(19) United States
(12) Patent Application Publication
(10) Pub. No.: US 2016/0060260 A1
(43) Pub. Date: Mar. 3, 2016

(54) BROMODOMAIN INHIBITORS FOR TREATING DISEASE

(71) Applicant: Board of Regents, The University of Texas System, Austin, TX (US)

(72) Inventors: Wylie Palmer, Houston, TX (US); Philip Jones, Houston, TX (US); Gang Liu, Sugar Land, TX (US); Alessia Petrocchi, Houston, TX (US); Naphtali Reyna, Arlington, TX (US); Govindan Subrumanian, Belle Mead, NJ (US); Jay Theroff, Manvel, TX (US); Anne Yau, Houston, TX (US)

(73) Assignee: Board of Regents, The University of Texas System, Austin, TX (US)

(21) Appl. No.: 14/838,570

(22) Filed: Aug. 28, 2015

Related U.S. Application Data
(60) Provisional application No. 62/043,578, filed on Aug. 29, 2014.

(51) Int. Cl. C07D 471/04; A61K 31/498; A61K 31/437; A61K 31/454; A61K 31/4439; A61K 31/4184; C07D 401/14; C07D 403/12; C07D 235/26; A61N 5/10; C07D 401/12; C07D 405/12; C07D 405/14; A61K 45/06; A61B 18/02; A61N 7/00; A61K 31/5377

(52) U.S. Cl. CPC C07D 471/04; A61K 31/5377; A61K 31/498; A61K 31/437; A61K 31/454; A61K 31/4439; A61K 31/4184; C07D 401/14; C07D 403/12; C07D 235/26; A61N 5/10; C07D 401/12; C07D 405/12; C07D 405/14; A61K 45/06; A61B 18/02; A61N 7/00; A61B 2018/00577

(57) ABSTRACT

Disclosed herein are compounds and compositions useful in the treatment of bromodomain-containing protein-mediated diseases, such as cancer, having the structure of Formula I:

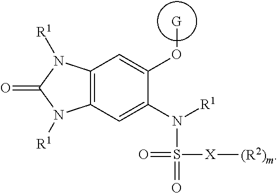

Methods of inhibiting activity of a bromodomain-containing protein in a human or animal subject are also provided.

This application claims the benefit of U.S. Provisional Application No. 62/043,578, filed Aug. 29, 2014, the disclosure of which is hereby incorporated by reference as if written herein in its entirety.

The present disclosure relates to new compounds and compositions, and their application as pharmaceuticals for the treatment of disease. Methods of inhibition of bromodomain-containing protein activity in a human or animal subject are also provided for the treatment of diseases such as cancer.

Acetylation of lysine residues is a post-translational modification (PTM) with broad relevance to cellular signaling and disease biology. Lysine acetylation, which is particularly abundant in nuclear macromolecular complexes, plays a key role in chromatin regulation and transcriptional control [Genes & Dev. 1998. 12: 599-606]. In cells, the principal ‘readers’ of the acetyl-lysine marks are the bromodomains (BRDs), which are a diverse family of evolutionary conserved protein-protein interaction modules that specifically recognizes and bind to acetylated lysine residues. The bromodomains, together with the enzymes that ‘write’ (Histone acetyl transferases, HATs) and ‘erase’ (histone deacetylases, HDACs) acetylated lysine residues on histone and non-histone proteins, critically control the regulation of gene expression and thereby cell phenotype including proliferation, cell differentiation and metabolism. Besides chromatin, many other proteins are also post-translationally modified such as P53, which could also be potentially recognized by bromodomain proteins. Because chromatin-mediated processes are often deregulated in cancer [Mol Oncol. December 2012; 6(6): 611-619], targeting epigenetic reader proteins like BET (dual-BRD4 containing proteins), CREBBP, ATAD2A, SMARCA2/4 and Tripartite Motif-containing 24 (TRIM24) represent promising targets for drug discovery. As illustrated by the development of selective inhibitors of the BET family of bromodomains, the conserved BRD fold represents a promising pocket for the development of small pharmaceutically active molecules [Nature Rev. Drug Discov., 2014, 337]. Indeed, small molecule bromodomain inhibitors have recently entered clinical trials [SciBX 7(15); doi:10.1038/scibx.2014.420] and objective responses have been reported in some leukemia patients during the Phase I dose-escalation trials [OncoEthics AACR abstract 2014].

The TRIM24 protein belongs to the TRIM/RBCC protein family, characterized by a conserved, N-terminal tripartite motif—namely, a RING domain, B-box zinc-fingers, and a coiled-coil region—as well as variable carboxy-terminal domains. In case of TRIM24, the variable C-terminal domain consists of an LXXLL nuclear receptor interaction motif, a plant homeodomain (PHD) and a bromodomain (Bromo) region. The TRIM24 PHD/bromodomains function combinatorially as a single entity to recognize dual histone marks of unmodified H3K4 and acetylated H3K23 (H3K23ac) within the same histone tail [Nature, 2010, 468, 927-32].

TRIM24 is a potent co-activator of ERα and overexpressed in many cancers as evident from both IHC [PLoS ONE 7(5): e37657; PLoS ONE 7(5): e37657; Am. J. Pathol., 2011, 178(4), 1461-9] and genomic analysis (4000+TCGA tumors). Specifically, protein expression in a cohort of 128 breast cancer patients revealed that 70% of these patients expressed aberrantly high levels of TRIM24, which strongly correlated with poor survival [Nature, 2010, 468, 927-32]. In contrast, those patients with undetectable or low expression of TRIM24, comparable to normal mammary epithelial cells, had good survival (p<0.003). Genome wide profiling of estrogen receptor (ERα), TRIM24 and histone signatures identified a distinct class of ER/TRIM24 regulated genes in breast cancer cells (MCF7) associated with proliferation and cell cycle biology. Moreover, depletion of TRIM24 led to reduced survival and proliferation of MCF7 breast cancer cells, and is highly additive with 4-OH tamoxifen, an inhibitor of Erα [Nature, 2010, 468, 927-932]. Independent work by Cavaillis et al. showed that high levels of TRIM24 mRNA significantly correlated with markers of poor prognosis, and were associated with worse overall patient survival [Am. J. Pathol., 2011, 178(4), 1461-9].

TRIM24 has also been shown to act as a ubiquitin E3-ligase for the tumor suppressor p53 [Proc Natl Acad Sci., USA 2009, 106:11612-11616] and negatively regulates p53 levels, suggesting Trim24 as a therapeutic target to restore tumor suppression by p53 [Proc Natl Acad Sci., USA 2014, Apr. 28, 1320428111].

Knock-down of TRIM24 in cancer cells results in anti-proliferative phenotypes as reported for non-small cell lung cancer [PLoS ONE., 2012; 7(5):e37657], prostate [Am. J. Pathol., 2011, 178(4), 1461-9] and head & neck cancer [Yan Lu, AACR Annual Meeting 2012, poster 1144]. In normal human embryonic stem cells, Trim24 knock-down by siRNA induces spontaneous differentiation suggesting a possible use of TRIM24 inhibitors in ‘differentiation therapy’ of cancer stem cells [PLoS Biol., 2012; 10(2):e1001268].

Mechanistic studies also suggest that inhibition of TRIM24 may synergize with DNA-damaging agents in general due to a possible role of TRIM24 in DNA repair and centromer biology [Nature 2010, 468, 927-32]. Consistent with this, we also find that TRIM24 expression is up regulated following exposure to DNA damage agents (72 hrs).

The bromodomain PHD finger protein 1 (BRPF1) acts as a scaffold for the assembly of a tetrameric histone acetyltransferase (HAT) complex, which play a role in acute myeloid leukemia (AML) [Int J Hematol., 2014 January; 99(1):21-31]. The HAT activity of this complex is encoded by the monocytic leukemia zinc finger (MOZ) gene, which is involved in recurrent translocations in hematopoietic cancers [Cancer Sci, 2008; 99(8), 1532-1527]. Deregulation of the BRPF1/MOZ complex, exemplified by 8p11 translocations resulting in MOZ-CBP, MOZ-p300 and MOZ-TIF2 fusion, are potent oncogenic drivers with expression of these fusion proteins in mouse bone marrow cells being sufficient to drive leukemogenesis in vivo [J Cell Physiol., 2014 Mar. 14. doi: 10.1002]. The 8p11 myeloproliferative syndrome is a rare, aggressive myeloproliferative neoplasm carrying fibroblast growth factor receptor-1 (FGFR1) fusion genes, and are clinically characterized by eosinophilia, a poor prognosis and frequent association with T- or B-cell lymphoma. FGFR1 rearrangements involving TRIM24 have been described [Haematologica. 2013; 98(1):103-6]. Thus, targeting the BRPF1/2/3 and or TRIM24 bromodomain(s) in an effort to normalize gene expression and the deregulated transcriptional programs represents an attractive strategy for therapeutic intervention in human disease [J Cell Physiol., 2014 Mar. 14. doi: 10.1002].

Thus, highly selective therapeutic agents directed against this emerging class of gene regulatory proteins promise new approaches to the treatment of human diseases.

### SUMMARY

Accordingly, the inventors herein disclose new compositions and methods for inhibiting bromodomain-containing protein activity.

Provided is a compound of structural Formula I

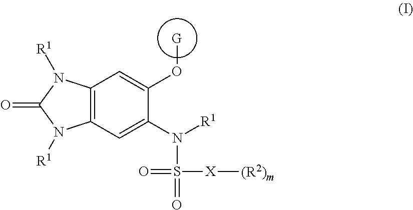

or a salt thereof, wherein: m is chosen from the integers 0, 1, 2, and 3; X is chosen from alkyl, aryl, cycloalkyl, heteroaryl, heteroarylalkyl, heterocycloalkyl, and heterocycloalkylalkyl; each R¹ is independently chosen from alkenyl, alkyl, H, and haloalkyl each R² is independently chosen from alkenyl, alkoxy, alkyl, aryl, arylalkyl, cyano, cycloalkyl, H, halo, haloalkoxy, heteroaryl, heteroarylalkyl, heterocycloalkyl, hydroxyl, N(R³)₂, C(O)OH, N(R³)C(O)C(R³)₃, N(R³)C(O)OC(R³)₃, C(O)OC(R³)₃, and C(O)N(R³)₂, wherein two R² groups together with the atoms to which they are attached optionally form an aryl, cycloalkyl, heteroaryl, or heterocycloalkyl ring, which may be optionally substituted with between 0 and 3 R³ groups; each R³ is independently chosen from alkenyl, alkoxy, alkyl, aminoalkyl, aryl, arylalkyl, cyano, cycloalkyl, cycloalkylalkyl, cycloalkylalkoxy, H, halo, haloalkyl, heteroaryl, heteroarylalkyl, heterocycloalkyl, heterocycloalkylalkyl, and hydroxyl; and G is phenyl, which may be optionally substituted.

Provided is a composition comprising a compound of Formula I and a pharmaceutically acceptable carrier, adjuvant, or vehicle.

Provided is a method for inhibiting activity of a bromodomain-containing protein, or a mutant thereof, in a biological sample comprising contacting the biological sample with a compound of Formula I.

Provided is a method for treating a bromodomain-containing protein-mediated disorder in a subject in need thereof, comprising the step of administering to the subject a compound of Formula I.

Provided is method of treating a bromodomain-containing protein-mediated disorder in a subject in need thereof, comprising the sequential or co-administration of a compound of Formula I or a pharmaceutically acceptable salt thereof, and another therapeutic agent.

Provided is a compound of any of Formula I for use in human therapy.

Provided is a compound of any of Formula I for use in treating a bromodomain-containing protein-mediated disease.

Provided is a use of a compound of Formula I for the manufacture of a medicament to treat a bromodomain-containing protein-mediated disease.

### DETAILED DESCRIPTION

### Abbreviations and Definitions

To facilitate understanding of the disclosure, a number of terms and abbreviations as used herein are defined below as follows:

When introducing elements of the present disclosure or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein when referring to a measurable value such as an amount of a compound, dose, time, temperature, and the like, is meant to encompass variations of 20%, 10%, 5%, 1%, 0.5%, or even 0.1% from the specified amount.

The term “acyl,” as used herein, alone or in combination, refers to a carbonyl attached to an alkenyl, alkyl, aryl, cycloalkyl, heteroaryl, heterocycle, or any other moiety were the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon radical having one or more double bonds and containing from 2 to 20 carbon atoms. In certain embodiments, the alkenyl will comprise from 2 to 6 carbon atoms. The term “alkenylene” refers to a carbon-carbon double bond system attached at two or more positions such as ethenylene [(—CH═CH—),(—C::C—)]. Examples of suitable alkenyl radicals include ethenyl, propenyl, 2-methylpropenyl, 1,4-butadienyl and the like. Unless otherwise specified, the term “alkenyl” may include “alkenylene” groups.

The term “alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl radical containing from 1 to 20 carbon atoms. In certain embodiments, the alkyl will comprise from 1 to 10 carbon atoms. In further embodiments, the alkyl will comprise from 1 to 6 carbon atoms. Alkyl groups may be optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like.

The term “alkylidene,” as used herein, alone or in combination, refers to an alkenyl group in which one carbon atom of the carbon-carbon double bond belongs to the moiety to which the alkenyl group is attached.

The term “alkylthio,” as used herein, alone or in combination, refers to an alkyl thioether (R—S—) radical wherein the term alkyl is as defined above and wherein the sulfur may be singly or doubly oxidized. Examples of suitable alkyl thioether radicals include methylthio, ethylthio, n-propylthio, isopropylthio, n-butylthio, iso-butylthio, sec-butylthio, tert-butylthio, methanesulfonyl, ethanesulfinyl, and the like.

The term “alkynyl,” as used herein, alone or in combination, refers to a straight-chain or branched chain hydrocarbon radical having one or more triple bonds and containing from 2 to 20 carbon atoms. In certain embodiments, the alkynyl comprises from 2 to 6 carbon atoms. In further embodiments, the alkynyl comprises from 2 to 4 carbon atoms. The term “alkynylene” refers to a carbon-carbon triple bond attached at two positions such as ethynylene (—C:::C—, —C≡C—). Examples of alkynyl radicals include ethynyl, propynyl, hydroxypropynyl, butyn-1-yl, butyn-2-yl, pentyn-1-yl, 3-methylbutyn-1-yl, hexyn-2-yl, and the like. Unless otherwise specified, the term “alkynyl” may include “alkynylene” groups.

The terms “amido” and “carbamoyl” as used herein, alone or in combination, refer to an amino group as described below attached to the parent molecular moiety through a carbonyl group, or vice versa. The term “C-amido” as used herein, alone or in combination, refers to a —C(O)N(RR′) group with R and R′ as defined herein or as defined by the specifically enumerated “R” groups designated. The term “N-amido” as used herein, alone or in combination, refers to a RC(O)N(R′)— group, with R and R′ as defined herein or as defined by the specifically enumerated “R” groups designated. The term “acylamino” as used herein, alone or in combination, embraces an acyl group attached to the parent moiety through an amino group. An example of an “acylamino” group is acetylamino (CH₃C(O)NH—).

The term “amino,” as used herein, alone or in combination, refers to —NRR′, wherein R and R′ are independently selected from the group consisting of hydrogen, alkyl, acyl, heteroalkyl, aryl, cycloalkyl, heteroaryl, and heterocycloalkyl, any of which may themselves be optionally substituted. Additionally, R and R′ may combine to form heterocycloalkyl, either of which may be optionally substituted.

The term “aryl,” as used herein, alone or in combination, means a carbocyclic aromatic system containing one, two or three rings wherein such polycyclic ring systems are fused together. The term “aryl” embraces aromatic groups such as phenyl, naphthyl, anthracenyl, and phenanthryl.

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term “arylalkynyl” or “aralkynyl,” as used herein, alone or in combination, refers to an aryl group attached to the parent molecular moiety through an alkynyl group.

The term “arylalkanoyl” or “aralkanoyl” or “aroyl,” as used herein, alone or in combination, refers to an acyl radical derived from an aryl-substituted alkanecarboxylic acid such as benzoyl, naphthoyl, phenylacetyl, 3-phenylpropionyl (hydrocinnamoyl), 4-phenylbutyryl, (2-naphthyl)acetyl, 4-chlorohydrocinnamoyl, and the like.

The term aryloxy as used herein, alone or in combination, refers to an aryl group attached to the parent molecular moiety through an oxy.

The terms “benzo” and “benz,” as used herein, alone or in combination, refer to the divalent radical C₆H₄=derived from benzene. Examples include benzothiophene and benzimidazole.

The term “carbamate,” as used herein, alone or in combination, refers to an ester of carbamic acid (—NHCOO—) which may be attached to the parent molecular moiety from either the nitrogen or acid end, and which may be optionally substituted as defined herein.

The term “O-carbamyl” as used herein, alone or in combination, refers to a —OC(O)NRR′, group, with R and R′ as defined herein.

The term “N-carbamyl” as used herein, alone or in combination, refers to a ROC(O)NR′— group, with R and R′ as defined herein.

The term “carbonyl,” as used herein, when alone includes formyl [—C(O)H] and in combination is a —C(O)— group.

The term “carboxyl” or “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl group wherein each cyclic moiety contains from 3 to 12 carbon atom ring members and which may optionally be a benzo fused ring system which is optionally substituted as defined herein. In certain embodiments, the cycloalkyl will comprise from 5 to 7 carbon atoms. Examples of such cycloalkyl groups include cyclopropyl, cyclobutyl, cyclopentyl, cyclohexyl, cycloheptyl, tetrahydronapthyl, indanyl, octahydronaphthyl, 2,3-dihydro-1H-indenyl, adamantyl and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or cyclic hydrocarbon radical, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Up to two heteroatoms may be consecutive, such as, for example, —CH₂—NH—OCH₃.

The term “heteroaryl,” as used herein, alone or in combination, refers to a 3 to 15 membered unsaturated heteromonocyclic ring, or a fused monocyclic, bicyclic, or tricyclic ring system in which at least one of the fused rings is aromatic, which contains at least one atom selected from the group consisting of O, S, and N. In certain embodiments, the heteroaryl will comprise from 5 to 7 carbon atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

The terms “heterocycloalkyl” and, interchangeably, “heterocycle,” as used herein, alone or in combination, each refer to a saturated, partially unsaturated, or fully unsaturated monocyclic, bicyclic, or tricyclic heterocyclic group containing at least one heteroatom as a ring member, wherein each the heteroatom may be independently selected from the group consisting of nitrogen, oxygen, and sulfur In certain embodiments, the heterocycloalkyl will comprise from 1 to 4 heteroatoms as ring members. In further embodiments, the heterocycloalkyl will comprise from 1 to 2 heteroatoms as ring members. In certain embodiments, the heterocycloalkyl will comprise from 3 to 8 ring members in each ring. In further embodiments, the heterocycloalkyl will comprise from 3 to 7 ring members in each ring. In yet further embodiments, the heterocycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihydropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited.

The term “hydrazinyl” as used herein, alone or in combination, refers to two amino groups joined by a single bond, i.e., —N—N—.

The term “hydroxy,” or “hydroxyl,” as used herein, alone or in combination, refers to —OH.

The term “hydroxyalkyl,” as used herein, alone or in combination, refers to a hydroxy group attached to the parent molecular moiety through an alkyl group.

The term “imino,” as used herein, alone or in combination, refers to ═N—.

The term “iminohydroxy,” as used herein, alone or in combination, refers to ═N(OH) and ═N—O—.

The phrase “in the main chain” refers to the longest contiguous or adjacent chain of carbon atoms starting at the point of attachment of a group to the compounds of any one of the formulas disclosed herein.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “lower,” as used herein, alone or in a combination, where not otherwise specifically defined, means containing from 1 to and including 6 carbon atoms.

The term “lower aryl,” as used herein, alone or in combination, means phenyl or naphthyl, either of which may be optionally substituted as provided.

The term “lower heteroaryl,” as used herein, alone or in combination, means either 1) monocyclic heteroaryl comprising five or six ring members, of which between one and four the members may be heteroatoms selected from the group consisting of O, S, and N, or 2) bicyclic heteroaryl, wherein each of the fused rings comprises five or six ring members, comprising between them one to four heteroatoms selected from the group consisting of O, S, and N.

The term “lower cycloalkyl,” as used herein, alone or in combination, means a monocyclic cycloalkyl having between three and six ring members. Lower cycloalkyls may be unsaturated. Examples of lower cycloalkyl include cyclopropyl, cyclobutyl, cyclopentyl, and cyclohexyl.

The term “lower heterocycloalkyl,” as used herein, alone or in combination, means a monocyclic heterocycloalkyl having between three and six ring members, of which between one and four may be heteroatoms selected from the group consisting of O, S, and N. Examples of lower heterocycloalkyls include pyrrolidinyl, imidazolidinyl, pyrazolidinyl, piperidinyl, piperazinyl, and morpholinyl. Lower heterocycloalkyls may be unsaturated.

The term “lower amino,” as used herein, alone or in combination, refers to —NRR′, wherein R and R′ are independently selected from the group consisting of hydrogen, lower alkyl, and lower heteroalkyl, any of which may be optionally substituted. Additionally, the R and R′ of a lower amino group may combine to form a five- or six-membered heterocycloalkyl, either of which may be optionally substituted.

The term “mercaptyl” as used herein, alone or in combination, refers to an RS— group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The terms “sulfonate,” “sulfonic acid,” and “sulfonic,” as used herein, alone or in combination, refer to the —SO₃H group and its anion as the sulfonic acid is used in salt formation.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —S(O)₂—.

The term “N-sulfonamido” refers to a RS(═O)₂NR′— group with R and R′ as defined herein.

The term “S-sulfonamido” refers to a —S(═O)₂NRR′, group, with R and R′ as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC(S)NR′— group, with R and R′ as defined herein.

The term “O-thiocarbamyl” refers to a —OC(S)NRR′, group with R and R′ as defined herein.

The term “thiocyanato” refers to a —CNS group.

The term “trihalomethanesulfonamido” refers to a X₃CS(O)₂NR— group with X is a halogen and R as defined herein.

The term “trihalomethanesulfonyl” refers to a X₃CS(O)₂— group where X is a halogen.

The term “trihalomethoxy” refers to a X₃CO— group where X is a halogen.

The term “trisubstituted silyl,” as used herein, alone or in combination, refers to a silicone group substituted at its three free valences with groups as listed herein under the definition of substituted amino. Examples include trimethysilyl, tert-butyldimethylsilyl, triphenylsilyl and the like.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that the group is absent.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, pyridinyl, thiophenyl, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety selected from the group consisting of hydrogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and Rn where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. Thus, by way of example only, an unsymmetrical group such as —C(O)N(R)— may be attached to the parent moiety at either the carbon or the nitrogen.

Asymmetric centers exist in the compounds disclosed herein. These centers are designated by the symbols “R” or “S,” depending on the configuration of substituents around the chiral carbon atom. It should be understood that the disclosure encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present disclosure includes all cis, trans, syn, anti, entgegen (E), and zusammen (Z) isomers as well as the appropriate mixtures thereof. Additionally, compounds may exist as tautomers; all tautomeric isomers are provided by this disclosure. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered part of larger substructure. A bond may be single, double, or triple unless otherwise specified. A dashed line between two atoms in a drawing of a molecule indicates that an additional bond may be present or absent at that position.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

Bromodomain-containing protein inhibitor is used herein to refer to a compound that exhibits an IC₅₀ with respect to bromodomain-containing protein activity of no more than about 100 μM and more typically not more than about 50 μM, as measured in the bromodomain-containing protein enzyme assay described generally herein below. IC₅₀ is that concentration of inhibitor that reduces the activity of the bromodomain-containing protein to half-maximal level. Certain compounds disclosed herein have been discovered to exhibit inhibition against the bromodomain-containing protein. In certain embodiments, compounds will exhibit an IC₅₀ with respect to the bromodomain-containing protein of no more than about 10 μM; in further embodiments, compounds will exhibit an IC₅₀ with respect to the bromodomain-containing protein of no more than about 5 μM; in yet further embodiments, compounds will exhibit an IC₅₀ with respect to the bromodomain-containing protein of not more than about 1 μM; in yet further embodiments, compounds will exhibit an IC₅₀ with respect to the bromodomain-containing protein of not more than about 200 nM, as measured in the bromodomain-containing protein binding assay described herein.

The phrase “therapeutically effective” is intended to qualify the amount of active ingredients used in the treatment of a disease or disorder or on the effecting of a clinical endpoint.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

As used herein, reference to “treatment” of a patient is intended to include prophylaxis. Treatment may also be preemptive in nature, i.e., it may include prevention of disease. Prevention of a disease may involve complete protection from disease, for example as in the case of prevention of infection with a pathogen, or may involve prevention of disease progression. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease.

The term “patient” is generally synonymous with the term “subject” and includes all mammals including humans. Examples of patients include humans, livestock (farm animals) such as cows, goats, sheep, pigs, and rabbits, and companion animals such as dogs, cats, rabbits, and horses. Preferably, the patient is a human.

The term “prodrug” refers to a compound that is made more active in vivo. Certain compounds disclosed herein may also exist as prodrugs, as described in the art. Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

The compounds disclosed herein can exist as therapeutically acceptable salts. The present disclosure includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present disclosure contemplates sodium, potassium, magnesium, and calcium salts of the compounds disclosed herein, and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

A salt of a compound can be made by reacting the appropriate compound in the form of the free base with the appropriate acid.

### Compounds

The present disclosure provides a compound of structural Formula I

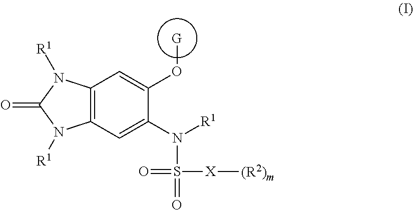

or a salt thereof, wherein: m is chosen from the integers 0, 1, 2, and 3; X is chosen from alkyl, aryl, cycloalkyl, heteroaryl, heteroarylalkyl, heterocycloalkyl, and heterocycloalkylalkyl; each R¹ is independently chosen from alkenyl, alkyl, H, and haloalkyl; each R² is independently chosen from alkenyl, alkoxy, alkyl, aryl, arylalkyl, cyano, cycloalkyl, H, halo, haloalkoxy, heteroaryl, heteroarylalkyl, heterocycloalkyl, hydroxyl, N(R³)₂, C(O)OH, N(R³)C(O)C(R³)₃, N(R³)C(O)OC(R³)₃, C(O)OC(R³)₃, and C(O)N(R³)₂, wherein two R² groups together with the atoms to which they are attached optionally form an aryl, cycloalkyl, heteroaryl, or heterocycloalkyl ring, which may be optionally substituted with between 0 and 3 R³ groups; each R³ is independently chosen from alkenyl, alkoxy, alkyl, aminoalkyl, aryl, arylalkyl, cyano, cycloalkyl, cycloalkylalkyl, cycloalkylalkoxy, H, halo, haloalkyl, heteroaryl, heteroarylalkyl, heterocycloalkyl, heterocycloalkylalkyl, and hydroxyl; and G is phenyl, which may be optionally substituted.

In some embodiments, the compound, or a pharmaceutically acceptable salt thereof, has Formula II

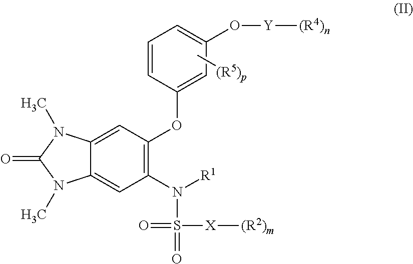

wherein: m, n, and q are independently chosen from the integers 0, 1, 2, and 3; p is chosen from the integers 0, 1, 2, 3, and 4; X, Y, and Z are independently chosen from alkyl, aryl, cycloalkyl, heteroaryl, heteroarylalkyl, heterocycloalkyl, and heterocycloalkylalkyl; R¹ is chosen from alkenyl, alkyl, H, and haloalkyl; each R², R⁴, and R⁶ is independently chosen from alkenyl, alkoxy, alkyl, aryl, arylalkyl, cyano, cycloalkyl, H, halo, haloalkoxy, heteroaryl, heteroarylalkyl, heterocycloalkyl, hydroxyl, N(R³)₂, C(O)OH, N(R³)C(O)C(R³)₃, N(R³)C(O)OC(R³)₃, C(O)OC(R³)₃, and C(O)N(R³)₂, wherein two R² groups together with the atoms to which they are attached optionally form an aryl, cycloalkyl, heteroaryl, or heterocycloalkyl ring, which may be optionally substituted with between 0 and 3 R³ groups, wherein two R⁴ groups together with the atoms to which they are attached optionally form an aryl, cycloalkyl, heteroaryl, or heterocycloalkyl ring, which may be optionally substituted with between 0 and 3 R³ groups, wherein two R⁶ groups together with the atoms to which they are attached optionally form an aryl, cycloalkyl, heteroaryl, or heterocycloalkyl ring, which may be optionally substituted with between 0 and 3 R³ groups; each R³ is independently chosen from alkenyl, alkoxy, alkyl, aminoalkyl, aryl, arylalkyl, cyano, cycloalkyl, cycloalkylalkyl, cycloalkylalkoxy, H, halo, haloalkyl, heteroaryl, heteroarylalkyl, heterocycloalkyl, heterocycloalkylalkyl, and hydroxyl; and each R⁵ is independently chosen from O—Z—(R⁶)_{q}, alkyl, cyano, H, and halo.

In some embodiments, the compound, or a pharmaceutically acceptable salt thereof, has Formula III

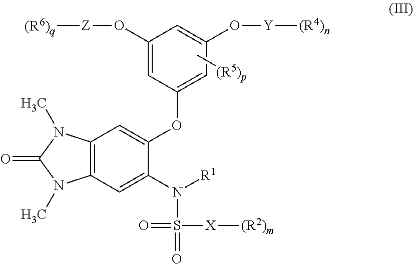

wherein: m, n, p, and q are independently chosen from the integers 0, 1, 2, and 3; X, Y, and Z are independently chosen from alkyl, aryl, cycloalkyl, heteroaryl, heteroarylalkyl, heterocycloalkyl, and heterocycloalkylalkyl; R¹ is chosen from alkenyl, alkyl, H, and haloalkyl; each R², R⁴, and R⁶ is independently chosen from alkenyl, alkoxy, alkyl, aryl, arylalkyl, cyano, cycloalkyl, H, halo, haloalkoxy, heteroaryl, heteroarylalkyl, heterocycloalkyl, hydroxyl, N(R³)₂, C(O)OH, N(R³)C(O)C(R³)₃, N(R³)C(O)OC(R³)₃, C(O)OC(R³)₃, and C(O)N(R³)₂, wherein two R² groups together with the atoms to which they are attached optionally form an aryl, cycloalkyl, heteroaryl, or heterocycloalkyl ring, which may be optionally substituted with between 0 and 3 R³ groups, wherein two R⁴ groups together with the atoms to which they are attached optionally form an aryl, cycloalkyl, heteroaryl, or heterocycloalkyl ring, which may be optionally substituted with between 0 and 3 R³ groups, wherein two R⁶ groups together with the atoms to which they are attached optionally form an aryl, cycloalkyl, heteroaryl, or heterocycloalkyl ring, which may be optionally substituted with between 0 and 3 R³ groups; each R³ is independently chosen from alkenyl, alkoxy, alkyl, aminoalkyl, aryl, arylalkyl, cyano, cycloalkyl, cycloalkylalkyl, cycloalkylalkoxy, H, halo, haloalkyl, heteroaryl, heteroarylalkyl, heterocycloalkyl, heterocycloalkylalkyl, and hydroxyl; and each R⁵ is independently chosen from H and halo.

In certain embodiments, X is chosen from imidazole, phenyl, pyridine, and pyrazole.

In certain embodiments, Y and Z are alkyl.

In certain embodiments, R¹ is H.

In certain embodiments, R² is chosen from alkyl and alkoxy.

In certain embodiments, R⁴ and R⁶ are independently chosen from alkoxy, H, halo, and N(R³)₂.

Also provided are embodiments wherein any of embodiment above in paragraphs [0011] and [0112]-[0119] above may be combined with any one or more of these embodiments, provided the combination is not mutually exclusive. As used herein, two embodiments are “mutually exclusive” when one is defined to be something which cannot overlap with the other. For example, an embodiment wherein Y is alkyl is mutually exclusive with an embodiment wherein Y is heteroaryl. However, an embodiment wherein Y is alkyl and X is imidazole is not mutually exclusive with an embodiment wherein R¹ is H.

### Pharmaceutical Compositions

While it may be possible for the compounds of the subject disclosure to be administered as the raw chemical, it is also possible to present them as a pharmaceutical formulation. Accordingly, provided herein are pharmaceutical formulations which comprise one or more of certain compounds disclosed herein, or one or more pharmaceutically acceptable salts, esters, prodrugs, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. Typically, these methods include the step of bringing into association a compound of the subject disclosure or a pharmaceutically acceptable salt, ester, amide, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Compounds described herein can be administered as follows:

### Oral Administration

The compounds of the present invention may be administered orally, including swallowing, so the compound enters the gastrointestinal tract, or is absorbed into the blood stream directly from the mouth, including sublingual or buccal administration.

Suitable compositions for oral administration include solid formulations such as tablets, pills, cachets, lozenges and hard or soft capsules, which can contain liquids, gels, powders, or granules.

In a tablet or capsule dosage form the amount of drug present may be from about 0.05% to about 95% by weight, more typically from about 2% to about 50% by weight of the dosage form.

In addition, tablets or capsules may contain a disintegrant, comprising from about 0.5% to about 35% by weight, more typically from about 2% to about 25% of the dosage form. Examples of disintegrants include methyl cellulose, sodium or calcium carboxymethyl cellulose, croscarmellose sodium, polyvinylpyrrolidone, hydroxypropyl cellulose, starch and the like.

Suitable binders, for use in a tablet, include gelatin, polyethylene glycol, sugars, gums, starch, hydroxypropyl cellulose and the like. Suitable diluents, for use in a tablet, include mannitol, xylitol, lactose, dextrose, sucrose, sorbitol and starch.

Suitable surface active agents and glidants, for use in a tablet or capsule, may be present in amounts from about 0.1% to about 3% by weight, and include polysorbate 80, sodium dodecyl sulfate, talc and silicon dioxide.

Suitable lubricants, for use in a tablet or capsule, may be present in amounts from about 0.1% to about 5% by weight, and include calcium, zinc or magnesium stearate, sodium stearyl fumarate and the like.

Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with a liquid diluent. Dyes or pigments may be added to tablets for identification or to characterize different combinations of active compound doses.

Liquid formulations can include emulsions, solutions, syrups, elixirs and suspensions, which can be used in soft or hard capsules. Such formulations may include a pharmaceutically acceptable carrier, for example, water, ethanol, polyethylene glycol, cellulose, or an oil. The formulation may also include one or more emulsifying agents and/or suspending agents.

Compositions for oral administration may be formulated as immediate or modified release, including delayed or sustained release, optionally with enteric coating.

In another embodiment, a pharmaceutical composition comprises a therapeutically effective amount of a compound of Formula (I) or a pharmaceutically acceptable salt thereof, and a pharmaceutically acceptable carrier.

### Parenteral Administration

Compounds of the present invention may be administered directly into the blood stream, muscle, or internal organs by injection, e.g., by bolus injection or continuous infusion.

Suitable means for parenteral administration include intravenous, intra-muscular, subcutaneous intra-arterial, intraperitoneal, intrathecal, intracranial, and the like. Suitable devices for parenteral administration include injectors (including needle and needle-free injectors) and infusion methods. The formulations may be presented in unit-dose or multi-dose containers, for example sealed ampoules and vials.

Most parenteral formulations are aqueous solutions containing excipients, including salts, buffering, suspending, stabilizing and/or dispersing agents, antioxidants, bacteriostats, preservatives, and solutes which render the formulation isotonic with the blood of the intended recipient, and carbohydrates.

Parenteral formulations may also be prepared in a dehydrated form (e.g., by lyophilization) or as sterile non-aqueous solutions. These formulations can be used with a suitable vehicle, such as sterile water. Solubility-enhancing agents may also be used in preparation of parenteral solutions.

Compositions for parenteral administration may be formulated as immediate or modified release, including delayed or sustained release. Compounds may also be formulated as depot preparations. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

### Topical Administration

Compounds of the present invention may be administered topically (for example to the skin, mucous membranes, ear, nose, or eye) or transdermally. Formulations for topical administration can include, but are not limited to, lotions, solutions, creams, gels, hydrogels, ointments, foams, implants, patches and the like. Carriers that are pharmaceutically acceptable for topical administration formulations can include water, alcohol, mineral oil, glycerin, polyethylene glycol and the like. Topical administration can also be performed by, for example, electroporation, iontophoresis, phonophoresis and the like.

Typically, the active ingredient for topical administration may comprise from 0.001% to 10% w/w (by weight) of the formulation. In certain embodiments, the active ingredient may comprise as much as 10% w/w; less than 5% w/w; from 2% w/w to 5% w/w; or from 0.1% to 1% w/w of the formulation.

Compositions for topical administration may be formulated as immediate or modified release, including delayed or sustained release.

### Rectal, Buccal, and Sublingual Administration

Suppositories for rectal administration of the compounds of the present invention can be prepared by mixing the active agent with a suitable non-irritating excipient such as cocoa butter, synthetic mono-, di-, or triglycerides, fatty acids, or polyethylene glycols which are solid at ordinary temperatures but liquid at the rectal temperature, and which will therefore melt in the rectum and release the drug.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

### Administration by Inhalation

For administration by inhalation, compounds may be conveniently delivered from an insufflator, nebulizer pressurized packs or other convenient means of delivering an aerosol spray or powder. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the disclosure may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Other carrier materials and modes of administration known in the pharmaceutical art may also be used. Pharmaceutical compositions of the invention may be prepared by any of the well-known techniques of pharmacy, such as effective formulation and administration procedures. Preferred unit dosage formulations are those containing an effective dose, as herein recited, or an appropriate fraction thereof, of the active ingredient. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. In addition, the route of administration may vary depending on the condition and its severity. The above considerations concerning effective formulations and administration procedures are well known in the art and are described in standard textbooks.

### Methods of Treatment

The present disclosure provides compounds and pharmaceutical compositions that inhibit bromodomain-containing protein activity, and are thus useful in the treatment or prevention of disorders associated with bromodomain-containing protein activity. Compounds and pharmaceutical compositions of the present disclosure selectively modulate bromodomain-containing protein activity and are thus useful in the treatment or prevention of a range of disorders associated with bromodomain-containing protein activity and include, but are not limited to, cancer and other proliferative disorders, inflammatory diseases, sepsis, autoimmune disease, and viral diseases associated with bromodomain-containing protein activity.

Provided is a method for inhibiting activity of a bromodomain-containing protein, or a mutant thereof, in a biological sample comprising the step of contacting said biological sample with a compound of the present disclosure.

In some embodiments, the bromodomain-containing protein is chosen from BRPF1, BRPF2, BRPF3, and TRIM24. BRPF2 is often referred to as BRD1.

In certain embodiments, the bromodomain-containing protein is TRIM24.

Provided is a method for treating a bromodomain-containing protein-mediated disorder in a subject in need thereof, comprising the step of administering to said subject a compound of the present disclosure.

In some embodiments, the subject is a human.

In certain embodiments, the bromodomain-containing protein is chosen from BRPF1, BRPF2, BRPF3, and TRIM24. BRPF2 is often refered to as BRD1.

In particular embodiments, the bromodomain-containing protein is TRIM24.

In some embodiments, the bromodomain-containing protein-mediated disorder is a proliferative disorder, inflammatory disease, sepsis, neurological disease, autoimmune disease, or viral infection.

### Neurological Disorders

In some embodiments, the compounds and pharmaceutical compositions of the present disclosure may be useful in the treatment or prevention of neurological diseases.

Studies report important functions of epigenetic processes and bromodomain-containing proteins in several neurological diseases. Therefore, in certain embodiments, the compounds may be used for the treatment or prevention of neurological diseases. In certain embodiments, the compounds may be used for the treatment or prevention of Alzheimer's disease, Parkinson's disease, Huntington disease, bipolar disorder, schizophrenia, Rubinstein-Taybi syndrome, or epilepsy.

### Autoimmune and Inflammatory Disorders

In some embodiments, the compounds and pharmaceutical compositions of the present disclosure may be useful in the treatment or prevention of autoimmune and inflammatory diseases.

In certain embodiments, the autoimmune and inflammatory diseases involve an inflammatory response to infections with bacteria, viruses, fungi, parasites or their toxins.

In certain embodiments, the autoimmune and inflammatory diseases or conditions are chosen from sepsis, sepsis syndrome, septic shock, endotoxemia, systemic inflammatory response syndrome (SIRS), multi-organ dysfunction syndrome, toxic shock syndrome, acute lung injury, ARDS (adult respiratory distress syndrome), acute renal failure, fulminant hepatitis, burns, acute pancreatitis, post-surgical syndromes, sarcoidosis, Herxheimer reactions, encephalitis, myelitis, meningitis, malaria and SIRS associated with viral infections such as influenza, herpes zoster, herpes simplex and coronavirus.

### Cancer

In some embodiments, the compounds and pharmaceutical compositions of the present disclosure may be useful in the treatment or prevention of cancer.

In certain embodiments, the cancer is chosen from adenocarcinoma, adult T-cell leukemia/lymphoma, bladder cancer, blastoma, bone cancer, breast cancer, brain cancer, carcinoma, myeloid sarcoma, cervical cancer, colorectal cancer, esophageal cancer, gastrointestinal cancer, glioblastoma multiforme, glioma, gallbladder cancer, gastric cancer, head and neck cancer, Hodgkin's lymphoma, non-Hodgkin's lymphoma, intestinal cancer, kidney cancer, laryngeal cancer, leukemia, lung cancer, lymphoma, liver cancer, small cell lung cancer, non-small cell lung cancer, mesothelioma, multiple myeloma, ocular cancer, optic nerve tumor, oral cancer, ovarian cancer, pituitary tumor, primary central nervous system lymphoma, prostate cancer, pancreatic cancer, pharyngeal cancer, renal cell carcinoma, rectal cancer, sarcoma, skin cancer, spinal tumor, small intestine cancer, stomach cancer, T-cell lymphoma, testicular cancer, thyroid cancer, throat cancer, urogenital cancer, urothelial carcinoma, uterine cancer, vaginal cancer, and Wilms' tumor.

In certain embodiments, the cancer is chosen from acute myelogenous leukemia and Burkitt's lymphoma.

### Combinations and Combination Therapy

The compounds of the present invention can be used, alone or in combination with other pharmaceutically active compounds, to treat conditions such as those previously described hereinabove. The compound(s) of the present invention and other pharmaceutically active compound(s) can be administered simultaneously (either in the same dosage form or in separate dosage forms) or sequentially. Accordingly, in one embodiment, the present invention comprises methods for treating a condition by administering to the subject a therapeutically-effective amount of one or more compounds of the present invention and one or more additional pharmaceutically active compounds.

In another embodiment, there is provided a pharmaceutical composition comprising one or more compounds of the present invention, one or more additional pharmaceutically active compounds, and a pharmaceutically acceptable carrier.

In another embodiment, the one or more additional pharmaceutically active compounds is selected from the group consisting of anti-cancer drugs, anti-proliferative drugs, and anti-inflammatory drugs.

Bromodomain-containing protein inhibitor compositions described herein are also optionally used in combination with other therapeutic reagents that are selected for their therapeutic value for the condition to be treated. In general, the compounds described herein and, in embodiments where combination therapy is employed, other agents do not have to be administered in the same pharmaceutical composition and, because of different physical and chemical characteristics, are optionally administered by different routes. The initial administration is generally made according to established protocols and then, based upon the observed effects, the dosage, modes of administration and times of administration subsequently modified. In certain instances, it is appropriate to administer a bromodomain-containing protein inhibitor compound, as described herein, in combination with another therapeutic agent. By way of example only, the therapeutic effectiveness of a bromodomain-containing protein inhibitor is enhanced by administration of another therapeutic agent (which also includes a therapeutic regimen) that also has therapeutic benefit. Regardless of the disease, disorder or condition being treated, the overall benefit experienced by the patient is either simply additive of the two therapeutic agents or the patient experiences an enhanced (i.e., synergistic) benefit. Alternatively, if a compound disclosed herein has a side effect, it may be appropriate to administer an agent to reduce the side effect; or the therapeutic effectiveness of a compound described herein may be enhanced by administration of an adjuvant.

Therapeutically effective dosages vary when the drugs are used in treatment combinations. Methods for experimentally determining therapeutically effective dosages of drugs and other agents for use in combination treatment regimens are documented methodologies. Combination treatment further includes periodic treatments that start and stop at various times to assist with the clinical management of the patient. In any case, the multiple therapeutic agents (one of which is a bromodomain-containing protein inhibitor as described herein) may be administered in any order, or simultaneously. If simultaneously, the multiple therapeutic agents are optionally provided in a single, unified form, or in multiple forms (by way of example only, either as a single pill or as two separate pills).

In some embodiments, one of the therapeutic agents is given in multiple doses, or both are given as multiple doses. If not simultaneous, the timing between the multiple doses optionally varies from more than zero weeks to less than twelve weeks.

In addition, the combination methods, compositions and formulations are not to be limited to the use of only two agents, the use of multiple therapeutic combinations are also envisioned. It is understood that the dosage regimen to treat, prevent, or ameliorate the condition(s) for which relief is sought, is optionally modified in accordance with a variety of factors. These factors include the disorder from which the subject suffers, as well as the age, weight, sex, diet, and medical condition of the subject. Thus, the dosage regimen actually employed varies widely, in some embodiments, and therefore deviates from the dosage regimens set forth herein.

The pharmaceutical agents which make up the combination therapy disclosed herein are optionally a combined dosage form or in separate dosage forms intended for substantially simultaneous administration. The pharmaceutical agents that make up the combination therapy are optionally also administered sequentially, with either agent being administered by a regimen calling for two-step administration. The two-step administration regimen optionally calls for sequential administration of the active agents or spaced-apart administration of the separate active agents. The time between the multiple administration steps ranges from a few minutes to several hours, depending upon the properties of each pharmaceutical agent, such as potency, solubility, bioavailability, plasma half-life and kinetic profile of the pharmaceutical agent.

In another embodiment, a bromodomain-containing protein inhibitor is optionally used in combination with procedures that provide additional benefit to the patient. A bromodomain-containing protein inhibitor and any additional therapies are optionally administered before, during or after the occurrence of a disease or condition, and the timing of administering the composition containing a bromodomain-containing protein inhibitor varies in some embodiments. Thus, for example, a bromodomain-containing protein inhibitor is used as a prophylactic and is administered continuously to subjects with a propensity to develop conditions or diseases in order to prevent the occurrence of the disease or condition. A bromodomain-containing protein inhibitor and compositions are optionally administered to a subject during or as soon as possible after the onset of the symptoms. While embodiments of the present invention have been shown and described herein, it will be obvious to those skilled in the art that such embodiments are provided by way of example only. Numerous variations, changes, and substitutions will now occur to those skilled in the art without departing from the invention. It should be understood that in some embodiments of the invention various alternatives to the embodiments described herein are employed in practicing the invention.

A bromodomain-containing protein inhibitor can be used in combination with anti-cancer drugs, including but not limited to the following classes: alkylating agents, anti-metabolites, plant alkaloids and terpenoids, topoisomerase inhibitors, cytotoxic antibiotics, angiogenesis inhibitors and tyrosine kinase inhibitors.

For use in cancer and neoplastic diseases a bromodomain-containing protein inhibitor may be optimally used together with one or more of the following non-limiting examples of anti-cancer agents: (1) alkylating agents, including but not limited to cisplatin (PLATIN), carboplatin (PARAPLATIN), oxaliplatin (ELOXATIN), streptozocin (ZANOSAR), busulfan (MYLERAN) and cyclophosphamide (ENDOXAN); (2) anti-metabolites, including but not limited to mercaptopurine (PURINETHOL), thioguanine, pentostatin (NIPENT), cytosine arabinoside (ARA-C), gemcitabine (GEMZAR), fluorouracil (CARAC), leucovorin (FUSILEV) and methotrexate (RHEUMATREX); (3) plant alkaloids and terpenoids, including but not limited to vincristine (ONCOVIN), vinblastine and paclitaxel (TAXOL); (4) topoisomerase inhibitors, including but not limited to irinotecan (CAMPTOSAR), topotecan (HYCAMTIN) and etoposide (EPOSIN); (5) cytotoxic antibiotics, including but not limited to actinomycin D (COSMEGEN), doxorubicin (ADRIAMYCIN), bleomycin (BLENOXANE) and mitomycin (MITOSOL); (6) angiogenesis inhibitors, including but not limited to sunitinib (SUTENT) and bevacizumab (AVASTIN); and (7) tyrosine kinase inhibitors, including but not limited to imatinib (GLEEVEC), erlotinib (TARCEVA), lapatininb (TYKERB) and axitinib (INLYTA).

Where a subject is suffering from or at risk of suffering from an inflammatory condition, a bromodomain-containing protein inhibitor compound described herein is optionally used together with one or more agents or methods for treating an inflammatory condition in any combination. Therapeutic agents/treatments for treating an autoimmune and/or inflammatory condition include, but are not limited to any of the following examples: (1) corticosteroids, including but not limited to cortisone, dexamethasone, and methylprednisolone; (2) nonsteroidal anti-inflammatory drugs (NSAIDs), including but not limited to ibuprofen, naproxen, acetaminophen, aspirin, fenoprofen (NALFON), flurbiprofen (ANSAID), ketoprofen, oxaprozin (DAYPRO), diclofenac sodium (VOLTAREN), diclofenac potassium (CATAFLAM), etodolac (LODINE), indomethacin (INDOCIN), ketorolac (TORADOL), sulindac (CLINORIL), tolmetin (TOLECTIN), meclofenamate (MECLOMEN), mefenamic acid (PONSTEL), nabumetone (RELAFEN) and piroxicam (FELDENE); (3) immunosuppressants, including but not limited to methotrexate (RHEUMATREX), leflunomide (ARAVA), azathioprine (IMURAN), cyclosporine (NEORAL, SANDIMMUNE), tacrolimus and cyclophosphamide (CYTOXAN); (4) CD20 blockers, including but not limited to rituximab (RITUXAN); (5) Tumor Necrosis Factor (TNF) blockers, including but not limited to etanercept (ENBREL), infliximab (REMICADE) and adalimumab (HUMIRA); (6) interleukin-1 receptor antagonists, including but not limited to anakinra (KINERET); (7) interleukin-6 inhibitors, including but not limited to tocilizumab (ACTEMRA); (8) interleukin-17 inhibitors, including but not limited to AIN457; (9) Janus kinase inhibitors, including but not limited to tasocitinib; and (10) syk inhibitors, including but not limited to fostamatinib.

### Compound Synthesis

Compounds of the present invention can be prepared using methods illustrated in general synthetic schemes and experimental procedures detailed below. General synthetic schemes and experimental procedures are presented for purposes of illustration and are not intended to be limiting. Starting materials used to prepare compounds of the present invention are commercially available or can be prepared using routine methods known in the art.

### LIST OF ABBREVIATIONS

Ac₂O=acetic anhydride; AcCl=acetyl chloride; AcOH=acetic acid; AIBN=azobisisobutyronitrile; aq.=aqueous; br-s=broad singlet; Bu₃SnH=tributyltin hydride; CD₃OD=deuterated methanol; CDCl₃=deuterated chloroform; CDI=1,1′-Carbonyldiimidazole; DBU=1,8-diazabicyclo[5.4.0]undec-7-ene; DCM=dichloromethane; DEAD=diethyl azodicarboxylate; DIBAL-H=di-iso-butyl aluminium hydride; DIEA=DIPEA=N,N-diisopropylethylamine; DMAP=4-dimethylaminopyridine; DMF=N,N-dimethylformamide; DMSO-d₆=deuterated dimethyl sulfoxide; DMSO=dimethyl sulfoxide; DPPA=diphenylphosphoryl azide; EDC.HCl=EDCI.HCl=1-ethyl-3-(3-dimethylaminopropyl)carbodiimide hydrochloride; Et₂O=diethyl ether; EtOAc=ethyl acetate; EtOH=ethanol; h=hour; HATU=2-(1H-7-azabenzotriazol-1-yl)-1,1,3,3-tetramethyl uronium hexafluorophosphate methanaminium; HMDS=hexamethyldisilazane; HOBT=1-hydroxybenzotriazole; i-PrOH=isopropanol; LAH=lithium aluminiumhydride; LiHMDS=Lithium bis(trimethylsilyl)amide; MCPBA=meta-chloroperbenzoic acid; MeCN=acetonitrile; MeOH=methanol; MP-carbonate resin=macroporous triethylammonium methylpolystyrene carbonate resin; MsCl=mesyl chloride; MTBE=methyl tertiary butyl ether; n-BuLi=n-butyllithium; NaHMDS=Sodium bis(trimethylsilyl)amide; NaOMe=sodium methoxide; NaOtBu=sodium tert-butoxide; NBS=N-bromosuccinimide; NCS=N-chlorosuccinimide; NMP=N-Methyl-2-pyrrolidone; Pd(Ph₃)₄=tetrakis(triphenylphosphine)palladium(0); Pd₂(dba)₃=tris(dibenzylideneacetone)dipalladium(0); PdCl₂(PPh₃)2=bis(triphenylphosphine)palladium(II) dichloride; PG=protecting group; prep-HPLC=preparative high-performance liquid chromatography; PyBop=(benzotriazol-1-yloxy)tripyrrolidinophosphonium hexafluorophosphate; Pyr=pyridine; RT=room temperature; RuPhos=2-dicyclohexylphosphino-2′,6′-diisopropoxybiphenyl; sat.=saturated; ss=saturated solution; t-BuOH=tert-butanol; T3P=Propylphosphonic anhydride; TEA=Et₃N=triethylamine; TFA=trifluoroacetic acid; TFAA=trifluoroacetic anhydride; THF=tetrahydrofuran; Tol=toluene; TsCl=tosyl chloride; XPhos=2-dicyclohexylphosphino-2′,4′,6′-triisopropylbiphenyl.

### General Methods for Preparing Compounds

The following schemes can be used to practice the present invention. Additional structural groups, including but not limited to those defined elsewhere in the specification and not shown in the compounds described in the schemes can be incorporated to give various compounds disclosed herein, or intermediate compounds which can, after further manipulations using techniques known to those skilled in the art, be converted to compounds of the present invention.

### SYNTHESIS OF INTERMEDIATES

### N-(6-bromo-1,3-dimethyl-2-oxo-2,3-dihydro-1H-benzo[d]imidazol-5-yl)-2,2,2-trifluoroacetamide (Intermediate 1)

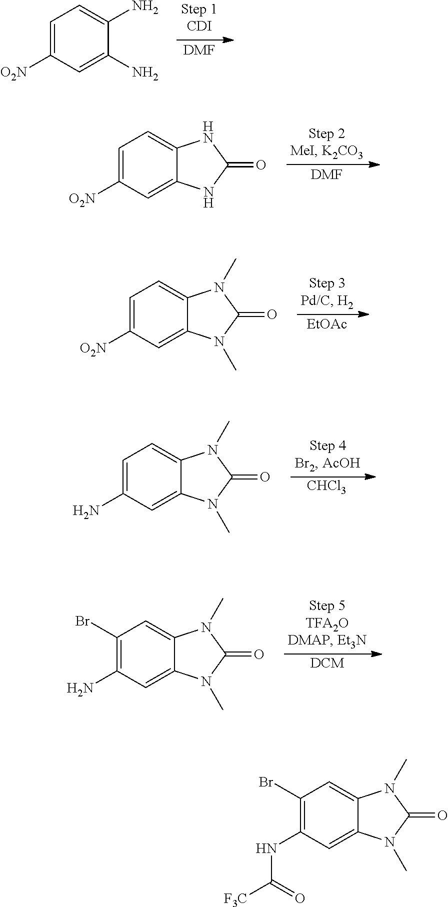

### Step 1: 5-nitro-1H-benzo[d]imidazol-2(3H)-one

To a 0° C. solution of 4-nitrobenzene-1,2-diamine (44 g, 285 mmol) in 80 mL of DMF was added 1,1′-carbonyldiimidazole (70 g, 428 mmol). The reaction mixture was stirred at RT for 4 h, then water (250 mL) was added. The resulting suspension was filtered, and the collected solids were washed with water (200 mL) and dried to give 5-nitro-1H-benzo[d]imidazol-2(3H)-one as a yellow solid (45 g, 88%). MS (ES+) C₇H₅N₃O₃ requires: 179. found: 180 [+H]⁺.

### Step 2: 1,3-dimethyl-5-nitro-1H-benzo[d]imidazol-2(3H)-one

To a solution of 5-nitro-1H-benzo[d]imidazol-2(3H)-one (55 g, 309 mmol) in 150 mL of DMF was added K₂CO₃ (85 g, 618 mmol), the reaction mixture was cooled to 0° C., then iodomethane (109 g, 772 mmol) was slowly added. The reaction mixture was stirred at RT overnight, then water was added to the reaction mixture. The resulting suspension was filtered and the collected solids were washed with water (200 mL) and dried to give 1,3-dimethyl-5-nitro-1H-benzo[d]imidazol-2(3H)-one as a yellow solid (55 g, 86%). MS (ES+) C₉H₉N₃O₃ requires: 207. found: 208 [M+H]⁺.

### Step 3: 5-amino-1,3-dimethyl-1H-benzo[d]imidazol-2(3H)-one

To a solution of 1,3-dimethyl-5-nitro-1H-benzo[d]imidazol-2(3H)-one (50 g, 240 mmol) in 200 mL of EtOAc under an inert atmosphere was added 10% palladium on activated carbon (5 g, 24 mmol). The reaction mixture was then charged with hydrogen and stirred at RT under an H₂ atmosphere overnight. The reaction mixture was filtered through a pad of celite then concentrated to give 5-amino-1,3-dimethyl-1H-benzo[d]imidazol-2(3H)-one as a yellow solid (32 g, 68%). MS (ES+) C₉H₁₁N₃O requires: 177. found: 178 [M+H]⁺.

### Step 4: 5-amino-6-bromo-1,3-dimethyl-1H-benzo[d]imidazol-2(3H)-one

To a 0° C. solution of 5-amino-1,3-dimethyl-1H-benzo[d]imidazol-2(3H)-one (4 g, 22.6 mmol) in 25 mL of CHCl₃ and 25 mL of AcOH was slowly added dropwise bromine (3.5 g, 22.6 mmol). The mixture was stirred at RT for 30 min, then concentrated and purified by silica gel chromatography (1:1 EtOAc/hexanes) to afford 5-amino-6-bromo-1,3-dimethyl-1H-benzo[d]imidazol-2(3H)-one as a yellow solid (3.2 g, 69%). MS (ES+) C₉H₁₀BrN₃O requires: 256. found: 257 [M+H]⁺.

### Step 5: N-(6-bromo-1,3-dimethyl-2-oxo-2,3-dihydro-1H-benzo[d]imidazol-5-yl)-2,2,2-trifluoroacetamide

To a 0° C. solution of 5-amino-6-bromo-1,3-dimethyl-1H-benzo[d]imidazol-2(3H)-one (1.50 g, 5.9 mmol) in DCM (45 ml) was added DMAP (72 mg, 0.59 mmol), triethylamine (1.63 ml, 11.7 mmol) and trifluoroacetic anhydride (0.91 ml, 6.4 mmol). The reaction mixture was stirred for 2 h and warmed to RT. The reaction mixture was then quenched with water and the organic phase was washed with brine, dried over sodium sulfate, filtered and concentrated to give N-(6-bromo-1,3-dimethyl-2-oxo-2,3-dihydro-1H-benzo[d]imidazol-5-yl)-2,2,2-trifluoroacetamide (Intermediate 1) as a yellow solid (2.20 g, 100%). MS (ES+) C₁₁H₉BrF₃N₃O₂ requires: 352. found 353 [M+H]⁺.

### 5-amino-6-(3-hydroxyphenoxy)-1,3-dimethyl-1H-benzo[d]imidazol-2(3H)-one (Intermediate 2, Route A)

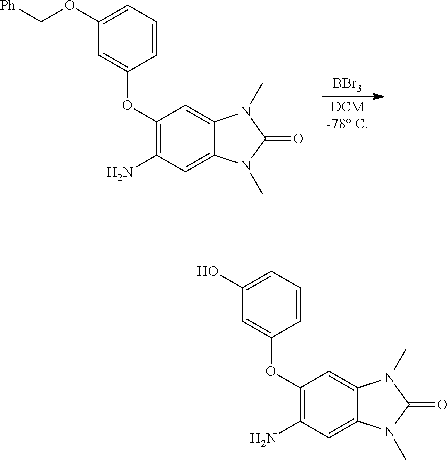 [SMILES: Cn1c(=O)n(C)c2cc(Oc3cccc(OCc4ccccc4)c3)c(N)cc21 ; "BBr3" ; "DCM-78° C." ; Cn1c(=O)n(C)c2cc(Oc3cccc(O)c3)c(N)cc21]

To a mixture of 5-amino-6-(3-(benzyloxy)phenoxy)-1,3-dimethyl-1H-benzo[d]imidazol-2(3H)-one (400 mg, 1.07 mmol) in DCM (20 mL) at −78° C. was added tribromoborane (5.3 mL, 5.3 mmol). The mixture was warmed up to room temperature gradually, then quenched by methanol dropwise, concentrated, and purified by column chromatography (20-100% EtOAc/hexanes and then 0-40% methanol/EtOAc) to give 5-amino-6-(3-hydroxyphenoxy)-1,3-dimethyl-1H-benzo[d]imidazol-2(3H)-one as a solid (240 mg, 79%). MS (ES+) C₁₅H₁₅N₃O₃ requires: 285. found: 286 [M+H]⁺.

### 5-amino-6-(3-hydroxyphenoxy)-1,3-dimethyl-1H-benzo[d]imidazol-2(3H)-one (Intermediate 2, Route B)

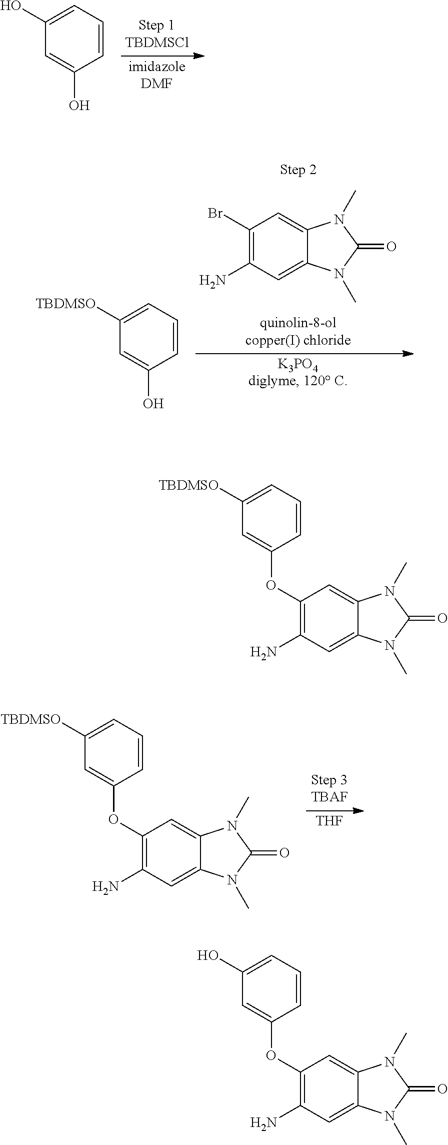

### Step 1: 3-[(tert-butyldimethylsilyl)oxy]phenol

A mixture of 1H-imidazole (2.25 g, 33.1 mmol), tert-butylchlorodimethylsilane (3.83 g, 25.4 mmol) and resorcinol (5.6 g, 51 mmol) in THF (30 ml) was stirred at 80° C. for 5 h. The resulting suspension of the cooled reaction mixture was filtered and the collected filtrate was concentrated and purified by silica-gel chromatography (20:80 to 0:100, EtOAc/hexanes) to give 3-((tert-butyldimethylsilyl)oxy)phenol (2.78 g, 49%). MS (ES+) C₁₂H₂₀O₂Si requires: 224. found 225 [M+H]⁺.

### Step 2: 5-amino-6-(3-((tert-butyldimethylsilyl)oxy)phenoxy)-1,3-dimethyl-1H-benzo[d]imidazol-2(3H)-one

A mixture of 3-((tert-butyldimethylsilyl)oxy)phenol (1.39 g, 6.20 mmol), quinolin-8-ol (79 mg, 0.55 mmol), copper(I) chloride (20 mg, 0.21 mmol), potassium phosphate (526 mg, 2.48 mmol) and 5-amino-6-bromo-1,3-dimethyl-1H-benzo[d]imidazol-2(3H)-one (529 mg, 2.07 mmol) in diglyme (20 ml) in a 100 mL round-bottom flask was degassed under a nitrogen atmosphere and heated to 120° C. for 24 h. To the cooled reaction mixture was added silica gel, stirred for 2 min, then the mixture was filtered through a pad of silica gel. The collected filtrate was concentrated and purified by column chromatography (20:80 to 0:100, EtOAc/hexanesthen 0:100 to 40:60, MeOH/EtOAc) to give 5-amino-6-(3-((tert-butyldimethylsilyl)oxy)phenoxy)-1,3-dimethyl-1H-benzo[d]imidazol-2(3H)-one (521 mg, 63%). MS (ES+) C₂₁H₂₉N₃O₃Si requires: 399. found 400 [M+H]⁺.

### Step 3: 5-amino-6-(3-hydroxyphenoxy)-1,3-dimethyl-1H-benzo[d]imidazol-2(3H)-one

To a 0° C. solution of 5-amino-6-(3-((tert-butyldimethylsilyl)oxy)phenoxy)-1,3-dimethyl-1H-benzo[d]imidazol-2(3H)-one (623 mg, 1.56 mmol) in THF was added a solution of tetra-butylammonium fluoride (0.90 mL, 3.1 mmol) in THF, the reaction mixture was allowed to warm up to RT and then stirred for 1-2 h. The reaction mixture was quenched with 1 M hydrogen chloride (0.10 mL, 3.1 mmol) and then partitioned between EtOAc and water. The seperated organic layer was washed with water twice, then concentrated and purified by column chromatography (20-80% EtOAc/hexanes and 0-40% MeOH/DCM) to give 5-amino-6-(3-hydroxyphenoxy)-1,3-dimethyl-1H-benzo[d]imidazol-2(3H)-one (120 mg, 27%) as a solid. MS (ES+) C₁₅H₁₅N₃O₃ requires: 285. found 286 [M+H]⁺.

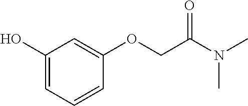

### 2-(3-hydroxyphenoxy)-N,N-dimethylacetamide (Intermediate 3)

A solution of resorcinol (250 mg, 2.27 mmol) in DMF (5 ml) was treated with potassium carbonate (628 mg, 4.54 mmol) and 2-bromo-N,N-dimethylacetamide (377 mg, 2.27 mmol). The reaction mixture was stirred for 16 h at ambient temperature, then diluted with water and carefully quenched with 1N HCl. EtOAc was added and the seperated organic layer was washed with brine, dried over sodium sulfate, filtered, concentrated, then the crude residue was then purified by column chromatography (EtOAc/hexanes from 30:70 to 100% EtOAc) to give 2-(3-hydroxyphenoxy)-N,N-dimethylacetamide as a yellow solid (105 mg, 24%). MS (ES+) C₁₀H₁₃NO₃ requires: 195. found 196 [M+H]⁺.

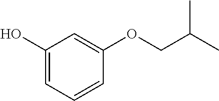

### 3-isobutoxyphenol (Intermediate 4)

A mixture of resorcinol (1.0 g, 9.1 mmol), 1-bromo-2-methylpropane (1.24 g, 9.08 mmol), and potassium carbonate (1.88 g, 13.6 mmol) in DMF (23 ml) was stirred at ambient temperature for 3 hrs, starting material was still observed by LCMS, so an additional amount of potassium carbonate (1.8 g) was added and the reaction mixture was heated to 80° C. for 16 hrs. The cooled reaction mixture was carefully quenched with 1M HCl, then partitioned between EtOAc and water. The seperated organic layer was washed with brine, dried over sodium sulfate, filtered, and concentrated. The crude residue was purified by column chromatography (20:80 to 80:20 EtOAc/hexanes) to give 3-isobutoxyphenol as a liquid (500 mg, 33%). MS (ES+) C₁₀H₁₄O₂ requires: 166. found 167 [M+H]⁺.

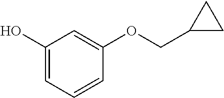 [SMILES: Oc1cccc(OCC2CC2)c1]

### 3-(cyclopropylmethoxy)phenol (Intermediate 5)

To a solution of resorcinol (2.0 g, 18 mmol) in DMF (8 mL) were added (bromomethyl)cyclopropane (0.817 g, 6.05 mmol) and potassium carbonate (2.51 g, 18.2 mmol) and the resulting mixture was stirred at 25° C. for 12 h. The reaction mixture was quenched with 1M aq. HCl (36 mL, 36 mmol) until the solution was acidic. Water (100 mL) was added, and the layers were separated. The aqueous phase was extracted with EtOAc (3×100 mL), and the combined organic layers were washed with water, concentrated under reduced pressure then purified by column chromatography (0-100% EtOAc/hexanes) to give 3-(cyclopropylmethoxy)phenol (700 mg, 70%). MS (ES+) C₁₀H₁₂O₂ requires: 164. found: 165 [M+H]⁺.

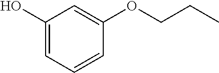

### 3-propoxyphenol (Intermediate 6)

To a solution of resorcinol (10.7 g, 98 mmol) in DMF (100 ml) was added potassium carbonate powder (13.5 mg, 98 mmol) and 1-bromopropane (2.96 mL, 32.5 mmol). The reaction mixture was stirred at 50° C. for 72 h. The cooled reaction mixture was then diluted with water (300 mL), and adjusted the pH until acidic by carefully quenching with 1N HCl. The product was extracted with EtOAc (2×300 mL), washed the organic layer with brine, dried over sodium sulfate, filtered, concentrated and purified by column chromatography (10-100% EtOAc/hexanes) to give 3-propoxyphenol as a colorless liquid (3.86 g, 78%). MS (ES+) C₉H₁₂O₂ requires: 152. found 153 [M+H]⁺.

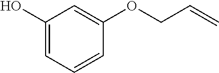

### 3-(allyloxy)phenol (Intermediate 7)

A suspension of potassium carbonate (17.1 g, 124 mmol) and resorcinol (13.65 g, 124 mmol) in DMF (80 mL) was treated with 3-bromoprop-1-ene (5 g, 41.3 mmol) and the reaction mixture was stirred for 2 days at RT. The reaction mixture was carefully neutralized with aq. HCl (21 mL, 250 mmol) washed with 20-30% EtOAc in hexanes (2×300 mL) and water (800 mL). The combined organic layers were concentrated and purified by column chromatography (0-10% EtOAc/hexanes) to give 3-(allyloxy)phenol (4.5 g, 73%). MS (ES+) C₉H₁₀O₂ requires: 150. found 151 [M+H]⁺.

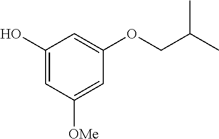

### 3-isobutoxy-5-methoxyphenol (Intermediate 8)

A solution of 5-methoxybenzene-1,3-diol (500 mg, 3.57 mmol) in DMF (5 ml) was treated with potassium carbonate (986 mg, 7.14 mmol) and 1-bromo-2-methylpropane (0.34 ml, 3.6 mmol). The reaction mixture was stirred at 50° C. for 16 h. The cooled reaction mixture was then diluted with water, neutralized with 1N HCl and extracted with EtOAc. The seperated organic layer was washed with brine, dried over sodium sulfate, concentrated, then purified by column chromatography (1:1 EtOAc/hexanes) to give 3-isobutoxy-5-methoxyphenol as a yellow liquid (200 mg, 29%). MS (ES+) C₁₁H₁₆O₃ requires: 196. found 197 [M+H]⁺.

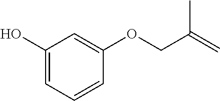

### 3-((2-methylallyl)oxy)phenol (Intermediate 9)

To a suspension of potassium carbonate (9.0 g, 65 mmol) and resorcinol (7.17 g, 65.1 mmol) in DMF (50 mL) was added 3-bromo-2-methylprop-1-ene (2.5 mL, 24.80 mmol) (dropwise). The reaction mixture was stirred for 60 mins. LC-MS showed the mono-alkylation and bis alkylation 1:1 ratio. The reaction mixture was then quenched with aq. HCl (10 mL, 120 mmol), washed with EtOAc (500 mL) and water (2×500 mL). The combined organic layers were concentrated, then purified by column chromatography (0-100% EtOAc/hexanes) to give 3-((2-methylallyl)oxy)phenol (1.4 g, 39%) as a liquid. MS (ES+) C₁₀H₁₂O₂ requires: 164. found 165 [M+H]⁺.

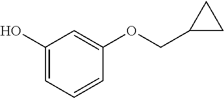 [SMILES: Oc1cccc(OCC2CC2)c1]

### 3-(cyclopropylmethoxy)phenol (Intermediate 10)

To a solution of resorcinol (2.0 g, 18.1 mmol) in DMF (8 mL) were added (bromomethyl)cyclopropane (0.817 g, 6.05 mmol) and potassium carbonate (2.51 g, 18.2 mmol) and the resulting mixture was stirred at 25° C. for 12 h. The reaction mixture was quenched with 1 M aq. HCl (36.3 mL) until the solution was acidic. Water (100 mL) was added, and the organic layers were separated. The aqueous phase was extracted with EtOAc (3×100 mL), the combined organic layers were washed with water, concentrated under reduced pressure, and purified by column chromatography (0-100% EtOAc/hexanes) to give 3-(cyclopropylmethoxy)phenol (700 mg, 70%). MS (ES+) C₁₀H₁₂O₂ requires: 164. found: 165 [M+H]⁺.

### 5-propoxybenzene-1,3-diol (Intermediate 11) and 3,5-dipropoxyphenol (Intermediate 12)

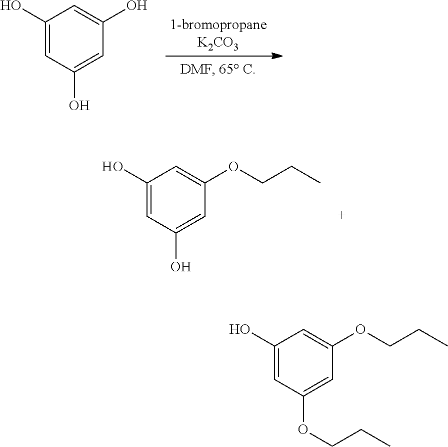

A solution of benzene-1,3,5-triol (500 mg, 3.96 mmol) in DMF (5 ml) was treated with potassium carbonate powder (1.70 g, 12.3 mmol) and 1-bromopropane (0.76 ml, 8.3 mmol), the mixture was stirred at 65° C. for 16 h. The cooled reaction mixture was diluted with water, carefully quenched with 1N HCl until acidic, then extracted with EtOAc. The organic phase was then washed with brine and dried over sodium sulfate, concentrated, then the residue was purified by column chromatography (1:1 EtOAc/hexanes) to give 5-propoxybenzene-1,3-diol (96 mg, 14%) MS (ES+) C₉H₁₂O₃ requires: 168. found: 169 [M+H]⁺and 3,5-dipropoxyphenol (160 mg, 19%) as liquids. MS (ES+) C₁₂H₁₈O₃ requires: 210. found: 211 [M+H]⁺.

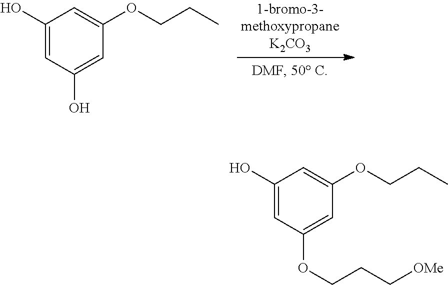 [SMILES: CCCOc1cc(O)cc(O)c1 ; CCCOc1cc(O)cc(OCCCOC)c1]

### 3-(3-methoxypropoxy)-5-propoxyphenol (Intermediate 13)

A solution of 5-propoxybenzene-1,3-diol (96 mg, 0.57 mmol) in DMF (1 ml) was treated with potassium carbonate (245 mg, 1.77 mmol) and 1-bromo-3-methoxypropane (0.078 ml, 0.69 mmol), the mixture was stirred overnight at 50° C. The cooled reaction mixture was diluted with water, neutralized with 1N HCl and extracted with EtOAc. The organic phase was then washed with brine, dried over sodium sulfate, concentrated then the crude residue was purified by column chromatography (1:1 EtOAc/hexanes) to give 3-(3-methoxypropoxy)-5-propoxyphenol as a yellow liquid (44 mg, 32%). MS (ES+) C₁₃H₂₀O₄ requires: 240. found 241 [M+H]⁺.

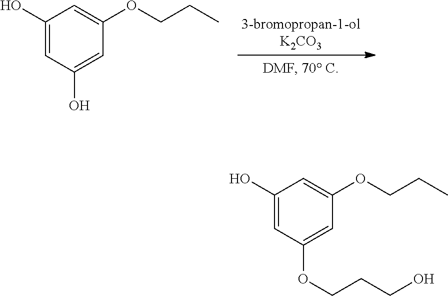 [SMILES: CCCOc1cc(O)cc(O)c1 ; CCCOc1cc(O)cc(OCCCO)c1]

### 3-(3-hydroxypropoxy)-5-propoxyphenol (Intermediate 14)

To a solution of 3-bromopropan-1-ol (136 mg, 0.981 mmol) in DMF (1.5 ml) were added 3-bromopropan-1-ol (136 mg, 0.981 mmol) and potassium carbonate (247 mg, 1.78 mmol) and the resulting mixture was stirred at 70° C. overnight. The reaction mixture was diluted with water and carefully quenched with 1N HCl, EtOAc was added and the seperated organic phase was then washed with brine, dried over sodium sulfate, filtered, concentrated, then the residue was purified by column chromatography (1:1 EtOAc/hexanes) to give 3-(3-hydroxypropoxy)-5-propoxyphenol (23 mg, 11%). MS (ES+) C₂₃H₁₈O₄ requires: 226. found 227 [M+H]⁺.

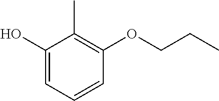

### 2-methyl-3-propoxyphenol (Intermediate 15)

To a suspension of potassium carbonate (2.23 g, 16.1 mmol) and 2-methylbenzene-1,3-diol (2 g, 16.1 mmol) in DMF (20 mL) was added 1-bromopropane (0.489 mL, 5.37 mmol). The reaction mixture was stirred for 4 days at RT, then quenched with 1N aq. HCl (11 mL) until the mixture was acidic then diluted with 100 mL of water. The resulting mixture was extracted with EtOAc (2×100 mL) and the combined organic layers were concentrated and purified by column chromatography (0-100% EtOAc/hexanes) to give 2-methyl-3-propoxyphenol (670 mg, 75%). MS (ES+) C₁₀H₁₄O₂ requires: 166. found 167 [M+H]⁺.

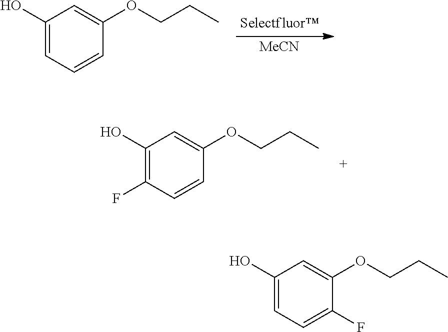

### 2-fluoro-5-propoxyphenol (Intermediate 16) and 4-fluoro-4-propoxyphenol (Intermediate 17)

To a solution of 3-propoxyphenol (300 mg, 1.971 mmol) in acetonitrile (4.5 ml) was added Selectfluor™ (698 mg, 1.97 mmol) and the resulting mixture was stirred at 25° C. overnight. The reaction mixture was quenched with methanol, concentrated, then purified by silica gel chromatography (0-50% EtOAc/hexanes) to give 2-fluoro-5-propoxyphenol (90 mg, 27%) as a yellow liquid: MS (ES+) C₉H₁₁FO₂: 170. found: 171 [M+H]⁺; Rt=1.78 min. ¹H NMR (500 MHz CDCl₃) δ: 6.89 (dd, J=11.2, 8.7 Hz, 1H), 6.51 (s, 1H), 6.50 (dd, J=7.2, 2.9 Hz, 1H), 6.34-6.31 (m, 1H), 3.93 (t, J=6.7 Hz, 2H), 1.85-1.78 (m, 2H). 1.02 (t, J=7.5 Hz, 3H); and 4-fluoro-3-propoxyphenol (220 mg, 66%) as a yellow liquid. MS (ES+) C₉H₁₁FO₂: 170. found: 171 [M+H]⁺; Rt=1.69 min. ¹H NMR (500 MHz CDCl₃) δ: 6.95 (dd, J=10.2, 9.0, Hz, 1H), 6.56 (dd, J=7.3, 2.9, Hz 1H), 6.38-6.34 (m, 1H), 5.43 (d, J=3.9 Hz, 1H), 3.85 (t, J=6.5 Hz, 2H), 1.81-1.74 (m, 2H), 1.01 (t, J=7.5 Hz, 3H). Regiochemistry was assigned comparing the NMR spectral data (COSY and NOESY experiments) of Examples 104 and 105.

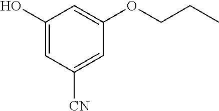

### 3-hydroxy-5-propoxybenzonitrile (Intermediate 18)

To a solution of 1-bromopropane (0.14 ml, 1.63 mmol) in DMF (2 ml) were added 1-bromopropane (0.15 ml, 1.6 mmol) and potassium carbonate (225 mg, 1.63 mmol) and the resulting mixture was stirred at 40° C. for 2 days. The cooled reaction mixture was diluted with water, quenched with 1N HCl and taken up into EtOAc. The seperated organic layer was then washed with brine and dried over sodium sulfate, filtered and concentrated then purified by column chromatography (3:7 EtOAc/hexanes) to give 3-hydroxy-5-propoxybenzonitrile as a white solid (113 mg, 43%). MS (ES+) C₁₀H₁₁NO₂ requires: 177. found: 178 [M+H]⁺.

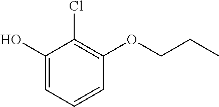

### 2-chloro-3-propoxyphenol (Intermediate 19)

A mixture of 1-bromopropane (255 mg, 2.08 mmol), potassium carbonate (287 mg, 2.08 mmol), and 2-chlorobenzene-1,3-diol (300 mg, 2.08 mmol) in DMF (5 ml) was stirred for 8 h. The mixture was diluted with EtOAc and washed with water. The organic layer was then concentrated and purified by column chromatography (20-100% EtOAc/hexanes then 0-60% MeOH/EtOAc) to give the title compound (168 mg, 43%). MS (ES+) C₉H₁₁³⁵ClO₂ requires: 186. found: 187 [M+H]⁺.

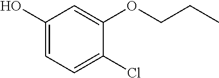

### 4-chloro-3-propoxyphenol (Intermediate 20)

To a 0° C. solution of 3-propoxyphenol (300 mg, 1.971 mmol) in chloroform (1 ml) was added sulfuryl chloride (0.18 ml, 2.2 mmol) dropwise over 15 min. The reaction was then heated to 65° C. for 2 h. The solution was allowed to warm up to room temperature, then the reaction mixture was quenched with methanol, concentrated, and the crude material was purified by column chromatography (20-100% EtOAc/hexanes, then 0-35% MeOH/EtOAc) to give the 4-chloro-3-propoxyphenol (200 mg, 54%). MS (ES+) C₉H₁₁³⁵ClO₂ requires: 186. found: 187 [M+H]⁺. Regiochemistry was assigned using spectral NMR data (COSY and NOESY experiments) for Example 112 which correlated with Example 105.

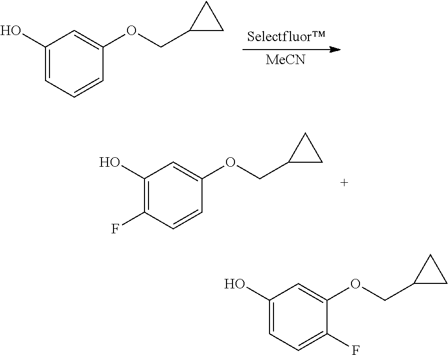 [SMILES: "MeCN" ; Oc1cccc(OCC2CC2)c1 ; Oc1cc(OCC2CC2)ccc1F ; Oc1ccc(F)c(OCC2CC2)c1]

### 5-(cyclopropylmethoxy)-2-fluorophenol (Intermediate 21) and 3-(cyclopropylmethoxy)-4-fluorophenol (Intermediate 22)

To a solution of 3-(cyclopropylmethoxy)phenol (350 mg, 2.13 mmol) in acetonitrile (8 ml) were added Selectfluor™ (906 mg, 2.56 mmol) and the resulting mixture was stirred at 25° C. overnight. The reaction mixture was treated with methanol and the resulting mixture was filtered and purified by silica gel chromatography (0-100% EtOAc/hexanes) to give an inseperable mixture of 5-(cyclopropylmethoxy)-2-fluorophenol and 3-(cyclopropylmethoxy)-4-fluorophenol (150 mg, 39%) as a yellow liquid. MS (ES+) C₁₀H₁₁FO₂ requires: 182. found: 183 [M+H]⁺. Regiochemistry was assigned comparing the NMR spectral data (COSY and NOESY experiments) of Example 129 with Example 112.

### 4-methoxy-3-propoxyphenol and 2-methoxy-5-propoxyphenol (Intermediate 23)

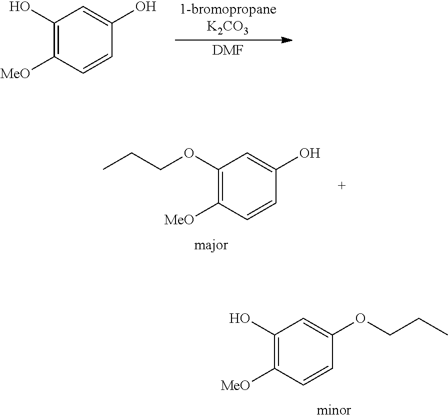

A mixture of 1-bromopropane (439 mg, 3.57 mmol), potassium carbonate (493 mg, 3.57 mmol), and 4-methoxybenzene-1,3-diol (500 mg, 3.57 mmol) in DMF (5 mL) was stirred for 8 h. The mixture was diluted with EtOAc and washed with water. The organic layer was then concentrated and purified by column chromatography (20-100% EtOAc/hexanes and then 0-60% MeOH/EtOAc) to give a mixture of 2-methoxy-5-propoxyphenol (minor) and 4-methoxy-3-propoxyphenol (major) (180 mg, 28%). MS (ES+) C₁₀H₁₄O₃ requires: 182. found: 183 [M+H]⁺. Regiochemistry was assigned comparing the NMR spectral data (COSY and NOESY experiments) of Example 113 with Example 114.

### 5-(cyclopropylmethoxy)benzene-1,3-diol (Intermediate 24) and 3,5-bis(cyclopropylmethoxy)phenol (Intermediate 25)

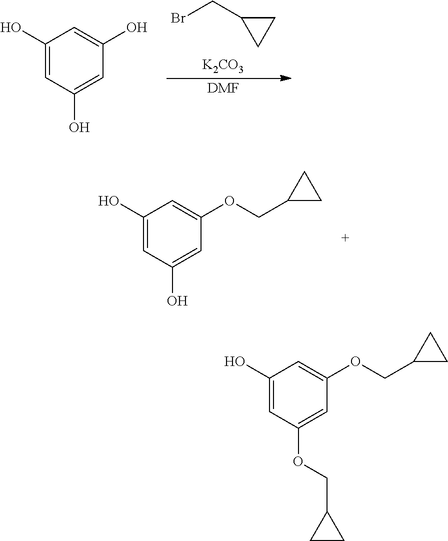

A solution of benzene-1,3,5-triol (280 mg, 2.22 mmol) in DMF (3 ml) was treated with potassium carbonate (614 mg, 4.44 mmol) and (bromomethyl)cyclopropane (0.2 ml, 2.2 mmol), the mixture was stirred at RT for 4 days then was diluted with EtOAc and washed with 1M HCl. The separated organic layer was washed with sat. aq. NaCl, dried over sodium sulfate, filtered and concentrated under reduced pressure. The residue was purified via silica gel chromatography (20-100% EtOAc/hexanes) to give 5-(cyclopropylmethoxy)benzene-1,3-diol (120 mg, 30%) as an off-white solid MS (ES+) C₁₀H₁₂O₃ requires: 180. found: 181 [M+H]⁺; and 3,5-bis(cyclopropylmethoxy)phenol (30 mg, 6%) as a colorless liquid. MS (ES+) C₁₄H₁₈O₃ requires: 234. found: 235 [M+H]⁺.

### 3-(cyclopropylmethoxy)-5-propoxyphenol (Intermediate 26)

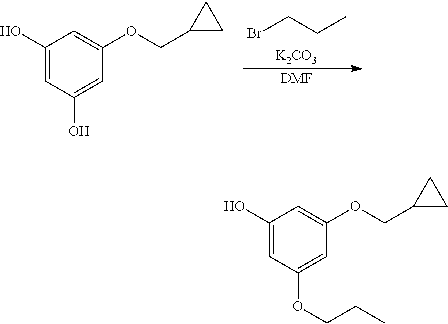

To a solution of 5-(cyclopropylmethoxy)benzene-1,3-diol (120 mg, 0.67 mmol) in DMF (2 ml) was added potassium carbonate (184 mg, 1.33 mmol) and 1-bromopropane (0.061 ml, 0.67 mmol) and the reaction mixture was stirred at 40° C. for 1 day. The cooled reaction mixture was neutralized with 1M HCl, diluted with EtOAc, and the seperated organic layer was washed with sat. aq. NaCl solution, dried over sodium sulfate, filtered and concentrated under reduced pressure. The residue was purified via silica gel chromatography (20-100% EtOAc/hexanes) to give 3-(cyclopropylmethoxy)-5-propoxyphenol (34 mg, 23%) as an off-white solid. MS (ES+) C₁₃H₁₈O₃ requires: 222. found: 223 [M+H]⁺.

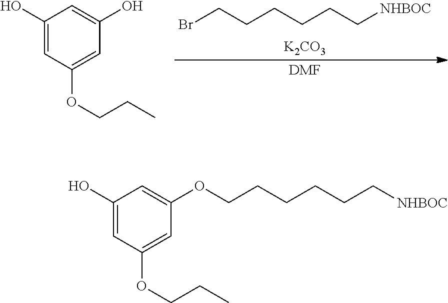

### tert-butyl (6-(3-hydroxy-5-propoxyphenoxy)hexyl)carbamate (Intermediate 27)

To a solution of 5-propoxybenzene-1,3-diol (344 mg, 2.05 mmol), tert-butyl(6-bromohexyl)carbamate (268 mg, 0.956 mmol) in 3 mL of DMF was added potassium carbonate (326 mg, 2.36 mmol) and the resulting mixture was stirred at 50° C. for 16 h. The cooled reaction mixture was neutralized with 1M HCl, diluted with EtOAc, and the seperated organic layer was washed with sat. aq. NaCl solution, dried over sodium sulfate, filtered and concentrated under reduced pressure. The residue was purified via silica gel chromatography (1:9 to 1:1 EtOAc/hexanes) to give tert-butyl (6-(3-hydroxy-5-propoxyphenoxy)hexyl)carbamate (174 mg, 23%) as a light-brown viscous liquid. MS (ES+) C₂₀H₃₃NO₅ requires: 367. found: 368 [M+H]⁺.

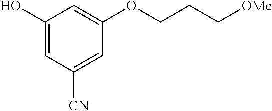 [SMILES: COCCCOc1cc(O)cc(C#N)c1]

### 3-hydroxy-5-(3-methoxypropoxy)benzonitrile (Intermediate 28)

To a solution of 1-bromo-3-methoxypropane (0.18 ml, 1.6 mmol) in DMF (2 ml) were added 1-bromo-3-methoxypropane (0.18 ml, 1.63 mmol) and potassium carbonate (225 mg, 1.63 mmol) and the resulting mixture was stirred at 40° C. for 3 h. The mixture was diluted with water quenched with 1N HCl and taken up into EtOAc. The seperated organic phase was washed with brine, dried over sodium sulfate, filtered, concentrated then purified by column chromatography (3:7 EtOAc/DCM) to give 3-hydroxy-5-(3-methoxypropoxy)benzonitrile as a yellow liquid (105 mg, 34%). MS (ES+) C₁₁H₁₃NO₃ requires: 207. found: 176 [M—OCH₃]⁺.

### 5-(3-methoxypropoxy)benzene-1,3-diol and 3,5-bis(3-methoxypropoxy)phenol (Intermediate 29)

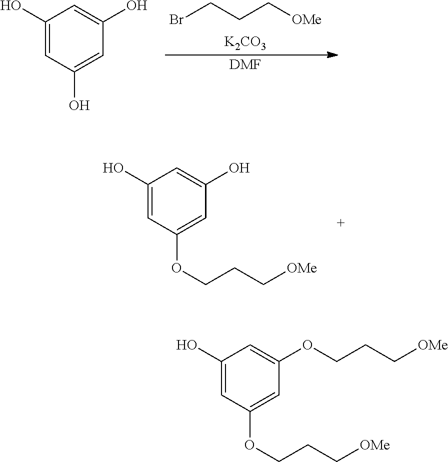

To a solution of benzene-1,3,5-triol (500 mg, 3.96 mmol) in DMF (25 ml) was added potassium carbonate (1.10 g, 7.9 mmol) and 1-bromo-3-methoxypropane (0.66 ml, 5.9 mmol). The mixture was stirred at 50° C. for 72 h, then diluted with water (50 mL) and adjusted the pH of the mixture until acidic with 1N aq. HCl. The product was extracted with EtOAc (2×40 mL), washed with brine (30 mL), dried over sodium sulfate, and concentrated. Purification by silica gel chromatography (1:9 to 100% EtOAc/hexanes) gave 5-(3-methoxypropoxy)benzene-1,3-diol (235 mg, 30% yield) MS (ES+) C₁₀H₁₄O₄ requires: 198. found 199 [M+H]⁺ and 3,5-bis(3-methoxypropoxy)phenol (185 mg, 17% yield) MS (ES+) C₁₄H₂₂O₅ requires: 270. found 271 [M+H]⁺, both as colorless liquids.

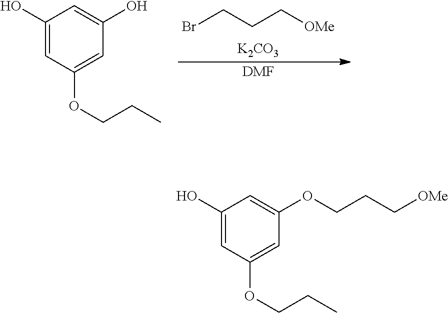

### 3-(3-methoxypropoxy)-5-propoxyphenol (Intermediate 30)

To a solution of 5-propoxybenzene-1,3-diol (1.28 g, 7.59 mmol) in DMF (8 ml) were added potassium carbonate (2.1 g, 15 mmol) and 1-bromo-3-methoxypropane (0.93 ml, 8.4 mmol) and the resulting mixture was stirred at 50° C. for 3 h. The cooled reaction mixture was neutralized with 1M aq. HCl, and extracted with EtOAc. The seperated organic layer was washed with sat. aq. NaCl, dried over sodium sulfate, filtered and concentrated under reduced pressure. The residue was purified via silica gel chromatography (1:9 to 6:4 EtOAc/hexanes) to give 3-(3-methoxypropoxy)-5-propoxyphenol (580 mg, 32%) as a yellow liquid. MS (ES+) C₁₃H₂₀O₄ requires: 240. found: 241 [M+H]⁺.

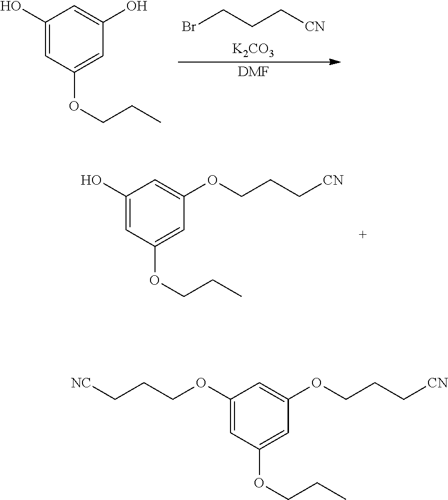

### 4-(3-hydroxy-5-propoxyphenoxy)butanenitrile (Intermediate 31)

To a solution of 5-propoxybenzene-1,3-diol (200 mg, 1.189 mmol) in DMF (8 mL) were added 4-bromobutanenitrile (59 mg, 0.40 mmol) and potassium carbonate (164 mg, 1.19 mmol) and the resulting mixture was stirred at 25° C. for 12 h. The reaction mixture was quenched with 1N aq. HCl (2.4 mL, 2.4 mmol) until the solution was acidic, water (20 mL) was added, and the layers were separated. The aqueous phase was extracted with EtOAc (3×20 mL), and the combined organic layers were washed with water, concentrated under reduced pressure, then purified by column chromatography (0-100% EtOAc/hexanes) to give an inseperable mixture of 4-(3-hydroxy-5-propoxyphenoxy)butanenitrile MS (ES+) C₁₄H₂₂O₅ requires: 235. found: 236 [M+H]⁺ and 4,4′-((5-propoxy-1,3-phenylene)bis(oxy))dibutanenitrile (216 mg). MS (ES+) C₁₇H₂₂N₂O₃ requires: 302. found: 303 The mixture was used directly in the next step.

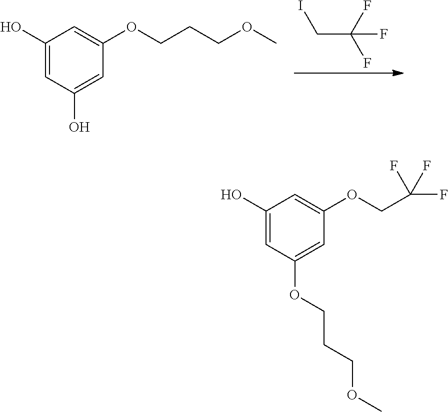

### 3-(3-methoxypropoxy)-5-(2,2,2-trifluoroethoxy)phenol (Intermediate 32)

To a solution of 5-(3-methoxypropoxy)benzene-1,3-diol (250 mg, 1.26 mmol) in DMF (3 ml) were added potassium carbonate (349 mg, 2.52 mmol) and 1,1,1-trifluoro-2-iodoethane (0.25 ml, 2.5 mmol) and the resulting mixture was stirred overnight at 80° C. No product formation was observed, so an additional 6 eq. of iodide was added and the mixture was stirred overnight at 80° C. The reaction mixture was diluted with EtOAc, quenched with water and 1N HCl. The layers were separated, and the organic layer was washed with sat NaCl, dried over sodium sulfate, filtered and concentrated under reduced pressure. The residue was purified via silica gel chromatography (20-100% EtOAc/hexanes) to give 3-(3-methoxypropoxy)-5-(2,2,2-trifluoroethoxy)phenol (44 mg, 12%). MS (ES+) C₁₂H₁₅F₃O₄ requires: 280. found: 281 [M+H]⁺.

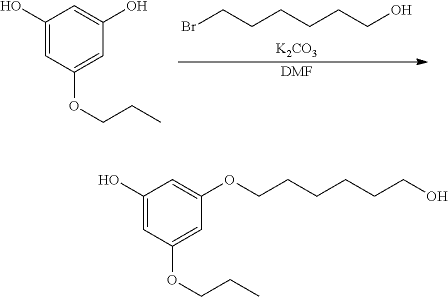

### 3-((6-hydroxyhexyl)oxy)-5-propoxyphenol (Intermediate 33)

To a suspension of 5-propoxybenzene-1,3-diol (300 mg, 1.78 mmol) in DMF (5 ml) were added potassium carbonate (518 mg, 3.75 mmol) 6-bromohexan-1-ol (0.28 ml, 2.14 mmol) was added and the resulting mixture was stirred at 80° C. for 4 h. The cooled reaction mixture was diluted with EtOAc and neutralized with 1N HCl. The separated organic layer was washed with sat. aq. NaCl, dried over sodium sulfate, filtered and concentrated under reduced pressure. The residue was purified via silica gel chromatography (20-100% EtOAc/hexanes) to give 3-((6-hydroxyhexyl)oxy)-5-propoxyphenol (188 mg, 39%) as a viscous brown liquid. MS (ES+) C₁₅H₂₄O₄ requires: 268. found: 269 [M+H]⁺.

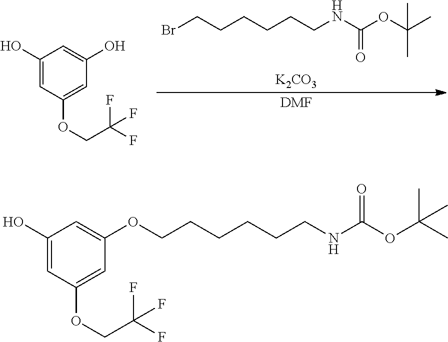

### tert-butyl (6-(3-hydroxy-5-(2,2,2-trifluoroethoxy)phenoxy)hexyl)carbamate (Intermediate 34)

To a suspension of 5-(2,2,2-trifluoroethoxy)benzene-1,3-diol (474 mg, 2.28 mmol) in DMF (4 ml) were added potassium carbonate (315 mg, 2.28 mmol) tert-butyl(6-bromohexyl)carbamate (213 mg, 0.76 mmol) was added and the resulting mixture was stirred at 50° C. overnight. The cooled reaction mixture was diluted with water and acidified with 1N HCl. The mixture was neutralized with 1N HCl and twice extracted with EtOAc. The combined organic layers were concentrated, dried over sodium sulfate then purified via silica gel chromatography (20-100% EtOAc/hexanes) to give tert-butyl (6-(3-hydroxy-5-(2,2,2-trifluoroethoxy)phenoxy)hexyl)carbamate as liquid (210 mg, 68%). MS (ES+) C₁₉H₂₈F₃NO₅ requires: 407. found: 408 [M+H]⁺, 352 [M—C₄H₈]⁺.

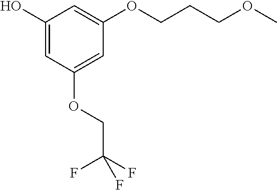 [SMILES: COCCCOc1cc(O)cc(OCC(F)(F)F)c1]

### 3-(3-methoxypropoxy)-5-(2,2,2-trifluoroethoxy)phenol (Intermediate 35)

To a suspension of 5-(2,2,2-trifluoroethoxy)benzene-1,3-diol (250 mg, 1.20 mmol) in DMF (3 ml) were added potassium carbonate (349 mg, 2.52 mmol), 1-bromo-3-methoxypropane (0.13 ml, 1.20 mmol) and the resulting mixture was stirred at 50° C. for 4 h. The cooled reaction mixture was then acidified with 1N HCl, and extracted with EtOAc. The seperated organic phase was washed with water, and sat. aq. NaCl solution, then dried over sodium sulfate, filtered and concentrated under reduced pressure. The residue was purified via silica gel chromatography (20-100% EtOAc/hexanes) to give 3-(3-methoxypropoxy)-5-(2,2,2-trifluoroethoxy)phenol (80 mg, 24%) as a viscous yellow liquid. MS (ES+) C₁₂H₁₅F₃O₄ requires: 280. found: 281 [M+H]⁺.

### 3-ethoxy-5-isobutoxyphenol (Intermediate 36)

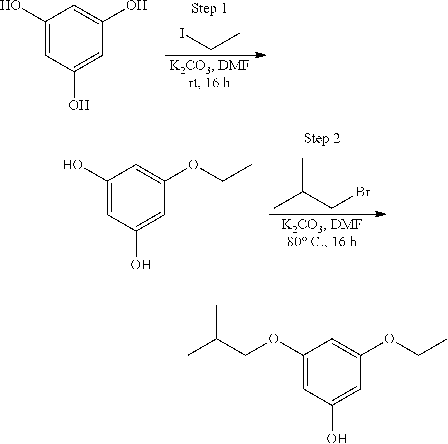

### Step 1: 5-ethoxybenzene-1,3-diol

To a solution of benzene-1,3,5-triol (5.0 g, 39.6 mmol) in DMF (50 mL) was added K₂CO₃ (10.9 g, 79.2 mmol), and iodoethane (6.1 g, 39.6 mmol). The reaction mixture was stirred at RT for 16 h, then added water (50 ml), extracted with EtOAc (2 x 50 ml), washed with brine (2 x 50 ml), dried over Na₂SO₄, filtered and concentrated. The crude product was purified by column chromatography (0:100 to 15:85 MeOH/DCM) to give 5-ethoxybenzene-1,3-diol as a yellow liquid (2.5 g, 41%). MS (ES+) C₈H₁₀O₃ requires: 154. found: 155 [M+H]⁺.

### Step 2: 3-ethoxy-5-isobutoxyphenol

To a solution of 5-ethoxybenzene-1,3-diol (2.3 g, 14.9 mmol) in DMF (22 mL) was added K₂CO₃ (6.2 g, 44.9 mmol), and 1-bromo-2-methylpropane (2.0 g, 14.9 mmol). The reaction mixture was stirred at 80° C. for 16 h. The cooled reaction mixture was diluted with water (50 ml), extracted with EtOAc (2×50 ml), washed with brine (2×50 ml), dried over Na₂SO₄, filtered and concentrated. The crude product was purified by column chromatography (0:100 to 40:60 EtOAc/hexanes) to give 3-ethoxy-5-isobutoxyphenol as a yellow liquid (900 mg, 33%). MS (ES+) C₁₂H₁₈O₃ requires: 210. found: 211 [M+H]⁺.

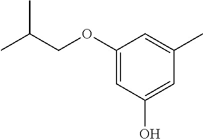

### 3-isobutoxy-5-methylphenol (Intermediate 37)

To a solution of 5-methylbenzene-1,3-diol (2.0 g, 16 mmol) in 20 mL of DMF was added 1-bromo-2-methylpropane (2.7 g, 19 mmol) and K₂CO₃ (2.0 g, 24.5 mmol). The reaction mixture was heated to 75° C. and stirred overnight. To the cooled reaction mixture was added water and the product was extracted with EtOAc (3×10 mL). The combined organic layers were washed with water (10 mL), brine (10 mL), then dried over anhydrous sodium sulfate, filtered, and concentrated under reduced pressure. The residue was purified by column chromatography (1:2 EtOAc/hexanes) to afford 3-isobutoxy-5-methylphenol as a yellow solid (1.3 g, 45%). MS (ES+) Cl₁H₁₆O₂ requires: 180. found: 181 [M+H]⁺.

### 3-ethyl-5-isobutoxyphenol (Intermediate 38)

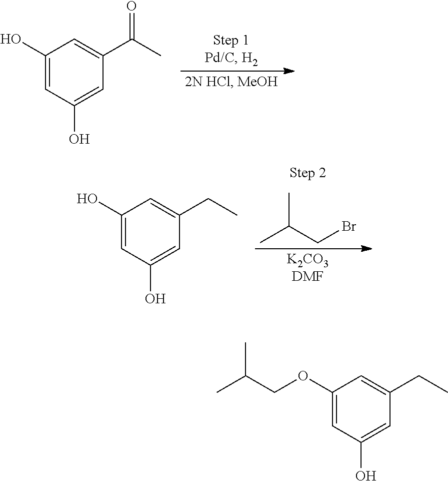

### Step 1: 5-ethylbenzene-1,3-diol

To a solution of 1-(3,5-dihydroxyphenyl)ethanone (2.5 g, 16.4 mmol) in MeOH (120 mL) and 2N HCl (120 ml) was added Pd/C (250 mg) under an inert atmosphere. The reaction mixture was then charged with hydrogen gas and stirred at RT for 16 h under an N₂ atmosphere. The reaction mixture was filtered, concentrated and the residue was taken up into EtOAc (50 ml), washed with brine (2×50 ml), dried over Na₂SO₄, filtered and concentrated. The crude product was purified by column chromatography (0:100 to 40:60 EtOAc/hexanes) to give 5-ethylbenzene-1,3-diol as a yellow liquid (1.8 g, 79%). MS (ES+) C₈H₁₀O₂ requires: 138. found: 139 [M+H]⁺.

### Step 2: 3-ethyl-5-isobutoxyphenol

To a solution of 1-(3,5-dihydroxyphenyl)ethanone (1.8 g, 13.0 mmol) in DMF (20 mL) was added K₂CO₃ (5.4 g, 39.1 mmol), 3-ethyl-5-isobutoxyphenol (1.8 g, 13.3 mmol) and the reaction mixture was stirred at 80° C. for 16 h. To the cooled reaction mixture was added water (50 ml), and extracted with EtOAc (2×50 ml), washed with brine (2×50 ml), dried over Na₂SO₄, filtered and concentrated. The crude product was purified by column chromatography (0:100 to 40:60 EtOAc/hexanes) to give the product as a yellow liquid (600 mg, 24%). MS (ES+) C₁₂H₁₈O₂ requires: 194. found: 195 [M+H]⁺.

### 3-chloro-5-isobutoxyphenol (Intermediate 39)

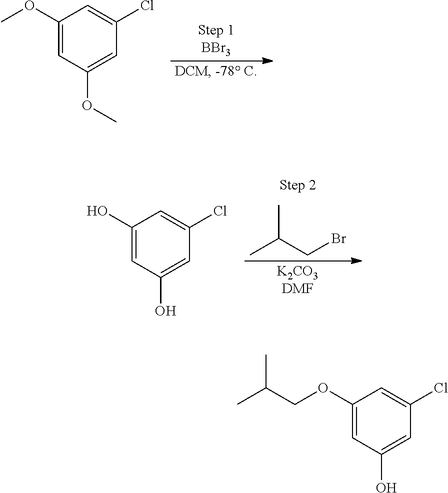

### Step 1: 5-chlorobenzene-1,3-diol

To a −78° C. solution of 1-chloro-3,5-dimethoxybenzene (1.73 g, 10 mmol) in 10 mL of CH₂Cl₂ was added 10 mL of BBr₃/CH₂Cl₂. The reaction mixture was stirred at −78° C. for 2 h, then allowed to warm up to RT overnight. To the mixture was added water and the product was extracted with EtOAc (3×10 mL). The combined organic layers were washed with water (10 mL) and brine (10 mL), dried over anhydrous sodium sulfate, filtered and concentrated under reduced pressure to afford a crude product. The residue was purified by flash chromatography (1:1 EtOAc/hexanes) to give 3-chloro-5-isobutoxyphenol as a yellow solid (1.23 g, 85%). MS (ES+) C₆H₅ClO₂ requires: 144. found: 145 [M+H]⁺.

### Step 2: 3-chloro-5-isobutoxyphenol

To a solution of 5-chlorobenzene-1,3-diol (1.3 g, 9.0 mmol) in 20 mL of DMF was added 1-bromo-2-methylpropane (1.5 g, 11 mmol) and K₂CO₃ (6.2 g, 45 mmol). The reaction mixture was heated to 75° C. and stirred overnight. To the cooled reaction mixture was added water and the product was extracted with EtOAc (3×10 mL). The combined organic layers were washed with water (10 mL) and brine (10 mL), dried over anhydrous sodium sulfate, filtered and concentrated under reduced pressure to afford a crude product. The residue was purified by flash chromatography (1:1 EtOAc/hexanes) to give 3-chloro-5-isobutoxyphenol as a yellow liquid (0.9 g, 52%). MS (ES+) C₁₀H₁₃ClO₂ requires: 200. found: 201 [M+H]⁺.

### 3-fluoro-5-isobutoxyphenol (Intermediate 40)

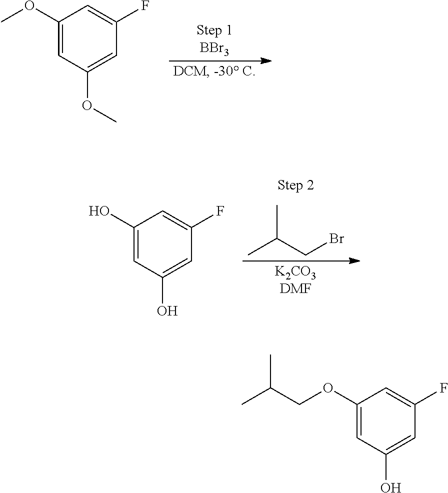

### Step 1: 5-fluorobenzene-1,3-diol

To a −30° C. solution of 1-fluoro-3,5-dimethoxybenzene (1.56 g, 10 mmol) in 10 mL of CH₂Cl₂ was added 10 mL of BBr₃/CH₂Cl₂ at while the solution was stirring. The reaction mixture was stirred at −30° C. for 2 h and then allowed to warm up to RT overnight. To the reaction mixture was added water and the product was extracted with EtOAc (3×10 mL). The organic layers were washed with water (10 mL) and brine (10 mL), dried over anhydrous sodium sulfate, then the combined organic layers were concentrated under reduced pressure to afford a crude product. The residue was purified by flash chromatography (1:1 EtOAc/hexanes) to give 5-fluorobenzene-1,3-diol as a yellow solid (1.2 g, 93%). MS (ES+) C₆H₅FO₂ requires: 128. found: 129 [M+H]⁺.

### Step 2: 3-fluoro-5-isobutoxyphenol

To a solution of 5-fluorobenzene-1,3-diol (1.2 g, 9.3 mmol) in 20 mL of DMF was added 1-bromo-2-methylpropane (1.6 g, 11.2 mmol) and K₂CO₃ (6.5 g, 46.5 mmol). The reaction mixture was heated to 75° C. and stirred overnight. To the cooled reaction mixture was added water and the product was extracted with EtOAc (3×10 mL). The combined organic layers were washed with water (10 mL) and brine (10 mL), dried over anhydrous sodium sulfate, filtered and under reduced pressure. The residue was purified by flash chromatography (1:1 EtOAc/hexanes) to give 3-fluoro-5-isobutoxyphenol as a yellow liquid (0.94 g, 55%). MS (ES+) C₁₀H₁₃FO₂ requires: 184. found: 185 [M+H]⁺.

### General Synthetic Methods and Examples

### Method A

### Example 1

### N-(6-(3-(benzyloxy)phenoxy)-1,3-dimethyl-2-oxo-2,3-dihydro-1H-benzo[d]imidazol-5-yl)-3,4-dimethoxybenzenesulfonamide

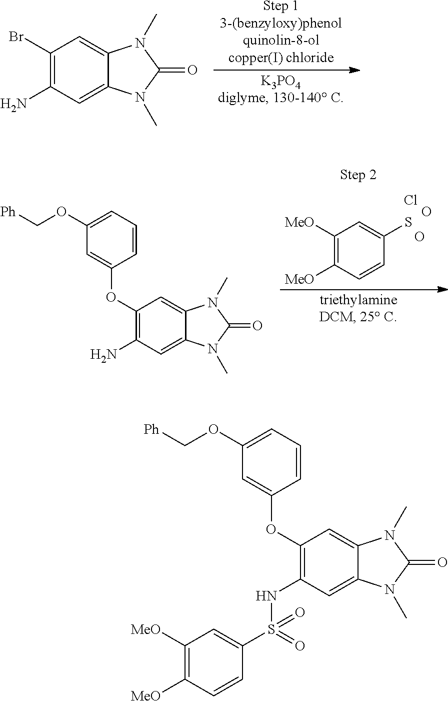

### Step 1: 5-amino-6-(3-(benzyloxy)phenoxy)-1,3-dimethyl-1H-benzo[d]imidazol-2(3H)-one

A mixture of 3-(benzyloxy)phenol (156 mg, 0.78 mmol), quinolin-8-ol (30 mg, 0.21 mmol), copper(I) chloride (10 mg, 0.10 mmol), potassium phosphate (200 mg, 0.94 mmol) and 5-amino-6-bromo-1,3-dimethyl-1H-benzo[d]imidazol-2(3H)-one (200 mg, 0.78 mmol) in diglyme (10 mL) was degassed under a nitrogen atmosphere, then the reaction mixture was heated to 130° C. for 72 h. Silica gel was then added to the cooled reaction mixture and the mixture was filtered through a pad of silica gel. The collected filtrate was then concentrated and purified by column chromatography (0-100% EtOAc/hexanesthen 0-40% MeOH/EtOAc) to give 5-amino-6-(3-(benzyloxy)phenoxy)-1,3-dimethyl-1H-benzo[d]imidazol-2(3H)-one as a solid (213 mg, 73%). MS (ES+) C₂₂H₂₁N₃O₃ requires: 375. found: 376 [M+H]⁺.

### Step 2

A solution of 5-amino-6-(3-(benzyloxy)phenoxy)-1,3-dimethyl-1H-benzo[d]imidazol-2(3H)-one (80 mg, 0.21 mmol) and triethylamine (43.1 mg, 0.426 mmol) in DCM (0.5 mL) was treated with 3,4-dimethoxybenzene-1-sulfonyl chloride (76 mg, 0.32 mmol). The reaction mixture was stirred at room temperature for 30 mins then the mixture was quenched with methanol, concentrated, and purified by prep-HPLC (mobile phase: A=0.1% TFA/H₂O, B=0.1% TFA/MeCN; gradient: B=40%-80% in 12 min; column: C18) to give 5-amino-6-(3-(benzyloxy)phenoxy)-1,3-dimethyl-1H-benzo[d]imidazol-2(3H)-one as a solid (56 mg, 46%). MS (ES+) C₃₀H₂₉N₃O₇S requires: 576. found: 577 [M+H]⁺. ¹H NMR (600 MHz, CDCl₃) δ 9.53 (s, 1H), 7.37 (m, 4H), 7.31 (m, 1H), 7.17 (m, 2H), 7.10 (s, 1H), 7.07 (t, J=8.0 Hz, 1H), 6.68 (d, J=8.0 Hz, 1H), 6.70 (s, 1H), 6.65 (dd, J=8.0, 2.0 Hz, 1H), 6.12 (m, 2H), 4.97 (s, 2H), 3.72 (s, 3H), 3.56 (s, 3H), 3.30 (s, 3H), 3.17 (s, 3H).

### Method A

### Example 2

### N-(6-(3-(benzyloxy)phenoxy)-1,3-dimethyl-2-oxo-2,3-dihydro-1H-benzo[d]imidazol-5-yl)-3,5-dimethyl-1H-pyrazole-4-sulfonamide

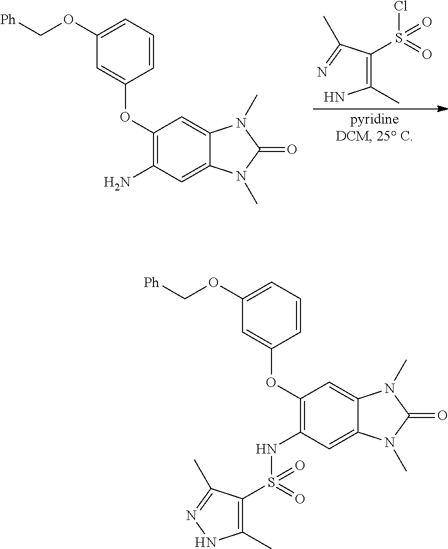

To a mixture of 5-amino-6-(3-(benzyloxy)phenoxy)-1,3-dimethyl-1H-benzo[d]imidazol-2(3H)-one (20 mg, 0.053 mmol) and 3,5-dimethyl-1H-pyrazole-4-sulfonyl chloride (16 mg, 0.080 mmol) in DCM (1 mL) was added pyridine (12 uL, 0.16 mmol). The mixture was stirred at 45° C. for 60 min, then quenched with methanol, concentrated and purified by prep-HPLC (mobile phase: A=0.1% TFA/H₂O, B=0.1% TFA/MeCN; gradient: B=40%-80% in 12 min; column: C18) to give the title compound as a solid (11 mg, 37%). MS (ES+) C₂₇H₂₇N₅O₅S requires: 533. found 534 [M+H]⁺. ¹H NMR (600 MHz, DMSO-d6) δ: 9.24 (s, 1H), 7.36 (m, 5H), 7.17 (t, J=8 Hz, 1H), 7.09 (s, 1H), 6.71 (d, J=8 Hz, 1H), 6.67 (s, 1H), 6.24 (m, 2H), 5.02 (s, 2H), 3.30 (s, 3H), 3.18 (s, 3H), 3.17 (s, 1H), 2.01 (s, 6H).

### Method B

### Example 3

### N-(6-(3-((6-chloropyridin-3-yl)methoxy)phenoxy)-1,3-dimethyl-2-oxo-2,3-dihydro-1H-benzo[d]imidazol-5-yl)-1-methyl-1H-imidazole-4-sulfonamide

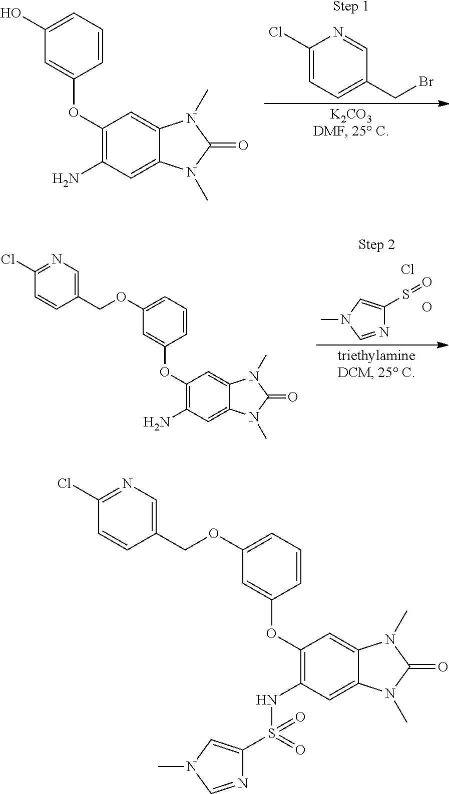

### Step 1: 5-amino-6-(3-((6-chloropyridin-3-yl)methoxy)phenoxy)-1,3-dimethyl-1H-benzo[d]imidazol-2(3H)-one

A mixture of potassium carbonate powder (48 mg, 0.35 mmol), 5-amino-6-(3-hydroxyphenoxy)-1,3-dimethyl-1H-benzo[d]imidazol-2(3H)-one (50 mg, 0.18 mmol, Intermediate 2) and 2-chloro-5-(chloromethyl)pyridine (142 mg, 0.88 mmol) in DMF (1 mL) was stirred for 30 mins. The reaction mixture was then quenched with methanol, concentrated and purified by column chromatography (20-100% EtOAc/hexanes then 0-40% MeOH/EtOAc) to give 5-amino-6-(3-((6-chloropyridin-3-yl)methoxy)phenoxy)-1,3-dimethyl-1H-benzo[d]imidazol-2(3H)-one as a solid (56 mg, 78%). MS (ES+) C₂₁H₁₉ClN₄O₃ requires: 410. found: 411 [M+H]⁺.

### Step 2: N-(6-(3-((6-chloropyridin-3-yl)methoxy)phenoxy)-1,3-dimethyl-2-oxo-2,3-dihydro-1H-benzo[d]imidazol-5-yl)-1-methyl-1H-imidazole-4-sulfonamide

A mixture of triethylamine (10 mg, 0.097 mmol), 4-methoxybenzene-1-sulfonyl chloride (15 mg, 0.073 mmol), and 5-amino-6-(3-((6-chloropyridin-3-yl)methoxy)phenoxy)-1,3-dimethyl-1H-benzo[d]imidazol-2(3H)-one (20 mg, 0.049 mmol) in DCM (1 mL) was stirred at room temperature for 30 mins. The reaction mixture was quenched with methanol, concentrated, then purified by prep-HPLC (mobile phase: A=0.1% TFA/H₂O, B=0.1% TFA/MeCN; gradient: B=40%-80% in 12 min; column: C18) to give the title compound as a solid (11 mg, 41%). MS (ES+) C₂₁H₁₉ClN₄O₃ requires: 555. found: 556 [M+H]⁺. ¹H NMR (600 MHz, CDCl₃) δ 8.36 (d, J=2.4 Hz, 1H), 8.00 (d, J=1.4 Hz, 1H), 7.87 (d, J=0.4 Hz, 1H), 7.73 (dd, J=8.5, 2.6 Hz, 1H), 7.41 (d, J=8.0 Hz, 1H), 7.31 (t, J=8.5 Hz, 1H), 6.84 (s, 1H), 6.80 (dd, J=8.5, 2.6, Hz, 1H), 6.75 (dd, J=8.5, 2.6, Hz, 1H), 6.59 (s, 1H), 6.57 (t, J=8.5 Hz, 1H), 5.3 (br-s, 1H), 4.36 (s, 2H), 3.67 (s, 3H), 3.23 (s, 3H), 3.18 (s, 3H).

### Method B

### Example 4

### N-(6-(3-(isopentyloxy)phenoxy)-1,3-dimethyl-2-oxo-2,3-dihydro-1H-benzo[d]imidazol-5-yl)-1,2-dimethyl-1H-imidazole-4-sulfonamide

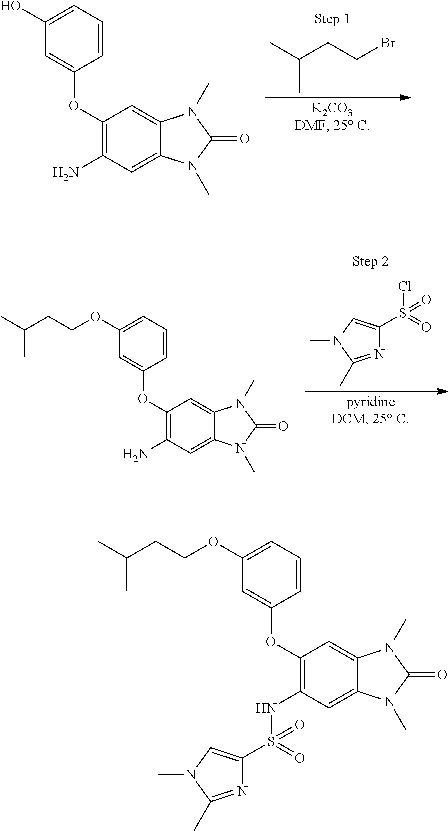

### Step 1: 5-amino-6-(3-(isopentyloxy)phenoxy)-1,3-dimethyl-1H-benzo[d]imidazol-2(3H)-one

To a solution of 5-amino-6-(3-hydroxyphenoxy)-1,3-dimethyl-1H-benzo[d]imidazol-2(3H)-one (50 mg, 0.18 mmol) in anhydrous DMF (1 ml) was added potassium carbonate (24 mg, 0.18 mmol) and 1-bromo-3-methylbutane (27 mg, 0.18 mmol). The reaction mixture was stirred at RT for 1 day, then the reaction mixture was diluted with water and extracted with EtOAc. The seperated organic layer was dried over sodium sulfate, filtered and concentrated to give the intermediate, 5-amino-6-(3-(isopentyloxy)phenoxy)-1,3-dimethyl-1H-benzo[d]imidazol-2(3H)-one, as a crude residue and carried onto the next step without further purification. MS (ES+) C₂₀H₂₅N₃O₃ requires: 355. found 356 [M+H]⁺.

### Step 2: N-(6-(3-(isopentyloxy)phenoxy)-1,3-dimethyl-2-oxo-2,3-dihydro-1H-benzo[d]imidazol-5-yl)-1,2-dimethyl-1H-imidazole-4-sulfonamide

To a solution of 5-amino-6-(3-(isopentyloxy)phenoxy)-1,3-dimethyl-1H-benzo[d]imidazol-2(3H)-one (62 mg, 0.18 mmol) and pyridine (28 uL, 0.35 mmol) in anhydrous DCM (1 ml) was added 1,2-dimethyl-1H-imidazole-4-sulfonyl chloride (34 mg, 0.18 mmol) in a vial. The reaction mixture was stirred for 2 days at RT, then quenched with methanol, concentrated and purified by prep-HPLC to give the title product as a solid (5 mg, 6% yield over 2-steps). MS (ES+) C₂₅H₃₁N₅O₅S requires: 513. found 514 [M+H]⁺. ¹H NMR (600 MHz d₆-DMSO) δ: 9.32 (s, 1H), 7.50 (s, 1H), 7.18-7.15 (m, 2H), 6.78 (s, 1H), 6.65-6.63 (m, 1H), 6.26-6.25 (m, 2H), 3.96-3.94 (t, J=6.6 Hz, 2H), 3.46 (s, 3H), 3.30 (s, 3H), 3.21 (s, 3H), 2.10 (s, 3H), 1.79-1.72 (m, 1H), 1.61-1.57 (q, J=6.7 Hz, 2H), 0.92 (s, 3H), 0.91 (s, 3H).

### Method B

### Example 5

### N-(6-(3-(4-aminobutoxy)phenoxy)-1,3-dimethyl-2-oxo-2,3-dihydro-1H-benzo[d]imidazol-5-yl)-1-methyl-1H-imidazole-4-sulfonamide

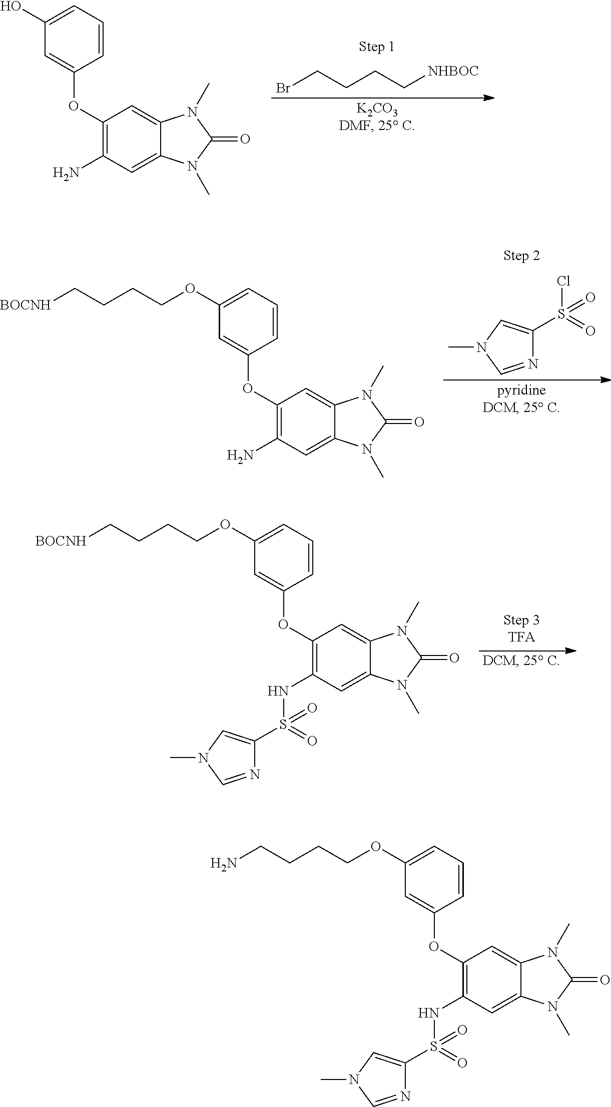

### Step 1: tert-butyl (4-(3-((6-amino-1,3-dimethyl-2-oxo-2,3-dihydro-1H-benzo[d]imidazol-5-yl)oxy)phenoxy)butyl)carbamate

To a solution of 5-amino-6-(3-hydroxyphenoxy)-1,3-dimethyl-1H-benzo[d]imidazol-2(3H)-one (51 mg, 0.18 mmol) in anhydrous DMF (1 ml) were added potassium carbonate (25 mg, 0.18 mmol) and tert-butyl(4-bromobutyl)carbamate (45.1 mg, 0.179 mmol). The reaction mixture was stirred at RT for 3 days. The reaction mixture was diluted with water, then extracted with 3 portions of EtOAc. The combined organic layers were concentrated to give tert-butyl (4-(3-((6-amino-1,3-dimethyl-2-oxo-2,3-dihydro-1H-benzo[d]imidazol-5-yl)oxy)phenoxy)butyl)carbamate, which was used directly in the next step without further purification. MS (ES+) C₂₄H₃₂N₄O₅ requires: 456. found 457 [M+H]⁺.

### Step 2: tert-butyl (4-(3-((1,3-dimethyl-6-(1-methyl-1H-imidazole-5-sulfonamido)-2-oxo-2,3-dihydro-1H-benzo[d]imidazol-5-yl)oxy)phenoxy)butyl)carbamate

To a solution of tert-butyl (4-(3-((6-amino-,3-dimethyl-2-oxo-2,3-dihydro-1H-benzo[d]imidazol-5-yl)oxy)phenoxy)butyl)carbamate (82 mg, 0.18 mmol) in anhydrous DCM (1 ml) was added pyridine (29 uL, 0.36 mmol), and 1-methyl-1H-imidazole-4-sulfonyl chloride (32 mg, 0.18 mmol) in a vial. The reaction mixture was stirred at RT for 16 h, then concentrated and purified by prep-HPLC to give tert-butyl (4-(3-((1,3-dimethyl-6-(1-methyl-1H-imidazole-5-sulfonamido)-2-oxo-2,3-dihydro-1H-benzo[d]imidazol-5-yl)oxy)phenoxy)butyl)carbamate. MS (ES+) C₂₈H₃₆N₆O₇S requires: 600. found 601 [M+H]⁺.

### Step 3: N-(6-(3-(4-aminobutoxy)phenoxy)-1,3-dimethyl-2-oxo-2,3-dihydro-1H-benzo[d]imidazol-5-yl)-1-methyl-1H-imidazole-4-sulfonamide 2,2,2-trilfluoroacetate

To a solution of tert-butyl (4-(3-((1,3-dimethyl-6-(1-methyl-1H-imidazole-4-sulfonamido)-2-oxo-2,3-dihydro-1H-benzo[d]imidazol-5-yl)oxy)phenoxy)butyl)carbamate (100 mg, 0.166 mmol) in DCM (1 ml) was added 2,2,2-trifluoroacetic acid (0.019 ml, 0.25 mmol) and the resulting mixture was stirred at 25° C. for 2 h. The reaction mixture was quenched with methanol, filtered and purified prep-HPLC to give N-(6-(3-(4-aminobutoxy)phenoxy)-1,3-dimethyl-2-oxo-2,3-dihydro-1H-benzo[d]imidazol-5-yl)-1-methyl-1H-imidazole-4-sulfonamide 2,2,2-trilfluoroacetate (26.2 mg, 32% yield). MS (ES+) C₂₃H₂₈N₆O₅S requires: 500. found 501 [M+H]⁺. ¹H NMR (600 MHz d₆-DMSO) δ: 9.31 (s, 1H), 7.68 (br-s, 1H), 7.61 (s, 1H), 7.59 (s, 1H), 7.19-7.16 (t, J=8.3 Hz, 1H), 7.08 (s, 1H), 6.78 (s, 1H), 6.63-6.61 (d, J=8.2 Hz, 1H), 6.31-6.30 (m, 2H), 3.94-3.92 (t, J=6.1 Hz, 2H), 3.60 (s, 3H), 3.37 (s, 3H), 3.21 (s, 3H), 2.88-2.82 (m, 2H), 1.77-1.72 (m, 2H), 1.70-1.65 (m, 2H).

### Method C

### Example 6

### N-(6-(3-ethoxyphenoxy)-1,3-dimethyl-2-oxo-2,3-dihydro-1H-benzo[d]imidazol-5-yl)-1,2-dimethyl-1H-imidazole-4-sulfonamide

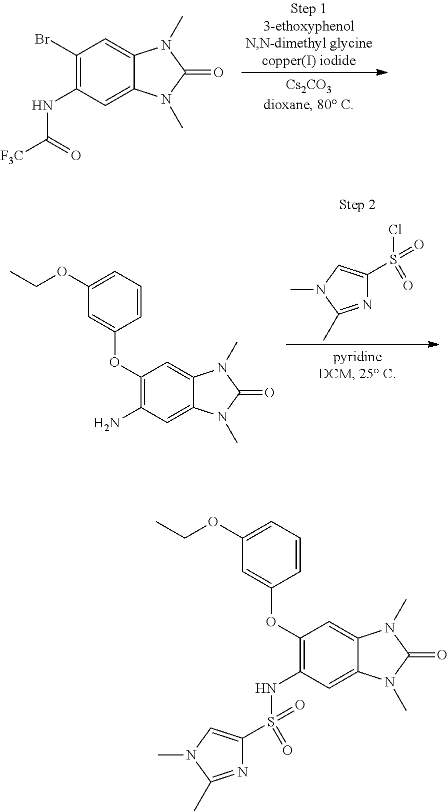 [SMILES: Cn1c(=O)n(C)c2cc(NC(=O)C(F)(F)F)c(Br)cc21 ; CCOc1cccc(Oc2cc3c(cc2N)n(C)c(=O)n3C)c1 ; Cc1nc(S(=O)(=O)Cl)cn1C ; CCOc1cccc(Oc2cc3c(cc2NS(=O)(=O)c2cn(C)c(C)n2)n(C)c(=O)n3C)c1]

### Step 1: 5-amino-6-(3-ethoxyphenoxy)-1,3-dimethyl-1H-benzo[d]imidazol-2(3H)-one

A mixture of N-(6-bromo-1,3-dimethyl-2-oxo-2,3-dihydro-1H-benzo[d]imidazol-5-yl)-2,2,2-trifluoroacetamide (50 mg, 0.14 mmol), 2-(dimethylamino)acetic acid (15 mg, 0.14 mmol), 3-ethoxyphenol (29 mg, 0.21 mmol), copper(I) iodide (8 mg, 0.043 mmol) and cesium carbonate (139 mg, 0.43 mmol) were charged in a flask with dioxane (1 ml). The reaction mixture was heated to 80° C. and stirred for 16 h. The reaction was monitored for complete de-protection of trifluoroacetamide (addition of methanol can be used to facilitate this step). The cooled reaction mixture was diluted in methanol, filtered and the collected filtrate was concentrated and purified by column chromatography (EtOAc/hexanes 1:1) to give 5-amino-6-(3-ethoxyphenoxy)-1,3-dimethyl-1H-benzo[d]imidazol-2(3H)-one as an orange solid (13 mg, 29%). MS (ES+) C₁₇H₁₉N₃O₃ requires: 313. found 314 [M+H]⁺.

### Step 2: N-(6-(3-ethoxyphenoxy)-1,3-dimethyl-2-oxo-2,3-dihydro-1H-benzo[d]imidazol-5-yl)-1,2-dimethyl-1H-imidazole-4-sulfonamide

A solution of 5-amino-6-(3-ethoxyphenoxy)-1,3-dimethyl-1H-benzo[d]imidazol-2(3H)-one (13 mg, 0.041 mmol) in DCM (0.5 ml) was treated with pyridine (7 μl, 0.08 mmol) and 1,2-dimethyl-1H-imidazole-4-sulfonyl chloride (12 mg, 0.062 mmol). The reaction mixture was stirred at ambient temperature for 1 day, then concentrated and purified by prep-HPLC to give the title compound as a solid (7 mg, 36%). MS (ES+) C₂₂H₂₅N₅O₅S requires: 471. found 472 [M+H]⁺. ¹H NMR (d₆-DMSO) δ 9.34 (br. s., 1H), 7.50 (s, 1H), 7.18-7.13 (m, 2H), 6.76 (1H, s), 6.61 (dd, J=8.2, 2.2 Hz, 1H), 6.26 (dd, J=8.2, 2.2 Hz, 1H), 6.20 (t, J=2.2 Hz, 1H), 3.96 (q, J=6.9 Hz, 2H), 3.45 (s, 3H), 3.29 (s, 3H), 3.20 (s, 3H), 2.09 (s, 3H), 1.30 (t, J=6.9 Hz, 3H).

### Method D

### Example 7

### N-(6-(3-(4-(N-(6-(3-((6-aminohexyl)oxy)-5-propoxyphenoxy)-1,3-dimethyl-2-oxo-2,3-dihydro-1H-benzo[d]imidazol-5-yl)-3,4-dimethoxybenzenesulfonamide 2,2,2-trifluoroacetate

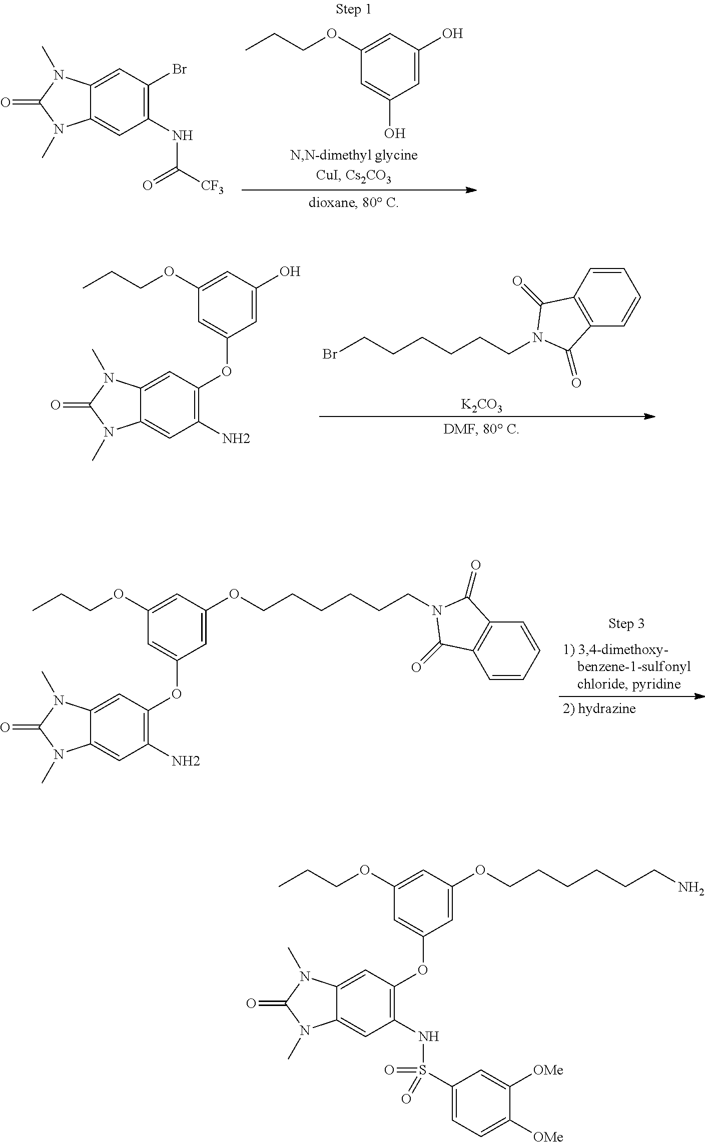

### Step 1: 5-amino-6-(3-hydroxy-5-propoxyphenoxy)-1,3-dimethyl-1H-benzo[d]imidazol-2(3H)-one

A flask was charged with N-(6-bromo-1,3-dimethyl-2-oxo-2,3-dihydro-1H-benzo[d]imidazol-5-yl)-2,2,2-trifluoroacetamide (2.44 g, 6.94 mmol), 2-(dimethylamino)acetic acid (0.715 g, 6.94 mmol), copper(I) iodide (0.396 g, 2.08 mmol), 5-propoxybenzene-1,3-diol (1.4 g, 8.3 mmol), cesium carbonate (6.78 g, 20.8 mmol) and dioxanes (30 ml). The reaction mixture was stirred at 80° C. for 18 h, then methanol (30 ml) was added and the mixture was stirred at 80° C. for an additional 4 h. The cooled reaction mixture was then concentrated under reduced pressure then the residue was dissolved in DCM with 5% MeOH and filtered through a pad of celite. The collected filtrate was concentrated, added saturated aq. NH₄Cl (50 mL) and the organic layer was separated. The aqueous phase was extracted with EtOAc (3×50 mL) and the combined organic layers were washed with sat NH₄Cl and concentrated under reduced pressure. The residue was purified by silica gel chromatography (0:100 to 40:60, MeOH/DCM) to give 5-amino-6-(3-hydroxy-5-propoxyphenoxy)-1,3-dimethyl-1H-benzo[d]imidazol-2(3H)-one (790 mg, 43%) as a thick dark-brown solid. MS (ES+) C₁₈H₂₁N₃O₄ requires: 343. found 344 [M+H]⁺.

### Step 2: 2-(6-(3-((6-amino-1,3-dimethyl-2-oxo-2,3-dihydro-1H-benzo[d]imidazol-5-yl)oxy)-5-propoxyphenoxy)hexyl)isoindoline-1,3-dione

To a solution of 5-amino-6-(3-hydroxy-5-propoxyphenoxy)-1,3-dimethyl-1H-benzo[d]imidazol-2(3H)-one (800 mg, 2.33 mmol) in anhydrous DMF (60 ml) was added potassium carbonate (1.29 g, 9.32 mmol) and 2-(6-bromohexyl)isoindoline-1,3-dione (2.89 g, 9.32 mmol) in a vial. The reaction mixture was stirred at 80° C. for 1 hr. To the cooled reaction mixture was added water (50 mL), and the layers were separated. The aqueous phase was extracted with EtOAc (3×50 mL), the combined organic layers were concentrated under reduced pressure and the residue was purified by silica gel chromatography (20:80 to 100:0, EtOAc/hexanes) to give 2-(6-(3-((6-amino-1,3-dimethyl-2-oxo-2,3-dihydro-1H-benzo[d]imidazol-5-yl)oxy)-5-propoxyphenoxy)hexyl)isoindoline-1,3-dione as a thick brown liquid (800 mg, 60%). MS (ES+) C₃₂H₃₆N₄O₆ requires: 572 found: 573 [M+H]⁺.

### Step 3: N-(6-(3-((6-aminohexyl)oxy)-5-propoxyphenoxy)-1,3-dimethyl-2-oxo-2,3-dihydro-1H-benzo[d]imidazol-5-yl)-3,4-dimethoxybenzenesulfonamide

To a vial containing a solution of 2-(6-(3-((6-amino-1,3-dimethyl-2-oxo-2,3-dihydro-1H-benzo[d]imidazol-5-yl)oxy)-5-propoxyphenoxy)hexyl)isoindoline-1,3-dione (150 mg, 0.26 mmol) in anhydrous DCM (1 ml), was added pyridine (0.042 ml, 0.52 mmol) and 3,4-dimethoxybenzene-1-sulfonyl chloride (74.4 mg, 0.314 mmol). The reaction mixture was stirred at RT for 1 h, then added methanol and hydrazine (8 μl, 0.26 mmol). The reaction was monitored for completion, then the mixture was concentrated and purified by mass-triggered preparative HPLC (Mobile phase: A=0.1% TFA/H₂O, B=0.1% TFA/MeCN; Gradient: B=20-60%; 12 min; Column: C18) to give N-(6-(3-((6-aminohexyl)oxy)-5-propoxyphenoxy)-1,3-dimethyl-2-oxo-2,3-dihydro-1H-benzo[d]imidazol-5-yl)-3,4-dimethoxybenzenesulfonamide 2,2,2-trifluoroacetate (50 mg, 30%) as a white solid. MS (ES+) C₃₂H₄₂N₄O₈S requires: 642. found: 643 [M+H]⁺. ¹H NMR (600 MHz, CDCl₃) δ: 9.27 (s, 1H), 7.62 (s, 1H), 7.60 (s, 1H), 7.59 (s, 2H), 7.06 (s, 1H), 6.82 (s, 1H), 6.17 (t, J=2 Hz, 1H), 5.86 (d, J=2 Hz, 2H), 3.88 (t, J=7 Hz, 2H), 3.83 (t, J=6 Hz, 2H), 3.60 (s, 3H), 3.26 (s, 3H), 3.22 (s, 3H), 2.78 (m, 2H), 1.70 (m, 4H), 1.36 (m, 4H), 1.54 (m, 2H), 0.94 (t, J=7 Hz, 3H).

### Method E

### Example 8

### N-(6-(3-((6-aminohexyl)oxy)-5-propoxyphenoxy)-1,3-dimethyl-2-oxo-2,3-dihydro-1H-benzo[d]imidazol-5-yl)-1-methyl-1H-imidazole-4-sulfonamide 2,2,2-trifluoroacetate

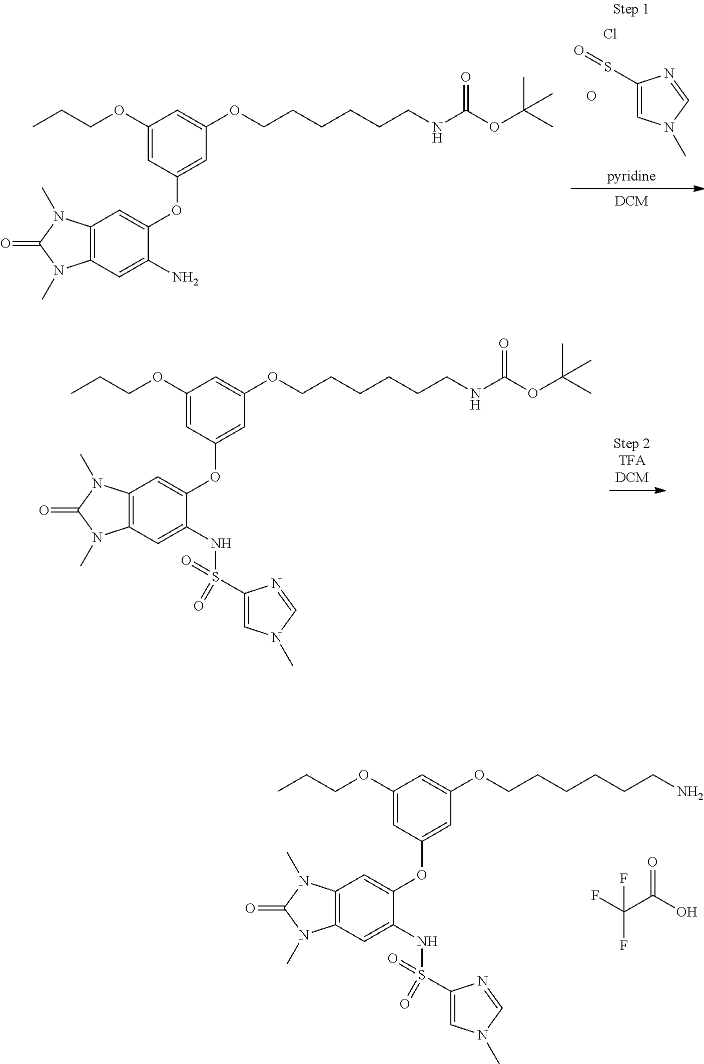

### Step 1: tert-Butyl (6-(3-((1,3-dimethyl-6-(1-methyl-1H-imidazole-4-sulfonamido)-2-oxo-2,3-dihydro-1H-benzo[d]imidazol-5-yl)oxy)-5-propoxyphenoxy)hexyl)carbamate

To a solution of tert-butyl (6-(3-((6-amino-,3-dimethyl-2-oxo-2,3-dihydro-1H-benzo[d]imidazol-5-yl)oxy)-5-propoxyphenoxy)hexyl)carbamate (67 mg, 0.12 mmol) in DCM (2 ml) were added 1-methyl-1H-imidazole-4-sulfonyl chloride (46 mg, 0.26 mmol) and pyridine (100 μL, 1.24 mmol), the sulfonyl chloride was not fully soluble, so dioxanes (2 mL) was added and the resulting mixture was stirred at 60° C. for 21 h. The resulting suspension was concentrated, then purified by silica-gel chromatography (1:5-1:1 EtOAc/hexanes then 100% EtOAc) and dried to give tert-butyl (6-(3-((1,3-dimethyl-6-(1-methyl-1H-imidazole-4-sulfonamido)-2-oxo-2,3-dihydro-1H-benzo[d]imidazol-5-yl)oxy)-5-propoxyphenoxy)hexyl)carbamate as a red-colored amorphous solid (58 mg, 68%). MS (ES+) C₃₃H₄₆N₆O₈S requires: 686. found 687 [M+H]⁺. ¹H NMR (600 MHz, CDCl₃) δ: 7.97 (br-s, 1H), 7.60 (s, 1H, overlapped with CHCl₃), 7.35 (s, 1H), 7.31 (s, 1H), 7.20 (s, 1H), 6.57 (s, 1H), 6.14 (s, 1H), 5.77 (br-s, 1H), 5.72 (br-s, 1H), 3.86 (t, J=6.4 Hz, 2H), 3.83 (t, J=6.5 Hz, 2H), 3.62 (s, 3H), 3.45 (s, 3H), 3.31 (s, 3H), 3.12 (br-t, J=6.3 Hz, 2H), 1.76 (m, 4H), 1.51 (m, 2H), 1.46 (m, 2H), 1.44 (s, 9H), 1.37 (m, 2H), 1.01 (t, J=7.3 Hz, 3H).

### Step 2: N-(6-(3-((6-aminohexyl)oxy)-5-propoxyphenoxy)-1,3-dimethyl-2-oxo-2,3-dihydro-1H-benzo[d]imidazol-5-yl)-1-methyl-1H-imidazole-4-sulfonamide 2,2,2-trifluoroacetate

To a solution of tert-butyl (6-(3-((1,3-dimethyl-6-(1-methyl-1H-imidazole-4-sulfonamido)-2-oxo-2,3-dihydro-1H-benzo[d]imidazol-5-yl)oxy)-5-propoxyphenoxy)hexyl)carbamate (18 mg, 0.026 mmol) in DCM (4 ml) were added TFA (500 μl, 6.49 mmol) and the resulting mixture was stirred at 25° C. for 16 h. The reaction mixture was concentrated to give the title compound as a brown-colored viscous liquid (17 mg, 93%). MS (ES+) C₂₈H₃₈N₆O₆S requires: 586. found 587 [M+H]⁺. ¹H NMR (d₆-DMSO) δ: 8.52 (br-s, 1H), 7.94 (br-s, 3H), 7.48 (br-s, 1H), 7.24 (s, 1H), 7.18 (s, 1H), 6.56 (s, 1H), 6.11 (s, 1H), 5.85 (s, 1H), 5.56 (s, 1H), 3.89 (t, J=6.1 Hz, 2H), 3.79 (t, J=6.6 Hz, 2H), 3.61 (s, 3H), 3.41 (s, 3H), 3.28 (s, 3H), 2.97 (br-s, 2H), 1.77-1.65 (m, 6H), 1.46 (m, 2H), 1.42 (m, 2H), 0.98 (t, J=7.3 Hz, 3H).

### Method F

### Example 9

### N-(6-(3-(4-(dimethylamino)butoxy)phenoxy)-1,3-dimethyl-2-oxo-2,3-dihydro-1H-benzo[d]imidazol-5-yl)-1-methyl-1H-imidazole-4-sulfonamide 2,2,2-trifluoroacetate

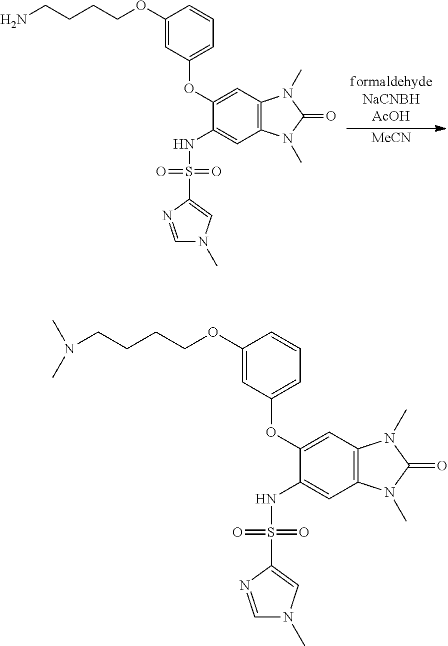 [SMILES: "MeCN" ; Cn1cnc(S(=O)(=O)Nc2cc3c(cc2Oc2cccc(OCCCCN)c2)n(C)c(=O)n3C)c1 ; CN(C)CCCCOc1cccc(Oc2cc3c(cc2NS(=O)(=O)c2cn(C)cn2)n(C)c(=O)n3C)c1]

To a solution of N-(6-(3-(4-aminobutoxy)phenoxy)-1,3-dimethyl-2-oxo-2,3-dihydro-1H-benzo[d]imidazol-5-yl)-1-methyl-1H-imidazole-4-sulfonamide (10 mg, 0.020 mmol) in acetonitrile (1 ml) was added formaldehyde (1.7 μl, 0.06 mmol) and stirred for 30 min at 25° C. The reaction mixture was then treated with sodium cyanoborohydride (1.9 mg, 0.030 mmol), a drop of acetic acid and then stirred at 25° C. for another 30 min. The reaction mixture was then quenced with methanol and concentrated under reduced pressure. The residue was purified by prep-HPLC to obtain the title compound (4.1 mg, 39%). MS (ES+) C₂₅H₃₂N₆O₅S requires 528. found: 529 [M+H]⁺. ¹H NMR (600 MHz, d₆-DMSO) δ: 9.31 (s, 1H), 9.22 (br-s, 1H), 7.62 (s, 1H), 7.59 (d, J=1.2 Hz, 1H), 7.18 (m, 1H), 7.08 (s, 1H), 6.78 (s, 1H), 6.63 (dd, J=8.2, 2.1 Hz, 1H), 6.31 (m, 2H), 3.95 (t, J=5.6 Hz, 2H), 3.61 (s, 3H), 3.27 (s, 3H), 3.21 (s, 3H), 3.11-3.09 (m, 2H), 2.78 (s, 3H), 2.77 (s, 3H), 1.78-1.70 (m, 4H).

### Method F

### Example 10

### N-(6-(3-(4-(dimethylamino)butoxy)-5-propoxyphenoxy)-1,3-dimethyl-2-oxo-2,3-dihydro-1H-benzo[d]imidazol-5-yl)-3,4-dimethoxybenzenesulfonamide 2,2,2-trifluoroacetate

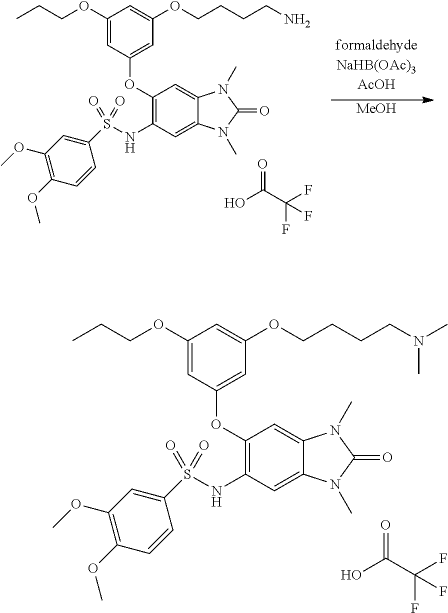

To a solution of N-(6-(3-(4-aminobutoxy)-5-propoxyphenoxy)-1,3-dimethyl-2-oxo-2,3-dihydro-1H-benzo[d]imidazol-5-yl)-3,4-dimethoxybenzenesulfonamide 2,2,2-trifluoroacetate (180 mg, 0.247 mmol) in methanol (3.0 ml) was added triethylamine (0.034 ml, 0.25 mmol), acetic acid (0.028 ml, 0.49 mmol), formaldehyde (0.054 ml, 2.0 mmol), and sodium triacetoxyborohydride (131 mg, 0.618 mmol). The reaction mixture was stirred at room temperature and checked by LCMS every 30 minutes. After 3 h the reaction was complete by LCMS. The reaction was quenched with a few drops of TFA and concentrated under reduced pressure. The residue was purified by prep-HPLC using a gradient of 20-60% ACN/water containing 0.1% TFA to afford N-(6-(3-(4-(dimethylamino)butoxy)-5-propoxyphenoxy)-1,3-dimethyl-2-oxo-2,3-dihydro-1H-benzo[d]imidazol-5-yl)-3,4-dimethoxybenzenesulfonamide 2,2,2-trifluoroacetate (106 mg, 57%) as a white solid. MS (ES+) C₃₂H₄₂N₄O₈S requires: 642. found 643 [M+H]⁺. ¹H NMR (600 MHz, DMSO-d₆) δ 9.46 (s, 1H), 9.30 (br-s, 1H), 7.19 (m, 2H), 7.07 (s, 1H), 6.90 (d, J=9.0 Hz, 1H), 6.75 (s, 1H), 6.13 (t, J=2.2 Hz, 1H), 5.71 (t, J=2.0 Hz, 1H), 5.67 (t, J=2.0 Hz, 1H), 3.84 (t, J=5.9 Hz, 2H), 3.77 (m, 5H), 3.62 (s, 3H), 3.29 (s, 3H), 3.20 (s, 3H), 3.12-3.05 (m, 2H), 2.78 (d, J=4.7 Hz, 6H), 1.77-1.63 (m, 6H), 0.95 (t, J=7.3 Hz, 3H).The following examples were prepared using the general methods A through F as previously described:

TABLE 1
Synthesized Examples

| | | | | [M + | |
|---|---|---|---|---|---|
| No. | Structure | Name | MW | H]⁺ | Method |
| 1 | 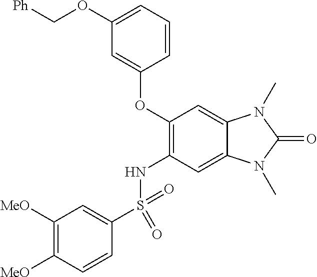 [SMILES: COc1ccc(S(=O)(=O)Nc2cc3c(cc2Oc2cccc(OCc4ccccc4)c2)n(C)c(=O)n3C)cc1OC] | N-{6-[3- (benzyloxy) phenoxy]-1,3-dimethyl- 2-oxo-2,3- dihydro-1H-1,3- benzodiazol-5- yl}-3,4- dimethoxybenzene- 1-sulfonamide | 576 | 577 | A, see text for detailed descrip- tion |
| 2 | 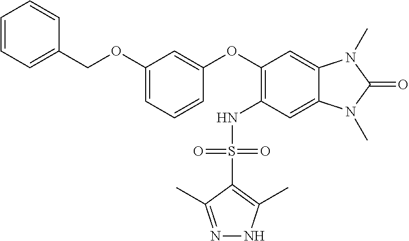 [SMILES: Cc1n[nH]c(C)c1S(=O)(=O)Nc1cc2c(cc1Oc1cccc(OCc3ccccc3)c1)n(C)c(=O)n2C] | N-{6-[3- (benzyloxy) phenoxy]-1,3-dimethyl- 2-oxo-2,3- dihydro-1H-1,3- benzodiazol-5- yl}-3,5-dimethyl-0 1H-pyrazole-4- sulfonamide | 533 | 534 | A, see text for detailed descrip- tion |
| 3 | 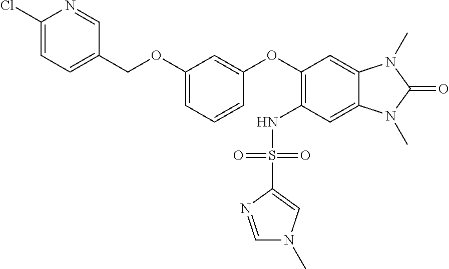 [SMILES: Cn1cnc(S(=O)(=O)Nc2cc3c(cc2Oc2cccc(OCc4ccc(Cl)nc4)c2)n(C)c(=O)n3C)c1] | N-(6-(3-((6- chloropyridin-3- yl)methoxy) phenoxy)-1,3- dimethyl-2-oxo- 2,3-dihydro-1H- benzo[d]imidazol- 5-yl)-1-methyl- 1H-imidazole-4- sulfonamide | 555 | 556 | B, see text for detailed descrip- tion |
| 4 | 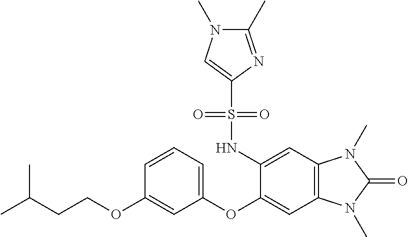 [SMILES: Cc1nc(S(=O)(=O)Nc2cc3c(cc2Oc2cccc(OCCC(C)C)c2)n(C)c(=O)n3C)cn1C] | N-(6-(3- (isopentyloxy) phenoxy)-1,3- dimethyl-2-oxo- 2,3-dihydro-1H- benzo[d]imidazol- 5-yl)-1,2- dimethyl-1H- imidazole-4- sulfonamide | 513 | 514 | B, see text for detailed descrip- tion |
| 5 | 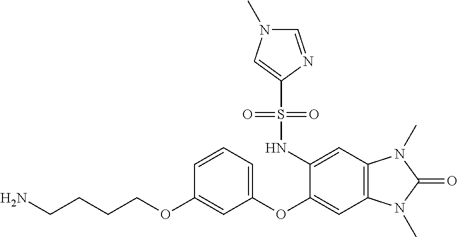 [SMILES: Cn1cnc(S(=O)(=O)Nc2cc3c(cc2Oc2cccc(OCCCCN)c2)n(C)c(=O)n3C)c1] | N-(6-(3-(4- aminobutoxy) phenoxy)-1,3- dimethyl-2-oxo- 2,3-dihydro-1H- benzo[d]imidazol- 5-yl)-1-methyl- 1H-imidazole-4- sulfonamide | 500 | 501 | B, see text for detailed descrip- tion |
| 6 | 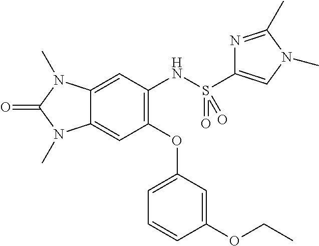 [SMILES: CCOc1cccc(Oc2cc3c(cc2NS(=O)(=O)c2cn(C)c(C)n2)n(C)c(=O)n3C)c1] | N-[6-(3- ethoxyphenoxy)- 1,3-dimethyl-2- oxo-2,3-dihydro- 1H-1,30 benzodiazol-5- yl]-1,2-dimethyl- 1H-imidazole-4- sulfonamide | 471 | 472 | C, see text for detailed descrip- tion |
| 7 | 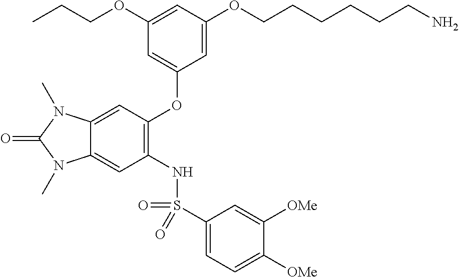 [SMILES: CCCOc1cc(OCCCCCCN)cc(Oc2cc3c(cc2NS(=O)(=O)c2ccc(OC)c(OC)c2)n(C)c(=O)n3C)c1] | N-(6-{3-[(6- aminohexyl)oxy]- 5- propoxyphenoxy}- 1,3-dimethyl-2- oxo-2,3-dihydro- 1H-1,3- benzodiazol-5- yl)-3,4- dimethoxybenzene- 1-sulfonamide | 642 | 643 | D, see text for detailed descrip- tion |
| 8 | 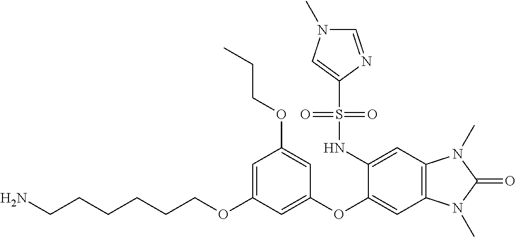 [SMILES: CCCOc1cc(OCCCCCCN)cc(Oc2cc3c(cc2NS(=O)(=O)c2cn(C)cn2)n(C)c(=O)n3C)c1] | N-(6-(3-((6- aminohexyl)oxy)- 5- propoxyphenoxy)- 1,3-dimethyl-2- oxo-2,3-dihydro- 1H- benzo[d]imidazol- 5-yl)-1-methyl- 1H-imidazole-4- sulfonamide | 586 | 587 | E, see text for detailed descrip- tion |
| 9 | 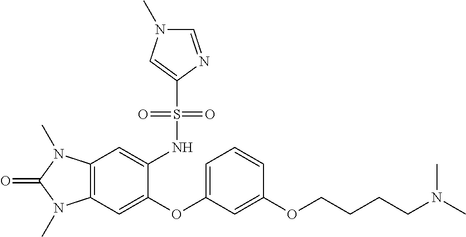 [SMILES: CN(C)CCCCOc1cccc(Oc2cc3c(cc2NS(=O)(=O)c2cn(C)cn2)n(C)c(=O)n3C)c1] | N-(6-(3-(4- (dimethylamino) butoxy)phenoxy)- 1,3-dimethyl-2- oxo-2,3-dihydro- 1H- benzo[d]imidazol- 5-yl)-1-methyl- 1H-imidazole-4- sulfonamide | 528 | 529 | F, see text for detailed descrip- tion |
| 10 | 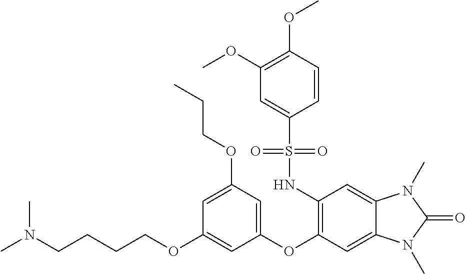 [SMILES: CCCOc1cc(OCCCCN(C)C)cc(Oc2cc3c(cc2NS(=O)(=O)c2ccc(OC)c(OC)c2)n(C)c(=O)n3C)c1] | N-(6-(3-(4- (dimethylamino) butoxy)-5- propoxyphenoxy)- 1,3-dimethyl-2- oxo-2,3-dihydro- 1H- benzo[d]imidazol- 5-yl)-3,4- dimethoxybenzene- sulfonamide | 642 | 643 | F, see text for detailed descrip- tion |
| 11 | 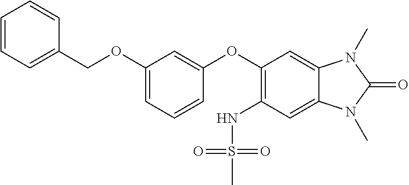 [SMILES: Cn1c(=O)n(C)c2cc(Oc3cccc(OCc4ccccc4)c3)c(NS(C)(=O)=O)cc21] | N-(6-3- (benzyloxy) phenoxy)-1,3-dimethyl- 2-oxo-2,3- dihydro-1H- benzo[d]imidazol- 5- yl)methanesulfonamide | 453 | 454 | A |
| 12 | 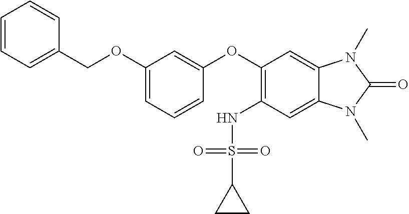 [SMILES: Cn1c(=O)n(C)c2cc(Oc3cccc(OCc4ccccc4)c3)c(NS(=O)(=O)C3CC3)cc21] | N-(6-(3- (benzyloxy) phenoxy)-1,3-dimethyl- 2-oxo-2,3- dihydro-1H- benzo[d]imidazol- 5- yl)cyclopropane- sulfonamide | 479 | 480 | A |
| 13 | 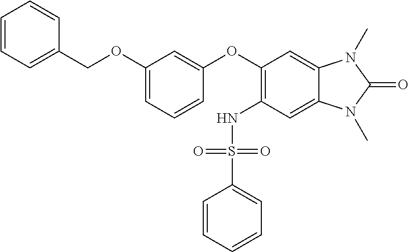 [SMILES: Cn1c(=O)n(C)c2cc(Oc3cccc(OCc4ccccc4)c3)c(NS(=O)(=O)c3ccccc3)cc21] | N-(6-(3- (benzyloxy) phenoxy)-1,3-dimethyl- 2-oxo-2,3- dihydro-1H- benzo[d]imidazol- 5- yl)benzenesulfonamide | 515 | 516 | A |
| 14 | 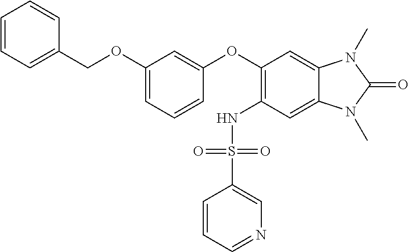 [SMILES: Cn1c(=O)n(C)c2cc(Oc3cccc(OCc4ccccc4)c3)c(NS(=O)(=O)c3cccnc3)cc21] | N-(6-(3- (benzyloxy) phenoxy)-1,3-dimethyl- 2-oxo-2,3- dihydro-1H- benzo[d]imidazol- 5-yl)pyridine-3- sulfonamide | 516 | 517 | A |
| 15 | 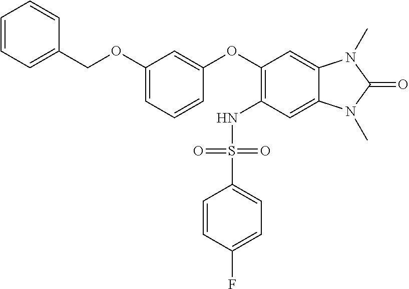 [SMILES: Cn1c(=O)n(C)c2cc(Oc3cccc(OCc4ccccc4)c3)c(NS(=O)(=O)c3ccc(F)cc3)cc21] | N-(6-(3- (benzyloxy) p[henoxy)-1,3-dimethyl- 2-oxo-2,3- dihydro-1H- benzo[d]imidazol- 5-yl)-4- fluorobenzene- sulfonamide | 533 | 534 | A |
| 16 | 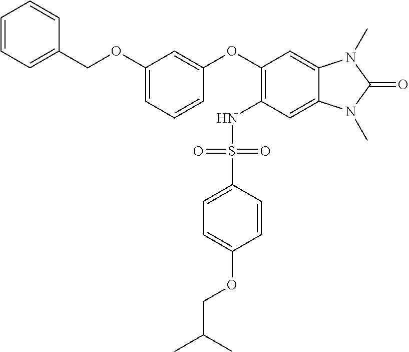 [SMILES: CC(C)COc1ccc(S(=O)(=O)Nc2cc3c(cc2Oc2cccc(OCc4ccccc4)c2)n(C)c(=O)n3C)cc1] | N-(6-(3- (benzyloxy) phenoxy)-1,3-dimethyl- 2-oxo-2,3- dihydro-1H- benzo[d]imidazol- 5-yl)-4- isobutoxybenzene- sulfonamide | 587 | 588 | A |
| 17 | 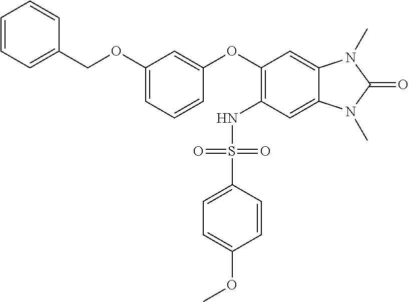 [SMILES: COc1ccc(S(=O)(=O)Nc2cc3c(cc2Oc2cccc(OCc4ccccc4)c2)n(C)c(=O)n3C)cc1] | N-(6-(3- (benzyloxy) phenoxy)-1,3-dimethyl- 2-oxo-2,3- dihydro-1H- benzo[d]imidazol- 5-yl)-4- methoxybenzene- sulfonamide | 545 | 546 | A |
| 18 | 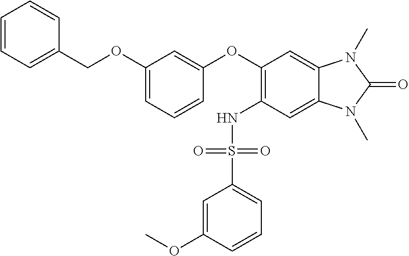 [SMILES: COc1cccc(S(=O)(=O)Nc2cc3c(cc2Oc2cccc(OCc4ccccc4)c2)n(C)c(=O)n3C)c1] | N-(6-(3- (benzyloxy) phenoxy)-1,3-dimethyl- 2-oxo-2,3- ddihydro-1H- benzo[d]imidazol- 5-yl)-3- methoxybenzene- sulfonamide | 545 | 546 | A |
| 19 | 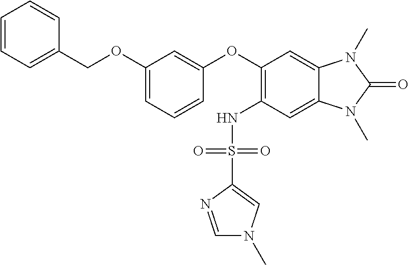 [SMILES: Cn1cnc(S(=O)(=O)Nc2cc3c(cc2Oc2cccc(OCc4ccccc4)c2)n(C)c(=O)n3C)c1] | N-(6-(3- (benzyloxy) phenoxy)-1,3-dimethyl- 2-oxo-2,3- dihydro-1H- benzo[d]imidazol- 5-yl)-1-methyl- 1H-imidazole-4- sulfonamide | 519 | 520 | A |
| 20 | 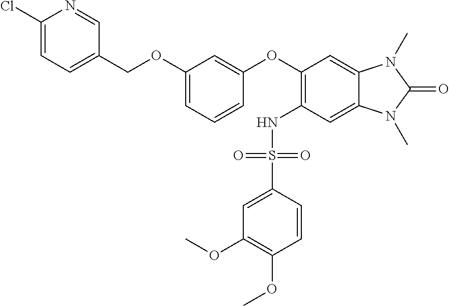 [SMILES: COc1ccc(S(=O)(=O)Nc2cc3c(cc2Oc2cccc(OCc4ccc(Cl)nc4)c2)n(C)c(=O)n3C)cc1OC] | N-(6-(3-((6- chloropyridin-3- yl)methoxy) phenoxy)-1,3- dimethyl-2-oxo- 2,3-dihydro-1H- benzo[d]imidazol- 5-yl)-3,4- dimethoxybenzene- sulfonamide | 610 | 611 | B |
| 21 | 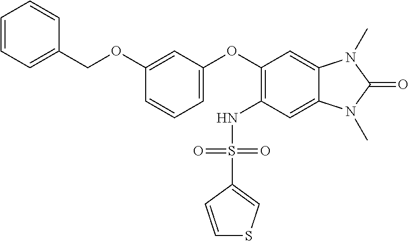 [SMILES: Cn1c(=O)n(C)c2cc(Oc3cccc(OCc4ccccc4)c3)c(NS(=O)(=O)c3ccsc3)cc21] | N-(6-(3- (benzyloxy) phenoxy)-1,3-dimethyl- 2-oxo-2,3- dihydro-1H- benzo[d]imidazol- 5-yl)thiophene- 3-sulfonamide | 521 | 522 | A |
| 22 | 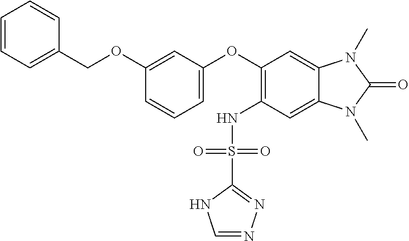 [SMILES: Cn1c(=O)n(C)c2cc(Oc3cccc(OCc4ccccc4)c3)c(NS(=O)(=O)c3nnc[nH]3)cc21] | N-(6-(3- (benzyloxy) phenoxy)-1,3-dimethyl- 2-oxo-2,3- dihydro-1H- benzo[d]imidazol- 5-yl)-4H-1,2,4- triazole-3- sulfonamide | 506 | 507 | A |
| 23 | 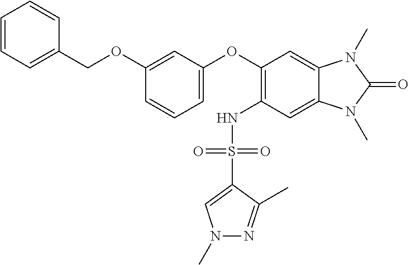 [SMILES: Cc1nn(C)cc1S(=O)(=O)Nc1cc2c(cc1Oc1cccc(OCc3ccccc3)c1)n(C)c(=O)n2C] | N-(6-(3- (benzyloxy) phenoxy)-1,3-dimethyl- 2-oxo-2,3- dihydro-1H- benzo[d]imidazol- 5-yl)-1,3- dimethyl-1H- pyrazole-4- sulfonamide | 533 | 534 | A |
| 24 | 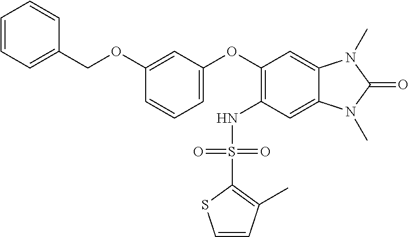 [SMILES: Cc1ccsc1S(=O)(=O)Nc1cc2c(cc1Oc1cccc(OCc3ccccc3)c1)n(C)c(=O)n2C] | N-(6-(3- (benzyloxy) phenoxy)-1,3-dimethyl- 2-oxo-2,3- dihydro-1H- benzo[d]imidazol- 5-yl)-3- methylthiophene- 2-sulfonamide | 535 | 536 | A |
| 25 | 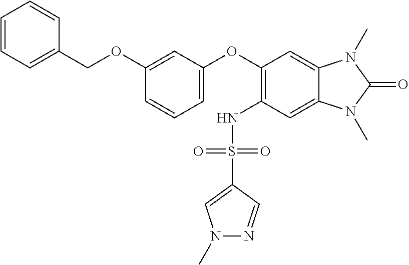 [SMILES: Cn1cc(S(=O)(=O)Nc2cc3c(cc2Oc2cccc(OCc4ccccc4)c2)n(C)c(=O)n3C)cn1] | N-(6-(3- (benzyloxy) phenoxy)-1,3-dimethyl- 2-oxo-2,3- dihydro-1H- benzo[d]imidazol- 5-yl)-1-methyl- 1H-pyrazole-4- sulfonamide | 519 | 520 | A |
| 26 | 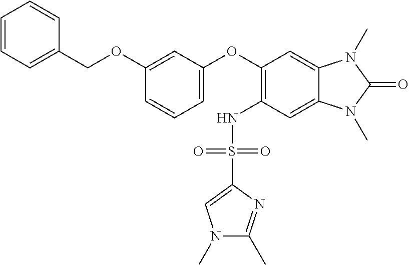 [SMILES: Cc1nc(S(=O)(=O)Nc2cc3c(cc2Oc2cccc(OCc4ccccc4)c2)n(C)c(=O)n3C)cn1C] | N-(6-(3- (benzyloxy) phenoxy)-1,3-dimethyl- 2-oxo-2,3- dihydro-1H- benzo[d]imidazol- 5-yl)-1,2- dimethyl-1H- imidazole-4- sulfonamide | 533 | 534 | A |
| 27 | 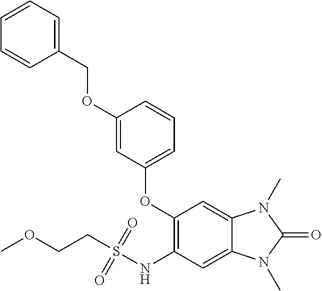 [SMILES: COCCS(=O)(=O)Nc1cc2c(cc1Oc1cccc(OCc3ccccc3)c1)n(C)c(=O)n2C] | N-(6-(3- (benzyloxy) phenoxy)-1,3-dimethyl- 2-oxo-2,3- dihydro-1H- benzo[d]imidazol- 5-yl)-2- methoxyethane-1- sulfonamide | 497 | 498 | A |
| 28 | 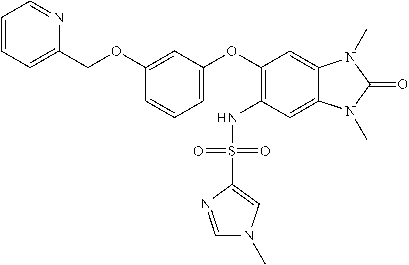 [SMILES: Cn1cnc(S(=O)(=O)Nc2cc3c(cc2Oc2cccc(OCc4ccccn4)c2)n(C)c(=O)n3C)c1] | N-(1,3-dimethyl- 2-oxo-6-(3- (pyridin-2- ylmethoxy) phenoxy)-2,3-dihydro- 1H- benzo[d]imidazol- 5-yl)-1-methyl- 1H-imidazole-4- sulfonamide | 520 | 521 | B |
| 29 | 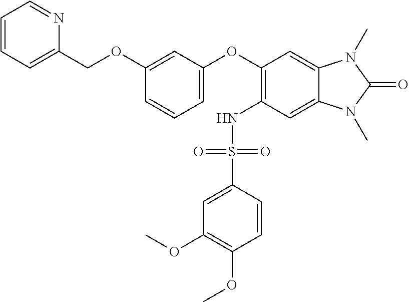 [SMILES: COc1ccc(S(=O)(=O)Nc2cc3c(cc2Oc2cccc(OCc4ccccn4)c2)n(C)c(=O)n3C)cc1OC] | N-(1,3-dimethyl- 2-oxo-6-(3- (pyridin-2- ylmethoxy) phenoxy)-2,3-dihydro- 1H- benzo[d]imidazol- 5-yl)-3,4- dimethoxybenzene- sulfonamide | 576 | 577 | B |
| 30 | 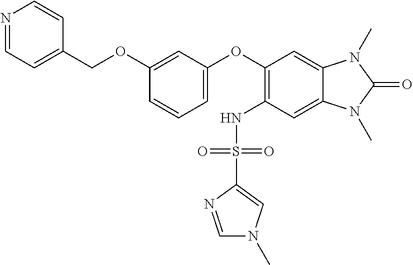 [SMILES: Cn1cnc(S(=O)(=O)Nc2cc3c(cc2Oc2cccc(OCc4ccncc4)c2)n(C)c(=O)n3C)c1] | N-(1,3-dimethyl- 2-oxo-6-(3- (pyridin-4- ylmethoxy) phenoxy)-2,3-dihydro- 1H- benzo[d]imidazol- 5-yl)-1-methyl- 1H-imidazole-4- sulfonamide | 520 | 521 | B |
| 31 | 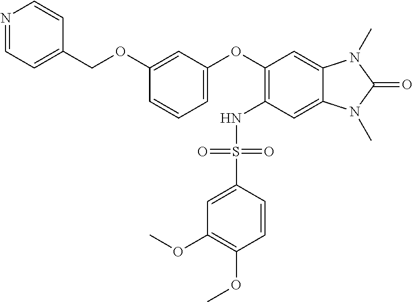 [SMILES: COc1ccc(S(=O)(=O)Nc2cc3c(cc2Oc2cccc(OCc4ccncc4)c2)n(C)c(=O)n3C)cc1OC] | N-(1,3-dimethyl- 2-oxo-6-(3- (pyridin-4- ylmethoxy) phenoxy)-2,3-dihydro- 1H- benzo[d]imidazol- 5-yl)-3,4- dimethoxybenzene- sulfonamide | 576 | 577 | B |
| 32 | 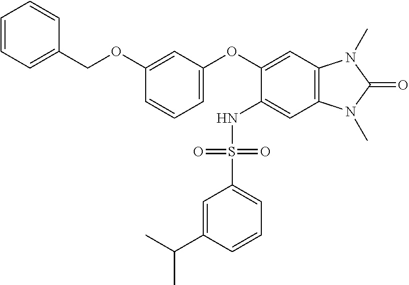 [SMILES: CC(C)c1cccc(S(=O)(=O)Nc2cc3c(cc2Oc2cccc(OCc4ccccc4)c2)n(C)c(=O)n3C)c1] | N-(6-(3- (benzyloxy) phenoxy)-1,3-dimethyl- 2-oxo-2,3- dihydro-1H- benzo[d]imidazol- 5-yl)-3- isopropylbenzene- sulfonamide | 557 | 558 | A |
| 33 | 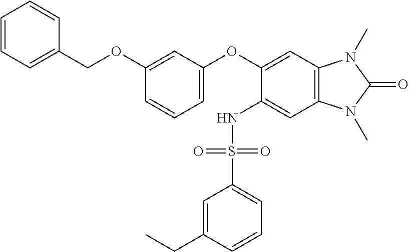 [SMILES: CCc1cccc(S(=O)(=O)Nc2cc3c(cc2Oc2cccc(OCc4ccccc4)c2)n(C)c(=O)n3C)c1] | N-(6-(3- (benzyloxy) phenoxy)-1,3-dimethyl- 2-oxo-2,3- dihydro-1H- benzo[d]imidazol- 5-yl)-3- ethylbenzene- sulfonamide | 543 | 544 | A |
| 34 | 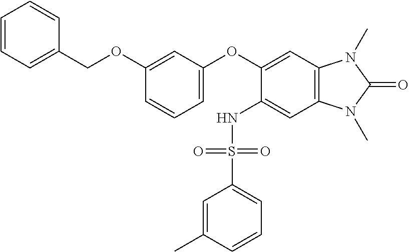 [SMILES: Cc1cccc(S(=O)(=O)Nc2cc3c(cc2Oc2cccc(OCc4ccccc4)c2)n(C)c(=O)n3C)c1] | N-(6-(3- (benzyloxy) phenoxy)-1,3-dimethyl- 2-oxo-2,3- dihydro-1H- benzo[d]imidazol- 5-yl)-3- methylbenzene- sulfonamide | 529 | 530 | A |
| 35 | 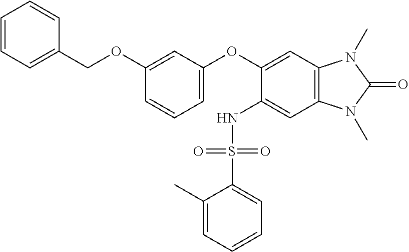 [SMILES: Cc1ccccc1S(=O)(=O)Nc1cc2c(cc1Oc1cccc(OCc3ccccc3)c1)n(C)c(=O)n2C] | N-(6-(3- (benzyloxy) phenoxy)-1,3-dimethyl- 2-oxo-2,3- dihydro-1H- benzo[d]imidazol- 5-yl)-2- methylbenzene- sulfonamide | 529 | 530 | A |
| 36 | 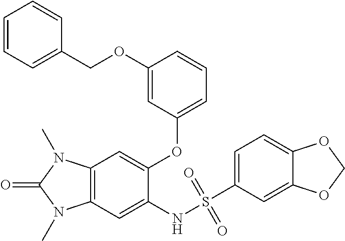 [SMILES: Cn1c(=O)n(C)c2cc(Oc3cccc(OCc4ccccc4)c3)c(NS(=O)(=O)c3ccc4c(c3)OCO4)cc21] | N-(6-(3- (benzyloxy) phenoxy)-1,3-dimethyl- 2-oxo-2,3- dihydro-1H- benzo[d]imidazol- 5- yl)benzo[d][1,3] dioxole-5- sulfonamide | 559 | 560 | A |
| 37 | 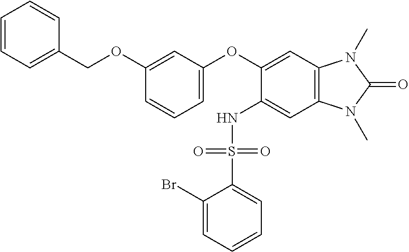 [SMILES: Cn1c(=O)n(C)c2cc(Oc3cccc(OCc4ccccc4)c3)c(NS(=O)(=O)c3ccccc3Br)cc21] | N-(6-(3- (benzyloxy) phenoxy)-1,3-dimethyl- 2-oxo-2,3- dihydro-1H- benzo[d]imidazol- 5-yl)-2- bromobenzene- sulfonamide | 593 | 594 | A |
| 38 | 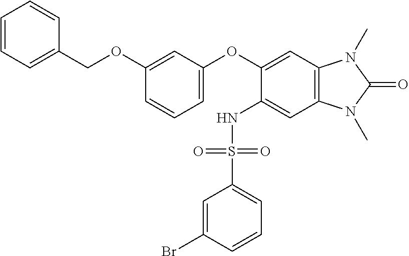 [SMILES: Cn1c(=O)n(C)c2cc(Oc3cccc(OCc4ccccc4)c3)c(NS(=O)(=O)c3cccc(Br)c3)cc21] | N-(6-(3- (benzyloxy) phenoxy)-1,3-dimethyl- 2-oxo-2,3- dihydro-1H- benzo[d]imidazol- 5-yl)-3- bromobenzene- sulfonamide | 593 | 594 | A |
| 39 | 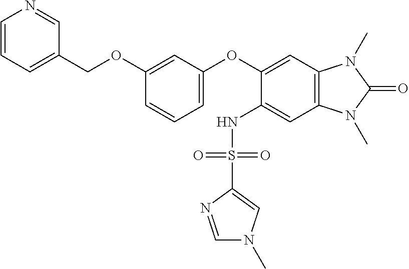 [SMILES: Cn1cnc(S(=O)(=O)Nc2cc3c(cc2Oc2cccc(OCc4cccnc4)c2)n(C)c(=O)n3C)c1] | N-(1,3-dimethyl- 2-oxo-6-(3- (pyridin-3- ylmethoxy) phenoxy)-2,3-dihydro- 1H- benzo[d]imidazol- 5-yl)-1-methyl- 1H-imidazole-4- sulfonamide | 520 | 521 | B |
| 40 | 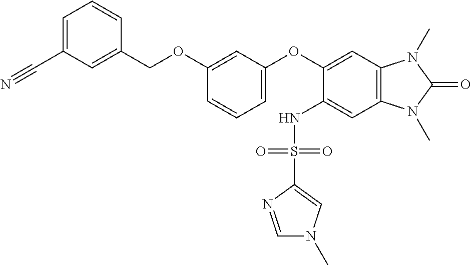 [SMILES: Cn1cnc(S(=O)(=O)Nc2cc3c(cc2Oc2cccc(OCc4cccc(C#N)c4)c2)n(C)c(=O)n3C)c1] | N-(6-(3-((3- cyanobenzyl)oxy) phenoxy)-1,3- dimethyl-2-oxo- 2,3-dihydro-1H- benzo[d]imidazol- 5-yl)-1-methyl- 1H-imidazole-4- sulfonamide | 544 | 545 | B |
| 41 | 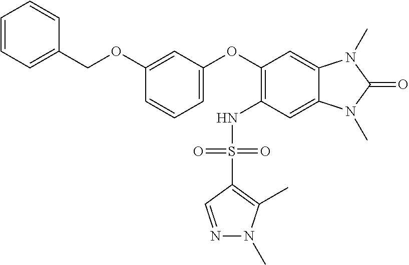 [SMILES: Cc1c(S(=O)(=O)Nc2cc3c(cc2Oc2cccc(OCc4ccccc4)c2)n(C)c(=O)n3C)cnn1C] | N-(6-(3- (benzyloxy) phenoxy)-1,3-dimethyl- 2-oxo-2,3- dihydro-1H- benzo[d]imidazol- 5-yl)-1,5- dimethyl-1H- pyrazole-4- sulfonamide | 533 | 534 | A |
| 42 | 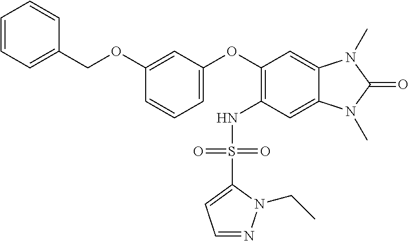 [SMILES: CCn1nccc1S(=O)(=O)Nc1cc2c(cc1Oc1cccc(OCc3ccccc3)c1)n(C)c(=O)n2C] | N-(6-(3- (benzyloxy) phenoxy)-1,3-dimethyl- 2-oxo-2,3- dihydro-1H- benzo[d]imidazol- 5-yl)-1-ethyl-1H- pyrazole-5- sulfonamide | 533 | 534 | A |
| 43 | 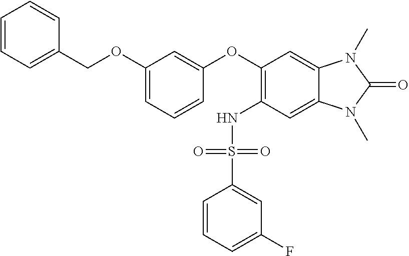 [SMILES: Cn1c(=O)n(C)c2cc(Oc3cccc(OCc4ccccc4)c3)c(NS(=O)(=O)c3cccc(F)c3)cc21] | N-(6-(3- (benzyloxy) phenoxy)-1,3-dimethyl- 2-oxo-2,3- dihydro-1H- benzo[d]imidazol- 5-yl)-3- fluorobenzene- sulfonamide | 533 | 534 | A |
| 44 | 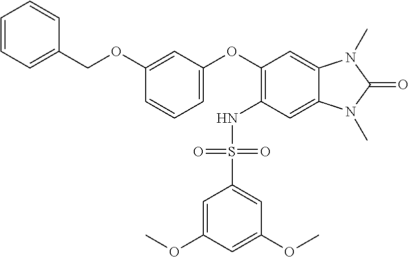 [SMILES: COc1cc(OC)cc(S(=O)(=O)Nc2cc3c(cc2Oc2cccc(OCc4ccccc4)c2)n(C)c(=O)n3C)c1] | N-(6-(3- (benzyloxy) phenoxy)-1,3-dimethyl- 2-oxo-2,3- dihydro-1H- benzo[d]imidazol- 5-yl)-3,5- dimethoxybenzene- sulfonamide | 575 | 576 | A |
| 45 | 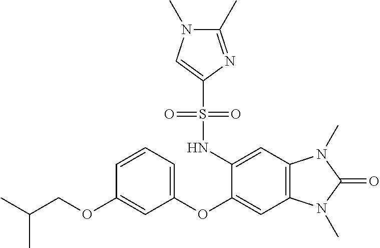 [SMILES: Cc1nc(S(=O)(=O)Nc2cc3c(cc2Oc2cccc(OCC(C)C)c2)n(C)c(=O)n3C)cn1C] | N-(6-(3- isobutoxyphenoxy)- 1,3-dimethyl- 2-oxo-2,3- dihydro-1H- benzo[d]imidazol- 5-yl)-1,2- dimethyl-1H- imidazole-4- sulfonamide | 499 | 500 | B |
| 46 | 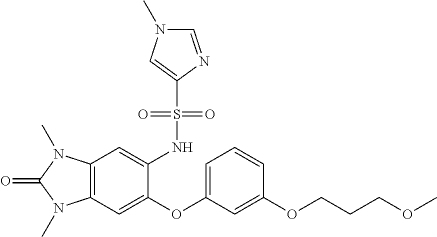 [SMILES: COCCCOc1cccc(Oc2cc3c(cc2NS(=O)(=O)c2cn(C)cn2)n(C)c(=O)n3C)c1] | N-(6-(3-(3- methoxypropoxy) phenoxy)-1,3- dimethyl-2-oxo- 2,3-dihydro-1H- benzo[d]imidazol- 5-yl)-1,2- dimethyl-1H- imidazole-4- sulfonamide | 515 | 516 | B |
| 47 | 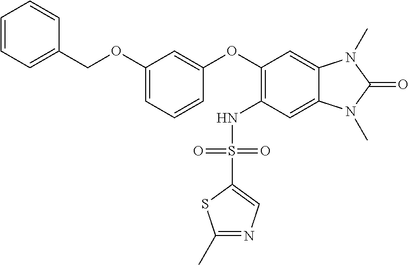 [SMILES: Cc1ncc(S(=O)(=O)Nc2cc3c(cc2Oc2cccc(OCc4ccccc4)c2)n(C)c(=O)n3C)s1] | N-(6-(3- (benzyloxy) phenoxy)-1,3-dimethyl- 2-oxo-2,3- dihydro-1H- benzo[d]imidazol- 5-yl)-2- methylthiazole-5- sulfonamide | 536 | 537 | A |
| 48 | 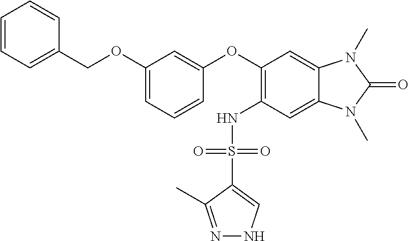 [SMILES: Cc1n[nH]cc1S(=O)(=O)Nc1cc2c(cc1Oc1cccc(OCc3ccccc3)c1)n(C)c(=O)n2C] | N-(6-(3- (benzyloxy) phenoxy)-1,3-dimethyl- 2-oxo-2,3- dihydro-1H- benzo[d]imidazol- 5-yl)-3-methyl- 1H-pyrazole-4- sulfonamide | 519 | 520 | A |
| 49 | 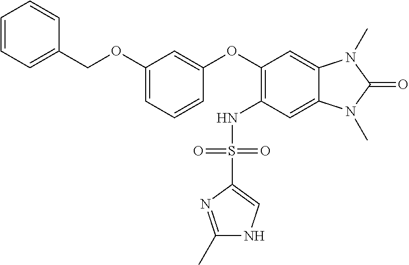 [SMILES: Cc1nc(S(=O)(=O)Nc2cc3c(cc2Oc2cccc(OCc4ccccc4)c2)n(C)c(=O)n3C)c[nH]1] | N-(6-(3- (benzyloxy) phenoxy)-1,3-dimethyl- 2-oxo-2,3- dihydro-1H- benzo[d]imidazol- 5-yl)-2-methyl- 1H-imidazole-4- sulfonamide | 519 | 520 | A |
| 50 | 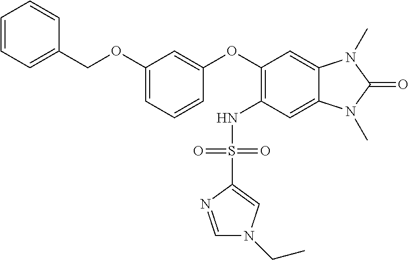 [SMILES: CCn1cnc(S(=O)(=O)Nc2cc3c(cc2Oc2cccc(OCc4ccccc4)c2)n(C)c(=O)n3C)c1] | N-(6-(3- (benzyloxy) phenoxy)-1,3-dimethyl- 2-oxo-2,3- dihydro-1H- benzo[d]imidazol- 5-yl)-1-ethyl-1H- imidazole-4- sulfonamide | 533 | 534 | A |
| 51 | 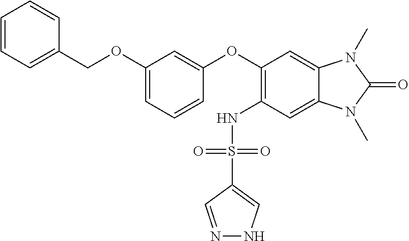 [SMILES: Cn1c(=O)n(C)c2cc(Oc3cccc(OCc4ccccc4)c3)c(NS(=O)(=O)c3cn[nH]c3)cc21] | N-(6-(3- (benzyloxy) phenoxy)-1,3-dimethyl- 2-oxo-2,3- dihydro-1H- benzo[d]imidazol- 5-yl)-1H- pyrazole-4- sulfonamide | 505 | 506 | A |
| 52 | 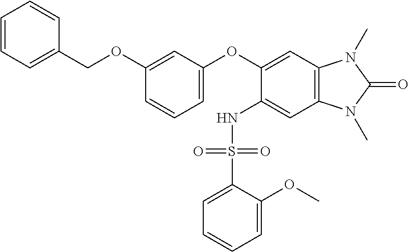 [SMILES: COc1ccccc1S(=O)(=O)Nc1cc2c(cc1Oc1cccc(OCc3ccccc3)c1)n(C)c(=O)n2C] | N-(6-(3- (benzyloxy) phenoxy)-1,3-dimethyl- 2-oxo-2,3- dihydro-1H- benzo[d]imidazol- 5-yl)-2- methoxybenzene- sulfonamide | 545 | 546 | A |
| 53 | 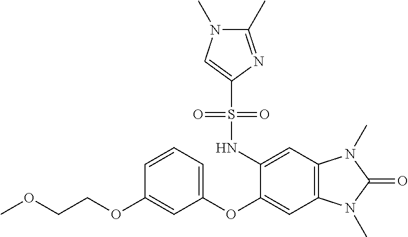 [SMILES: COCCOc1cccc(Oc2cc3c(cc2NS(=O)(=O)c2cn(C)c(C)n2)n(C)c(=O)n3C)c1] | N-(6-(3-(2- methoxyethoxy) phenoxy)-1,3- dimethyl-2-oxo- 2,3-dihydro-1H- benzo[d]imidazol- 5-yl)-1,2- dimethyl-1H- imidazole-4- sulfonamide | 501 | 502 | B |
| 54 | 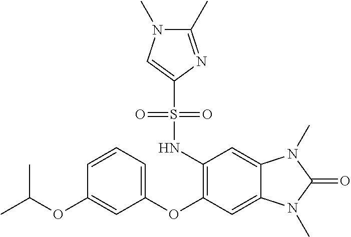 [SMILES: Cc1nc(S(=O)(=O)Nc2cc3c(cc2Oc2cccc(OC(C)C)c2)n(C)c(=O)n3C)cn1C] | N-(6-(3- isopropoxyphenoxy)- 1,3-dimethyl- 2-oxo-2,3- dihydro-1H- benzo[d]imidazol- 5-yl)-1,2- dimethyl-1H- imidazole-4- sulfonamide | 485 | 486 | C |
| 55 | 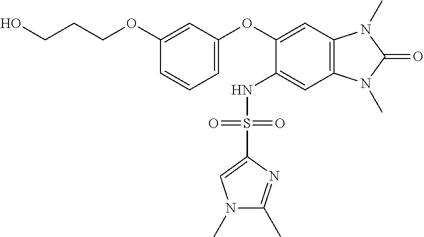 [SMILES: Cc1nc(S(=O)(=O)Nc2cc3c(cc2Oc2cccc(OCCCO)c2)n(C)c(=O)n3C)cn1C] | N-(6-(3-(3- hydroxypropoxy) phenoxy)-1,3- dimethyl-2-oxo- 2,3-dihydro-1H- benzo[d]imidazol- 5-yl)-1,2- dimethyl-1H- imidazole-4- sulfonamide | 501 | 502 | B |
| 56 | 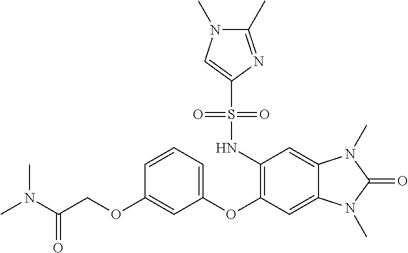 [SMILES: Cc1nc(S(=O)(=O)Nc2cc3c(cc2Oc2cccc(OCC(=O)N(C)C)c2)n(C)c(=O)n3C)cn1C] | 2-(3-((6-((1,2- dimethyl-1H- imidazole)-4- sulfonamido)-1,3- dimethyl-2-oxo- 2,3-dihydro-1H- benzo[d]imidazol- 5- yl)oxy)phenoxy)- N,N- dimethylacetamide | 528 | 529 | C |
| 57 | 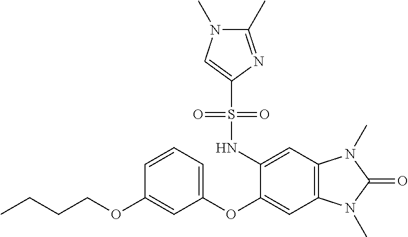 [SMILES: CCCCOc1cccc(Oc2cc3c(cc2NS(=O)(=O)c2cn(C)c(C)n2)n(C)c(=O)n3C)c1] | N-(6-(3- butoxyphenoxy)- 1,3-dimethyl-2- oxo-2,3-dihydro- 1H- benzo[d]imidazol- 5-yl)-1,2- dimethyl-1H- imidazole-4- sulfonamide | 499 | 500 | B |
| 58 | 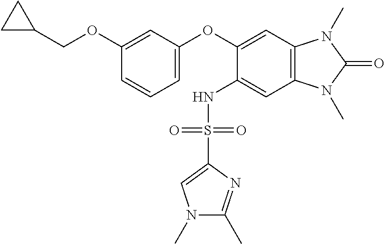 [SMILES: Cc1nc(S(=O)(=O)Nc2cc3c(cc2Oc2cccc(OCC4CC4)c2)n(C)c(=O)n3C)cn1C] | N-(6-(3- (cyclopropylmethoxy) phenoxy)- 1,3-dimethyl-2- oxo-2,3-dihydro- 1H- benzo[d]imidazol- 5-yl)-1,2- dimethyl-1H- imidazole-4- sulfonamide | 497 | 498 | B |
| 59 | 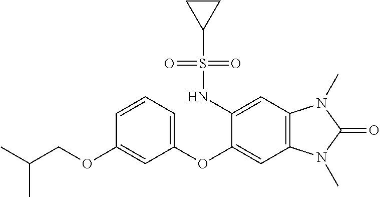 [SMILES: CC(C)COc1cccc(Oc2cc3c(cc2NS(=O)(=O)C2CC2)n(C)c(=O)n3C)c1] | N-(6-(3- isobutoxyphenoxy)- 1,3-dimethyl- 2-oxo-2,3- dihydro-1H- benzo[d]imidazol- 5- yl)cyclopropane- sulfonamide | 445 | 446 | C |
| 60 | 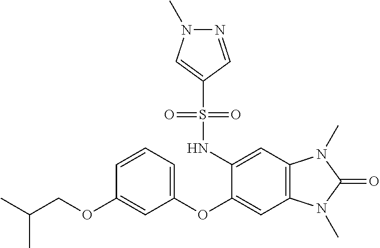 [SMILES: CC(C)COc1cccc(Oc2cc3c(cc2NS(=O)(=O)c2cnn(C)c2)n(C)c(=O)n3C)c1] | N-(6-(3- isobutoxyphenoxy)- 1,3-dimethyl- 2-oxo-2,3- dihydro-1H- benzo[d]imidazol- 5-yl)-1-methyl- 1H-pyrazole-4- sulfonamide | 485 | 486 | C |
| 61 | 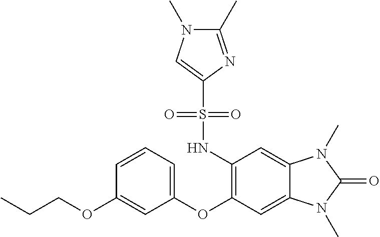 [SMILES: CCCOc1cccc(Oc2cc3c(cc2NS(=O)(=O)c2cn(C)c(C)n2)n(C)c(=O)n3C)c1] | N-(1,3-dimethyl- 2-oxo-6-(3- propoxyphenoxy)- 2,3-dihydro-1H- benzo[d]imidazol- 5-yl)-1,2- dimethyl-1H- imidazole-4- sulfonamide | 485 | 486 | C |
| 62 | 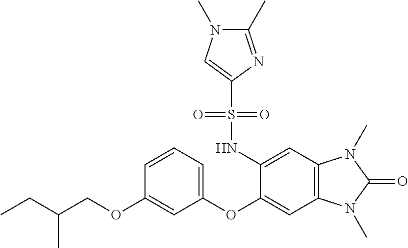 [SMILES: CCC(C)COc1cccc(Oc2cc3c(cc2NS(=O)(=O)c2cn(C)c(C)n2)n(C)c(=O)n3C)c1] | N-(1,3-dimethyl- 6-(3-(2- methylbutoxy) phenoxy)-2-oxo-2,3- dihydro-1H- benzo[d]imidazol- 5-yl)-1,2- dimethyl-1H- imidazole-4- sulfonamide | 513 | 514 | B |
| 63 | 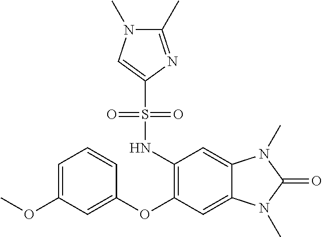 [SMILES: COc1cccc(Oc2cc3c(cc2NS(=O)(=O)c2cn(C)c(C)n2)n(C)c(=O)n3C)c1] | N-(6-(3- methoxyphenoxy)- 1,3-dimethyl-2- oxo-2,3-dihydro- 1H- benzo[d]imidazol- 5-yl)-1-methyl- 1H-imidazole-4- sulfonamide | 443 | 444 | A |
| 64 | 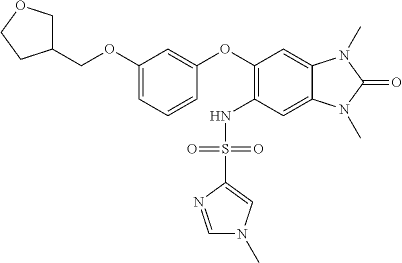 [SMILES: Cn1cnc(S(=O)(=O)Nc2cc3c(cc2Oc2cccc(OCC4CCOC4)c2)n(C)c(=O)n3C)c1] | N-(1,3-dimethyl- 2-oxo-6-(3- ((tetrahydrofuran- 3- yl)methoxy) phenoxy)-2,3-dihydro- 1H- benzo[d]imidazol- 5-yl)-1-methyl- 1H-imidazole-4- sulfonamide | 513 | 514 | B |
| 65 | 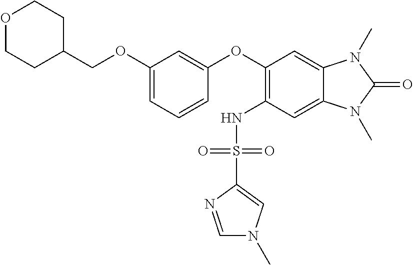 [SMILES: Cn1cnc(S(=O)(=O)Nc2cc3c(cc2Oc2cccc(OCC4CCOCC4)c2)n(C)c(=O)n3C)c1] | N-(1,3-dimethyl- 2-oxo-6-(3- ((tetrahydro-2H- pyran-4- yl)methoxy) phenoxy)-2,3-dihydro- 1H- benzo[d]imidazol- 5-yl)-1-methyl- 1H-imidazole-4- sulfonamide | 527 | 528 | B |
| 66 | 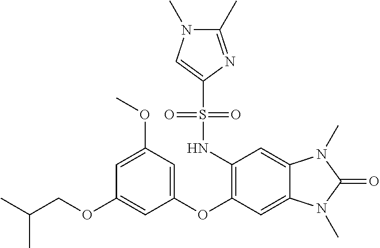 [SMILES: COc1cc(OCC(C)C)cc(Oc2cc3c(cc2NS(=O)(=O)c2cn(C)c(C)n2)n(C)c(=O)n3C)c1] | N-(6-(3- isobutoxy-5- methoxyphenoxy)- 1,3-dimethyl-2- oxo-2,3-dihydro- 1H- benzo[d]imidazol- 5-yl)-1,2- dimethyl-1H- imidazole-4- sulfonamide | 529 | 530 | C |
| 67 | 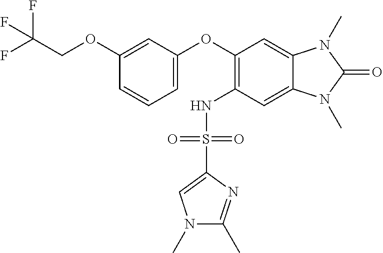 [SMILES: Cc1nc(S(=O)(=O)Nc2cc3c(cc2Oc2cccc(OCC(F)(F)F)c2)n(C)c(=O)n3C)cn1C] | N-(1,3-dimethyl- 2-oxo-6-(3-(2,2,2- trifluoroethoxy) phenoxy)-2,3- dihydro-1H- benzo[d]imidazol- 5-yl)-1,2- dimethyl-1H- imidazole-4- sulfonamide | 525 | 526 | C |
| 68 | 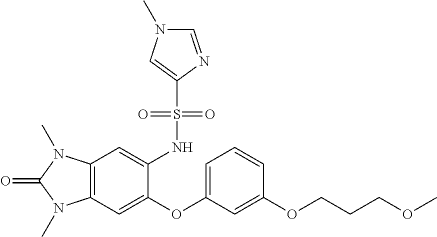 [SMILES: COCCCOc1cccc(Oc2cc3c(cc2NS(=O)(=O)c2cn(C)cn2)n(C)c(=O)n3C)c1] | N-(6-(3-(3- methoxypropoxy) phenoxy)-1,3- dimethyl-2-oxo- 2,3-dihydro-1H- benzo[d]imidazol- 5-yl)-1-methyl- 1H-imidazole-4- sulfonamide | 501 | 502 | B |
| 69 | 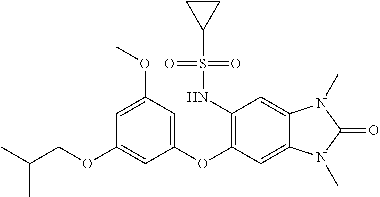 [SMILES: COc1cc(OCC(C)C)cc(Oc2cc3c(cc2NS(=O)(=O)C2CC2)n(C)c(=O)n3C)c1] | N-(6-(3- isobutoxy-5- methoxyphenoxy)- 1,3-dimethyl-2- oxo-2,3-dihydro- 1H- benzo[d]imidazol- 5- yl)cyclopropane- sulfonamide | 475 | 476 | C |
| 70 | 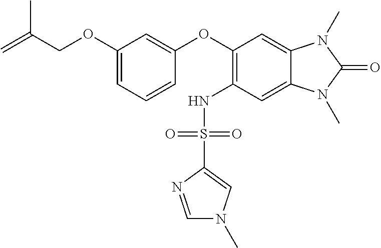 [SMILES: C=C(C)COc1cccc(Oc2cc3c(cc2NS(=O)(=O)c2cn(C)cn2)n(C)c(=O)n3C)c1] | N-(1,3-dimethyl- 6-(3-((2- methylallyl)oxy) phenoxy)-2-oxo- 2,3-dihydro-H- benzo[d]imidazol- 5-yl)-1-methyl- 1H-imidazole-4- sulfonamide | 483 | 484 | A |
| 71 | 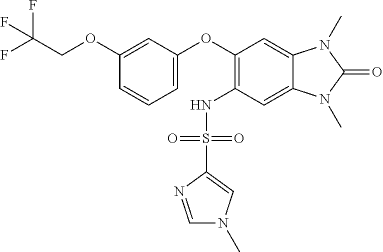 [SMILES: Cn1cnc(S(=O)(=O)Nc2cc3c(cc2Oc2cccc(OCC(F)(F)F)c2)n(C)c(=O)n3C)c1] | N-(1,3-dimethyl- 2-oxo-6-(3-(2,2,2- trifluoroethoxy) phenoxy)-2,3- dihydro-1H- benzo[d]imidazol- 5-yl)-1-methyl- 1H-imidazole-4- sulfonamide | 511 | 512 | C |
| 72 | 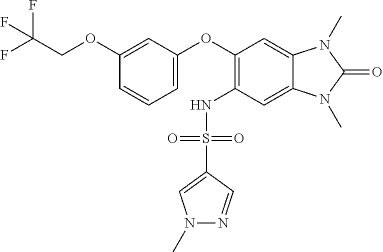 [SMILES: Cn1cc(S(=O)(=O)Nc2cc3c(cc2Oc2cccc(OCC(F)(F)F)c2)n(C)c(=O)n3C)cn1] | N-(1,3-dimethyl- 2-oxo-6-(3-(2,2,2- trifluoroethoxy) phenoxy)-2,3- dihydro-1H- benzo[d]imidazol- 5-yl)-1-methyl- 1H-pyrazole-4- sulfonamide | 511 | 512 | C |
| 73 | 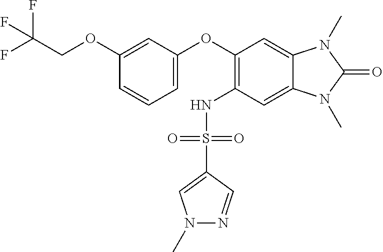 [SMILES: Cn1cc(S(=O)(=O)Nc2cc3c(cc2Oc2cccc(OCC(F)(F)F)c2)n(C)c(=O)n3C)cn1] | N-(1,3-dimethyl- 2-oxo-6-(3- (trifluoromethoxy) phenoxy)-2,3- dihydro-1H- benzo[d]imidazol- 5-yl)-1-methyl- 1H-pyrazole-4- sulfonamide | 497 | 498 | C |
| 74 | 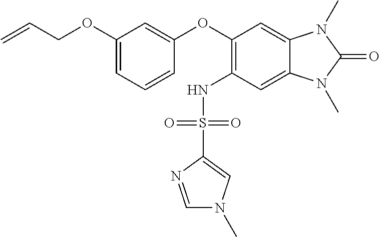 [SMILES: C=CCOc1cccc(Oc2cc3c(cc2NS(=O)(=O)c2cn(C)cn2)n(C)c(=O)n3C)c1] | N-(6-(3- (allyloxy)phenoxy)- 1,3-dimethyl- 2-oxo-2,3- dihydro-1H- benzo[d]imidazol- 5-yl)-1-methyl- 1H-imidazole-4- sulfonamide | 469 | 470 | A |
| 75 | 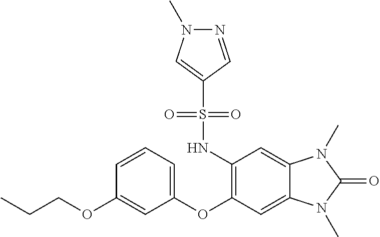 [SMILES: CCCOc1cccc(Oc2cc3c(cc2NS(=O)(=O)c2cnn(C)c2)n(C)c(=O)n3C)c1] | N-(1,3-dimethyl- 2-oxo-6-(3- propoxyphenoxy)- 2,3-dihydro-1H- benzo[d]imidazol- 5-yl)-1-methyl- 1H-pyrazole-4- sulfonamide | 471 | 472 | A |
| 76 | 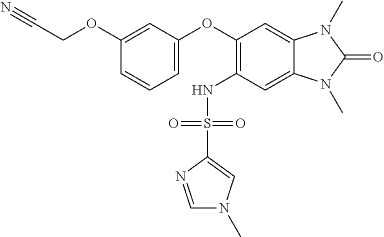 [SMILES: Cn1cnc(S(=O)(=O)Nc2cc3c(cc2Oc2cccc(OCC#N)c2)n(C)c(=O)n3C)c1] | N-(6-(3- (cyanomethoxy) phenoxy)-1,3- dimethyl-2-oxo- 2,3-dihydro-1H- benzo[d]imidazol- 5-yl)-1-methyl- 1H-imidazole-4- sulfonamide | 468 | 469 | A |
| 77 | 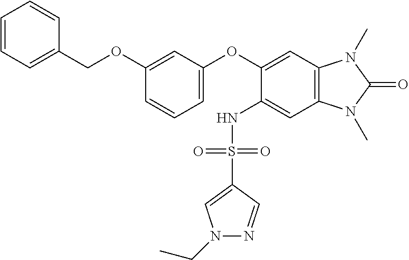 [SMILES: CCn1cc(S(=O)(=O)Nc2cc3c(cc2Oc2cccc(OCc4ccccc4)c2)n(C)c(=O)n3C)cn1] | N-(6-(3- (benzyloxy) phenoxy)-1,3-dimethyl- 2-oxo-2,3- dihydro-1H- benzo[d]imidazol- 5-yl)-1-ethyl-1H- pyrazole-4- sulfonamide | 533 | 534 | A |
| 78 | 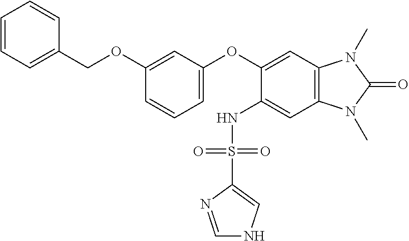 [SMILES: Cn1c(=O)n(C)c2cc(Oc3cccc(OCc4ccccc4)c3)c(NS(=O)(=O)c3c[nH]cn3)cc21] | N-(6-(3- (benzyloxy) phenoxy)-1,3-dimethyl- 2-oxo-2,3- dihydro-1H- benzo[d]imidazol- 5-yl)-1H- imidazole-4- sulfonamide | 505 | 506 | A |
| 79 | 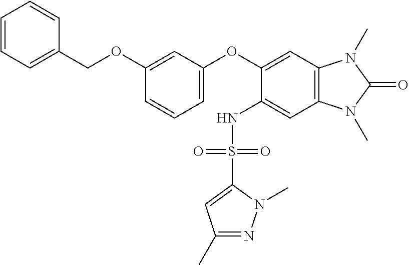 [SMILES: Cc1cc(S(=O)(=O)Nc2cc3c(cc2Oc2cccc(OCc4ccccc4)c2)n(C)c(=O)n3C)n(C)n1] | N-(6-(3- (benzyloxy) phenoxy)-1,3-dimethyl- 2-oxo-2,3- dihydro-1H- benzo[d]imidazol- 5-yl)-1,3- dimethyl-1H- pyrazole-5- sulfonamide | 533 | 534 | A |
| 80 | 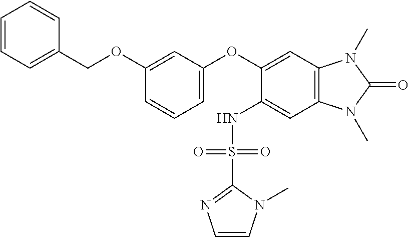 [SMILES: Cn1ccnc1S(=O)(=O)Nc1cc2c(cc1Oc1cccc(OCc3ccccc3)c1)n(C)c(=O)n2C] | N-(6-(3- (benzyloxy) phenoxy)-1,3-dimethyl- 2-oxo-2,3- dihydro-1H- benzo[d]imidazol- 5-yl)-1-methyl- 1H-imidazole-2- sulfonamide | 519 | 520 | A |
| 81 | 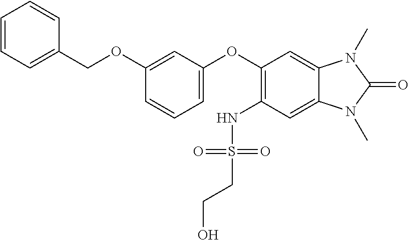 [SMILES: Cn1c(=O)n(C)c2cc(Oc3cccc(OCc4ccccc4)c3)c(NS(=O)(=O)CCO)cc21] | N-(6-(3- (benzyloxy) phenoxy)-1,3-dimethyl- 2-oxo-2,3- dihydro-1H- benzo[d]imidazol- 5-yl)-2- hydroxyethane-1- sulfonamide | 483 | 484 | A |
| 82 | 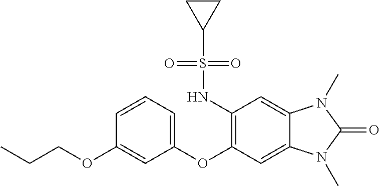 [SMILES: CCCOc1cccc(Oc2cc3c(cc2NS(=O)(=O)C2CC2)n(C)c(=O)n3C)c1] | N-(1,3-dimethyl- 2-oxo-6-(3- propoxyphenoxy)- 2,3-dihydro-1H- benzo[d]imidazol- 5- yl)cyclopropane- sulfonamide | 431 | 432 | A |
| 83 | 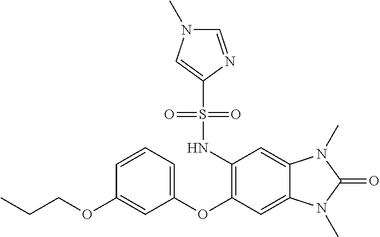 [SMILES: CCCOc1cccc(Oc2cc3c(cc2NS(=O)(=O)c2cn(C)cn2)n(C)c(=O)n3C)c1] | N-(1,3-dimethyl- 2-oxo-6-(3- propoxyphenoxy)- 2,3-dihydro-1H- benzo[d]imidazol- 5-yl)-1-methyl- 1H-imidazole-4- sulfonamide | 471 | 472 | A |
| 84 | 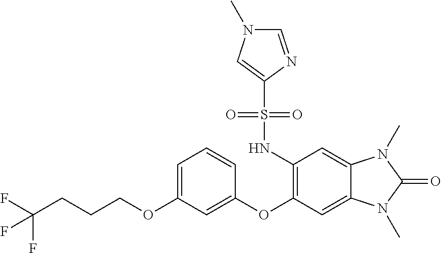 [SMILES: Cn1cnc(S(=O)(=O)Nc2cc3c(cc2Oc2cccc(OCCCC(F)(F)F)c2)n(C)c(=O)n3C)c1] | N-(1,3-dimethyl- 2-oxo-6-(3-(4,4,4- trifluorobutoxy) phenoxy)-2,3- dihydro-1H- benzo[d]imidazol- 5-yl)-1-methyl- 1H-imidazole-4- sulfonamide | 539 | 540 | B |
| 85 | 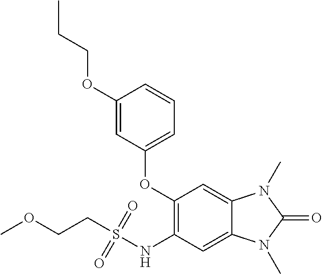 [SMILES: CCCOc1cccc(Oc2cc3c(cc2NS(=O)(=O)CCOC)n(C)c(=O)n3C)c1] | N-(1,3-dimethyl- 2-oxo-6-(3- propoxyphenoxy)- 2,3-dihydro-1H- benzo[d]imidazol- 5-yl)-2- methoxyethane-1- sulfonamide | 449 | 450 | A |
| 86 | 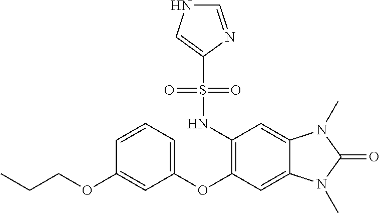 [SMILES: CCCOc1cccc(Oc2cc3c(cc2NS(=O)(=O)c2c[nH]cn2)n(C)c(=O)n3C)c1] | N-(1,3-dimethyl- 2-oxo-6-(3- propoxyphenoxy)- 2,3-dihydro-1H- benzo[d]imidazol- 5-yl)-1H- imidazole-4- sulfonamide | 457 | 458 | A |
| 87 | 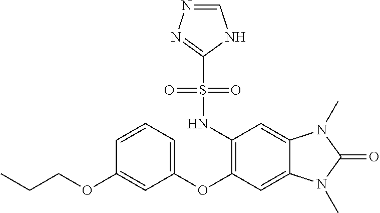 [SMILES: CCCOc1cccc(Oc2cc3c(cc2NS(=O)(=O)c2nnc[nH]2)n(C)c(=O)n3C)c1] | N-(1,3-diemthyl- 2-oxo-6-(3- propoxyphenoxy)- 2,3-dihydro-1H- benzo[d]imidazol- 5-yl)-4H-1,2,4- triazole-3- sulfonamide | 458 | 459 | A |
| 88 | 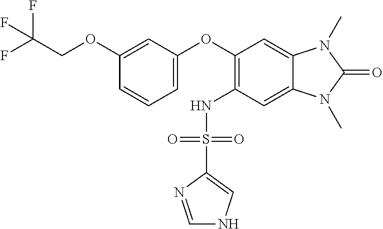 [SMILES: Cn1c(=O)n(C)c2cc(Oc3cccc(OCC(F)(F)F)c3)c(NS(=O)(=O)c3c[nH]cn3)cc21] | N-(1,3-dimethyl- 2-oxo-6-(3-(2,2,2- trifluoroethoxy) phenoxy)-2,3- dihydro-1H- benzo[d]imidazol- 5-yl)-1H- imidazole-4- sulfonamide | 497 | 498 | C |
| 89 | 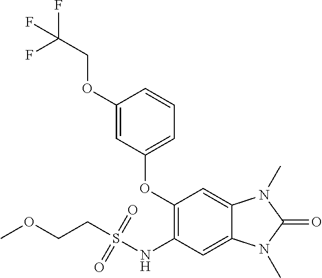 [SMILES: COCCS(=O)(=O)Nc1cc2c(cc1Oc1cccc(OCC(F)(F)F)c1)n(C)c(=O)n2C] | N-(1,3-dimethyl- 2-oxco-6-(3-(2,2,2- trifluoroethoxy) phenoxy)-2,3- dihydro-1H- benzo[d]imidazol- 5-yl)-2- methoxyethane-1- sulfonamide | 489 | 490 | C |
| 90 | 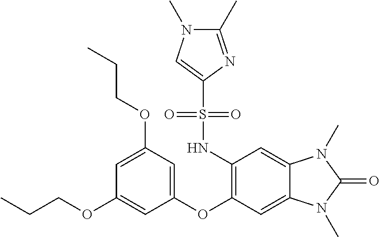 [SMILES: CCCOc1cc(OCCC)cc(Oc2cc3c(cc2NS(=O)(=O)c2cn(C)c(C)n2)n(C)c(=O)n3C)c1] | N-(6-(3,5- dipropoxyphenoxy)- 1,3-diemthyl- 2-oxo-2,3- dihydro-1H- benzo[d]imidazol- 5-yl)-1,2- dimethyl-1H- imidazole-4- sulfonamide | 543 | 544 | C |
| 91 | 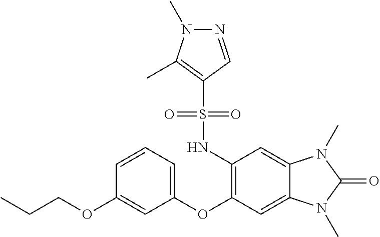 [SMILES: CCCOc1cccc(Oc2cc3c(cc2NS(=O)(=O)c2cnn(C)c2C)n(C)c(=O)n3C)c1] | N-(1,3-dimethyl- 2-oxo-6-(3- propoxyphenoxy)- 2,3-dihydro-1H- benzo[d]imidazol- 5-yl)-1,5- dimethyl-1H- pyrazole-4- sulfonamide | 485 | 486 | A |
| 92 | 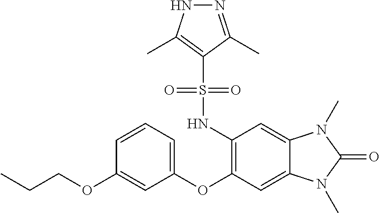 [SMILES: CCCOc1cccc(Oc2cc3c(cc2NS(=O)(=O)c2c(C)n[nH]c2C)n(C)c(=O)n3C)c1] | N-(1,3-dimethyl- 2-oxo-6-(3- propoxyphenoxy)- 2,3-dihydro-1H- benzo[d]imidazol- 5-yl)-3,5- dimethyl-1H- pyrazole-4- sulfonamide | 485 | 486 | A |
| 93 | 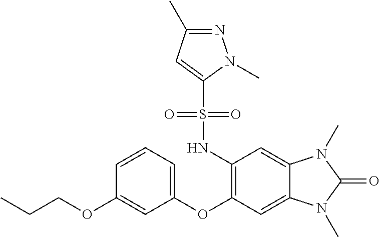 [SMILES: CCCOc1cccc(Oc2cc3c(cc2NS(=O)(=O)c2cc(C)nn2C)n(C)c(=O)n3C)c1] | N-(1,3-dimethyl- 2-oxo-6-(3- propoxyphenoxy)- 2,3-dihydro-1H- benzo[d]imidazol- 5-yl)-1,3- dimethyl-1H- pyrazole-5- sulfonamide | 485 | 486 | A |
| 94 | 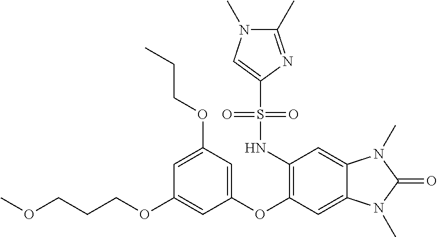 [SMILES: CCCOc1cc(OCCCOC)cc(Oc2cc3c(cc2NS(=O)(=O)c2cn(C)c(C)n2)n(C)c(=O)n3C)c1] | N-(6-(3-(3- methoxypropoxy)- 5- propoxyphenoxy)- 1,3-dimethyl-2- oxo-2,3-dihydro- 1H- benzo[d]imidazol- 5-yl)-1,2- dimethyl-1H- imidazole-4- sulfonamide | 573 | 574 | C |
| 95 | 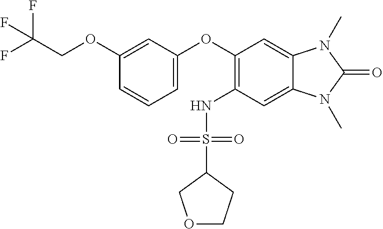 [SMILES: Cn1c(=O)n(C)c2cc(Oc3cccc(OCC(F)(F)F)c3)c(NS(=O)(=O)C3CCOC3)cc21] | N-(1,3-dimethyl- 2-oxo-6-(3-(2,2,2- trifluoroethoxy) phenoxy)-2,3- dihydro-1H- benzo[d]imidazol- 5- yl)tetrahydrofuran- 3-sulfonamide | 501 | 502 | C |
| 96 | 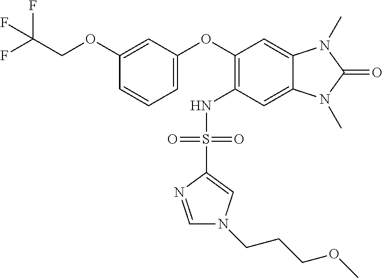 [SMILES: COCCCn1cnc(S(=O)(=O)Nc2cc3c(cc2Oc2cccc(OCC(F)(F)F)c2)n(C)c(=O)n3C)c1] | N-(1,3-dimethyl- 2-oxo-6-(3-(2,2,2- trifluoroethoxy) phenoxy)-2,3- dihydro-1H- benzo[d]imidazol- 5-yl)-1-(3- methoxypropyl)- 1H-imidazole-4- sulfonamide | 569 | 570 | C |
| 97 | 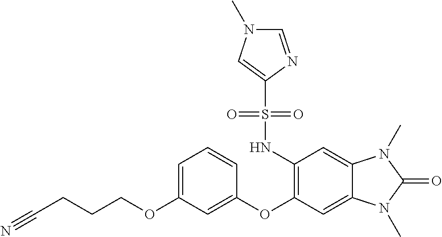 [SMILES: Cn1cnc(S(=O)(=O)Nc2cc3c(cc2Oc2cccc(OCCCC#N)c2)n(C)c(=O)n3C)c1] | N-(6-(3-(3- cyanopropoxy) phenoxy)-1,3- dimethyl-2-oxo- 2,3-dihydro-1H- benzo[d]imidazol- 5-yl)-1-methyl- 1H-imidazole-4- sulfonamide | 496 | 497 | B |
| 98 | 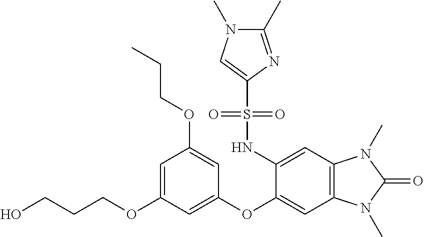 [SMILES: CCCOc1cc(OCCCO)cc(Oc2cc3c(cc2NS(=O)(=O)c2cn(C)c(C)n2)n(C)c(=O)n3C)c1] | N-(6-(3-(3- hydroxypropoxy)- 5- propoxyphenoxy)- 1,3-dimethyl-2- oxo-2,3-dihydro- 1H- benzo[d]imidazol- 5-yl)-1,2- dimethyl-1H- imidazole-4- sulfonamide | 559 | 560 | C |
| 99 | 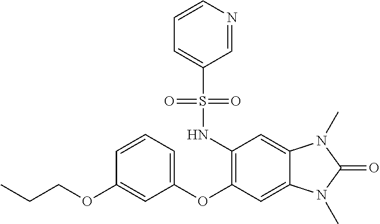 [SMILES: CCCOc1cccc(Oc2cc3c(cc2NS(=O)(=O)c2cccnc2)n(C)c(=O)n3C)c1] | N-(1,3-dimethyl- 2-oxo-6-(3- propoxyphenoxy)- 2,3-dihydro-1H- benzo[d]imidazol- 5-yl)pyridine-3- sulfonamide | 468 | 469 | A |
| 100 | 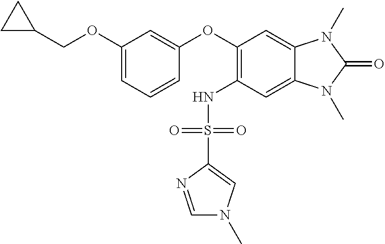 [SMILES: Cn1cnc(S(=O)(=O)Nc2cc3c(cc2Oc2cccc(OCC4CC4)c2)n(C)c(=O)n3C)c1] | N-(6-(3- (cyclopropylmethoxy) phenoxy)- 1,3-dimethyl-2- oxo-2,3-dihydro- 1H- benzo[d]imidazol- 5-yl)-1-methyl- 1H-imidazole-4- sulfonamide | 483 | 484 | B |
| 101 | 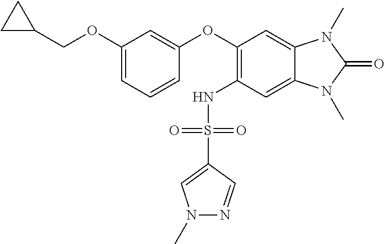 [SMILES: Cn1cc(S(=O)(=O)Nc2cc3c(cc2Oc2cccc(OCC4CC4)c2)n(C)c(=O)n3C)cn1] | N-(6-(3- (cyclopropylmethoxy) phenoxy)- 1,3-dimethyl-2- oxo-2,3-dihydro- 1H- benzo[d]imidazol- 5-yl)-1-methyl- 1H-pyrazole-4- sulfonamide | 483 | 484 | B |
| 102 | 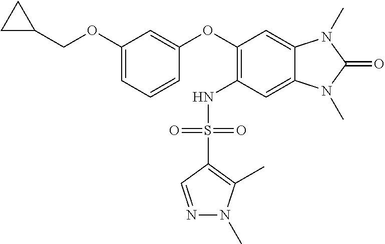 [SMILES: Cc1c(S(=O)(=O)Nc2cc3c(cc2Oc2cccc(OCC4CC4)c2)n(C)c(=O)n3C)cnn1C] | N-(6-(3- (cyclopropylmethoxy) phenoxy)- 1,3-dimethyl-2- oxo-2,3-dihydro- 1H- benzo[d]imidazol- 5-yl)-1,5- dimethyl-1H- pyrazole-4- sulfonamide | 497 | 498 | B |
| 103 | 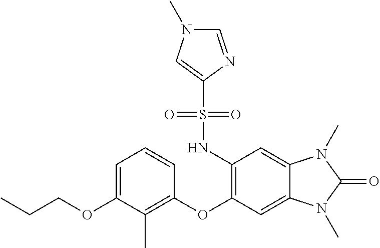 [SMILES: CCCOc1cccc(Oc2cc3c(cc2NS(=O)(=O)c2cn(C)cn2)n(C)c(=O)n3C)c1C] | N-(1,3-dimethyl- 6-(2-methyl-3- propoxyphenoxy)- 2-oxo-2,3- dihydro-1H- benzo[d]imidazol- 5-yl)-1-methyl- 1H-imidazole-4- sulfonamide | 485 | 486 | A |
| 104 | 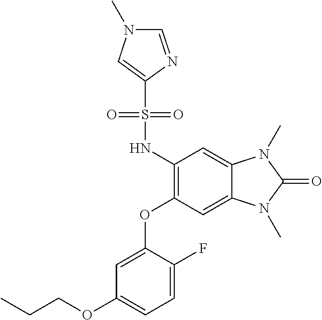 [SMILES: CCCOc1ccc(F)c(Oc2cc3c(cc2NS(=O)(=O)c2cn(C)cn2)n(C)c(=O)n3C)c1] | N-(6-(2-fluoro-5- propoxyphenoxy)- 1,3-dimethyl-2- oxo-2,3-dihydro- 1H- benzo[d]imidazol- 5-yl)-1-methyl- 1H-imidazole-4- sulfonamide | 489 | 490 | A |
| 105 | 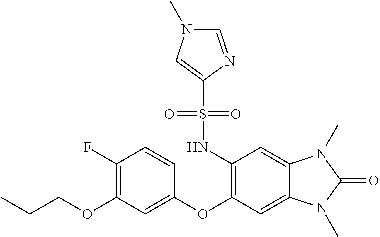 [SMILES: CCCOc1cc(Oc2cc3c(cc2NS(=O)(=O)c2cn(C)cn2)n(C)c(=O)n3C)ccc1F] | N-(6-(4-fluoro-3- propoxyphenoxy)- 1,3-dimethyl-2- oxo-2,3-dihydro- 1H- benzo[d]imidazol- 5-yl)-1-methyl- 1H-imidazole-4- sulfonamide | 489 | 490 | A |
| 106 | 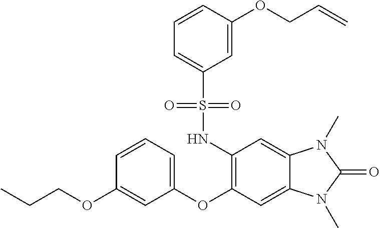 [SMILES: C=CCOc1cccc(S(=O)(=O)Nc2cc3c(cc2Oc2cccc(OCCC)c2)n(C)c(=O)n3C)c1] | 3-(allyloxy)-N- (1,3-dimethyl-2- oxo-6-(3- propoxyphenoxy)- 2,.3-dihydro-1H- benzo[d]imidazol- 5- yl)benzenesulfonamide | 523 | 524 | A |
| 107 | 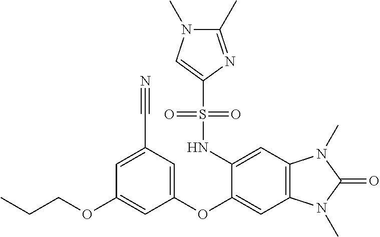 [SMILES: CCCOc1cc(C#N)cc(Oc2cc3c(cc2NS(=O)(=O)c2cn(C)c(C)n2)n(C)c(=O)n3C)c1] | N-(6-(3-cyano-5- propoxyphenoxy)- 1,3-dimethyl-2- oxo-2,3-dihydro- 1H- benzo[d]imidazol- 5-yl)-1,2- dimethyl-1H- imidazole-4- sulfonamide | 510 | 511 | C |
| 108 | 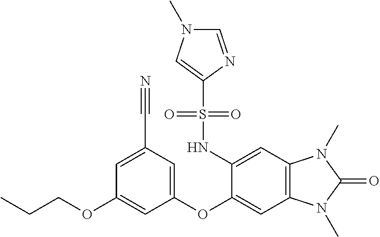 [SMILES: CCCOc1cc(C#N)cc(Oc2cc3c(cc2NS(=O)(=O)c2cn(C)cn2)n(C)c(=O)n3C)c1] | N-(6-(3-cyano-5- propoxyphenoxy)- 1,3-dimethyl-2- oxo-2,3-dihydro- 1H- benzo[d]imidazol- 5-yl)-1-methyl- 1H-imidazole-4- sulfonamide | 496 | 497 | C |
| 109 | 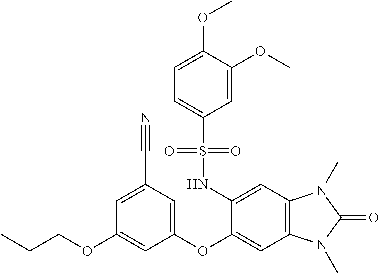 [SMILES: CCCOc1cc(C#N)cc(Oc2cc3c(cc2NS(=O)(=O)c2ccc(OC)c(OC)c2)n(C)c(=O)n3C)c1] | N-(6-(3-cyano-5- propoxyphenoxy)- 1,3-dimethyl-2- oxo-2,3-dihydro- 1H- benzo[d]imidazol- 5-yl)-3,4- dimethoxybenzene- sulfonamide | 552 | 553 | C |
| 110 | 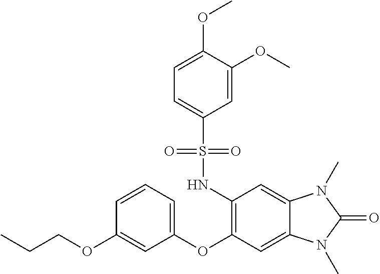 [SMILES: CCCOc1cccc(Oc2cc3c(cc2NS(=O)(=O)c2ccc(OC)c(OC)c2)n(C)c(=O)n3C)c1] | N-(1,3-dimethyl- 2-oxo-6-(3- propoxyphenoxy)- 2,3-dihydro-1H- benzo[d]imidazol- 5-yl)-3,4- dimethoxybenzene- sulfonamide | 527 | 528 | A |
| 111 | 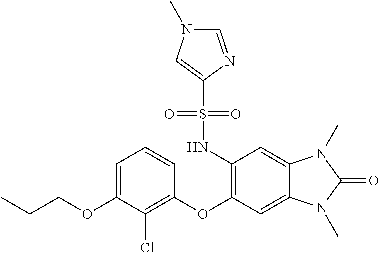 [SMILES: CCCOc1cccc(Oc2cc3c(cc2NS(=O)(=O)c2cn(C)cn2)n(C)c(=O)n3C)c1Cl] | N-(6-(2-chloro-3- propoxyphenoxy)- 1,3-dimethyl-2- oxo-2,3-dihydro- 1H- benzo[d]imidazol- 5-yl)-1-methyl- 1H-imidazole-4- sulfonamide | 505 | 506 | A |
| 112 | 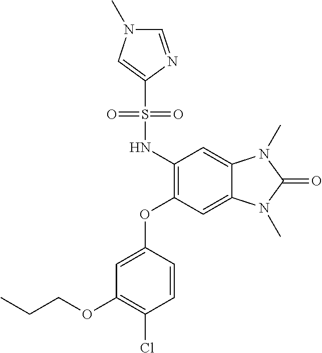 [SMILES: CCCOc1cc(Oc2cc3c(cc2NS(=O)(=O)c2cn(C)cn2)n(C)c(=O)n3C)ccc1Cl] | N-(6-(4-chloro-3- propoxyphenoxy)- 1,3-dimethyl-2- oxo-2,3-dihydro- 1H- benzo[d]imidazol- 5-yl)-1-methyl- 1H-imidazole-4- sulfonamide | 505 | 506 | A |
| 113 | 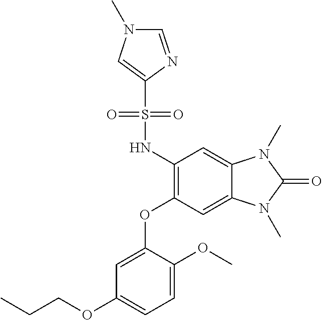 [SMILES: CCCOc1ccc(OC)c(Oc2cc3c(cc2NS(=O)(=O)c2cn(C)cn2)n(C)c(=O)n3C)c1] | N-(6-(2-methoxy- 5- propoxyphenoxy)- 1,3-dimethyl-2- oxo-2,3-dihydro- 1H- benzo[d]imidazol- 5-yl)-1-methyl- 1H-imidazole-4- sulfonamide | 501 | 502 | A |
| 114 | 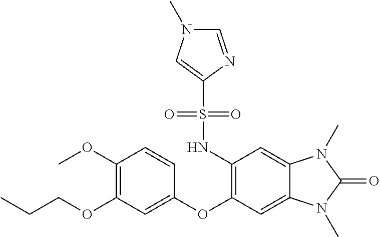 [SMILES: CCCOc1cc(Oc2cc3c(cc2NS(=O)(=O)c2cn(C)cn2)n(C)c(=O)n3C)ccc1OC] | N-(6-(4-methoxy- 5- propoxyphenoxy)- 1,3-dimethyl-2- oxo-2,3-dihydro- 1H- benzo[d]imidazol- 5-yl)-1-methyl- 1H-imidazole-4- sulfonamide | 501 | 502 | A |
| 115 | 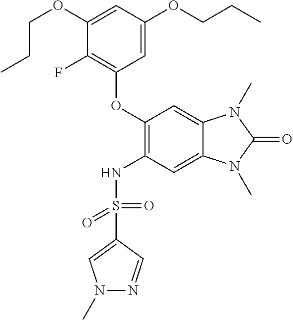 [SMILES: CCCOc1cc(OCCC)c(F)c(Oc2cc3c(cc2NS(=O)(=O)c2cnn(C)c2)n(C)c(=O)n3C)c1] 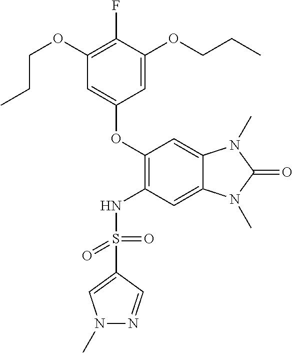 [SMILES: CCCOc1cc(Oc2cc3c(cc2NS(=O)(=O)c2cnn(C)c2)n(C)c(=O)n3C)cc(OCCC)c1F] | N-(6-(2-fluoro- 3,5- dipropoxyphenoxy)- 1,3-dimethyl- 2-oxo-2,3- ddihydro-1H- benzo[d]imidazol- 5-yl)-1-methyl- 1H-pyrazole-4- sulfonamide; N-(6-(4-fluoro-3,5- dipropoxyphenoxy)- 1,3-dimethyl- 2-oxo-2,3- dihydro-1H- benzo[d]imidazol- 5-yl)-1-methyl- 1H-pyrazole-4- sulfonamide 1:1 mixture of isomers | 547 | 548 | A |
| 116 | 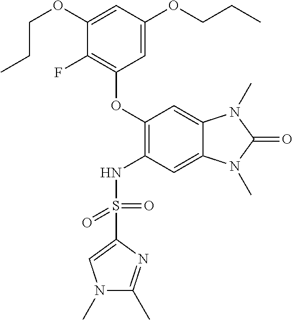 [SMILES: CCCOc1cc(OCCC)c(F)c(Oc2cc3c(cc2NS(=O)(=O)c2cn(C)c(C)n2)n(C)c(=O)n3C)c1] 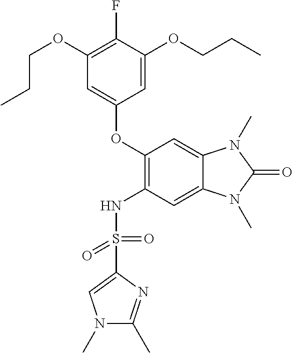 [SMILES: CCCOc1cc(Oc2cc3c(cc2NS(=O)(=O)c2cn(C)c(C)n2)n(C)c(=O)n3C)cc(OCCC)c1F] | N-(6-(2-fluoro- 3,5- dipropoxyphenoxy)- 1,3-dimethyl- 2-oxo-2,3- dihydro-1H- benzo[d]imidazol- 5-yl)-2- dimethyl-1H- imidazole-4- sulfonamide; N- (6-(4-fluoro-3,5- dipropoxyphenoxy)- 1,3-dimethyl- 2-oxo-2,3- dihydro-1H- benzo[d]imidazol- 5-yl)-1,2- dimethyl-1H- imidazole-4- sulfonamide 1:1 mixture of isomers | 561 | 562 | A |
| 117 | 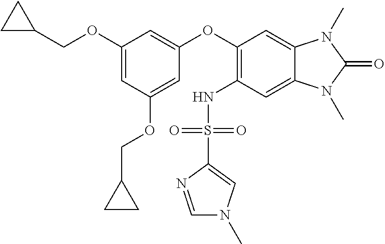 [SMILES: Cn1cnc(S(=O)(=O)Nc2cc3c(cc2Oc2cc(OCC4CC4)cc(OCC4CC4)c2)n(C)c(=O)n3C)c1] | N-(6-(3,5- bis(cyclopropyl- methoxy)phenoxy)- 1,3-dimethyl-2- oxo-2,3-dihydro- 1H- benzo[d]imidazol- 5-yl)-1-methyl- 1H-imidazole-4- sulfonamide | 553 | 554 | C |
| 118 | 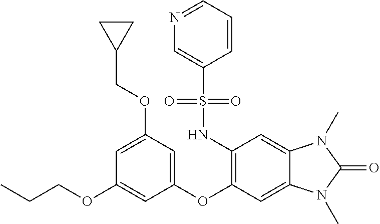 [SMILES: CCCOc1cc(OCC2CC2)cc(Oc2cc3c(cc2NS(=O)(=O)c2cccnc2)n(C)c(=O)n3C)c1] | N-(6-(3- (cyclopropylmethoxy)- 5- propoxyphenoxy)- 1,3-dimethyl-2- oxo-2,3-dihydro- 1H- benzo[d]imidazol- 5-yl)pyridine-3- sulfonamide | 538 | 539 | C |
| 119 | 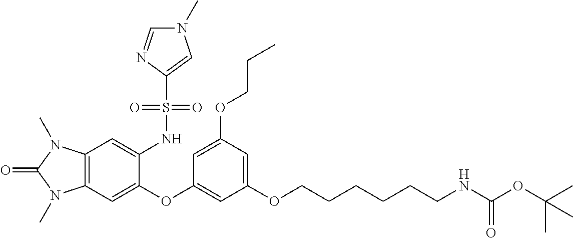 [SMILES: CCCOc1cc(OCCCCCCNC(=O)OC(C)(C)C)cc(Oc2cc3c(cc2NS(=O)(=O)c2cn(C)cn2)n(C)c(=O)n3C)c1] | tert-butyl (6-(3- ((1,3-dimethyl-6- ((1-methyl-1H- imidazole)-4- sulfonamido)-2- oxo-2,3-dihydro- 1H- benzo[d]imidazol- 5-yl)oxy)-5- propoxyphenoxy) hexyl)carbamate | 686 | 687 | C |
| 120 | 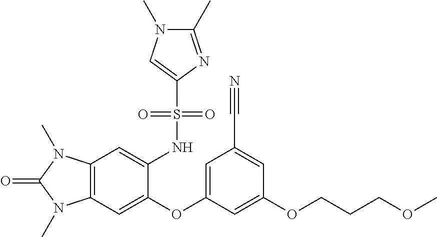 [SMILES: COCCCOc1cc(C#N)cc(Oc2cc3c(cc2NS(=O)(=O)c2cn(C)c(C)n2)n(C)c(=O)n3C)c1] | N-(6-(3-cyano-5- (3- methoxypropoxy) phenoxy)-1,3- dimethyl-2-oxo- 2,3-dihydro-1H- benzo[d]imidazol- 5-yl)-1,2- dimethyl-1H- imidazole-4- sulfonamide | 540 | 541 | C |
| 121 | 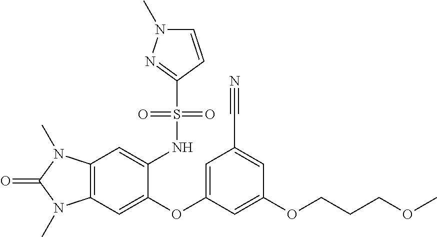 [SMILES: COCCCOc1cc(C#N)cc(Oc2cc3c(cc2NS(=O)(=O)c2ccn(C)n2)n(C)c(=O)n3C)c1] | N-(6-(3-cyano-5- (3- methoxypropoxy) phenoxy)-1,3- dimethyl-2-oxo- 2,3-dihydro-1H- benzo[d]imidazol- 5-yl)-1-methyl- 1H-pyrazole-3- sulfonamide | 526 | 527 | C |
| 122 | 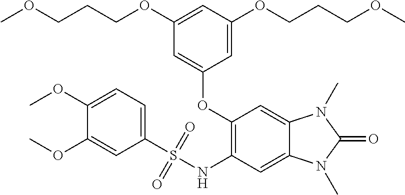 | N-(6-(3,5-bis(3- methoxypropoxy) phenoxy)-1,3- dimethyl-2-oxo- 2,3-dihydro-1H- benzo[d]imidazol- 5-yl)-3,4- dimethoxybenzene- sulfonamide | 645 | 646 | A |
| 123 | 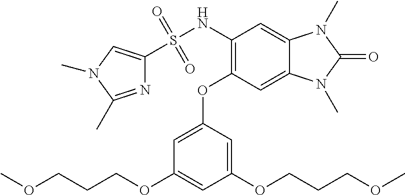 | N-(6-(3,5-bis(3- methoxypropoxy) phenoxy)-1,3- dimethyl-2-oxo- 2,3-dihydro-1H- benzo[d]imidazol- 5-yl)-1,2- dimethyl-1H- imidazole-4- sulfonamide | 603 | 604 | A |
| 124 | 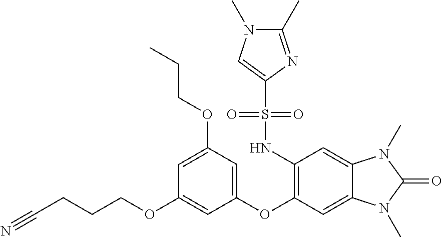 [SMILES: CCCOc1cc(OCCCC#N)cc(Oc2cc3c(cc2NS(=O)(=O)c2cn(C)c(C)n2)n(C)c(=O)n3C)c1] | N-(6-(3-(3- cyanopropoxy)-5- propoxyphenoxy)- 1,3-dimethyl-2- oxo-2,3-dihydro- 1H- benzo[d]imidazol- 5-yl)-1,2- dimethyl-1H- imidazole-4- sulfonamide | 568 | 569 | A |
| 125 | 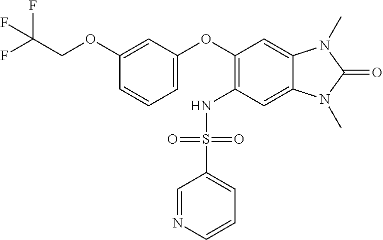 [SMILES: Cn1c(=O)n(C)c2cc(Oc3cccc(OCC(F)(F)F)c3)c(NS(=O)(=O)c3cccnc3)cc21] | N-(1,3-dimethyl- 2-oxo-6-(3-(2,2,2- trifluoroethoxy) phenoxy)-2,3- dihydro-1H- benzo[d]imidazol- 5-yl)pyridin-3- sulfonamide | 508 | 509 | C |
| 126 | 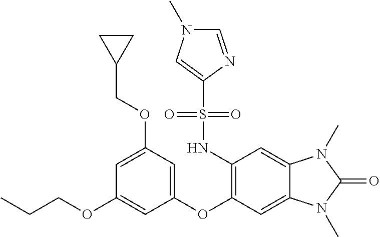 [SMILES: CCCOc1cc(OCC2CC2)cc(Oc2cc3c(cc2NS(=O)(=O)c2cn(C)cn2)n(C)c(=O)n3C)c1] | N-(6-(3- (cyclopropylmethoxy)- 5- propoxyphenoxy)- 1,3-dimethyl-2- oxo-2,3-dihydro- 1H- benzo[d]imidazol- 5-yl)-1-methyl- 1H-imidazole-4- sulfonamide | 541 | 542 | C |
| 127 | 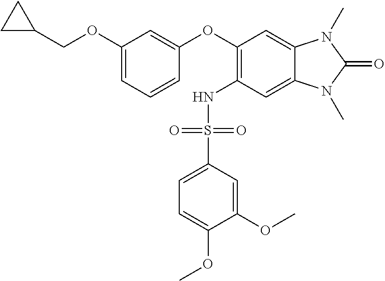 [SMILES: COc1ccc(S(=O)(=O)Nc2cc3c(cc2Oc2cccc(OCC4CC4)c2)n(C)c(=O)n3C)cc1OC] | N-(6-(3- (cyclopropylmethoxy) phenoxy)- 1,3-dimethyl-2- oxo-2,3-dihydro- 1H- benzo[d]imidazol- 5-yl)-3,4- dimethoxybenzene- sulfonamide | 539 | 540 | A |
| 128 | 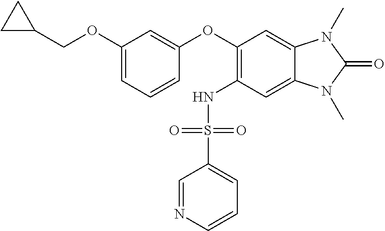 [SMILES: Cn1c(=O)n(C)c2cc(Oc3cccc(OCC4CC4)c3)c(NS(=O)(=O)c3cccnc3)cc21] | N-(6-(3- (cyclopropylmethoxy) phenoxy)- 1,3-dimethyl-2- oxo-2,3-dihydro- 1H- benzo[d]imidazol- 5-yl)pyridine-3- sulfonamide | 480 | 481 | A |
| 129 | 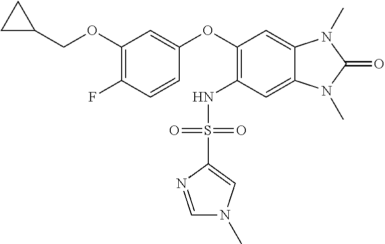 | N-(6-(3- (cyclopropylmethoxy)- 4- fluorophenoxy)- 1,3-dimethyl-2- oxo-2,3-dihydro- 1H- benzo[d]imidazol- 5-yl)-1-methyl- 1H-imidazole-4- sulfonamide | 515 | 516 | A |
| 130 | 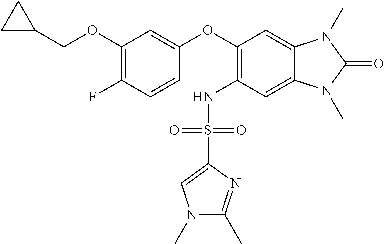 [SMILES: Cc1nc(S(=O)(=O)Nc2cc3c(cc2Oc2ccc(F)c(OCC4CC4)c2)n(C)c(=O)n3C)cn1C] | N-(6-(3- (cyclopropylmethoxy)- 4- fluorophenoxy)- 1,3-dimethyl-2- oxo-2,3-dihydro- 1H- benzo[d]imidazol- 5-yl)-1,2- dimethyl-1H- imidazole-4- sulfonamide | 515 | 516 | A |
| 131 | 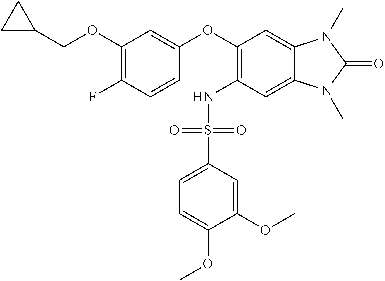 [SMILES: COc1ccc(S(=O)(=O)Nc2cc3c(cc2Oc2ccc(F)c(OCC4CC4)c2)n(C)c(=O)n3C)cc1OC] | N-(6-(3- (cyclopropylmethoxy)- 4- fluorophenoxy)- 1,3-dimethyl-2- oxo-2,3-dihydro- 1H- benzo[d]imidazol- 5-yl)-3,4- dimethoxybenzene- sulfonamide | 557 | 558 | A |
| 132 | 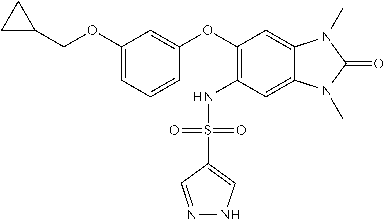 [SMILES: Cn1c(=O)n(C)c2cc(Oc3cccc(OCC4CC4)c3)c(NS(=O)(=O)c3cn[nH]c3)cc21] | N-(6-(3- (cyclopropylmethoxy) phenoxy)- 1,3-dimethyl-2- oxo-2,3-dihydro- 1H- benzo[d]imidazol- 5-yl)-1H- pyrazole-4- sulfonamide | 469 | 470 | A |
| 133 | 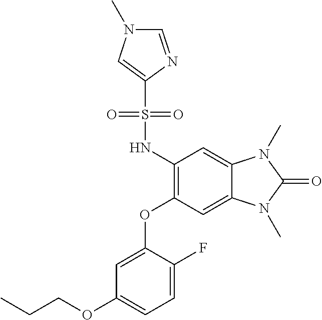 [SMILES: CCCOc1ccc(F)c(Oc2cc3c(cc2NS(=O)(=O)c2cn(C)cn2)n(C)c(=O)n3C)c1] | N-(6-(2-fluoro-5- propoxyphenoxy)- 1,3-dimethyl-2- oxo-2,3-ddihydro- 1H- benzo[d]imidazol- 5-yl)-1,2- dimethyl-1H- imidazole-4- sulfonamide | 503 | 504 | A |
| 134 | 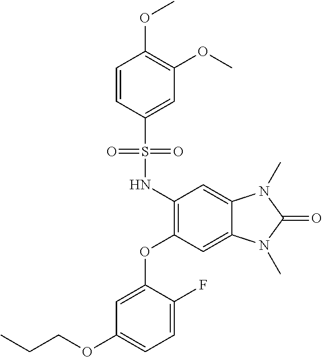 [SMILES: CCCOc1ccc(F)c(Oc2cc3c(cc2NS(=O)(=O)c2ccc(OC)c(OC)c2)n(C)c(=O)n3C)c1] | N-(6-(2-fluoro-5- propoxyphenoxy)- 1,3-dimethyl-2- oxo-2,3-dihydro- 1H- benzo[d]imidazol- 5-yl)-3,4- dimethoxybenzene- sulfonamide | 545 | 546 | A |
| 135 | 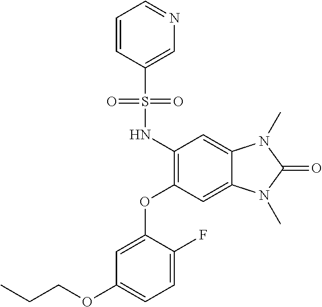 [SMILES: CCCOc1ccc(F)c(Oc2cc3c(cc2NS(=O)(=O)c2cccnc2)n(C)c(=O)n3C)c1] | N-(6-(2-fluoro-5- propoxyphenoxy)- 1,3-dimethyl-2- oxo-2,3-dihydro- 1H- benzo[d]imidazol- 5-yl)pyridine-3- sulfonamide | 486 | 487 | A |
| 136 | 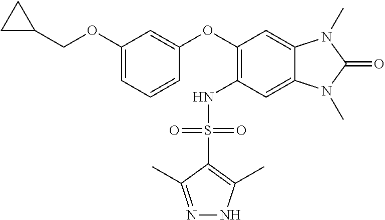 [SMILES: Cc1n[nH]c(C)c1S(=O)(=O)Nc1cc2c(cc1Oc1cccc(OCC3CC3)c1)n(C)c(=O)n2C] | N-(6-(3-(cyclopropyl- methoxy)phenoxy)- 1,3-dimethyl-2- oxo-2,3-dihydro- 1H- benzo[d]imidazol- 5-yl)-3,5- dimethyl-1H- pyrazole-4- sulfonamide | 497 | 498 | A |
| 137 | 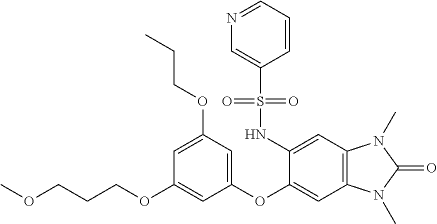 [SMILES: CCCOc1cc(OCCCOC)cc(Oc2cc3c(cc2NS(=O)(=O)c2cccnc2)n(C)c(=O)n3C)c1] | N-(6-(3-(3- methoxypropoxy)- 5- propoxyphenoxy)- 1,3-dimethyl-2- oxo-2,3-dihydro- 1H- benzo[d]imidazol- 5-yl)pyridine-3- sulfonamide | 536 | 537 | C |
| 138 | 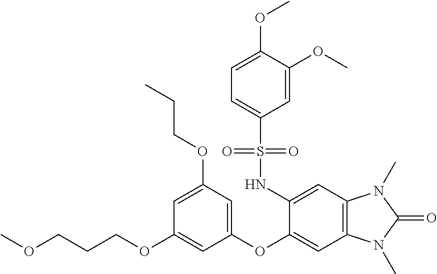 [SMILES: CCCOc1cc(OCCCOC)cc(Oc2cc3c(cc2NS(=O)(=O)c2ccc(OC)c(OC)c2)n(C)c(=O)n3C)c1] | 3,4-dimethoxy-N- (6-(3-(3- methoxypropoxy)- 5- propoxyphenoxy)- 1,3-dimethyl-2- oxo-2,3-dihydro- 1H- benzo[d]imidazol- 5- yl)benzene- sulfonamide | 615 | 616 | C |
| 139 | 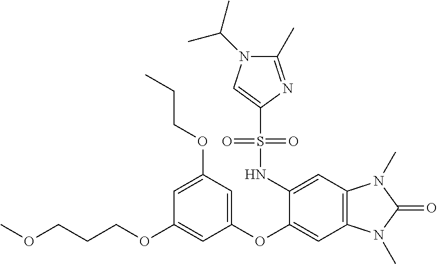 [SMILES: CCCOc1cc(OCCCOC)cc(Oc2cc3c(cc2NS(=O)(=O)c2cn(C(C)C)c(C)n2)n(C)c(=O)n3C)c1] | 1-isopropyl-N-(6- (3-(3- methoxypropoxy)- 5- propoxyphenoxy)- 1,3-dimethyl-2- oxo-2,3-dihydro- 1H- benzo[d]imidazol- 5-yl)-2-methyl- 1H-imidazole-4- sulfonamide | 601 | 602 | C |
| 140 | 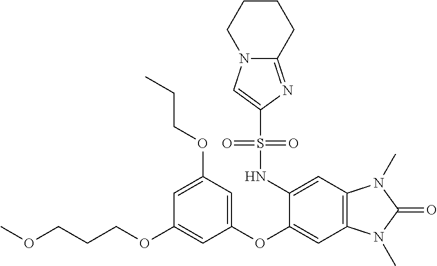 [SMILES: CCCOc1cc(OCCCOC)cc(Oc2cc3c(cc2NS(=O)(=O)c2cn4c(n2)CCCC4)n(C)c(=O)n3C)c1] | N-(6-(3-(3- methoxypropoxy)- 5- propoxyphenoxy)- 1,3-dimethyl-2- oxo-2,3-dihydro- 1H- benzo[d]imidazol- 5-yl)-5,6,7,8- tetrahydroimidazo [1,2-a]pyridine-2- sulfonamide | 599 | 600 | C |
| 141 | 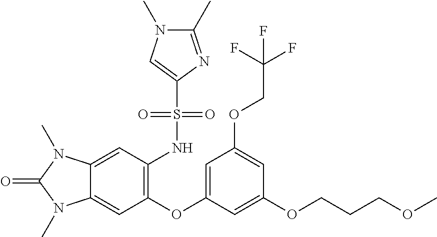 [SMILES: COCCCOc1cc(OCC(F)(F)F)cc(Oc2cc3c(cc2NS(=O)(=O)c2cn(C)c(C)n2)n(C)c(=O)n3C)c1] | N-(6-(3-(3- methoxypropoxy)- 5-(2,2,2- trifluoroethoxy) phenoxy)-1,3- dimethyl-2-oxo- 2,3-dihydro-1H- benzo[d]imidazol- 5-yl)-1,2- dimethyl-1H- imidazole-4- sulfonamide | 613 | 614 | C |
| 142 | 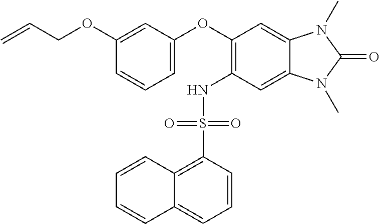 [SMILES: C=CCOc1cccc(Oc2cc3c(cc2NS(=O)(=O)c2cccc4ccccc24)n(C)c(=O)n3C)c1] | N-(6-(3- (allyloxy)phenoxy)- 1,3-dimethyl- 2-oxo-2,3- dihydro-1H- benzo[d]imidazol- 5- yl)naphthalene-1- sulfonamide | 515 | 516 | B |
| 143 | 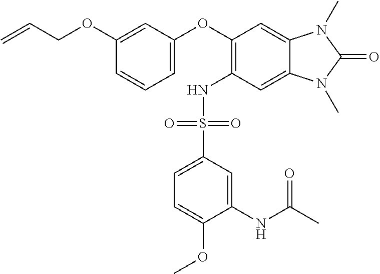 [SMILES: C=CCOc1cccc(Oc2cc3c(cc2NS(=O)(=O)c2ccc(OC)c(NC(C)=O)c2)n(C)c(=O)n3C)c1] | N-(5-(N-(6-(3- (allyloxy)phenoxy)- 1,3-dimethyl- 2-oxo-2,3- dihydro-1H- benzo[d]imidazol- 5-yl)sulfamoyl)- 2- methoxyphenyl) acetamide | 552 | 553 | B |
| 144 | 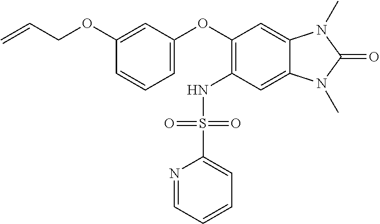 [SMILES: C=CCOc1cccc(Oc2cc3c(cc2NS(=O)(=O)c2ccccn2)n(C)c(=O)n3C)c1] | N-(6-(3- (allyloxy)phenoxy)- 1,3-dimethyl- 2-oxo-2,3- dihydro-1H- benzo[d]imidazol- 5-yl)pyridine-2- sulfonamide | 466 | 467 | B |
| 145 | 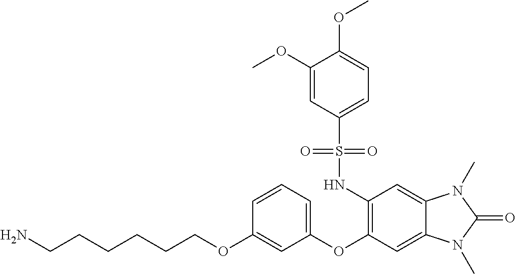 [SMILES: COc1ccc(S(=O)(=O)Nc2cc3c(cc2Oc2cccc(OCCCCCCN)c2)n(C)c(=O)n3C)cc1OC] | N-(6-(3-((6- aminohexyl)oxy) phenoxy)-1,3- dimethyl-2-oxo- 2,3-dihydro-1H- benzo[d]imidazol- 5-yl)-3,4- dimethoxybenzene- sulfonamide | 584 | 585 | E |
| 146 | 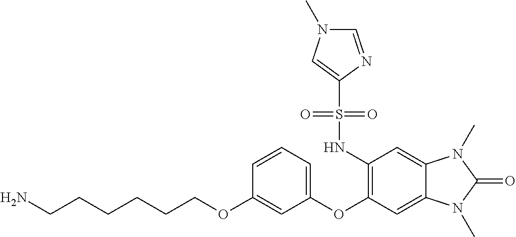 [SMILES: Cn1cnc(S(=O)(=O)Nc2cc3c(cc2Oc2cccc(OCCCCCCN)c2)n(C)c(=O)n3C)c1] | N-(6-(3-((6- aminohexyl)oxy) phenoxy)-1,3- dimethyl-2-oxo- 2,3-dimethyl-2-oxo- 2,3-dihydro-1H- benzo[d]imidazol- 5-yl)-1-methyl- 1H-imidazole-4- sulfonamide | 528 | 529 | E |
| 147 | 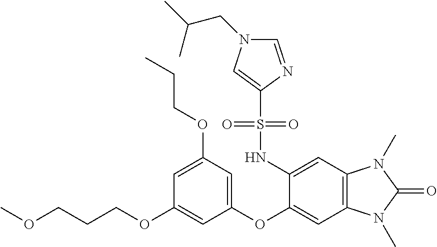 [SMILES: CCCOc1cc(OCCCOC)cc(Oc2cc3c(cc2NS(=O)(=O)c2cn(CC(C)C)cn2)n(C)c(=O)n3C)c1] | 1-isobutyl-N-(6- (3-(3- methoxypropoxy)- 5- propoxyphenoxy)- 1,3-dimethyl-2- oxo-2,3-dihydro- 1H- benzo[d]imidazol- 5-yl)-1H- imidazole-4- sulfonamide | 601 | 602 | C |
| 148 | 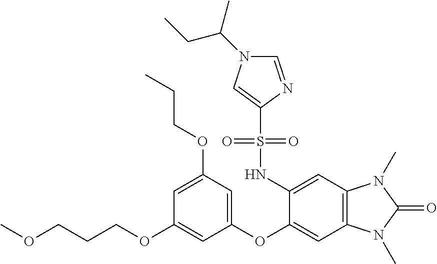 [SMILES: CCCOc1cc(OCCCOC)cc(Oc2cc3c(cc2NS(=O)(=O)c2cn(C(C)CC)cn2)n(C)c(=O)n3C)c1] | 1-(sec-butyl)-N- (6-(3-(3- methoxypropoxy)- 5- propoxyphenoxy)- 1,3-dimethyl-2- oxo-2,3-dihydro- 1H- benzo[d]imidazol- 5-yl)-1H- imidazole-4- sulfonamide | 601 | 602 | C |
| 149 | 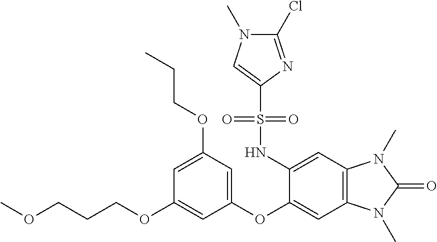 [SMILES: CCCOc1cc(OCCCOC)cc(Oc2cc3c(cc2NS(=O)(=O)c2cn(C)c(Cl)n2)n(C)c(=O)n3C)c1] | 2-chloro-N-(6-(3- (3- methoxypropoxy)- 5- propoxyphenoxy)- 1,3-dimethyl-2- oxo-2,3-dihydro- 1H- benzo[d]imidazol- 5-yl)-1-methyl- 1H-imidazole-4- sulfonamide | 593 | 594 | C |
| 150 | 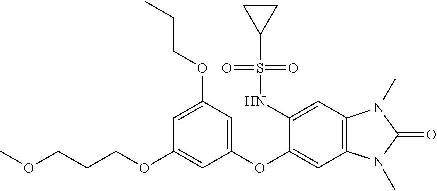 [SMILES: CCCOc1cc(OCCCOC)cc(Oc2cc3c(cc2NS(=O)(=O)C2CC2)n(C)c(=O)n3C)c1] | N-(6-(3-(3- methoxypropoxy)- 5- propoxyphenoxy)- 1,3-dimethyl-2- oxo-2,3-dihydro- 1H- benzo[d]imidazol- 5- yl)cyclopropane- sulfonamide | 519 | 520 | C |
| 151 | 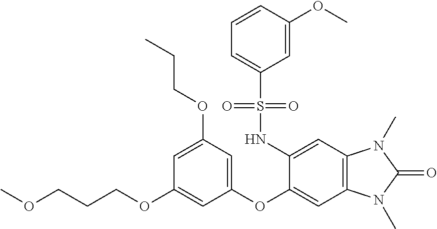 [SMILES: CCCOc1cc(OCCCOC)cc(Oc2cc3c(cc2NS(=O)(=O)c2cccc(OC)c2)n(C)c(=O)n3C)c1] | 3-methoxy-N-(6- (3-(3- methoxypropoxy)- 5- propoxyphenoxy)- 1,3-dimethyl-2- oxo-2,3-dihydro- 1H- benzo[d]imidazol- 5- yl)benzenesulfonamide | 585 | 586 | C |
| 152 | 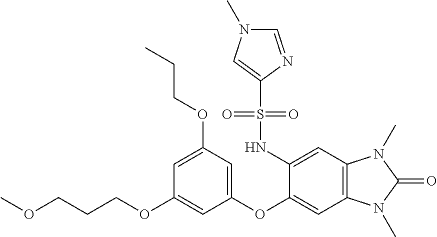 [SMILES: CCCOc1cc(OCCCOC)cc(Oc2cc3c(cc2NS(=O)(=O)c2cn(C)cn2)n(C)c(=O)n3C)c1] | N-(6-(3-(3- methoxypropoxy)- 5- propoxyphenoxy)- 1,3-dimethyl-2- oxo-2,3-dihydro- 1H- benzo[d]imidazol- 5-yl)-1-methyl- 1H-imidazole-4- sulfonamide | 559 | 560 | C |
| 153 | 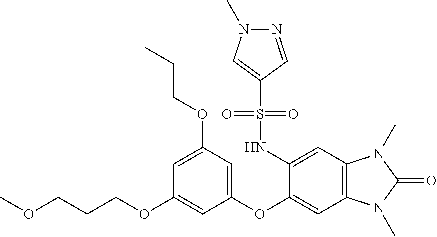 [SMILES: CCCOc1cc(OCCCOC)cc(Oc2cc3c(cc2NS(=O)(=O)c2cnn(C)c2)n(C)c(=O)n3C)c1] | N-(6-(3-(3- methoxypropoxy)- 5- propoxyphenoxy)- 1,3-dimethyl-2- oxo-2,3-dihydro- 1H- benzo[d]imidazol- 5-yl)-1-methyl- 1H-pyrazole-4- sulfonamide | 559 | 560 | C |
| 154 | 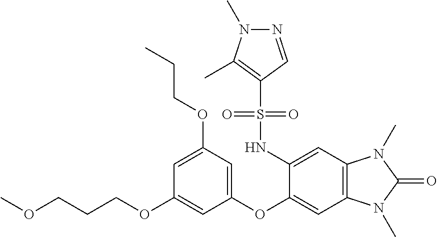 [SMILES: CCCOc1cc(OCCCOC)cc(Oc2cc3c(cc2NS(=O)(=O)c2cnn(C)c2C)n(C)c(=O)n3C)c1] | N-(6-(3-(3- methoxypropoxy)- 5- propoxyphenyl)- 1,3-dimethyl-2- oxo-2,3-dihydro- 1H- benzo[d]imidazol- 5-yl)-1,5- dimethyl-1H- pyrazole-4- sulfonamide | 573 | 574 | C |
| 155 | 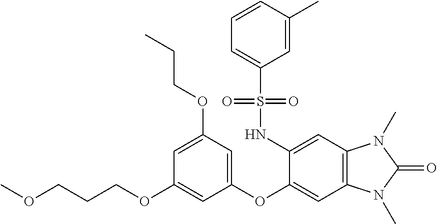 [SMILES: CCCOc1cc(OCCCOC)cc(Oc2cc3c(cc2NS(=O)(=O)c2cccc(C)c2)n(C)c(=O)n3C)c1] | N-(6-(3-(3- methoxypropoxy)- 5- propoxyphenoxy)- 1,3-dimethyl-2- oxo-2,3-dihydro- 1H- benzo[d]imidazol- 5-yl)-3- methylbenzene- sulfonamide | 569 | 570 | C |
| 156 | 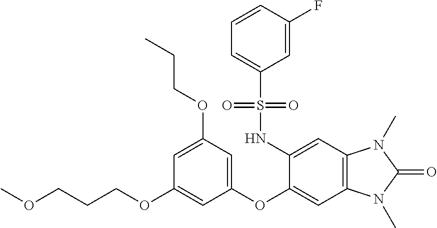 [SMILES: CCCOc1cc(OCCCOC)cc(Oc2cc3c(cc2NS(=O)(=O)c2cccc(F)c2)n(C)c(=O)n3C)c1] | 3-fluoro-N-(6-(3- (3- methoxypropoxy)- 5- propoxyphenoxy)- 1,3-dimethyl-2- oxo-2,3-dihydro- 1H- benzo[d]imidazol- 5- yl)benzenesulfonamide | 573 | 574 | C |
| 157 | 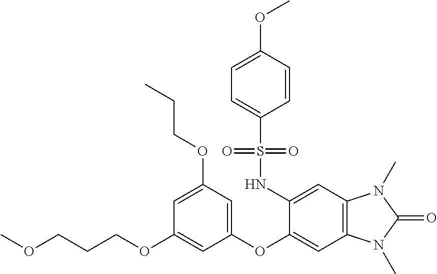 [SMILES: CCCOc1cc(OCCCOC)cc(Oc2cc3c(cc2NS(=O)(=O)c2ccc(OC)cc2)n(C)c(=O)n3C)c1] | 4-methoxy-N-(6- (3-(3- methoxypropoxy)- 5- propoxyphenoxy)- 1,3-dimethyl-2- oxo-2,3-dihydro- 1H- benzo[d]imidazol- 5- yl)benzenesulfonamide | 585 | 586 | C |
| 158 | 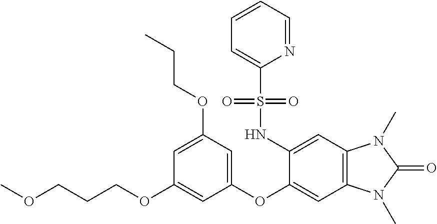 [SMILES: CCCOc1cc(OCCCOC)cc(Oc2cc3c(cc2NS(=O)(=O)c2ccccn2)n(C)c(=O)n3C)c1] | N-(6-(3-(3- methoxypropoxy)- 5- propoxyphenoxy)- 1,3-dimethyl-2- oxo-2,3-dihydro- 1H- benzo[d]imidazol- 5-yl)pyridine-2- sulfonamide | 556 | 557 | C |
| 159 | 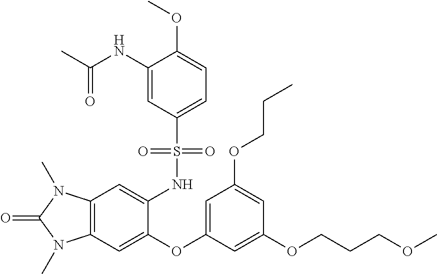 [SMILES: CCCOc1cc(OCCCOC)cc(Oc2cc3c(cc2NS(=O)(=O)c2ccc(OC)c(NC(C)=O)c2)n(C)c(=O)n3C)c1] | N-(2-methoxy-5- (N-(6-(3-(3- methoxypropoxy)- 5- propoxyphenoxy)- 1,3-dimethyl-2- oxo-2,3-dihydro- 1H- benzo[d]imidazol- 5- yl)sulfamoyl) phenyl)acetamide | 642 | 643 | C |
| 160 | 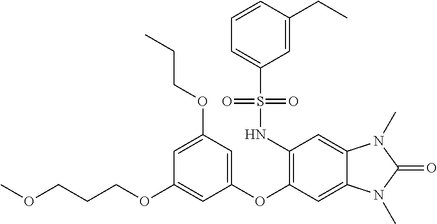 [SMILES: CCCOc1cc(OCCCOC)cc(Oc2cc3c(cc2NS(=O)(=O)c2cccc(CC)c2)n(C)c(=O)n3C)c1] | 3-ethyl-N-(6-(3- (3- methoxypropoxy)- 5- propoxyphenoxy)- 1,3-dimethyl-2- oxo-2,3-dihydro- 1H- benzo[d]imidazol- 5- yl)benzenesulfonamide | 583 | 584 | C |
| 161 | 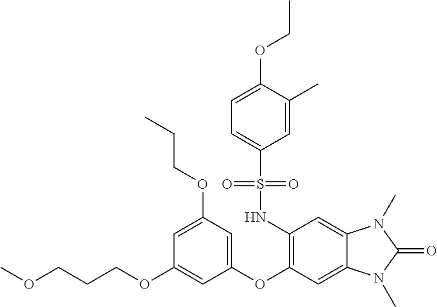 [SMILES: CCCOc1cc(OCCCOC)cc(Oc2cc3c(cc2NS(=O)(=O)c2ccc(OCC)c(C)c2)n(C)c(=O)n3C)c1] | 4-ehoxy-N-(6-(3- (3- methoxypropoxy)- 5- propoxyphenoxy)- 1,3-dimethyl-2- oxo-2,3-dihydro- benzo[d]imidazol- 5-yl)-3- methylbenzene- sulfonamide | 613 | 614 | C |
| 162 | 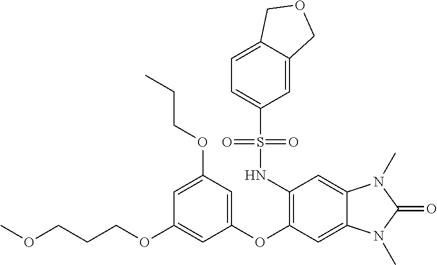 [SMILES: CCCOc1cc(OCCCOC)cc(Oc2cc3c(cc2NS(=O)(=O)c2ccc4c(c2)COC4)n(C)c(=O)n3C)c1] | N-(6-(3-(3- methoxypropoxy)- 5- propoxyphenoxy)- 1,3-dimethyl-2- oxo-2,3-dihydro- 1H- benzo[d]imidazol- 5-yl)-1,3- dihydroisobenzo- furan-5- sulfonamide | 597 | 598 | C |
| 163 | 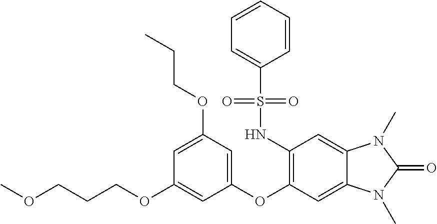 [SMILES: CCCOc1cc(OCCCOC)cc(Oc2cc3c(cc2NS(=O)(=O)c2ccccc2)n(C)c(=O)n3C)c1] | N-(6-(3-(3- methoxypropoxy)- 5- propoxyphenoxy)- 1,3-dimethyl-2- oxo-2,3-dihydro- 1H- benzo[d]imidazol- 5- yl)benzenesulfonamide | 555 | 556 | C |
| 164 | 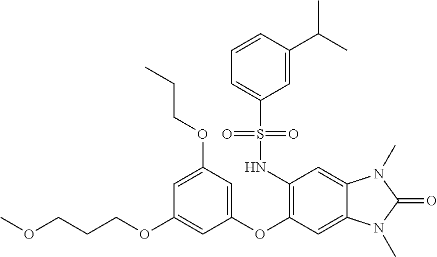 [SMILES: CCCOc1cc(OCCCOC)cc(Oc2cc3c(cc2NS(=O)(=O)c2cccc(C(C)C)c2)n(C)c(=O)n3C)c1] | 3-isopropyl-N-(6- (3-(3- methoxypropoxy)- 5- propoxyphenoxy)- 1,3-dimethyl-2- oxo-2,3-dihydro- 1H- benzo[d]imidazol- 5- yl)benzenesulfonamide | 597 | 598 | C |
| 165 | 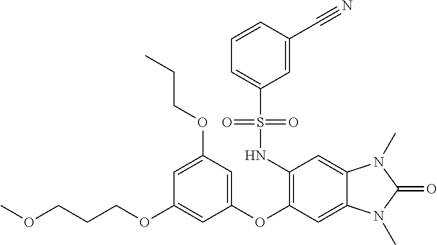 [SMILES: CCCOc1cc(OCCCOC)cc(Oc2cc3c(cc2NS(=O)(=O)c2cccc(C#N)c2)n(C)c(=O)n3C)c1] | 3-cyano-N-(6-(3- (3- methoxypropoxy)- 5- propoxyphenoxy)- 1,3-dimethyl-2- oxo-2,3-dihydro- 1H- benzo[d]imidazol- 5- yl)benzenesulfonamide | 580 | 581 | C |
| 166 | 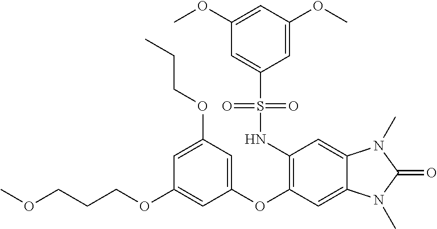 [SMILES: CCCOc1cc(OCCCOC)cc(Oc2cc3c(cc2NS(=O)(=O)c2cc(OC)cc(OC)c2)n(C)c(=O)n3C)c1] | 3,5-dimethoxy-N- (6-(3-(3- methoxypropoxy)- 5- propoxyphenoxy)- 1,3-dimethyl-2- oxo-2,3-dihydro- 1H- benzo[d]imidazol- 5- yl)benzenesulfonamide | 615 | 616 | C |
| 167 | 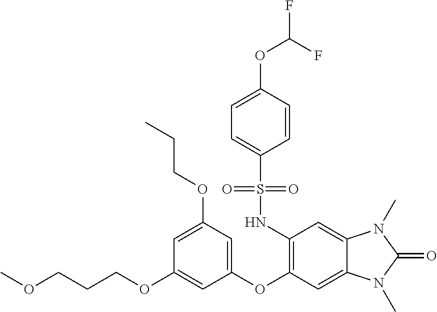 [SMILES: CCCOc1cc(OCCCOC)cc(Oc2cc3c(cc2NS(=O)(=O)c2ccc(OC(F)F)cc2)n(C)c(=O)n3C)c1] | 4- (difluoromethoxy)- N-(6-(3-(3- methoxypropoxy)- 5- propoxyphenoxy)- 1,3-dimethyl-2- oxo-2,3-dihydro- 1H- benzo[d]imidazol- 5- yl)benzenesulfonamide | 621 | 622 | C |
| 168 | 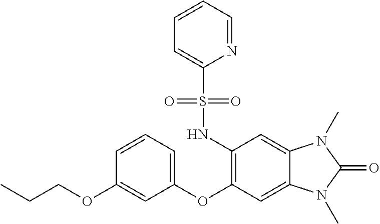 [SMILES: CCCOc1cccc(Oc2cc3c(cc2NS(=O)(=O)c2ccccn2)n(C)c(=O)n3C)c1] | N-(1,3-dimethyl- 2-oxo-6-(3- propoxyphenoxy)- 2,3-dihydro-1H- benzo[d]imidazol- 5-yl)pyridine-2- sulfonamide | 468 | 469 | A |
| 169 | 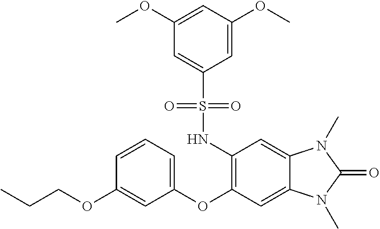 [SMILES: CCCOc1cccc(Oc2cc3c(cc2NS(=O)(=O)c2cc(OC)cc(OC)c2)n(C)c(=O)n3C)c1] | N-(1,3-dimethyl- 2-oxo-6-(3- propoxyphenoxy)- 2,3-dihydro-1H- benzo[d]imidazol- 5-yl)-3,5- dimethoxybenzene- sulfonamide | 527 | 528 | A |
| 170 | 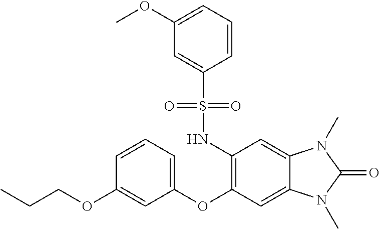 [SMILES: CCCOc1cccc(Oc2cc3c(cc2NS(=O)(=O)c2cccc(OC)c2)n(C)c(=O)n3C)c1] | N-(1,3-dimethyl- 2-oxo-6-(3- propoxyphenoxy)- 2,3-dihydro-1H- benzo[d]imidazol- 5-yl)-3- methoxybenzene- sulfonamide | 497 | 498 | A |
| 171 | 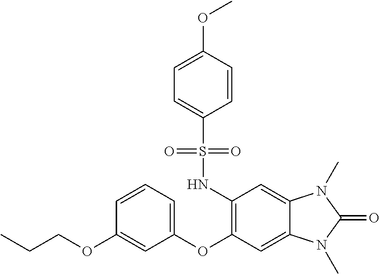 [SMILES: CCCOc1cccc(Oc2cc3c(cc2NS(=O)(=O)c2ccc(OC)cc2)n(C)c(=O)n3C)c1] | N-(1,3-dimethyl- 2-oxo-6-(3- propoxyphenoxy)- 2,3-dihydro-1H- benzo[d]imidazol- 5-yl)-4- methoxybenzene- sulfonamide | 497 | 498 | A |
| 172 | 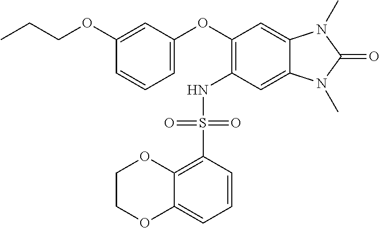 [SMILES: CCCOc1cccc(Oc2cc3c(cc2NS(=O)(=O)c2cccc4c2OCCO4)n(C)c(=O)n3C)c1] | N-(1,3-dimethyl- 2-oxo-6-(3- propoxyphenoxy)- 2,3-dihydro-1H- benzo[d]imidazol- 5-yl)-2,3- dihydrobenzo[b][1,4] dioxine-5- sulfonamide | 525 | 526 | A |
| 173 | 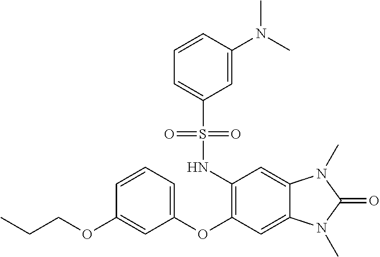 [SMILES: CCCOc1cccc(Oc2cc3c(cc2NS(=O)(=O)c2cccc(N(C)C)c2)n(C)c(=O)n3C)c1] | N-(1,3-dimethyl- 2-oxo-6-(3- propoxyphenoxy)- 2,3-dihydro-1H- benzo[d]imidazol- 5-yl)-3- (dimethylamino) benzenesulfonamide | 510 | 511 | A |
| 174 | 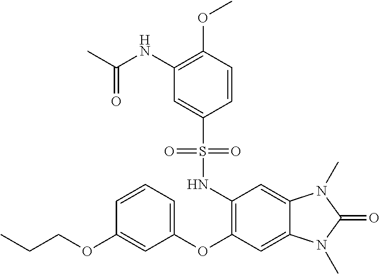 [SMILES: CCCOc1cccc(Oc2cc3c(cc2NS(=O)(=O)c2ccc(OC)c(NC(C)=O)c2)n(C)c(=O)n3C)c1] | N-(5-(N-(1,3- dimethyl-2-oxo- 6-(3- propoxyphenoxy)- 2,3-dihydro-1H- benzo[d]imidazol- 5-yl)sulfamoyl)- methoxyphenyl) acetamide | 554 | 555 | A |
| 175 | 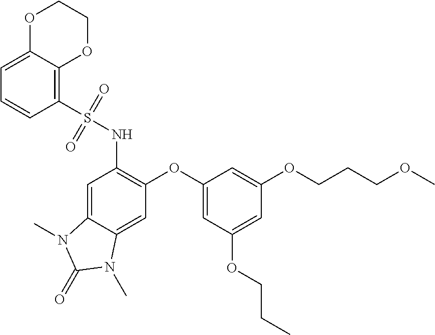 [SMILES: CCCOc1cc(OCCCOC)cc(Oc2cc3c(cc2NS(=O)(=O)c2cccc4c2OCCO4)n(C)c(=O)n3C)c1] | N-(6-(3-(3- methoxypropoxy)- 5- propoxyphenoxy)- 1,3-dimethyl-2- oxo-2,3-dihydro- 1H- benzo[d]imidazol- 5-yl)-2,3- dihydrobenzo[b][1,4] dioxine-5- sulfonamide | 613 | 614 | C |
| 176 | 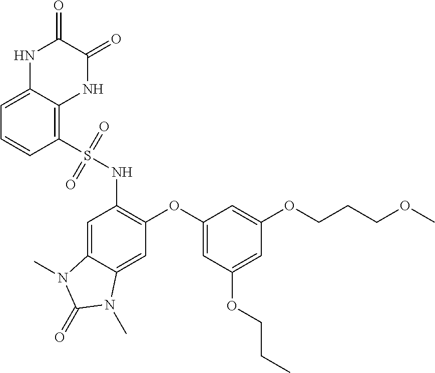 [SMILES: CCCOc1cc(OCCCOC)cc(Oc2cc3c(cc2NS(=O)(=O)c2cccc4[nH]c(=O)c(=O)[nH]c24)n(C)c(=O)n3C)c1] | N-(6-(3-(3- methoxypropoxy)- 5- propoxyphenoxy)- 1,3-dimethyl-2- oxo-2,3-dihydro- 1H- benzo[d]imidazol- 5-yl)-2,3-dioxo- 1,2,3,4- tetrahydroquin- oxaline-5- sulfonamide | 639 | 640 | C |
| 177 | 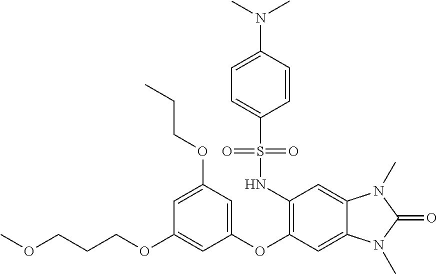 [SMILES: CCCOc1cc(OCCCOC)cc(Oc2cc3c(cc2NS(=O)(=O)c2ccc(N(C)C)cc2)n(C)c(=O)n3C)c1] | 4- (dimethylamino)- N-(6-(3-(3- methoxypropoxy)- 5- propoxyphenoxy)- 1,3-dimethyl-2- oxo-2,3-dihydro- benzo[d]imidazol- 5- yl)benzenesulfonamide | 598 | 599 | C |
| 178 | 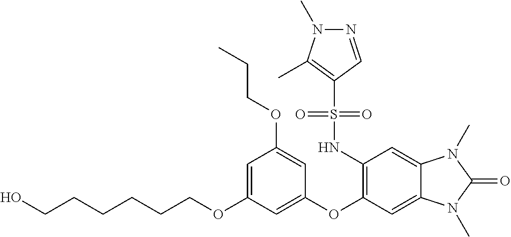 [SMILES: CCCOc1cc(OCCCCCCO)cc(Oc2cc3c(cc2NS(=O)(=O)c2cnn(C)c2C)n(C)c(=O)n3C)c1] | N-(6-(3-((6- hydroxyhexyl)oxy)- 5- propoxyphenoxy)- 1,3-dimethyl-2- oxo-2,3-dihydro- 1H- benzo[d]imidazol- 5-yl)-1,5- dimethyl-1H- pyrazole-4- sulfonamide | 601 | 602 | C |
| 179 | 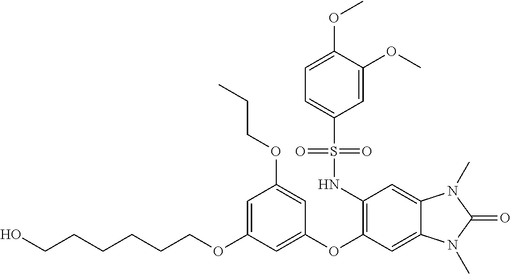 [SMILES: CCCOc1cc(OCCCCCCO)cc(Oc2cc3c(cc2NS(=O)(=O)c2ccc(OC)c(OC)c2)n(C)c(=O)n3C)c1] | N-(6-(3-((6- hydroxyhexyl)oxy)- 5- propoxyphenoxy)- 1,3-dimethyl-2- oxo-2,3-dihydro- 1H- benzo[d]imidazol- 5-yl)-3,4- dimethoxybenzene- sulfonamide | 643 | 644 | C |
| 180 | 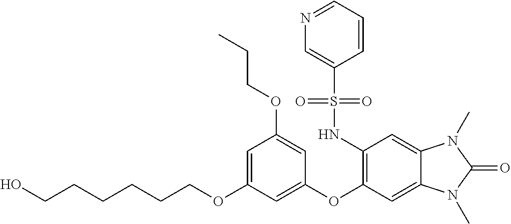 [SMILES: CCCOc1cc(OCCCCCCO)cc(Oc2cc3c(cc2NS(=O)(=O)c2cccnc2)n(C)c(=O)n3C)c1] | N-(6-(3-((6- hydroxyhexyl)oxy)- 5- propoxyphenoxy)- 1,3-dimethyl-2- oxo-2,3-dihydro- 1H- benzo[d]imidazol- 5-yl)pyridine-3- sulfonamide | 584 | 585 | C |
| 181 | 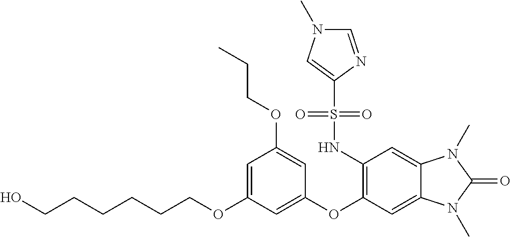 [SMILES: CCCOc1cc(OCCCCCCO)cc(Oc2cc3c(cc2NS(=O)(=O)c2cn(C)cn2)n(C)c(=O)n3C)c1] | N-(6-(3-((6- hydroxyhexyl)oxy)- 5- propoxyphenoxy)- 1,3-dimethyl-2- oxo-2,3-dihydro- 1H- benzo[d]imidazol- 5-yl)-1-methyl- 1H-imidazole-4- sulfonamide | 587 | 588 | C |
| 182 | 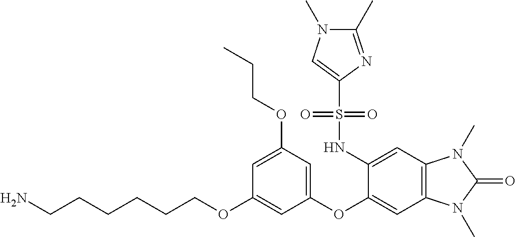 [SMILES: CCCOc1cc(OCCCCCCN)cc(Oc2cc3c(cc2NS(=O)(=O)c2cn(C)c(C)n2)n(C)c(=O)n3C)c1] | N-(6-(3-((6- aminohexyl)oxy)- 5- propoxyphenoxy)- 1,3-dimethyl-2- oxo-2,3-dihydro- 1H- benzo[d]imidazol- 5-yl)-1,2- dimethyl-1H- imidazole-4- sulfonamide | 600 | 601 | C |
| 183 | 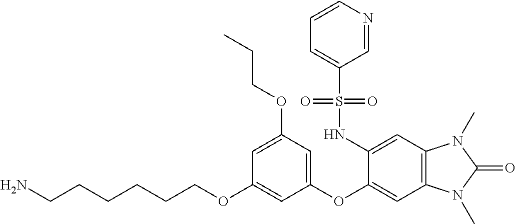 [SMILES: CCCOc1cc(OCCCCCCN)cc(Oc2cc3c(cc2NS(=O)(=O)c2cccnc2)n(C)c(=O)n3C)c1] | N-(6-(3-((6- aminohexyl)oxy)- 5- propoxyphenoxy)- 1,3-dimethyl-2- oxo-2,3-dihydro- 1H- benzo[d]imidazol- 5-yl)pyridine-3- sulfonamide | 583 | 584 | C |
| 184 | 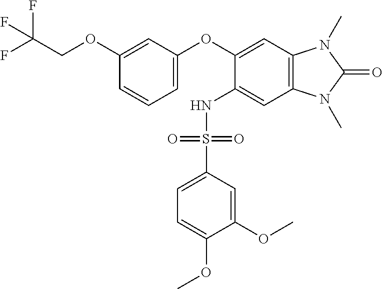 [SMILES: COc1ccc(S(=O)(=O)Nc2cc3c(cc2Oc2cccc(OCC(F)(F)F)c2)n(C)c(=O)n3C)cc1OC] | N-(1,3-dimethyl- 2-oxo-6-(3-(2,2,2- trifluoroethoxy) phenoxy)-2,3- dihydro-1H- benzo[d]imidazol- 5-yl)-3,4- dimethoxybenzene- sulfonamide | 567 | 568 | C |
| 185 | 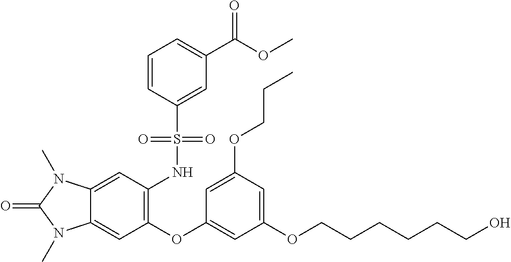 [SMILES: CCCOc1cc(OCCCCCCO)cc(Oc2cc3c(cc2NS(=O)(=O)c2cccc(C(=O)OC)c2)n(C)c(=O)n3C)c1] | methyl 3-(N-(6- (3-((6- hydroxynhexyl)oxy)- 5- propoxyphenoxy)- 1,3-dimethyl-2- oxo-2,3-dihydro- 1H- benzo[d]imidazol- 5- yl)sulfamoyl) benzoate | 641 | 642 | C |
| 186 | 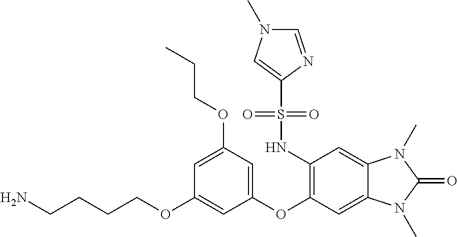 [SMILES: CCCOc1cc(OCCCCN)cc(Oc2cc3c(cc2NS(=O)(=O)c2cn(C)cn2)n(C)c(=O)n3C)c1] | N-(6-(3-(4- aminobutoxy)-5- propoxyphenoxy)- 1,3-dimethyl-2- oxo-2,3-dihydro- 1H- benzo[d]imidazol- 5-yl)-1-methyl- 1H-imidazole-4- sulfonamide | 558 | 559 | F |
| 187 | 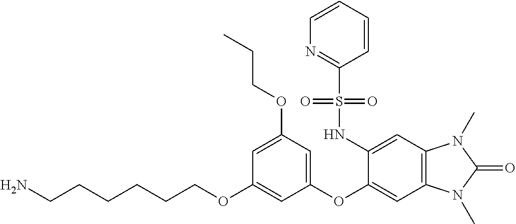 [SMILES: CCCOc1cc(OCCCCCCN)cc(Oc2cc3c(cc2NS(=O)(=O)c2ccccn2)n(C)c(=O)n3C)c1] | N-(6-(3-((6- aminohexyl)oxy)- 5- propoxyphenoxy)- 1,3-dimethyl-2- oxo-2,3-dihydro- 1H- benzo[d]imidazol- 5-yl)pyridine-2- sulfonamide | 583 | 584 | E |
| 188 | 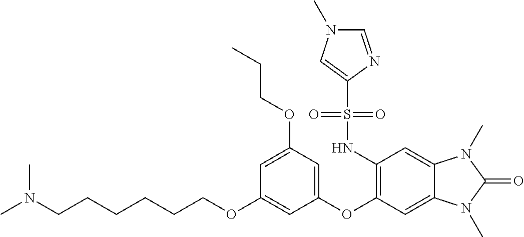 [SMILES: CCCOc1cc(OCCCCCCN(C)C)cc(Oc2cc3c(cc2NS(=O)(=O)c2cn(C)cn2)n(C)c(=O)n3C)c1] | N-(6-(3-((6- (dimethylamino) hexyl)oxy)-5- propoxyphenoxy)- 1,3-dimethyl-2- oxo-2,3-dihydro- 1H- benzo[d]imidazol- 5-yl)-1-methyl- 1H-imidazole-4- sulfonamide | 614 | 615 | F |
| 189 | 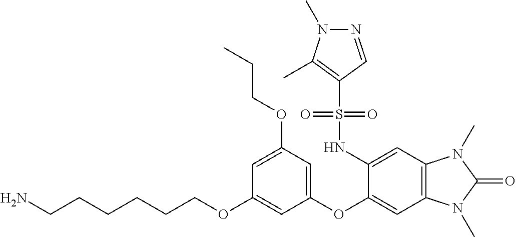 [SMILES: CCCOc1cc(OCCCCCCN)cc(Oc2cc3c(cc2NS(=O)(=O)c2cnn(C)c2C)n(C)c(=O)n3C)c1] | N-(6-(3-((6- aminohexyl)oxy)- 5- propoxyphenoxy)- 1,3-dimethyl-2- oxo-2,3-dihydro- 1H- benzo[d]imidazol- 5-yl)-1,5- dimethyl-1H- pyrazole-4- sulfonamide | 600 | 601 | E |
| 190 | 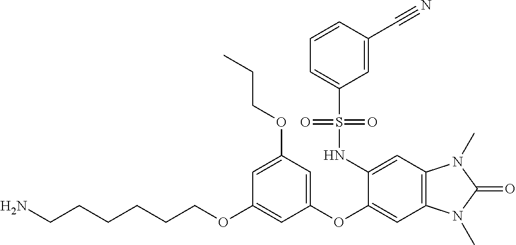 [SMILES: CCCOc1cc(OCCCCCCN)cc(Oc2cc3c(cc2NS(=O)(=O)c2cccc(C#N)c2)n(C)c(=O)n3C)c1] | N-(6-(3-((6- aminohexyl)oxy)- 5- propoxyphenoxy)- 1,3-dimethyl-2- oxo-2,3-dihydro- 1H- benzo[d]imidazol- 5-yl)-3- cyanobenzene- sulfonamide | 607 | 608 | E |
| 191 | 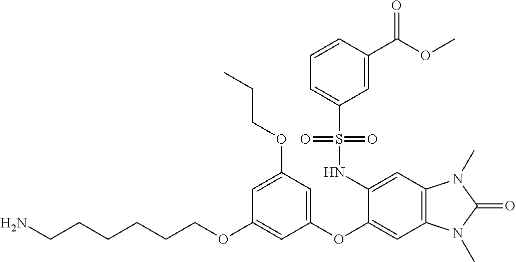 [SMILES: CCCOc1cc(OCCCCCCN)cc(Oc2cc3c(cc2NS(=O)(=O)c2cccc(C(=O)OC)c2)n(C)c(=O)n3C)c1] | methyl 3-(N-(6- (3-((6- aminohexyl)oxy)- 5- propoxyphenoxy)- 1,3-dimethyl-2- oxo-2,3-dihydro- 1H- benzo[d]imidazol- 5- yl)sulfamoyl) benzoate | 640 | 641 | E |
| 192 | 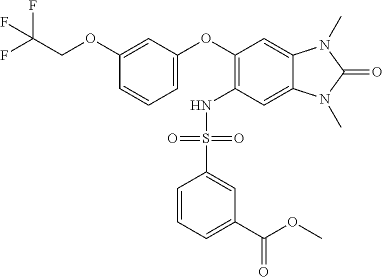 [SMILES: COC(=O)c1cccc(S(=O)(=O)Nc2cc3c(cc2Oc2cccc(OCC(F)(F)F)c2)n(C)c(=O)n3C)c1] | methyl 3-(N-(1,3- dimethyl-2-oxo- 6-(3-(2,2,2- trifluoroethoxy) phenoxy)-2,3- dihydro-1H- benzo[d]imidazol- 5- yl)sulfamoyl) benzoate | 565 | 566 | C |
| 193 | 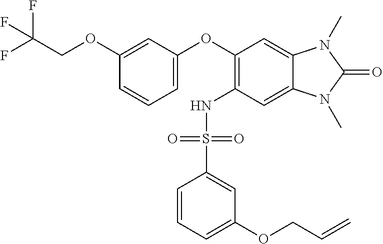 [SMILES: C=CCOc1cccc(S(=O)(=O)Nc2cc3c(cc2Oc2cccc(OCC(F)(F)F)c2)n(C)c(=O)n3C)c1] | 3-(allyloxy)-N- (1,3-dimethyl-2- oxo-6-(3-(2,2,2- trifluoroethoxy) phenoxy)-2,3- dihydro-1H- benzo[d]imidazol- 5- yl)benzenesulfonamide | 563 | 564 | C |
| 194 | 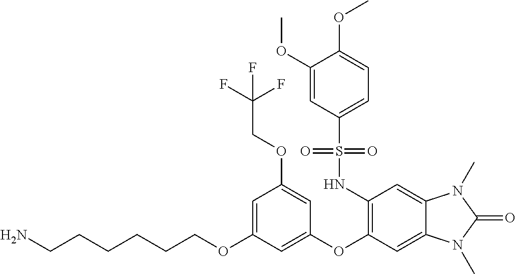 [SMILES: COc1ccc(S(=O)(=O)Nc2cc3c(cc2Oc2cc(OCCCCCCN)cc(OCC(F)(F)F)c2)n(C)c(=O)n3C)cc1OC] | N-(6-(3-((6- aminohexyl)oxy)- 5-(2,2,2- trifluoroethoxy) phenoxy)-1,3- dimethyl-2-oxo- 2,3-dihydro-1H- benzo[d]imidazol- 5-yl)-3,4- dimethoxybenzene- sulfonamide | 682 | 683 | E |
| 195 | 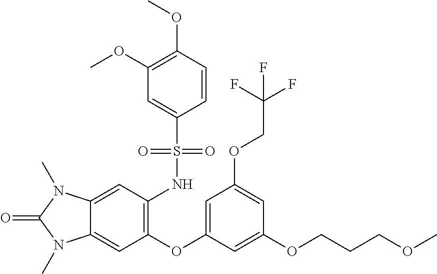 [SMILES: COCCCOc1cc(OCC(F)(F)F)cc(Oc2cc3c(cc2NS(=O)(=O)c2ccc(OC)c(OC)c2)n(C)c(=O)n3C)c1] | 3,4-dimethyl-N- (6-(3-(3- methoxypropoxy)- 5-(2,2,2- trifluoroethoxy) phenoxy)-1,3- dimethyl-2-oxo- 2,3-dihydro-1H- benzo[d]imidazol- 5- yl)benzenesulfonamide | 655 | 656 | C |
| 196 | 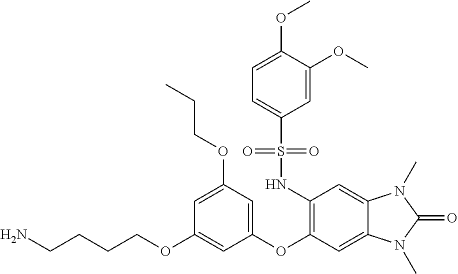 [SMILES: CCCOc1cc(OCCCCN)cc(Oc2cc3c(cc2NS(=O)(=O)c2ccc(OC)c(OC)c2)n(C)c(=O)n3C)c1] | N-(6-(3-(4- aminobutoxy)-5- propoxyphenoxy)- 1,3-dimethyl-2- oxo-2,3-dihydro- 1H- benzo[d]imidazol- 5-yl)-3,4- dimethoxybenzene- sulfonamide | 614 | 615 | D |
| 197 | 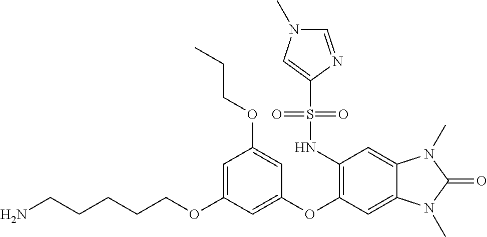 [SMILES: CCCOc1cc(OCCCCCN)cc(Oc2cc3c(cc2NS(=O)(=O)c2cn(C)cn2)n(C)c(=O)n3C)c1] | N-(6-(3-((5- aminopentyl)oxy)- 5- propoxyphenoxy)- 1,3-dimethyl-2- oxo-2,3-dihydro- 1H- benzo[d]imidazol- 5-yl)-1-methyl- 1H-imidazole-4- sulfonamide | 572 | 573 | D |
| 198 | 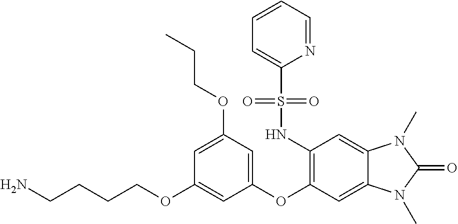 [SMILES: CCCOc1cc(OCCCCN)cc(Oc2cc3c(cc2NS(=O)(=O)c2ccccn2)n(C)c(=O)n3C)c1] | N-(6-(3-(4- aminobutoxy)-5- propoxyphenoxy)- 1,3-dimethyl-2- oxo-2,3-dihydro- 1H- benzo[d]imidazol- 5-yl)pyridine-2- sulfonamide | 555 | 556 | D |
| 199 | 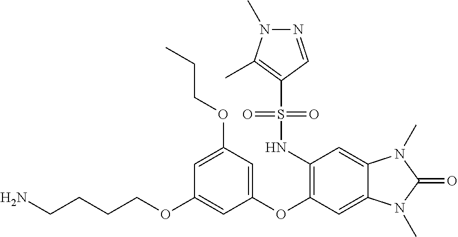 [SMILES: CCCOc1cc(OCCCCN)cc(Oc2cc3c(cc2NS(=O)(=O)c2cnn(C)c2C)n(C)c(=O)n3C)c1] | N-(6-(3-(4- aminobutoxy)-5- propoxyphenoxy)- 1,3-dimethyl-2- oxo-2,3-dihydro- 1H- benzo[d]imidazol- 5-yl)-1,5- dimethyl-1H- pyrazole-4- sulfonamide | 572 | 573 | D |
| 200 | 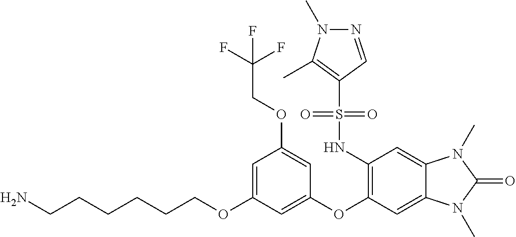 [SMILES: Cc1c(S(=O)(=O)Nc2cc3c(cc2Oc2cc(OCCCCCCN)cc(OCC(F)(F)F)c2)n(C)c(=O)n3C)cnn1C] | N-(6-(3-((6- aminohexyl)oxy)- 5-(2,2,2- trifluoroethoxy) phenoxy)-1,3- dimethyl-2-oxo- 2,3-dihydro-1H- benzo[d]imidazol- 5-yl)-1,5- dimethyl-1H- pyrazole-4- sulfonamide | 640 | 641 | E |
| 201 | 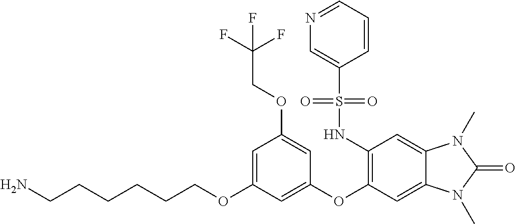 [SMILES: Cn1c(=O)n(C)c2cc(Oc3cc(OCCCCCCN)cc(OCC(F)(F)F)c3)c(NS(=O)(=O)c3cccnc3)cc21] | N-(6-(3-((6- aminohexyl)oxy)- 5-(2,2,2- trifluoroethoxy) phenoxy)-1,3- dimethyl-2-oxo- 2,3-dihydro-1H- benzo[d]imidazol- 5-yl)pyridine-3- sulfonamide | 623 | 624 | E |
| 202 | 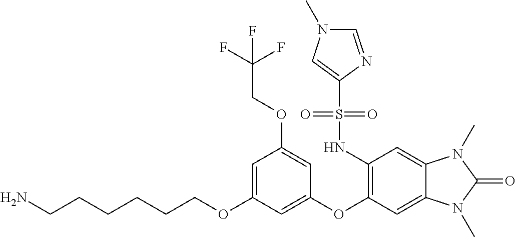 [SMILES: Cn1cnc(S(=O)(=O)Nc2cc3c(cc2Oc2cc(OCCCCCCN)cc(OCC(F)(F)F)c2)n(C)c(=O)n3C)c1] | N-(6-(3-((6- aminohexyl)oxy)- 5-(2,2,2- trifluoroethoxy) phenoxy)-1,3- dimethyl-2-oxo- 2,3-dihydro-1H- benzo[d]imidazol- 5-yl)-1-methyl- 1H-imidazole-4- sulfonamide | 626 | 627 | E |
| 203 | 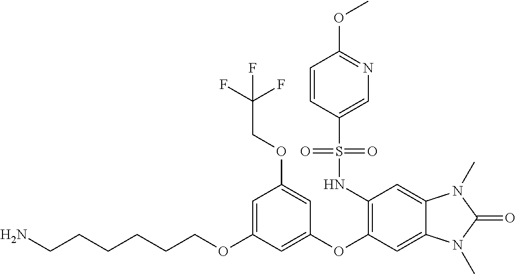 [SMILES: COc1ccc(S(=O)(=O)Nc2cc3c(cc2Oc2cc(OCCCCCCN)cc(OCC(F)(F)F)c2)n(C)c(=O)n3C)cn1] | N-(6-(3-((6- aminohexyl)oxy)- 5-(2,2,2- trifluoroethoxy) phenoxy)-1,3- dimethyl-2-oxo- 2,3-dihydro-1H- benzo[d]imidazol- 5-yl)-6- methoxypyridine- 3-sulfonamide | 653 | 654 | E |
| 204 | 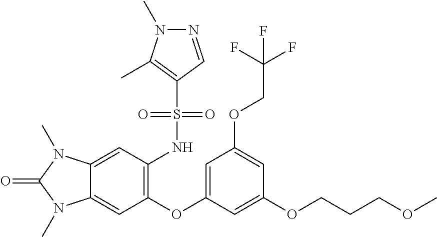 [SMILES: COCCCOc1cc(OCC(F)(F)F)cc(Oc2cc3c(cc2NS(=O)(=O)c2cnn(C)c2C)n(C)c(=O)n3C)c1] | N-(6-(3-(3- methoxypropoxy)- 5-(2,2,2- trifluoroethoxy) phenoxy)-1,3- dimethyl-2-oxo- 2,3-dihydro-1H- benzo[d]imidazol- 5-yl)-1,5- dimethyl-1H- pyrazole-4- sulfonamide | 613 | 614 | C |
| 205 | 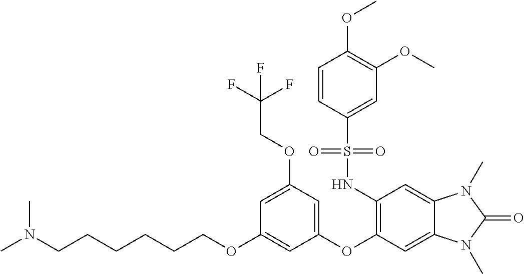 [SMILES: COc1ccc(S(=O)(=O)Nc2cc3c(cc2Oc2cc(OCCCCCCN(C)C)cc(OCC(F)(F)F)c2)n(C)c(=O)n3C)cc1OC] | N-(6-(3-((6- (dimethylamino) hexyl)oxy)-5- (2,2,2- trifluoroethoxy) phenoxy)-1,3- dimethyl-2-oxo- 2,3-dihydro-1H- benzo[d]imidazol- 5-yl)-3,4- dimethoxybenzene- sulfonamide | 710 | 711 | F |
| 206 | 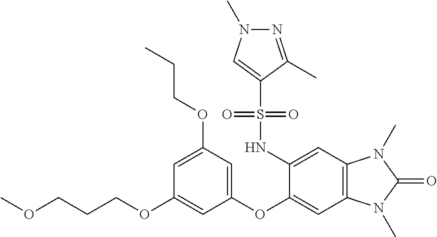 [SMILES: CCCOc1cc(OCCCOC)cc(Oc2cc3c(cc2NS(=O)(=O)c2cn(C)nc2C)n(C)c(=O)n3C)c1] | N-(6-(3-(3- methoxypropoxy)- 5- propoxyphenoxy)- 1,3-dimethyl-2- oxo-2,3-dihydro- 1H- benzo[d]imidazol- 5-yl)-1,3- dimethyl-1H- pyrazole-4- sulfonamide | 573 | 574 | B |
| 207 | 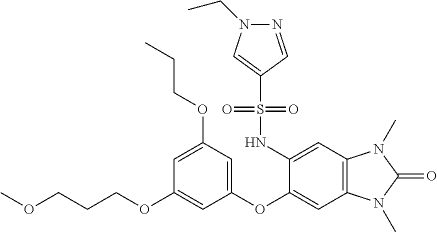 [SMILES: CCCOc1cc(OCCCOC)cc(Oc2cc3c(cc2NS(=O)(=O)c2cnn(CC)c2)n(C)c(=O)n3C)c1] | 1-ethyl-N-(6-(3- (3- methoxypropoxy)- 5- propoxyphenoxy)- 1,3-dimethyl-2- oxo-2,3-dihydro- 1H- benzo[d]imidazol- 5-yl)-1H- pyrazole-4- sulfonamide | 573 | 574 | B |
| 208 | 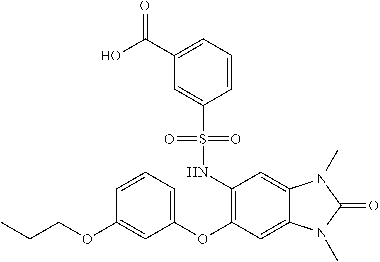 [SMILES: CCCOc1cccc(Oc2cc3c(cc2NS(=O)(=O)c2cccc(C(=O)O)c2)n(C)c(=O)n3C)c1] | 3-(N-(1,3- dimethyl-2-oxo- 6-(3- propoxyphenxoy)- 2,3-dihydro-1H- benzo[d]imidazol- 5- yl)sulfamoyl) benzoic acid | 511 | 512 | A |
| 209 | 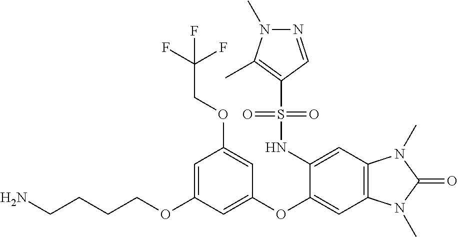 [SMILES: Cc1c(S(=O)(=O)Nc2cc3c(cc2Oc2cc(OCCCCN)cc(OCC(F)(F)F)c2)n(C)c(=O)n3C)cnn1C] | N-(6-(3-(4- aminobutoxy)-5- (2,2,2- trifluoroethoxy) phenoxy)-1,3- dimethyl-2-oxo- 2,3-dihydro-1H- benzo[d]imidazol- 5-yl)-1,5- dimethyl-1H- pyrazole-4- sulfonamide | 612 | 613 | D |
| 210 | 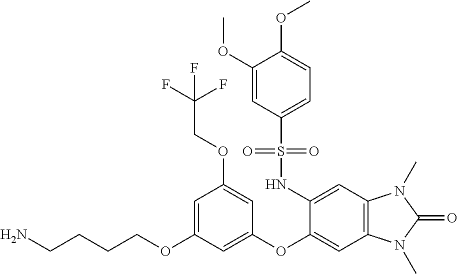 [SMILES: COc1ccc(S(=O)(=O)Nc2cc3c(cc2Oc2cc(OCCCCN)cc(OCC(F)(F)F)c2)n(C)c(=O)n3C)cc1OC] | N-(6-(3-(4- aminobutoxy)-5- (2,2,2- trifluoroerthoxy) phenoxy)-1,3- dimethyl-2-oxo- 2,3-dihydro-1H- benzo[d]imidazol- 5-yl)-3,4- dimethoxybenzene- sulfonamide | 654 | 655 | D |
| 211 | 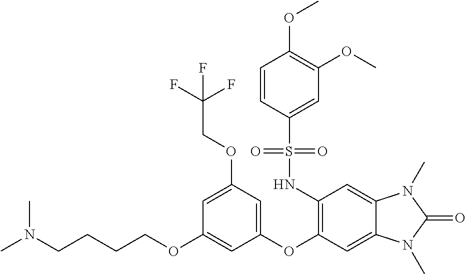 [SMILES: COc1ccc(S(=O)(=O)Nc2cc3c(cc2Oc2cc(OCCCCN(C)C)cc(OCC(F)(F)F)c2)n(C)c(=O)n3C)cc1OC] | N-(6-(3-(4- (dimethylamino) butoxy)-5-(2,2,2- trifluoroethoxy) phenoxy)-1,3- dimethyl-2-oxo- 2,3-dihydro-1H- benzo[d]imidazol- 5-yl)-3,4- dimethoxybenzene- sulfonamide | 682 | 683 | F |
| 212 | 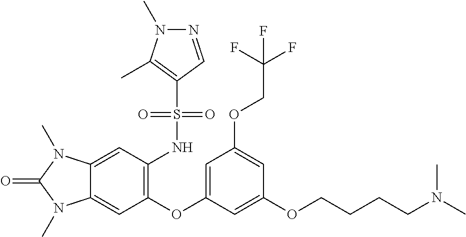 [SMILES: Cc1c(S(=O)(=O)Nc2cc3c(cc2Oc2cc(OCCCCN(C)C)cc(OCC(F)(F)F)c2)n(C)c(=O)n3C)cnn1C] | N-(6-(3-(4- (dimethylamino) butoxy)-5-(2,2,2- trifluoroethoxy) phenoxy)-1,3- dimethyl-2-oxo- 2,3-dihydro-1H- benzo[d]imidazol- 5-yl)-1,5- dimethyl-1H- pyrazole-4- sulfonamide | 640 | 641 | F |
| 213 | 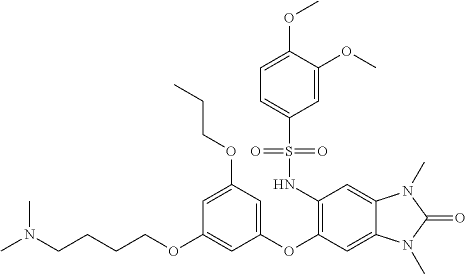 [SMILES: CCCOc1cc(OCCCCN(C)C)cc(Oc2cc3c(cc2NS(=O)(=O)c2ccc(OC)c(OC)c2)n(C)c(=O)n3C)c1] | N-(6-(3-((6- (dimethylamino) hexyl)oxy)-5- propoxyphenoxy)- 1,3-dimethyl-2- oxo-2,3-dihydro- 1H- benzo[d]imidazol- 5-yl)-3,4- dimethoxybenzene- sulfonamide | 670 | 671 | F |
| 214 | 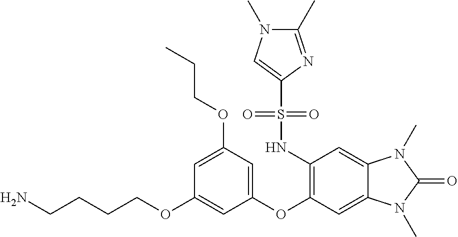 [SMILES: CCCOc1cc(OCCCCN)cc(Oc2cc3c(cc2NS(=O)(=O)c2cn(C)c(C)n2)n(C)c(=O)n3C)c1] | N-(6-3-(4- aminobutoxy)-5- propoxyphenoxy)- 1,3-dimethyl-2- oxo-2,3-dihydro- 1H- benzo[d]imidazol- 5-yl)-1,2- dimethyl-1H- imidazole-4- sulfonamide | 572 | 573 | D |
| 215 | 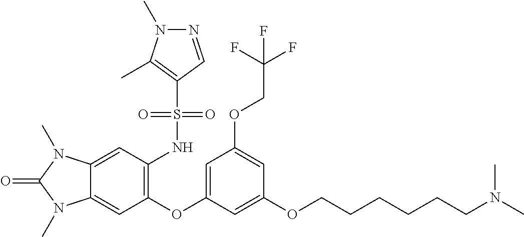 [SMILES: Cc1c(S(=O)(=O)Nc2cc3c(cc2Oc2cc(OCCCCCCN(C)C)cc(OCC(F)(F)F)c2)n(C)c(=O)n3C)cnn1C] | N-(6-(3-((6- (dimethylamino) hexyl)oxy)-5- (2,2,2- trifluoroethoxy) phenoxy)-1,3- dimethyl-2-oxo- 2-3,-dihydro-1H- benzo[d]imidazol- 5-yl)-1,5- dimethyl-1H- pyrazole-4- sulfonamide | 668 | 669 | F |
| 216 | 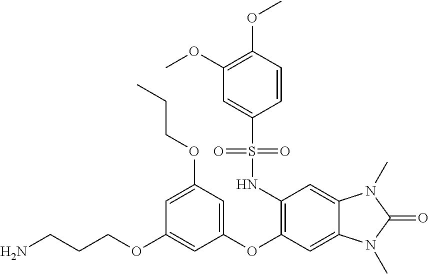 [SMILES: CCCOc1cc(OCCCN)cc(Oc2cc3c(cc2NS(=O)(=O)c2ccc(OC)c(OC)c2)n(C)c(=O)n3C)c1] | N-(6-(3-(3- aminopropoxy)-5- propoxyphenoxy)- 1,3-dimethyl-2- oxo-2,3-dihydro- 1H- benzo[d]imidazol- 5-yl)-3,4- dimethoxybenzene- sulfonamide | 600 | 601 | E |
| 217 | 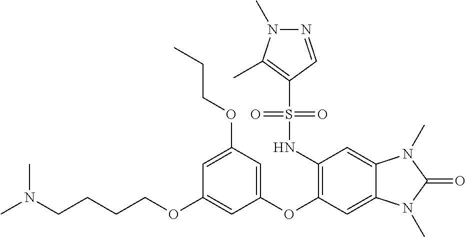 [SMILES: CCCOc1cc(OCCCCN(C)C)cc(Oc2cc3c(cc2NS(=O)(=O)c2cnn(C)c2C)n(C)c(=O)n3C)c1] | N-(6-(3-(4- (dimethylamino) butoxy)-5- propoxyphenoxy)- 1,3-dimethyl-2- oxo-2,3-dihydro- 1H- benzo[d]imidazol- 5-yl)-1,5- dimethyl-1H- pyrazole-4- sulfonamide | 600 | 601 | F |
| 218 | 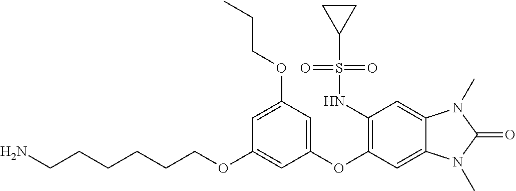 [SMILES: CCCOc1cc(OCCCCCCN)cc(Oc2cc3c(cc2NS(=O)(=O)C2CC2)n(C)c(=O)n3C)c1] | N-(6-(3-((6- aminohexyl)oxy)- 5- propoxyphenoxy)- 1,3-dimethyl-2- oxo-2,3-dihydro- 1H- benzo[d]imidazol- 5- yl)cyclopropane- sulfonamide | 546 | 547 | D |
| 219 | 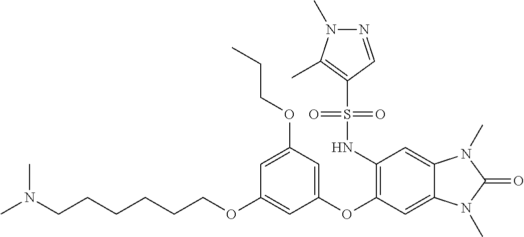 [SMILES: CCCOc1cc(OCCCCCCN(C)C)cc(Oc2cc3c(cc2NS(=O)(=O)c2cnn(C)c2C)n(C)c(=O)n3C)c1] | N-(6-(3-((6- (dimethylamino) hexyl)oxy)-5- propoxyphenoxy)- 1,3-dimethyl-2- oxo-2,3-dihydro- 1H- benzo[d]imidazol- 5-yl)-1,5- dimethyl-1H- pyrazole-4- sulfonamide | 628 | 629 | F |
| 220 | 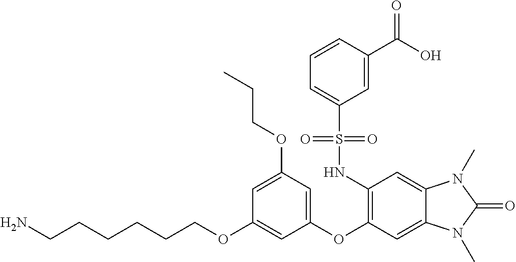 [SMILES: CCCOc1cc(OCCCCCCN)cc(Oc2cc3c(cc2NS(=O)(=O)c2cccc(C(=O)O)c2)n(C)c(=O)n3C)c1] | 3-(N-(6-(3-((6- aminohexyl)oxy)- 5- propoxyphenoxy)- 1,3-dimethyl-2- oxo-2,3-dihydro- 1H- benzo[d]imidazol- 5- yl)sulfamoyl) benzoic acid | 626 | 627 | D |
| 221 | 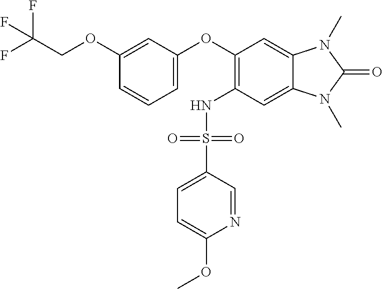 [SMILES: COc1ccc(S(=O)(=O)Nc2cc3c(cc2Oc2cccc(OCC(F)(F)F)c2)n(C)c(=O)n3C)cn1] | N-(1,3-dimethyl- 2-oxo-6-(3-(2,2,2- trifluoroethoxy) phenoxy)-2,3- dihydro-1H- benzo[d]imidazol- 5-yl)-6- methoxypyridine- 3-sulfonamide | 538 | 539 | C |
| 222 | 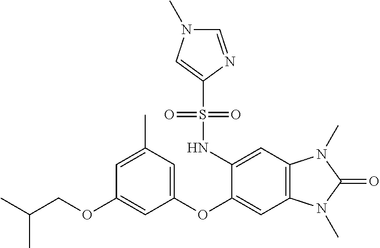 [SMILES: Cc1cc(OCC(C)C)cc(Oc2cc3c(cc2NS(=O)(=O)c2cn(C)cn2)n(C)c(=O)n3C)c1] | N-(6-(3- isobutoxy-5- methylphenoxy)- 1,3-dimethyl-2- oxo-2,3-dihydro- 1H- benzo[d]imidazol- 5-yl)-1-methyl- 1H-imidazole-4- sulfonamide | 499 | 500 | C |
| 223 | 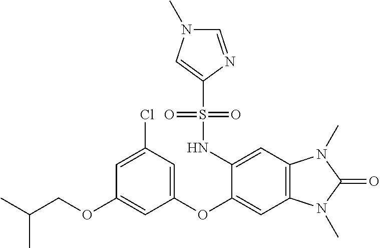 [SMILES: CC(C)COc1cc(Cl)cc(Oc2cc3c(cc2NS(=O)(=O)c2cn(C)cn2)n(C)c(=O)n3C)c1] | N-(6-(3-chloro-5- isobutoxyphenoxy)- 1,3-dimethyl- 2-oxo-2,3- dihydro-1H- benzo[d]imidazol- 5-yl)-1-methyl- 1H-imidazole-4- sulfonamide | 519 | 520 | C |
| 224 | 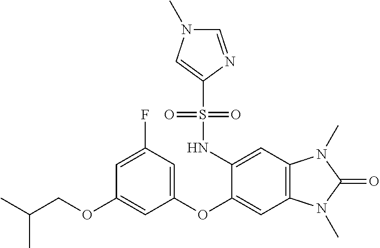 [SMILES: CC(C)COc1cc(F)cc(Oc2cc3c(cc2NS(=O)(=O)c2cn(C)cn2)n(C)c(=O)n3C)c1] | N-(6-(3-fluoro-5- isobutoxyphenoxy)- 1,3-dimethyl- 2-oxo-2,3- dihydro-1H- benzo[d]imidazol- 5-yl)-1-methyl- 1H-imidazole-4- sulfonamide | 503 | 504 | C |
| 225 | 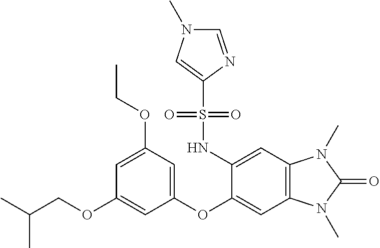 [SMILES: CCOc1cc(OCC(C)C)cc(Oc2cc3c(cc2NS(=O)(=O)c2cn(C)cn2)n(C)c(=O)n3C)c1] | N-(6-(3-ethoxy-5- isobutoxyphenoxy)- 1,3-dimethyl- 2-oxo-2,3- dihydro-1H- benzo[d]imidazol- 5-yl)-1-methyl- 1H-imidazole-4- sulfonamide | 529 | 530 | C |
| 226 | 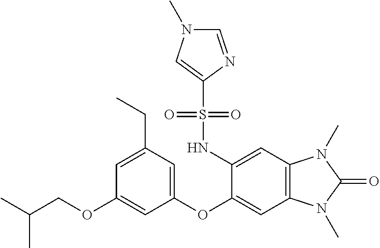 [SMILES: CCc1cc(OCC(C)C)cc(Oc2cc3c(cc2NS(=O)(=O)c2cn(C)cn2)n(C)c(=O)n3C)c1] | N-(6-(3-ethyl-5- isobutoxyphenoxy)- 1,3-dimethyl- 2-oxo-2,3- dihydro-1H- benzo[d]imidazol- 5-yl)-1-methyl- 1H-imidazole-4- sulfonamide | 513 | 514 | C |

### Example 227

### 5-(3-(6-amino-1,3-dimethyl-2-oxo-2,3-dihydro-1H-benzo[d]imidazol-5-yloxy)phenoxy)butanoic acid

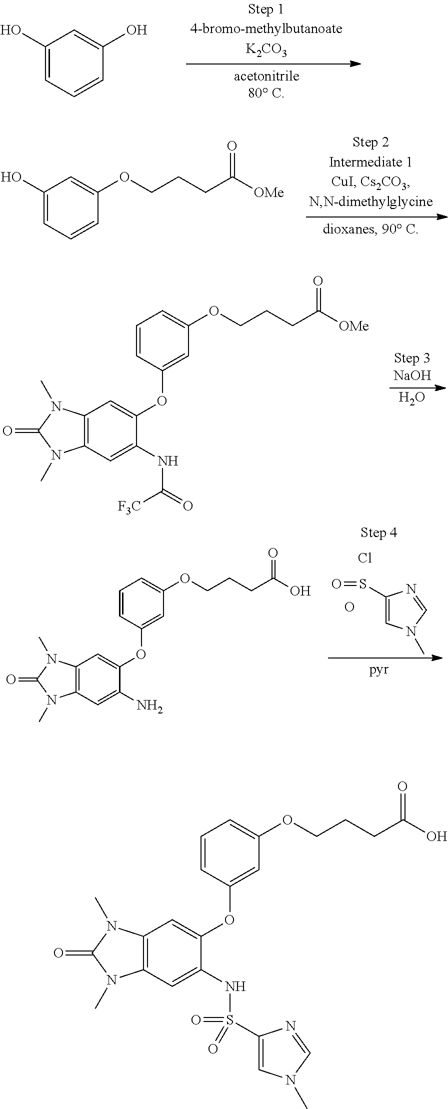

### Step 1: 4-(3-hydroxyphenoxy)butanoate

To a solution of resorcinol (1.0 g, 9.1 mmol) in 10 mL of acetonitrile and were added K₂CO₃ (1.3 g, 9.1 mmol) and methyl 4-bromobutanoate (1.6 g, 9.1 mmol). The mixture was heated to 86° C. and stirred overnight. The cooled reaction mixture was poured into 20 mL of H₂O and extracted with EtOAc (3×20 mL). The combined organic layer was washed with water (10 mL) and brine (10 mL), dried over anhydrous sodium sulfate, then concentrated under reduced pressure to afford a crude product. The residue was purified by flash chromatography (1:1 ACN/hexanes) to give 4-(3-hydroxyphenoxy)butanoate as a yellow solid (250 mg, 13%). MS (ES+) C₁₁H₁₄O₄ requires: 210. found: 211[M+H]⁺.

### Step 2: methyl 4-(3-(1,3-dimethyl-2-oxo-6-(2,2,2-trifluoroacetamido)-2,3-dihydro-1H-benzo[d]imidazol-5-yloxy)phenoxy)butanoate

To a solution of N-(6-bromo-1,3-dimethyl-2-oxo-2,3-dihydro-1H-benzo[d]imidazol-5-yl)-2,2,2-trifluoroacetamide (300 mg, 0.85 mmol) in 5 mL of 1,4-dioxanes were added CuI (48 mg, 0.26 mmol), Cs₂CO₃ (831 mg, 2.55 mmol), 2-(dimethylamino)acetic acid (87 mg, 0.85 mmol), methyl 4-(3-hydroxyphenoxy)butanoate (269 mg, 1.28 mmol). The reaction mixture was stirred at 90° C. overnight under a nitrogen atmosphere. The cooled reaction mixture was concentrated and purified by flash chromatography (1:1 ACN/hexanes) to afford methyl 4-(3-(1,3-dimethyl-2-oxo-6-(2,2,2-trifluoroacetamido)-2,3-dihydro-1H-benzo[d]imidazol-5-yloxy)phenoxy)butanoate as a black solid (200 mg, 48%). MS (ES+) C₂₂H₂₂F₃N₃O₆ requires: 481. found: 482[M+H]⁺.

### Step 3: 4-(3-(6-amino-1,3-dimethyl-2-oxo-2,3-dihydro-1H-benzo[d]imidazol-5-yloxy)phenoxy)butanoic acid

To a solution of methyl 4-(3-(1,3-dimethyl-2-oxo-6-(2,2,2-trifluoroacetamido)-2,3-dihydro-1H-benzo[d]imidazol-5-yloxy)phenoxy)butanoate (200 mg, 0.41 mmol) in 2 mL of water was added NaOH (33 mg, 0.82 mmol). The mixture was stirred at RT overnight, then concentrated and used to next step without further purification, to afford a black solid (100 mg, 62%). MS (ES+) C₁₉H₂₁N₃O₅ requires: 371. found: 372 [M+H]⁺.

### Step 4: 4-(3-(1,3-dimethyl-6-(1-methyl-1H-imidazole-4-sulfonamido)-2-oxo-2,3-dihydro-1H-benzo[d]imidazol-5-yloxy)phenoxy)butanoic acid

To a solution of 4-(3-(6-amino-1,3-dimethyl-2-oxo-2,3-dihydro-1H-benzo[d]imidazol-5-yloxy)phenoxy)butanoic acid (100 mg, 0.26 mmol) in 2 mL of DCM were added pyridine (41 mg, 0.52 mmol), and 1-methyl-1H-imidazole-4-sulfonyl chloride (70 mg, 0.39 mmol). The reaction mixture was stirred at RT for 1 h, then concentrated and purified by prep-HPLC (Mobile phase: A=0.01% TFA/H₂O, B=MeCN; Gradient: B=60%-95% in 18 min; Column: XBridge C18, 5 um, 30 mm×150 mm) to afford 4-(3-(1,3-dimethyl-6-(1-methyl-1H-imidazole-4-sulfonamido)-2-oxo-2,3-dihydro-1H-benzo[d]imidazol-5-yloxy)phenoxy)butanoic acid as a yellow solid (69 mg, 49%). MS (ES+) C₂₃H₂₅N₅O₇S requires: 515. found: 516 [M+H]⁺. ¹H NMR (500 MHz, d₆-DMSO): ¹H-NMR (500 MHz, CD₃OD) δ 7.42 (d, J=8.4 Hz, 3H), 7.16 (t, J=8.0 Hz, 1H), 6.74 (s, 1H), 6.66 (dd, J=8.0, 1.5 Hz, 1H), 6.31 (dd, J=8.0, 1.5 Hz, 1H), 6.24 (t, J=2.3 Hz, 1H), 4.00 (t, J=6.5 Hz, 2H), 3.60 (s, 3H), 3.46 (s, 3H), 3.30 (s, 3H), 2.48 (t, J=7.0 Hz, 2H), 2.07 (s, 2H).

### Example 228

### 5-(3-(6-amino-1,3-dimethyl-2-oxo-2,3-dihydro-1H-benzo[d]imidazol-5-yloxy)phenoxy)pentanoic acid

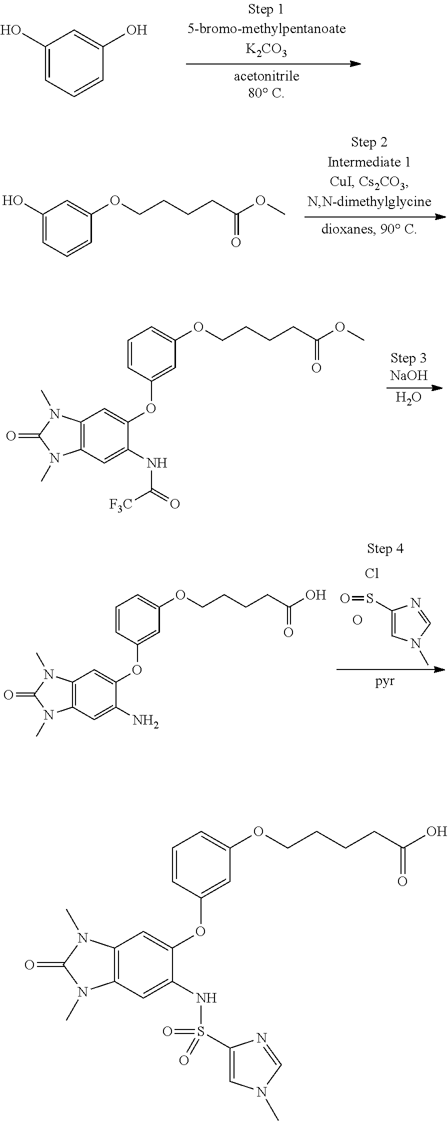

### Step 1: 4-(3-hydroxyphenoxy)butanoate

To a solution of resorcinol (1.0 g, 9.1 mmol) in 10 mL of acetonitrile and were added K₂CO₃ (1.2 g, 9.1 mmol) and methyl 5-bromopentanoate (1.7 g, 9.1 mmol). The mixture was heated to 86° C. and stirred overnight. The cooled reaction mixture was poured into 20 mL of water and extracted with EtOAc (3×20 mL). The combined organic layer was washed with water (10 mL) and brine (10 mL), dried over anhydrous sodium sulfate, filtered, concentrated under reduced then purified by flash chromatography (1:1 ACN/hexanes) to afford a yellow solid (400 mg, 20%). MS (ES+) C₁₂H₁₆O₄ requires: 224. found: 225 [M+H]⁺.

### Step 2: Methyl 5-(3-(1,3-dimethyl-2-oxo-6-(2,2,2-trifluoroacetamido)-2,3-dihydro-1H-benzo[d]imidazol-5-yloxy)phenoxy)pentanoate

To a solution of N-(6-bromo-1,3-dimethyl-2-oxo-2,3-dihydro-1H-benzo[d]imidazol-5-yl)-2,2,2-trifluoroacetamide (100 mg, 0.28 mmol) in 3 mL of 1,4-dioxane was added CuI (16 mg, 0.084 mmol), Cs₂CO₃ (274 mg, 0.84 mmol), 2-(dimethylamino)acetic acid (29 mg, 0.28 mmol), and methyl 4-(3-hydroxyphenoxy)butanoate (96 mg, 0.43 mmol) while the solution was stirring. The reaction mixture was stirred at 90° C. overnight under a nitrogen atmosphere. The cooled reaction mixture was concentrated and purified via flash chromatography (1:1 ACN/hexanes) to afford methyl 5-(3-(1,3-dimethyl-2-oxo-6-(2,2,2-trifluoroacetamido)-2,3-dihydro-1H-benzo[d]imidazol-5-yloxy)phenoxy)pentanoate as a black solid (100 mg, 45%). MS (ES+) C₂₃H₂₄F₃N₃O₅ requires: 495. found: 496 [M+H]⁺.

### Step 3: 5-(3-(6-amino-1,3-dimethyl-2-oxo-2,3-dihydro-1H-benzo[d]imidazol-5-yloxy)phenoxy)pentanoic acid

To a solution of methyl 5-(3-(1,3-dimethyl-2-oxo-6-(2,2,2-trifluoroacetamido)-2,3-dihydro-1H-benzo[d]imidazol-5-yloxy)phenoxy)pentanoate (300 mg, 0.61 mmol) in 5 mL of water was added NaOH (48 mg, 1.22 mmol). The mixture was stirred at RT overnight, then concentrated to afford a black solid (100 mg, 43%) which was used in the next step without further purification. MS (ES+) C₁₉H₂₁N₃O₅ requires: 385. found: 386 [M+H]⁺.

### Step 4: 5-(3-(6-amino-1,3-dimethyl-2-oxo-2,3-dihydro-1H-benzo[d]imidazol-5-yloxy)phenoxy)pentanoic acid

To a solution of 5-(3-(6-amino-1,3-dimethyl-2-oxo-2,3-dihydro-1H-benzo[d]imidazol-5-yloxy)phenoxy)pentanoic acid (200 mg, 0.52 mmol) in 2 mL of DCM was added pyridine (41 mg, 0.52 mmol), and 1-methyl-1H-imidazole-4-sulfonyl chloride (94 mg, 0.52 mmol). The solution was stirred at RT for 1 h. Then concentrated and purification via prep-HPLC (Mobile phase: A=0.01% TFA/H₂O, B=MeCN; Gradient: B=60%-95% in 18 min; Column: XBridge C18, 5 um, 30 mm×150 mm) to afford 5-(3-(6-amino-1,3-dimethyl-2-oxo-2,3-dihydro-1H-benzo[d]imidazol-5-yloxy)phenoxy)pentanoic acid as a white solid (29.6 mg, 9%). MS (ES+) C₂₄H₂₇N₅O₇S requires: 529. found: 530 [M+H]⁺. ¹H NMR (500 MHz, CD₃OD) δ 7.42 (d, J=6.0 Hz, 3H), 7.16 (t, J=8.3 Hz, 1H), 6.73 (s, 1H), 6.66 (dd, J=7.5, 1.5 Hz, 1H), 6.29 (dd, J=8.3, 2.1 Hz, 1H), 6.20 (t, J=2.0 Hz, 1H), 3.97 (t, J=6.3 Hz, 2H), 3.60 (s, 3H), 3.46 (s, 3H), 3.30 (s, 3H), 2.29 (t, J=6.8 Hz, 2H), 1.89-1.70 (m, 4H).

### Example 229

### N-(1,3-dimethyl-2-oxo-6-(3-(piperidin-4-ylmethoxy)phenoxy)-2,3-dihydro-1H-benzo[d]imidazol-5-yl)-1,2-dimethyl-1H-imidazole-4-sulfonamide

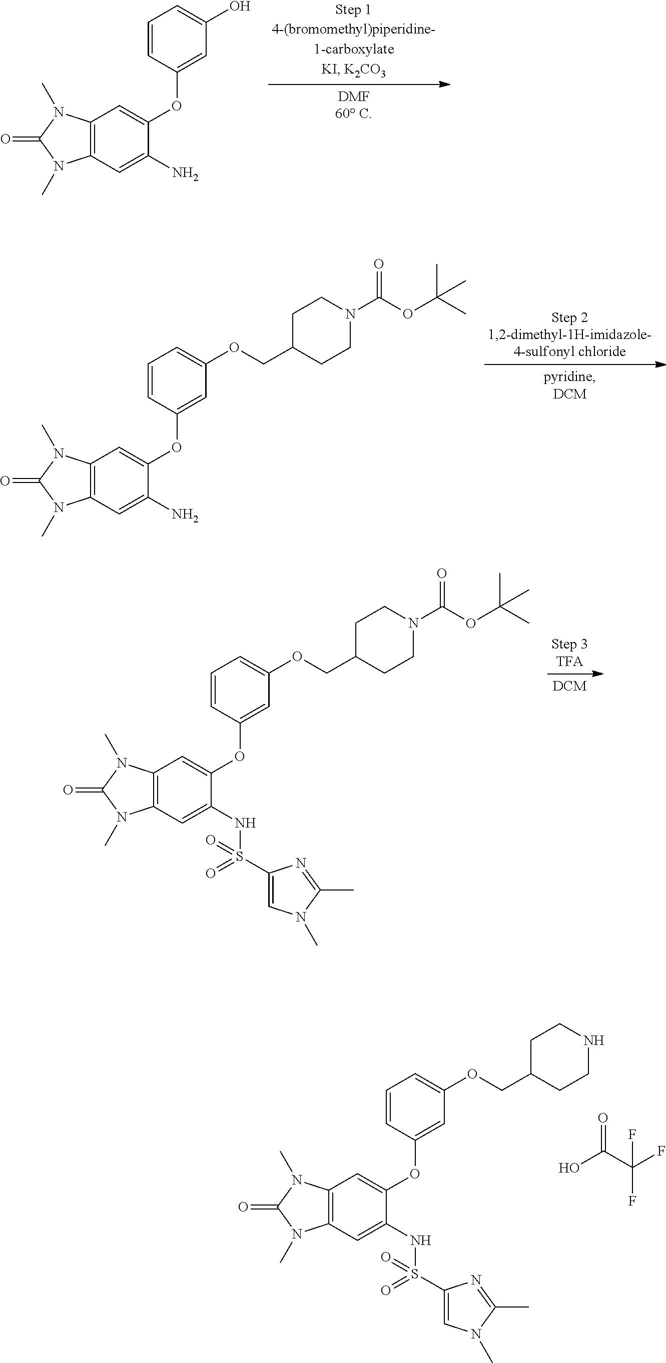

### Step 1: tert-butyl 4-((3-((6-amino-1,3-dimethyl-2-oxo-2,3-dihydro-1H-benzo[d]imidazol-5-yl)oxy)phenoxy)methyl)piperidine-1-carboxylate

To a solution of 5-amino-6-(3-hydroxyphenoxy)-1,3-dimethyl-1H-benzo[d]imidazol-2(3H)-one (45 mg, 0.16 mmol) in anhydrous DMF (1 ml) were added potassium carbonate (22 mg, 0.16 mmol), tert-butyl 4-(bromomethyl)piperidine-1-carboxylate (44 mg, 0.16 mmol), and potassium iodide (13 mg, 0.079 mmol). The reaction mixture stirred 60° C. for 30 min. The reaction mixture was then concentrated to give tert-butyl 4-((3-((6-amino-1,3-dimethyl-2-oxo-2,3-dihydro-1H-benzo[d]imidazol-5-yl)oxy)phenoxy)methyl)piperidine-1-carboxylate which was used in the next step without further purification. MS (ES+) C₂₆H₃₄N₄O₅ requires: 482. found 483 [M+H]⁺.

### Step 2: tert-butyl 4-((3-((6-(1,2-dimethyl-1H-imidazole-4-sulfonamido)-1,3-dimethyl-2-oxo-2,3-dihydro-1H-benzo[d]imidazol-5-yl)oxy)phenoxy)methyl)piperidine-1-carboxylate

To a solution of tert-butyl 4-((3-((6-amino-1,3-dimethyl-2-oxo-2,3-dihydro-1H-benzo[d]imidazol-5-yl)oxy)phenoxy)methyl)piperidine-1-carboxylate (76 mg, 0.16 mmol) in anhydrous DCM (1 ml) was added pyridine (25 uL, 0.32 mmol) and 1,2-dimethyl-1H-imidazole-4-sulfonyl chloride (31 mg, 0.16 mmol) in a vial. The reaction mixture was stirred at ambient temperature for 48 hrs before quenching with methanol, concentrated under reduced pressure and purified by prep HPLC to obtain tert-butyl 4-((3-((6-(1,2-dimethyl-1H-imidazole-4-sulfonamido)-1,3-dimethyl-2-oxo-2,3-dihydro-1H-benzo[d]imidazol-5-yl)oxy)phenoxy)methyl)piperidine-1-carboxylate (2.9 mg, 3% yield over 2 steps). MS (ES+) C₃₁H₄₀N₆O₇S requires: 640. found 641 [M+H]⁺.

### Step 3: N-(1,3-dimethyl-2-oxo-6-(3-(piperidin-4-ylmethoxy)phenoxy)-2,3-dihydro-1H-benzo[d]imidazol-5-yl)-1,2-dimethyl-1H-imidazole-4-sulfonamide trifluoro acetate

In a 5 mL vial containing tert-butyl 4-((3-((6-(1,2-dimethyl-1H-imidazole-4-sulfonamido)-1,3-dimethyl-2-oxo-2,3-dihydro-1H-benzo[d]imidazol-5-yl)oxy)phenoxy)methyl)piperidine-1-carboxylate (2.9 mg, 4.5 mol) was added 2,2,2-trifluoroacetic acid (0.52 μl, 6.8 μmol) in DCM (1 ml) to give a colorless solution. The de-protection was monitored by LCMS until completion then quenched with methanol, filtered purified by prep HPLC to yield N-(1,3-dimethyl-2-oxo-6-(3-(piperidin-4-ylmethoxy)phenoxy)-2,3-dihydro-1H-benzo[d]imidazol-5-yl)-1,2-dimethyl-1H-imidazole-4-sulfonamide 2,2,2-trifluoroacetate (1.8 mg, 74%). MS (ES+) C₂₆H₃₂N₆O₅S requires: 540. found 541 [M+H]⁺. 1H NMR (d₆-DMSO) δ: 9.23 (br-s, 1H), 8.50 (br-s, 1H), 8.17 (br-s, 1H), 7.49 (s, 1H), 7.18 (t, J=8.3 Hz, 1H), 7.15 (s, 1H), 6.77 (s, 1H), 6.63 (dd, J=8.3, 1.9 Hz, 1H), 6.32 (br-s, 1H), 6.28 (d, J=8.3 Hz, 1H), 3.82 (d, J=6.0 Hz, 2H), 3.46 (br-s, 3H), 3.31 (m, 2H), 3.29 (s, 3H), 3.21 (s, 3H), 2.90 (m, 2H), 2.12 (s, 3H), 2.03 (m, 1H), 1.89 (br-d, J=13.2 Hz, 2H), 1.39-1.49 (m, 2H).

### Example 230

### N-(6-(3-(3-hydroxy-2-methylpropoxy)phenoxy)-1,3-dimethyl-2-oxo-2,3-dihydro-1H-benzo[d]imidazol-5-yl)-1-methyl-1H-imidazole-4-sulfonamide

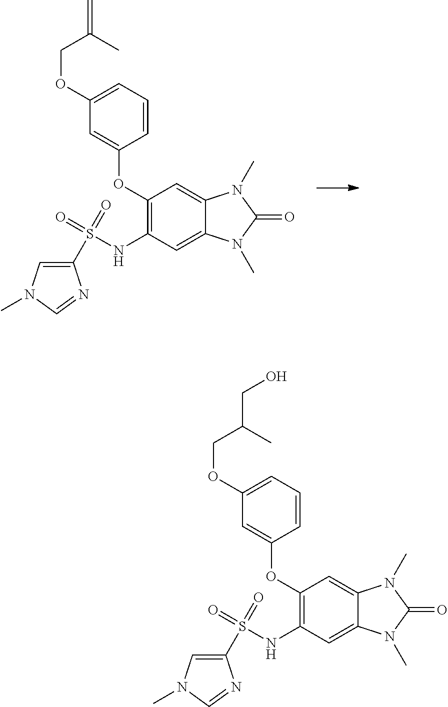 [SMILES: C=C(C)COc1cccc(Oc2cc3c(cc2NS(=O)(=O)c2cn(C)cn2)n(C)c(=O)n3C)c1 ; CC(CO)COc1cccc(Oc2cc3c(cc2NS(=O)(=O)c2cn(C)cn2)n(C)c(=O)n3C)c1]

To a solution of N-(1,3-dimethyl-6-(3-((2-methylallyl)oxy)phenoxy)-2-oxo-2,3-dihydro-1H-benzo[d]imidazol-5-yl)-1-methyl-1H-imidazole-4-sulfonamide (40 mg, 0.083 mmol) in THF (2 ml) was added borane (0.50 ml, 0.50 mmol) and the reaction mixture was stirred for 2 h at ambient temperature. The reaction mixture was then quenched with water (0.2 mL) and allowed to stir at 70° C. for 1 hour. LC-MS showed the major peak of 530 with the positive mode and 528 in the negative mode indicating the formation of the borane intermediate with MW=529. This was followed by addition of 10% NaOH aqueous solution (0.5 ml) and the mixture was stirred at room temperature for 30 min. LC-MS showed the major peak of 502 with the positive mode. The reaction mixture was then concentrated and purifed by prep-HPLC (Mobile phase: A=0.1% TFA/H₂O, B=0.1% TFA/MeCN; Gradient: B=10%-50% in 12 min; Column: C18) to give the title compound (3.7 mg, 9%). MS (ES+) C₂₃H₂₇N₅O₆S requires: 501. found 502 [M+H]⁺. ¹H-NMR (600 MHz, CD₃OD) δ ppm 7.48 (s, 1H), 7.43 (s, 1H), 7.38 (s, 1H), 7.14 (t, J=7.8 Hz, 1H), 6.71 (s, 1H), 6.65 (d, J=7.8 Hz, 1H), 6.27 (m, 2H), 3.92 (m, 1H), 3.82 (m, 1H), 3.57 (s, 3H), 3.56 (m, 2H), 3.43 (s, 3H), 3.28 (s, 3H), 2.05 (m, 1H), 1.03 (d, J=6.8 Hz, 3H).

### Example 231

### N-{6-[3-(2,3-dihydroxy-2-methylpropoxy)phenoxy]-1,3-dimethyl-2-oxo-2,3-dihydro-1H-1,3-benzodiazol-5-yl}-1-methyl-1H-imidazole-4-sulfonamide

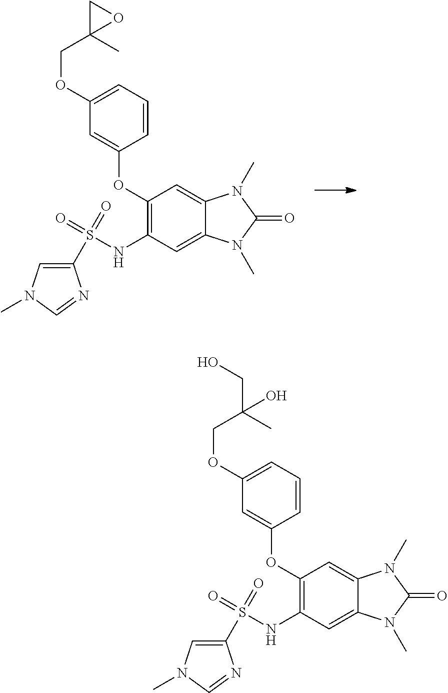 [SMILES: Cn1cnc(S(=O)(=O)Nc2cc3c(cc2Oc2cccc(OCC4(C)CO4)c2)n(C)c(=O)n3C)c1 ; Cn1cnc(S(=O)(=O)Nc2cc3c(cc2Oc2cccc(OCC(C)(O)CO)c2)n(C)c(=O)n3C)c1]

A mixture of N-(1,3-dimethyl-6-(3-((2-methyloxiran-2-yl)methoxy)phenoxy)-2-oxo-2,3-dihydro-1H-benzo[d]imidazol-5-yl)-1-methyl-1H-imidazole-4-sulfonamide (100 mg, 0.20 mmol) and sulfuric acid (39 mg, 0.40 mmol) in THF (2 ml) and water (2 ml) was stirred at 60° C. for 2 h. The reaction mixture was concentrated and purifed by prep-HPLC (Mobile phase: A=0.1% TFA/H2O, B=0.1% TFA/MeCN; Gradient: B=10%-30% in 12 min; Column: C18) to give the title compound (15 mg, 15%). MS (ES+) C₂₃H₂₇N₅O₇S requires: 517. found 518 [M+H]⁺. ¹H-NMR (600 MHz, d₆-DMSO) δ: ppm 9.32 (s, 1H), 7.58 (d, J=14.1 Hz, 2H), 7.16 (t, J=8.3 Hz, 1H), 7.10 (s, 1H), 6.77 (s, 1H), 6.66 (dd, J=2.3, 8.2 Hz, 1H), 6.32 (dd, J=2.3, 8.2 Hz, 1H), 6.24 (s, 1H), 3.74 (d, J=9.2 Hz, 1H), 3.68 (d, J=9.2 Hz, 1H), 3.58 (s, 3H), 3.37 (d, J=11.1 Hz, 1H), 3.28 (d, J=11.1 Hz, 1H), 3.27 (s, 3H), 3.20 (s, 3H), 1.11 (s, 3H).

### Examples 232 and 233

### N-{6-[3-(3-hydroxy-2-methoxy-2-methylpropoxy)phenoxy]-1,3-dimethyl-2-oxo-2,3-dihydro-1H-1,3-benzodiazol-5-yl}-1-methyl-1H-imidazole-4-sulfonamide (Example 232)

### N-{6-[3-(2-hydroxy-3-methoxy-2-methylpropoxy)phenoxy]-1,3-dimethyl-2-oxo-2,3-dihydro-1H-1,3-benzodiazol-5-yl}-1-methyl-1H-imidazole-4-sulfonamide (Example 233)

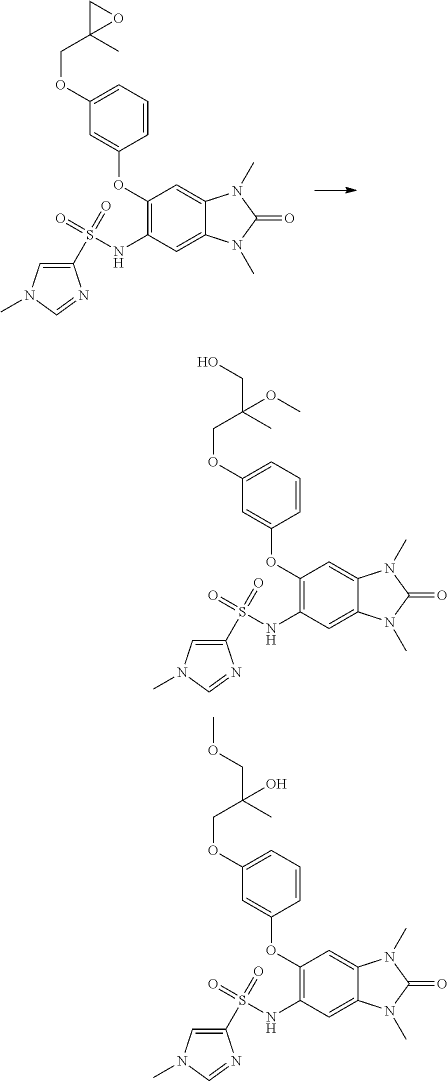

A solution of N-(1,3-dimethyl-6-(3-((2-methyloxiran-2-yl)methoxy)phenoxy)-2-oxo-2,3-dihydro-1H-benzo[d]imidazol-5-yl)-1-methyl-1H-imidazole-4-sulfonamide (10 mg, 0.020 mmol) in MeOH (1 mL) was treated with TFA (0.5 mL) and stirred at 60° C. for 12 h. The reaction mixture was concentrated and purified by prep-HPLC (Mobile phase: A=0.1% TFA/H₂O, B=0.1% TFA/MeCN; Gradient: B=10%-30% in 12 min; Column: C18) to give N-{6-[3-(3-hydroxy-2-methoxy-2-methylpropoxy)phenoxy]-1,3-dimethyl-2-oxo-2,3-dihydro-1H-1,3-benzodiazol-5-yl}-1-methyl-1H-imidazole-4-sulfonamide (1.7 mg, 16%) and N-{6-[3-(2-hydroxy-3-methoxy-2-methylpropoxy)phenoxy]-,3-dimethyl-2-oxo-2,3-dihydro-1H-1,3-benzodiazol-5-yl}-1-methyl-1H-imidazole-4-sulfonamide (1.0 mg, 9.4%).

N-{6-[3-(3-hydroxy-2-methoxy-2-methylpropoxy)phenoxy]-1,3-dimethyl-2-oxo-2,3-dihydro-1H-1,3-benzodiazol-5-yl}-1-methyl-1H-imidazole-4-sulfonamide (Example 232): MS (ES+) C₂₄H₂₉N₅O₇S requires: 531. found 532 [M+H]⁺. ¹H-NMR (600 MHz, d₆-DMSO) δ: ppm 9.32 (s, 1H), 7.58 (d, J=14.1 Hz, 2H), 7.16 (t, J=8.3 Hz, 1H), 7.10 (s, 1H), 6.78 (s, 1H), 6.64 (dd, J=2.3, 8.7 Hz, 1H), 6.29 (m, 2H), 3.89 (d, J=11.1 Hz, 1H), 3.79 (d, J=11.1 Hz, 1H), 3.58 (s, 3H), 3.51 (d, J=11 Hz, 1H), 3.50 (s, 1H), 3.34 (d, J=11 Hz, 1H), 3.27 (s, 3H), 3.20 (s, 3H), 3.18 (s, 3H), 1.12 (s, 3H).

N-{6-[3-(2-hydroxy-3-methoxy-2-methylpropoxy)phenoxy]-1,3-dimethyl-2-oxo-2,3-dihydro-1H-1,3-benzodiazol-5-yl}-1-methyl-1H-imidazole-4-sulfonamide (Example 233): MS (ES+) C₂₄H₂₉N₅O₇S requires: 531. found 532 [M+H]⁺. ¹H-NMR (600 MHz, d₆-DMSO) δ: ppm 9.32 (s, 1H), 7.58 (d, J=14.1 Hz, 2H), 7.15 (t, J=8.3 Hz, 1H), 7.10 (s, 1H), 6.78 (s, 1H), 6.60 (dd, J=2.3, 8.7 Hz, 1H), 6.30 (dd, J=2.3, 8.7 Hz, 1H), 6.26 (s, 1H), 3.76 (d, J=9.1 Hz, 1H), 3.68 (d, J=9.1 Hz, 1H), 3.58 (s, 3H), 3.50 (s, 1H), 3.32 (d, J=9.2 Hz, 1H), 3.27 (s, 3H), 3.26 (s, 3H), 3.22 (d, J=9.2 Hz, 1H), 3.20 (s, 3H), 1.13 (s, 3H).

### Example 234

### N-{6-[3-(3-amino-2-hydroxy-2-methylpropoxy)phenoxy]-1,3-dimethyl-2-oxo-2,3-dihydro-1H-1,3-benzodiazol-5-yl}-1-methyl-1H-imidazole-4-sulfonamide

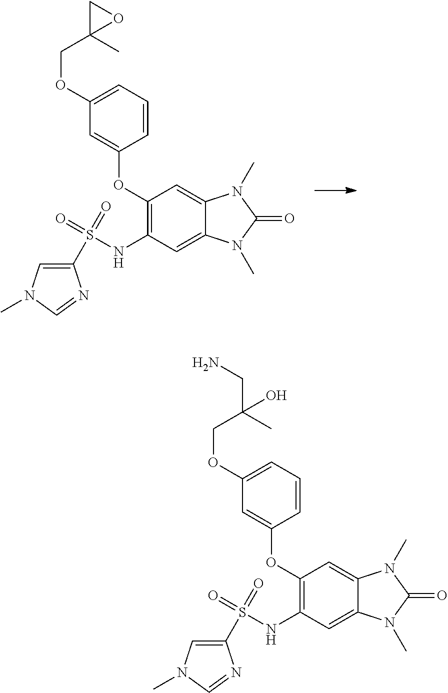 [SMILES: Cn1cnc(S(=O)(=O)Nc2cc3c(cc2Oc2cccc(OCC4(C)CO4)c2)n(C)c(=O)n3C)c1 ; Cn1cnc(S(=O)(=O)Nc2cc3c(cc2Oc2cccc(OCC(C)(O)CN)c2)n(C)c(=O)n3C)c1]

A mixture of N-(1,3-dimethyl-6-(3-((2-methyloxiran-2-yl)methoxy)phenoxy)-2-oxo-2,3-dihydro-1H-benzo[d]imidazol-5-yl)-1-methyl-1H-imidazole-4-sulfonamide (10 mg, 0.020 mmol) in ammonium hydroxide (140 mg, 4.0 mmol) was stirred at 60° C. for 12 h. The reaction mixture was concentrated and purified by prep-HPLC (Mobile phase: A=0.1% TFA/H₂O, B=0.1% TFA/MeCN; Gradient: B=10%-30% in 12 min; Column: C18) to give the title compound. (2.5 mg, 24%). MS (ES+) C₂₃H₂₈N₆O₆S requires: 516. found 517 [M+H]⁺. ¹H-NMR (600 MHz, d₆-DMSO) δ: ppm 9.30 (s, 1H), 7.73 (s, 3H), 7.62 (s, 1H), 7.59 (s, 1H), 7.20 (t, J=8.3 Hz, 1H), 7.08 (s, 1H), 6.77 (s, 1H), 6.64 (dd, J=8.7, 2.3 Hz, 1H), 6.35 (m, 2H), 3.81 (d, J=9.1 Hz, 1H), 3.78 (d, J=9.1 Hz, 1H), 3.60 (s, 3H), 3.27 (s, 3H), 3.21 (s, 3H), 3.29 (m, 1H), 2.84 (m, 1H), 1.25 (s, 3H).

### Example 235

### N-(6-{3-[2-hydroxy-2-methyl-3-(methylamino)propoxy]phenoxy}-1,3-dimethyl-2-oxo-2,3-dihydro-1H-1,3-benzodiazol-5-yl)-1-methyl-1H-imidazole-4-sulfonamide

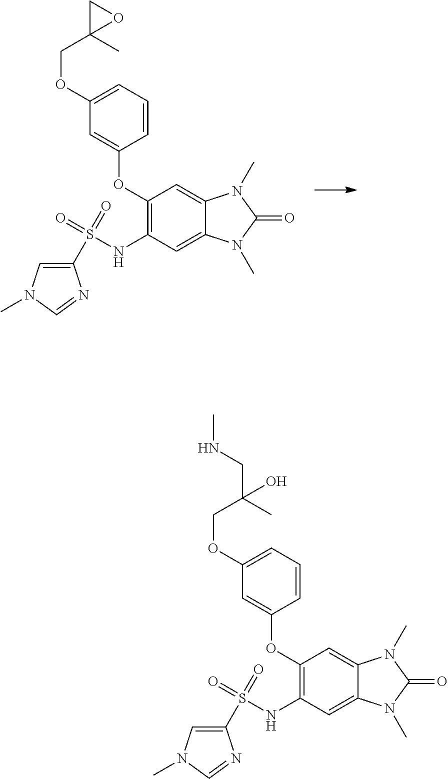 [SMILES: Cn1cnc(S(=O)(=O)Nc2cc3c(cc2Oc2cccc(OCC4(C)CO4)c2)n(C)c(=O)n3C)c1 ; CNCC(C)(O)COc1cccc(Oc2cc3c(cc2NS(=O)(=O)c2cn(C)cn2)n(C)c(=O)n3C)c1]

A mixture of N-(1,3-dimethyl-6-(3-((2-methyloxiran-2-yl)methoxy)phenoxy)-2-oxo-2,3-dihydro-1H-benzo[d]imidazol-5-yl)-1-methyl-1H-imidazole-4-sulfonamide (10 mg, 0.020 mmol) and methanamine (1.0 ml, 1.0 mmol) in MeOH was stirred at 60° C. for 12 h. The reaction mixture was concentrated and purified by prep-HPLC (Mobile phase: A=0.1% TFA/H₂O, B=0.1% TFA/MeCN; Gradient: B=10%-30% in 12 min; Column: C18) to give the title compound (1.8 mg, 17%). MS (ES+) C₂₄H₃₀N₆O₆S requires: 530. found 531 [M+H]⁺. ¹H-NMR (600 MHz, d₆-DMSO) δ: ppm 9.30 (s, 1H), 8.25 (s, 2H), 7.63 (s, 1H), 7.60 (s, 1H), 7.21 (t, J=8.3 Hz, 1H), 7.08 (s, 1H), 6.77 (s, 1H), 6.64 (dd, J=8.7, 2.3 Hz, 1H), 6.37 (m, 1H), 6.36 (dd, J=8.7, 2.3 Hz, 1H), 5.65 (s, 1H), 3.83 (d, J=9.1 Hz, 1H), 3.78 (d, J=9.1 Hz, 1H), 3.62 (s, 3H), 3.27 (s, 3H), 3.21 (s, 3H), 3.08 (m, 1H), 2.99 (m, 1H), 2.59 (t, J=5.9 Hz, 3H), 1.29 (s, 3H).

### Example 236

### N-(1,3-dimethyl-6-(3-((2-methyloxiran-2-yl)methoxy)phenoxy)-2-oxo-2,3-dihydro-1H-benzo[d]imidazol-5-yl)-1-methyl-1H-imidazole-4-sulfonamide

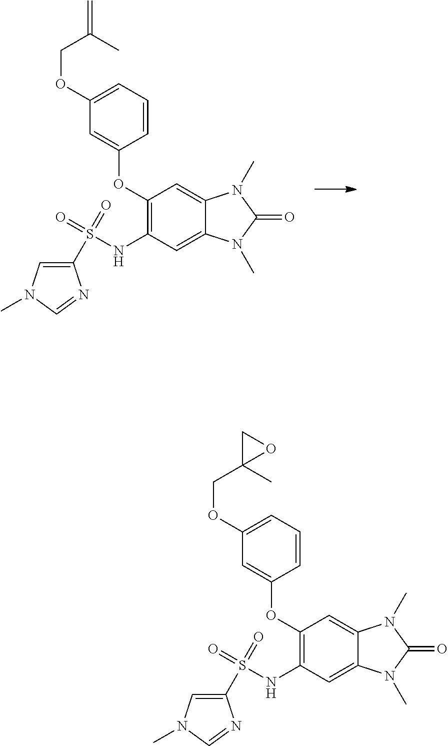 [SMILES: C=C(C)COc1cccc(Oc2cc3c(cc2NS(=O)(=O)c2cn(C)cn2)n(C)c(=O)n3C)c1 ; Cn1cnc(S(=O)(=O)Nc2cc3c(cc2Oc2cccc(OCC4(C)CO4)c2)n(C)c(=O)n3C)c1]

To a solution of N-(1,3-dimethyl-6-(3-((2-methylallyl)oxy)phenoxy)-2-oxo-2,3-dihydro-1H-benzo[d]imidazol-5-yl)-1-methyl-1H-imidazole-4-sulfonamide (75 mg, 0.155 mmol) in DCM (3 mL) was added MCPBA (191 mg, 0.776 mmol). The resulting mixture was stirred for 3 h. LC-MS showed clean conversion to the product. The mixture was concentrated and purified by column chromatography (0-100% EtOAc/hexanes then 0-45% methanol/EtOAc) to give the title compound (58 mg, 75%). MS (ES+) C₂₃H₂₅N₅O₆S requires: 499. found 500 [M+H]⁺. ¹H-NMR (600 MHz, d₆-DMSO) δ: ppm 9.32 (s, 1H), 7.56 (s, 2H), 7.16 (t, J=8.3 Hz, 1H), 7.10 (s, 1H), 6.78 (s, 1H), 6.64 (d, J=7.6 Hz, 1H), 6.30 (m, 2H), 4.10 (d, J=11.1 Hz, 1H), 3.80 (d, J=11.1 Hz, 1H), 3.58 (s, 3H), 3.27 (s, 3H), 3.20 (s, 3H), 2.78 (d, J=5.1 Hz, 1H), 2.68 (d, J=5.1 Hz, 1H), 1.36 (s, 3H).

### Example 237

### N-[6-(3-{3-[(2-aminoethyl)amino]-2-hydroxy-2-methylpropoxy}phenoxy)-1,3-dimethyl-2-oxo-2,3-dihydro-1H-1,3-benzodiazol-5-yl]-1-methyl-1H-imidazole-4-sulfonamide

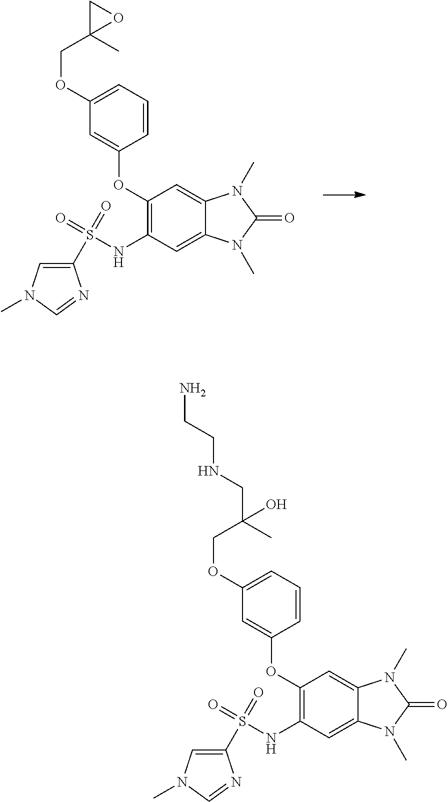 [SMILES: Cn1cnc(S(=O)(=O)Nc2cc3c(cc2Oc2cccc(OCC4(C)CO4)c2)n(C)c(=O)n3C)c1 ; Cn1cnc(S(=O)(=O)Nc2cc3c(cc2Oc2cccc(OCC(C)(O)CNCCN)c2)n(C)c(=O)n3C)c1]

A mixture of N-(1,3-dimethyl-6-(3-((2-methyloxiran-2-yl)methoxy)phenoxy)-2-oxo-2,3-dihydro-1H-benzo[d]imidazol-5-yl)-1-methyl-1H-imidazole-4-sulfonamide (20 mg, 0.040 mmol) and ethane-1,2-diamine (12 mg, 0.20 mmol) in THF (0.5 mL) was stirred at 60° C. overnight. The reaction mixture was concentrated and purified by prep-HPLC (Mobile phase: A=0.1% TFA/H₂O, B=0.1% TFA/MeCN; Gradient: B=10%-30% in 12 min; Column: C18) to give the title compound (1.7 mg, 7.6%). MS (ES+) C₂₅H₃₃N₇O₆S requires: 559. found 560 [M+H]⁺. ¹H-NMR (600 MHz, d₆-DMSO) δ: ppm 9.33 (s, 1H), 8.44 (s, 2H), 7.83 (s, 3H), 7.65 (s, 1H), 7.61 (s, 1H), 7.21 (t, J=8.3 Hz, 1H), 7.06 (s, 1H), 6.77 (s, 1H), 6.66 (dd, J=8.7, 2.3 Hz, 1H), 6.40 (s, 1H), 6.36 (dd, J=8.7, 2.3 Hz, 1H), 3.83 (d, J=9.1 Hz, 1H), 3.82 (d, J=9.1 Hz, 1H), 3.61 (s, 3H), 3.26 (s, 3H), 3.21 (s, 3H), 3.18 (m, 6H), 1.29 (s, 3H).

### Example 238

### N-{6-[3-(2-hydroxy-2-methylpropoxy)phenoxy]-1,3-dimethyl-2-oxo-2,3-dihydro-1H-1,3-benzodiazol-5-yl}-1-methyl-1H-imidazole-4-sulfonamide

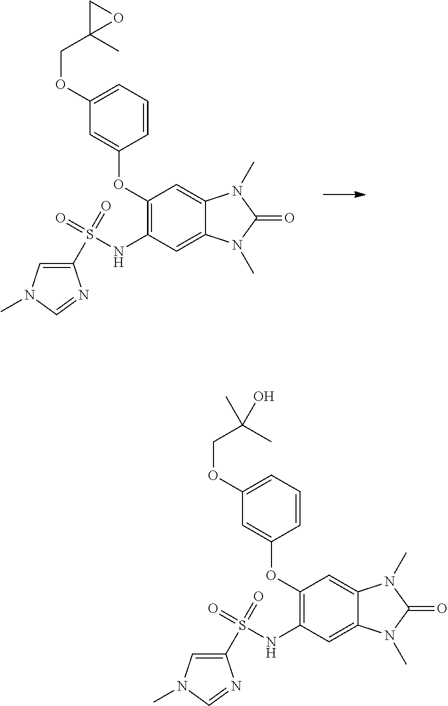 [SMILES: Cn1cnc(S(=O)(=O)Nc2cc3c(cc2Oc2cccc(OCC4(C)CO4)c2)n(C)c(=O)n3C)c1 ; Cn1cnc(S(=O)(=O)Nc2cc3c(cc2Oc2cccc(OCC(C)(C)O)c2)n(C)c(=O)n3C)c1]

A mixture of N-(1,3-dimethyl-6-(3-((2-methyloxiran-2-yl)methoxy)phenoxy)-2-oxo-2,3-dihydro-1H-benzo[d]imidazol-5-yl)-1-methyl-1H-imidazole-4-sulfonamide (20 mg, 0.040 mmol) and L-Selectride (0.12 mL, 0.12 mmol) in THF (0.5 mL) was stirred for 2 h at ambient temperature. The reaction mixture was quenched by TFA and concentrated, then the crude material was purified by prep-HPLC (Mobile phase: A=0.1% TFA/H₂O, B=0.1% TFA/MeCN; Gradient: B=20%-60% in 12 min; Column: C18) to give the title compound (3.5 mg, 17%). MS (ES+) C₂₃H₂₇N₅O₆S requires: 501. found 502 [M+H]⁺. ¹H-NMR (600 MHz, d₆-DMSO) δ: ppm 9.32 (s, 1H), 7.59 (s, 1H), 7.57 (s, 1H), 7.16 (t, J=8.3 Hz, 1H), 7.10 (s, 1H), 6.78 (s, 1H), 6.62 (dd, J=8.7, 2.3 Hz, 1H), 6.30 (dd, J=8.7, 2.3 Hz, 1H), 6.26 (s, 1H), 3.64 (s, 2H), 3.58 (s, 3H), 3.27 (s, 3H), 3.21 (s, 3H), 1.18 (s, 6H).

### Example 239

### 3-(3-{[1,3-dimethyl-6-(1-methyl-1H-imidazole-4-sulfonamido)-2-oxo-2,3-dihydro-1H-1,3-benzodiazol-5-yl]oxy}phenoxy)-2-methylpropyl 2-{[(tert-butoxy)carbonyl]amino}acetate

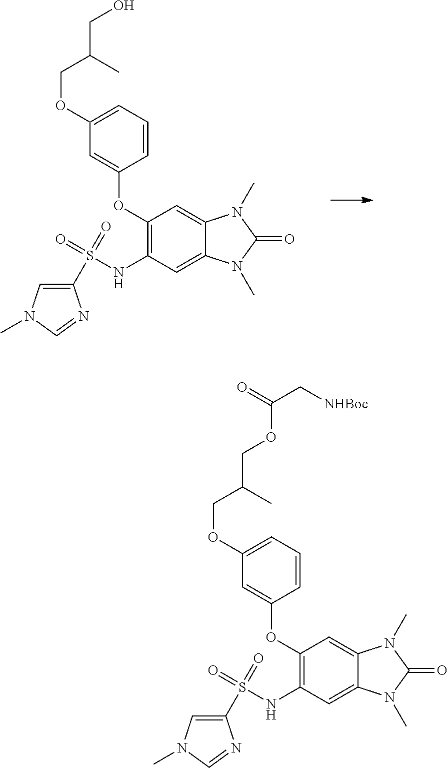 [SMILES: CC(CO)COc1cccc(Oc2cc3c(cc2NS(=O)(=O)c2cn(C)cn2)n(C)c(=O)n3C)c1 ; CC(COC(=O)CNC(=O)OC(C)(C)C)COc1cccc(Oc2cc3c(cc2NS(=O)(=O)c2cn(C)cn2)n(C)c(=O)n3C)c1]

A mixture of N-(6-(3-(3-hydroxy-2-methylpropoxy)phenoxy)-1,3-dimethyl-2-oxo-2,3-dihydro-1H-benzo[d]imidazol-5-yl)-1-methyl-1H-imidazole-4-sulfonamide (5.0 mg, 10 μmol) and 2-((tert-butoxycarbonyl)amino)acetic acid (1.7 mg, 10 μmol) in DMF (0.2 mL) was stirred overnight at ambient temperature. The reaction mixture was purified by prep-HPLC (Mobile phase: A=0.1% TFA/H2O, B=0.1% TFA/MeCN; Gradient: B=20%-60% in 12 min; Column: C18) to give the title compound (0.7 mg, 11%). MS (ES+) C₃₀H₃₈N₆O₉S requires: 658. found 659 [M+H]⁺. ¹H-NMR (600 MHz, CD₃OD) δ ppm 9.32 (s, 1H), 7.59 (s, 1H), 7.54 (s, 1H), 7.22 (s, 1H), 7.16 (t, J=8.3 Hz, 1H), 7.09 (s, 1H), 6.78 (s, 1H), 6.66 (dd, J=8.7, 2.3 Hz, 1H), 6.29 (s, 1H), 6.27 (dd, J=8.7, 2.3 Hz, 1H), 4.11 (m, 1H), 4.04 (m, 1H), 3.87 (m, 1H), 3.82 (m, 1H), 3.67 (d, J=9.1 Hz, 2H), 3.58 (s, 3H), 3.27 (s, 3H), 3.21 (s, 3H), 2.51 (m, 1H), 1.36 (s, 9H), 1.10 (d, J=7.2 Hz, 3H).

### Example 240

### N-(6-(3-(3-hydroxypropoxy)phenoxy)-1,3-dimethyl-2-oxo-2,3-dihydro-1H-benzo[d]imidazol-5-yl)-1-methyl-1H-imidazole-4-sulfonamide

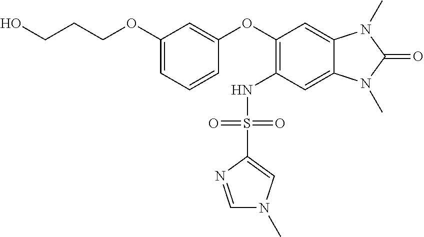 [SMILES: Cn1cnc(S(=O)(=O)Nc2cc3c(cc2Oc2cccc(OCCCO)c2)n(C)c(=O)n3C)c1]

To a solution of N-(6-(3-(allyloxy)phenoxy)-1,3-dimethyl-2-oxo-2,3-dihydro-1H-benzo[d]imidazol-5-yl)-1-methyl-1H-imidazole-4-sulfonamide (350 mg, 0.745 mmol) in THF (2 ml) was added borane (4.5 ml, 4.5 mmol) and the resulting mixture was stirred at 25° C. for 30 min. LC-MS showed the consumption of starting material. To the mixture, aq. NaOH (0.19 ml, 3.7 mmol) was added followed by hydrogen peroxide (423 mg, 3.73 mmol) and the resulting mixture was stirred at 25° C. The reaction was monitored for completion then quenched with 12 N HCl, water (100 mL) was added and the layers were separated. The aqueous phase was extracted with EtOAc (3×100 mL), the combined organic layers were washed with water, filtered and concentrated under reduced pressure. The aqueous phase was concentrated under reduced pressure and acetone was added and the resulting suspension was filtered to give a further amount of product. The combined product residues were purified by prep HPLC to obtain N-(6-(3-(3-hydroxypropoxy)phenoxy)-1,3-dimethyl-2-oxo-2,3-dihydro-1H-benzo[d]imidazol-5-yl)-1-methyl-1H-imidazole-4-sulfonamide (250 mg, 69%). MS (ES+) C₂₂H₂₅N₅O₆S requires: 487. found 488 [M+H]⁺. ¹H-NMR (600 MHz, CDCl₃) δ: 7.50 (s, 1H), 7.38 (d, J=5.3 Hz, 1H), 7.36 (d, J=7.7 Hz, 1H), 7.25 (d, J=4.4 Hz, 1H) 7.12 (m, 1H), 6.61 (m, 1H), 6.53 (s, 1H), 6.32 (t, J=8.5 Hz, 1H), 6.25 (s, 1H), 4.07 (t, J=6.0 Hz, 1H), 3.85 (t, J=5.8 Hz, 1H), 3.61 (s, 3H), 3,44 (s, 3H), 3.29 (s, 3H), 2.02 (m, 1H), 1.28 (d, J=6.4 Hz, 1H).

### Example 241

### N-(6-(3-(cyclopropylmethoxy)phenoxy)-1,3-dimethyl-2-oxo-2,3-dihydro-1H-benzo[d]imidazol-5-yl)-4-hydroxy-3-methoxybenzenesulfonamide

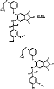 [SMILES: COc1ccc(S(=O)(=O)Nc2cc3c(cc2Oc2cccc(OCC4CC4)c2)n(C)c(=O)n3C)cc1OC ; [Li+] ; [Cl-] ; COc1cc(S(=O)(=O)Nc2cc3c(cc2Oc2cccc(OCC4CC4)c2)n(C)c(=O)n3C)ccc1O]

To a solution of N-(6-(3-(cyclopropylmethoxy)phenoxy)-1,3-dimethyl-2-oxo-2,3-dihydro-1H-benzo[d]imidazol-5-yl)-3,4-dimethoxybenzenesulfonamide (10 mg, 0.019 mmol) in DMF (1 ml) was added lithium chloride (2 mg, 0.04 mmol). The reaction mixture was heated to 110° C. and stirred for 12 h. The temperature of the reaction mixture was raised to 150° C. stirred for another 2 days. The cooled reaction mixture quenched with methanol, concentrated, filtered and purified by prep-HPLC to give N-(6-(3-(cyclopropylmethoxy)phenoxy)-1,3-dimethyl-2-oxo-2,3-dihydro-1H-benzo[d]imidazol-5-yl)-4-hydroxy-3-methoxybenzenesulfonamide (1.2 mg, 12%). MS (ES+) C₂₆H₂₇N₃O₇S requires: 525. found 526 [M+H]⁺. ¹H-NMR (600 MHz, d₆-DMSO) δ: 9.88 (s, 1H), 9.38 (s, 1H), 7.15 (s, 1H), 7.08-7.05 (m, 3H), 6.72 (m, 2H), 6.57 (m, 1H), 6.16-6.09 (m, 2H), 3.70 (d, J=7.1 Hz, 2H), 3.59 (s, 3H), 3.28 (s, 3H), 3.19 (s, 3H), 1.18 (m, 1H), 0.55 (m, 2H), 0.30 (m, 2H).

### Example 242

### N-(6-(3-((1,3-dimethyl-6-(1-methyl-1H-imidazole-4-sulfonamido)-2-oxo-2,3-dihydro-1H-benzo[d]imidazol-5-yl)oxy)-5-propoxyphenoxy)hexyl)acetamide

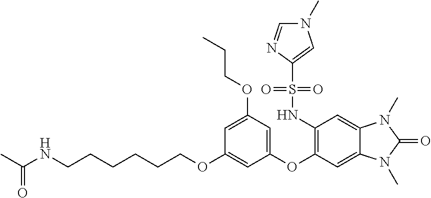 [SMILES: CCCOc1cc(OCCCCCCNC(C)=O)cc(Oc2cc3c(cc2NS(=O)(=O)c2cn(C)cn2)n(C)c(=O)n3C)c1]

To a solution of N-(6-(3-((6-aminohexyl)oxy)-5-propoxyphenoxy)-1,3-dimethyl-2-oxo-2,3-dihydro-1H-benzo[d]imidazol-5-yl)-1-methyl-1H-imidazole-4-sulfonamide (5 mg, 8.5 μmol) in DMF (0.5 mL) was added triethylamine (4 uL, 0.04 mmol) and acetic anhydride (87 mg, 0.85 mmol). The reaction mixture was stirred for 30 min, then diluted with EtOAc and the organic layer was washed with saturated aqueous NaHCO₃, concentrated, then the crude residue was purified by prep-HPLC (Mobile phase: A=0.1% TFA/H₂O, B=0.1% TFA/ACN; Gradient: B=10%-30% in 12 min; Column: C18) to give the title compound (2.4 mg, 45%). MS (ES+) C₃₀H₄₀N₆O₇S requires: 628. found 629 [M+H]⁺. ¹H-NMR (600 MHz, d₆-DMSO) δ 9.29 (s, 1H), 7.77 (s, 1H), 7.60 (s, 1H), 7.59 (s, 1H), 7.07 (s, 1H), 6.80 (s, 1H), 6.17 (t, J=2 Hz, H), 5.83 (d, J=2 Hz, 2H), 3.86 (t, J=7 Hz, 2H), 3.83 (t, J=6 Hz, 2H), 3.59 (s, 3H), 3.26 (s, 3H), 3.21 (s, 3H), 3.00 (m, 2H), 1.77 (s, 3H), 1.67 (m, 4H), 1.37 (m, 4H), 1.29 (m, 2H), 0.95 (t, J=7 Hz, 3H).

### Example 243

### (E)-3-((6-aminohex-2-en-1-yl)oxy)-N-(1,3-dimethyl-2-oxo-6-(3-propoxyphenoxy)-2,3-dihydro-1H-benzo[d]imidazol-5-yl)benzenesulfonamide 2,2,2-trifluoroacetate:

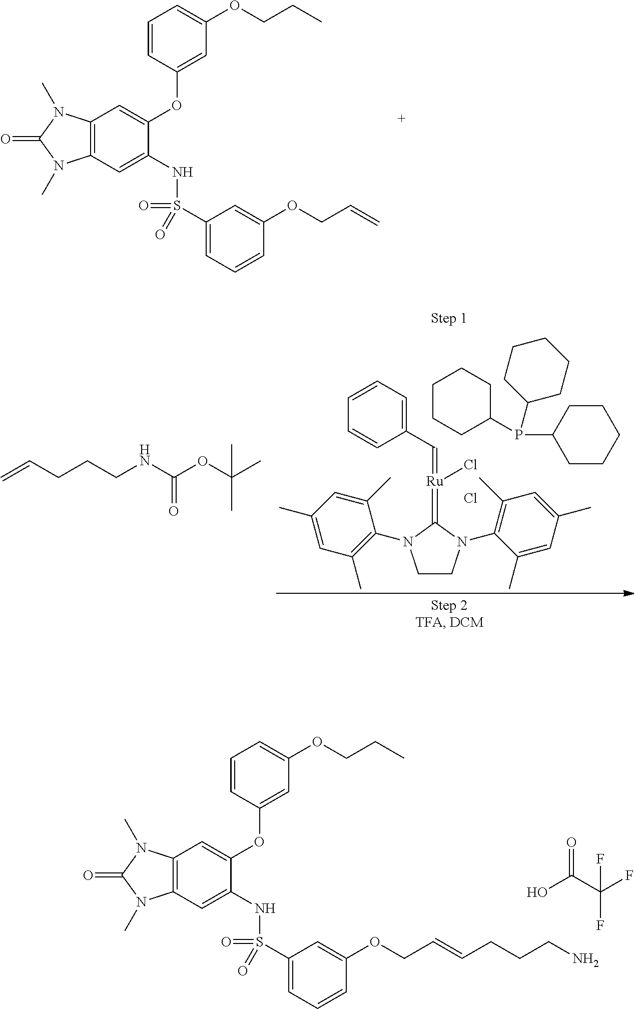

A solution of 3-(allyloxy)-N-(1,3-dimethyl-2-oxo-6-(3-propoxyphenoxy)-2,3-dihydro-1H-benzo[d]imidazol-5-yl)benzenesulfonamide (67 mg, 0.13 mmol), tert-butyl pent-4-en-1-ylcarbamate (218 mg, 1.178 mmol) in DCM (2 mL) was degassed with N₂ for 2 minutes. To the reaction mixture was added the Grubbs 2^{nd} generation catalyst (5 mg, 5.89 μmol) and the mixture was further degassed with N₂ and stirred overnight at ambient temperature. LCMS showed a small amount of product formation so the reaction mixture was heated to 35° C. overnight. Little to no change was observed, so the reaction mixture was concentrated and an additional 6 mg of catalyst was added, 2 mL of toluene and then heated to 60° C. for 2 days. The cooled reaction mixture was filtered through a pad of celite, concentrated and purified by column chromatography (1:1 EtOAc/hexanes) and dried to give (E)-tert-butyl (6-(3-(N-(1,3-dimethyl-2-oxo-6-(3-propoxyphenoxy)-2,3-dihydro-1H-benzo[d]imidazol-5-yl) sulfamoyl)phenoxy)hex-4-en-1-yl)carbamate as a light-brown film (36 mg, 40%). MS (ES+) C₃₅H₄₄N₄O₈S requires: 680. found 681 [M+H]⁺, 625 [M-C₄H₇]⁺, 100%.

Step 2: To a solution of (E)-tert-butyl (6-(3-(N-(1,3-dimethyl-2-oxo-6-(3-propoxyphenoxy)-2,3-dihydro-1H-benzo[d]imidazol-5-yl)sulfamoyl)phenoxy)hex-4-en-1-yl)carbamate (34 mg, 0.05 mmol) in DCM (2 ml) were added TFA (500 al, 6.49 mmol) and the resulting mixture was left standing at 25° C. for 2 days. The reaction mixture was concentrated to give a brown semi-solid then purified by HPLC to give (E)-3-((6-aminohex-2-en-1-yl)oxy)-N-(1,3-dimethyl-2-oxo-6-(3-propoxyphenoxy)-2,3-dihydro-1H-benzo[d]imidazol-5-yl)benzenesulfonamide 2,2,2-trifluoroacetate as a light-brown amorphous solid (22.6 mg, 65%). MS (ES+) C₃₀H₃₆N₄O₆S⁺ requires 580. found 581 [M+H]⁺. ¹H NMR (600 MHz, CDCl₃) δ: 7.92 (br-s, 3H), 7.34 (s, 1H), 7.25-7.17 (m, 3H), 7.12-7.03 (m, 2H), 6.98 (dt, J=7.0, 2.1 Hz, 1H), 6.59 (dd, J=6.3, 1.9 Hz, 1H), 6.41 (s, 1H), 6.09-6.02 (m, 2H), 5.71-5.46 (m, 2H), 4.23 (d, J=4.7 Hz, 2H), 3.81 (t, J=6.5 Hz, 2H), 3.42 (s, 3H), 3.24 (s, 3H), 3.02-2.88 (m, 2H), 2.12 (m, 2H), 1.78 (m, 4H), 1.02 (t, J=7.4 Hz, 3H).

### Example 244

### N,1-diallyl-N-(1,3-dimethyl-2-oxo-6-(3-propoxyphenoxy)-2,3-dihydro-1H-benzo[d]imidazol-5-yl)-1H-imidazole-4-sulfonamide

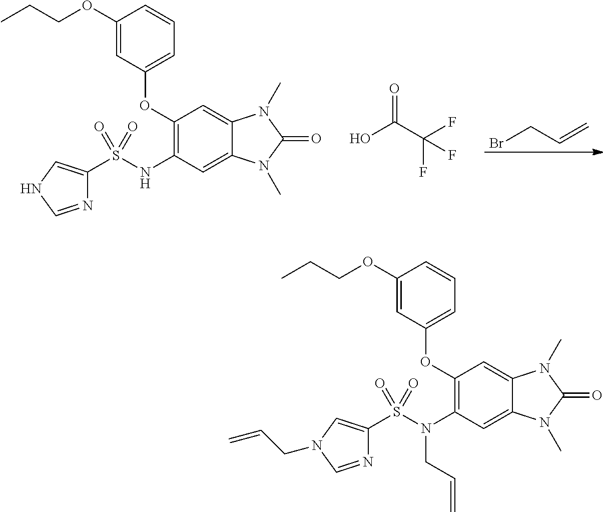

N-(1,3-dimethyl-2-oxo-6-(3-propoxyphenoxy)-2,3-dihydro-1H-benzo[d]imidazol-5-yl)-1H-imidazole-4-sulfonamide 2,2,2-trifluoroacetate (10 mg, 0.017 mmol) was dissolved in dioxanes (1.0 ml) and pyridine (0.013 ml, 0.18 mmol) was added followed by 3-bromoprop-1-ene (0.023 ml, 0.26 mmol). The reaction was heated in the microwave at 80° C. for 20 h. The cooled reaction mixture was concentrated and purified by prep-HPLC using a gradient of 30-70% ACN/water containing 0.1% TFA afforded a clean fraction of N,1-diallyl-N-(1,3-dimethyl-2-oxo-6-(3-propoxyphenoxy)-2,3-dihydro-1H-benzo[d]imidazol-5-yl)-1H-imidazole-4-sulfonamide (4 mg, 43%) as a white solid. MS (ES+) C₂₇H₃₁N₅O₅S requires: 537. found 538 [M+H]⁺. ¹H-NMR (600 MHz, d₆-DMSO) δ 10.98 (s, 1H), 9.30 (s, 1H), 8.26 (s, 1H), 7.24-7.18 (m, 2H), 6.83 (s, 1H), 6.68 (dd, J=8.2, 2.1 Hz, 1H), 6.29-6.23 (m, 2H), 6.03-5.91 (m, 2H), 5.45-5.33 (m, 3H), 5.30-5.23 (m, 1H), 4.91 (d, J=6.3 Hz, 2H), 4.73 (d, J=6.0 Hz, 2H), 3.89 (t, J=6.5 Hz, 2H), 3.34 (s, 3H), 3.22 (s, 3H), 1.75-1.67 (m, 2H), 0.97 (t, J=7.4 Hz, 3H).

### Example 245

### N-(6-(3-((6-aminohexyl)oxy)-5-propoxyphenoxy)-1,3-dimethyl-2-oxo-2,3-dihydro-1H-benzo[d]imidazol-5-yl)-3,4-dimethoxy-N-methylbenzenesulfonamide

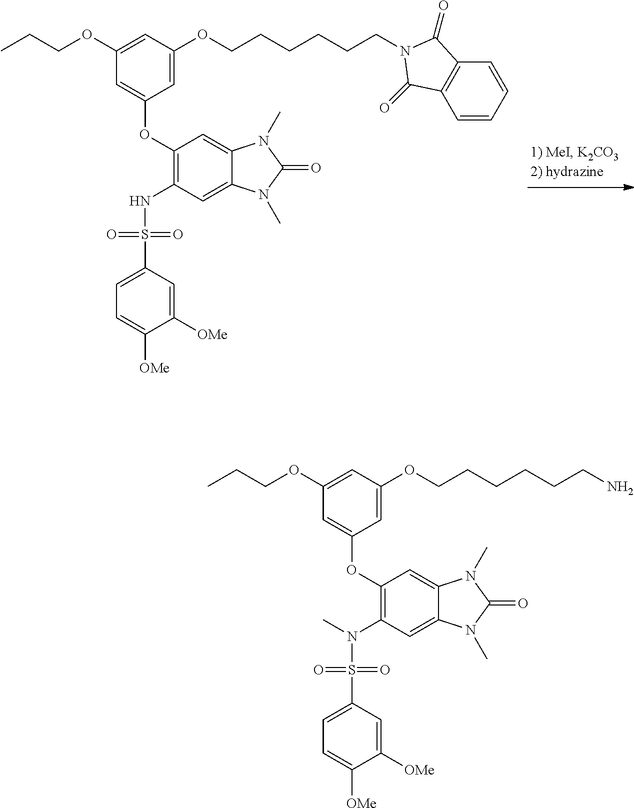 [SMILES: CCCOc1cc(OCCCCCCN2C(=O)c3ccccc3C2=O)cc(Oc2cc3c(cc2NS(=O)(=O)c2ccc(OC)c(OC)c2)n(C)c(=O)n3C)c1 ; CCCOc1cc(OCCCCCCN)cc(Oc2cc3c(cc2N(C)S(=O)(=O)c2ccc(OC)c(OC)c2)n(C)c(=O)n3C)c1]

To a solution of N-(6-(3-((6-(1,3-dioxoisoindolin-2-yl)hexyl)oxy)-5-propoxyphenoxy)-1,3-dimethyl-2-oxo-2,3-dihydro-1H-benzo[d]imidazol-5-yl)-3,4-dimethoxybenzenesulfonamide (50 mg, 0.065 mmol) in THF (2 ml) were added potassium carbonate (18 mg, 0.13 mmol) at 0° C. and methyl iodide (0.016 ml, 0.26 mmol) and the resulting mixture was stirred and gradually allowed to warm to RT. The reaction mixture was stirred at RT for 1 h then quenched with water and EtOAc. The organic layer concentrated and then treated with hydrazine (2.0 μl, 0.065 mmol) deprotect the phthalhydrazide, purification by preparative HPLC gave the TFA salt of N-(6-(3-((6-aminohexyl)oxy)-5-propoxyphenoxy)-1,3-dimethyl-2-oxo-2,3-dihydro-1H-benzo[d]imidazol-5-yl)-3,4-dimethoxy-N-methylbenzenesulfonamide (15 mg, 35%). MS (ES+) C₃₃H₄₄N₄O₈S requires: 656. found: 657 [M+H]⁺. ¹H NMR (d₆-DMSO) δ: 7.61 (s, 3H), 7.28 (d, J=8.6 Hz, 1H), 7.12 (d, J=6.9 Hz, 1H), 7.10 (s, 1H), 7.02 (s, 1H), 6.86 (s, 1H), 6.15 (s, 1H), 5.80 (s, 2H), 3.85-3.83 (m, 5H), 3.80 (t, J=6.0 Hz, 2H), 3.71 (s, 3H), 3.29 (s, 3H), 3.26 (s, 3H), 3.01 (s, 3H), 2.79-2.76 (m, 2H), 1.68-1.64 (m, 4H), 1.56-1.51 (m, 2H), 1.41-1.33 (m, 4H), 0.95-0.92 (t, J=7.4 Hz, 3H).

### Example 246 (Method G)

### N-(1,3-dimethyl-6-(3-(4-morpholinobutoxy)-5-propoxyphenoxy)-2-oxo-2,3-dihydro-1H-benzo[d]imidazol-5-yl)-3,4-dimethoxybenzenesulfonamide 2,2,2-trifluoroacetate

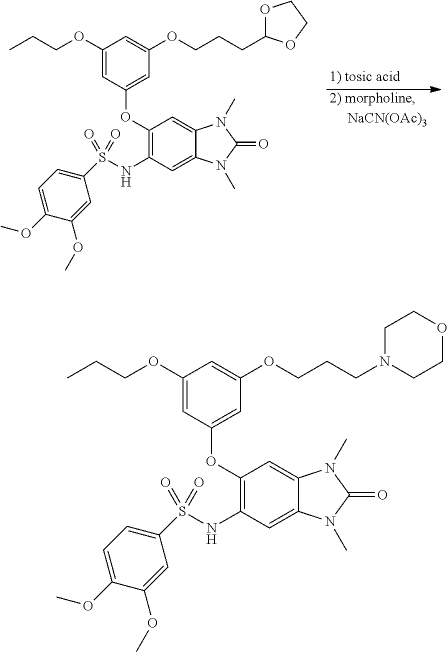

Step 1: N-(6-(3-(3-(1,3-dioxolan-2-yl)propoxy)-5-propoxyphenoxy)-1,3-dimethyl-2-oxo-2,3-dihydro-1H-benzo[d]imidazol-5-yl)-3,4-dimethoxybenzenesulfonamide (200 mg, 0.304 mmol) was dissolved in THF and water (5.00 ml). Tosic acid (500 mg, 2.63 mmol) was added and the reaction was stirred for 2 h at 50° C. The reaction was then allowed to cool to room temperature and was partitioned between water (15 mL) and EtOAc (15 mL). The layers were separated and the aqueous layer was extracted with EtOAc (15 mL). The combined organic layers were dried over Na₂SO₄, filtered, and concentrated. Purification by silica gel chromatography (0% EtOAc/hexane to 100% EtOAc/hexane) to afford N-(1,3-dimethyl-2-oxo-6-(3-(4-oxobutoxy)-5-propoxyphenoxy)-2,3-dihydro-1H-benzo[d]imidazol-5-yl)-3,4-dimethoxybenzenesulfonamide (138 mg, 74% yield) as a clear-colorless viscous liquid. MS (ES+) C₃₀H₃₅N₃O₉S requires: 613. found 614 [M+H]⁺.

Step 2: To a solution of N-(1,3-dimethyl-2-oxo-6-(3-(4-oxobutoxy)-5-propoxyphenoxy)-2,3-dihydro-1H-benzo[d]imidazol-5-yl)-3,4-dimethoxybenzenesulfonamide (45 mg, 0.073 mmol) in methanol (2.00 ml) was added acetic acid (8.40 μl, 0.147 mmol), morpholine (6.39 mg, 0.073 mmol), and sodium triacetoxyborohydride (38.9 mg, 0.183 mmol). The reaction was stirred at room temperature for 1 hour and then concentrated. Purification by prep-HPLC using a gradient of 30-70% ACN/water containing 0.1% TFA to afford N-(1,3-dimethyl-6-(3-(4-morpholinobutoxy)-5-propoxyphenoxy)-2-oxo-2,3-dihydro-1H-benzo[d]imidazol-5-yl)-3,4-dimethoxybenzenesulfonamide 2,2,2-trifluoroacetate (33 mg, 56% yield) as a white solid. MS (ES+) C₃₄H₄₄N₄O₉S requires: 684. found 685 [M+H]⁺. ¹H NMR (600 MHz, d₆-DMSO) δ 9.54 (br-s, 1H), 9.45 (s, 1H), 7.22-7.16 (m, 2H), 7.06 (s, 1H), 6.94-6.87 (m, 1H), 6.75 (s, 1H), 6.13 (s, 1H), 5.72 (s, 1H), 5.67 (s, 1H), 4.03-3.94 (m, 2H), 3.85 (t, J=5.9 Hz, 2H), 3.81-3.74 (m, 2H), 3.77 (s, 3H), 3.68-3.59 (m, 2H), 3.62 (s, 3H), 3.47-3.39 (m, 2H), 3.29 (s, 3H), 3.20 (s, 3H), 3.19-3.11 (m, 2H), 3.10-2.99 (m, 2H), 1.82-1.62 (m, 6H), 0.95 (t, J=7.4 Hz, 3H).

The following compounds were prepared in a similar manner as Example 246 (Method G).

| No. | Structure | Name | MW | [M + H]⁺ | Method |
|---|---|---|---|---|---|
| 247 | 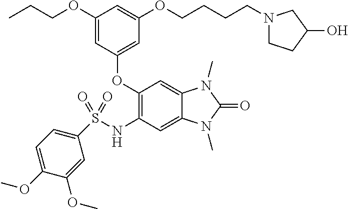 [SMILES: CCCOc1cc(OCCCCN2CCC(O)C2)cc(Oc2cc3c(cc2NS(=O)(=O)c2ccc(OC)c(OC)c2)n(C)c(=O)n3C)c1] | N-(6-{3-[4-(3- hydroxypyrrolidin- 1-yl)butoxy]-5- propoxyphenoxy}- 1,3-dimethyl-2- oxo-2,3-dihydro- 1H-1,3- benzodiazol-5- yl)-3,4- dimethoxybenzene- 1-sulfonamide | 684 | 685 | G |
| 248 | 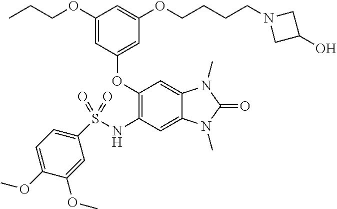 [SMILES: CCCOc1cc(OCCCCN2CC(O)C2)cc(Oc2cc3c(cc2NS(=O)(=O)c2ccc(OC)c(OC)c2)n(C)c(=O)n3C)c1] | N-(6-{3-[4-(3- hydroxyazetidin- 1-yl)butoxy]-5- propoxyphenoxy}- 1,3-dimethyl-2- oxo-2,3-dihydro- 1H-1,3- benzodiazol-5- yl)-3,4- dimethoxybenzene- 1-sulfonamide | 670 | 671 | G |
| 249 | 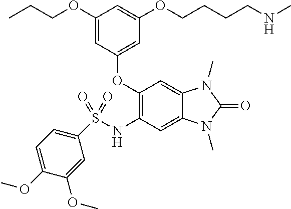 | N-(1,3-dimethyl- 6-{3-[4- (methylamino) butoxy]-5- propoxyphenoxy}- 2-oxo-2,3- dihydro-1H-1,3- benzodiazol-5- yl)-3,4- dimethoxybenzene- 1-sulfonamide | 628 | 629 | G |
| 250 | 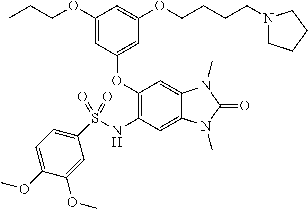 [SMILES: CCCOc1cc(OCCCCN2CCCC2)cc(Oc2cc3c(cc2NS(=O)(=O)c2ccc(OC)c(OC)c2)n(C)c(=O)n3C)c1] | N-(1,3-dimethyl- 2-oxo-6-{3- propoxy-5-[4- (pyrrolidin-1- yl)butoxy]phenoxy}- 2,3-dihydro- 1H-1,3- benzodiazol-5- yl)-3,4- dimethoxybenzene- 1-sulfonamide | 668 | 669 | G |
| 251 | 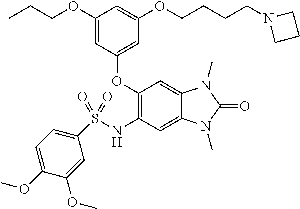 [SMILES: CCCOc1cc(OCCCCN2CCC2)cc(Oc2cc3c(cc2NS(=O)(=O)c2ccc(OC)c(OC)c2)n(C)c(=O)n3C)c1] | N-(6-{3-[4- (azetidin-1- yl)butoxy]-5- propoxyphenoxy}- 1,3-dimethyl-2- oxo-2,3-dihydro- 1H-1,3- benzodiazol-5- yl)-3,4- dimethoxybenzene- 1-sulfonamide | 654 | 655 | G |

### Example 252

### Methyl 3-({6-[3-(4-aminobutoxy)-5-propoxyphenoxy]-1,3-dimethyl-2-oxo-2,3-dihydro-1H-1,3-benzodiazol-5-yl}sulfamoyl)benzoate

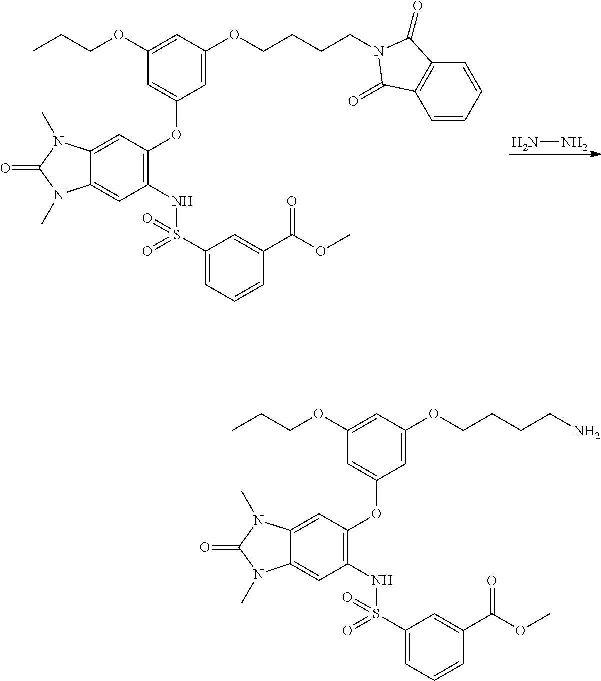

Was prepared in a similar manner to Example 10 (Method F) using methyl 3-(N-(6-(3-(4-(1,3-dioxoisoindolin-2-yl)butoxy)-5-propoxyphenoxy)-1,3-dimethyl-2-oxo-2,3-dihydro-1H-benzo[d]imidazol-5-yl)sulfamoyl)benzoate and hydrazine hydrate. The product was isolated by filtration of the resulting suspension to give the title compound as a pale-yellow solid (186 mg, 96%). MS (ES+) C₃₀H₃₆N₄O₈S requires: 612. found: 613 [M+H]⁺.

### Example 253

### 3-(N-(6-(3-(4-(dimethylamino)butoxy)-5-propoxyphenoxy)-1,3-dimethyl-2-oxo-2,3-dihydro-1H-benzo[d]imidazol-5-yl)sulfamoyl)benzoic acid

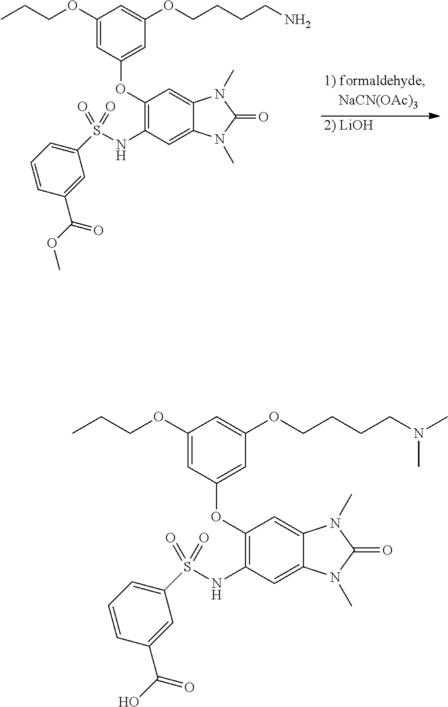

Step 1: To a solution of methyl 3-(N-(6-(3-(4-aminobutoxy)-5-propoxyphenoxy)-1,3-dimethyl-2-oxo-2,3-dihydro-1H-benzo[d]imidazol-5-yl)sulfamoyl)benzoate 2,2,2-trifluoroacetate (27 mg, 0.037 mmol) in methanol (3.000 ml) was added triethylamine (5.2 al, 0.037 mmol), acetic acid (4.3 al, 0.074 mmol), formaldehyde (8.2 al, 0.30 mmol), and sodium triacetoxyborohydride (19.7 mg, 0.093 mmol). The reaction was stirred at room temperature and checked by LCMS every 30 minutes. After 3 h the reaction was complete by LCMS. The reaction was quenched with a few drops of TFA and concentrated. Purification by prep-HPLC using a gradient of 20-60% AcN/water containing 0.1% TFA to afford methyl 3-(N-(6-(3-(4-(dimethylamino)butoxy)-5-propoxyphenoxy)-1,3-dimethyl-2-oxo-2,3-dihydro-1H-benzo[d]imidazol-5-yl)sulfamoyl)benzoate 2,2,2-trifluoroacetate (7 mg, 25% yield) as a yellow liquid. MS (ES+) C₃₂H₄₀N₄O₈S requires: 640. found 641 [M+H]⁺. ¹H NMR (600 MHz, CD₃OD) δ 8.28-8.25 (m, 1H), 8.04-7.99 (m, 1H), 7.82-7.77 (m, 1H), 7.42-7.37 (m, 1H), 7.38 (s, 1H), 6.64 (s, 1H), 6.09-6.05 (m, 1H), 5.63-5.59 (m, 1H), 5.55-5.52 (m, 1H), 3.87 (t, J=5.9 Hz, 2H), 3.86 (s, 3H), 3.76 (t, J=6.5 Hz, 2H), 3.02 (s, 3H), 3.26 (s, 3H), 3.24-3.17 (m, 2H), 2.91 (s, 6H), 1.94-1.86 (m, 2H), 1.86-1.79 (m, 2H), 1.79-1.70 (m, 2H), 1.02 (t, J=7.4 Hz, 3H).

Step 2: To a solution of methyl 3-(N-(6-(3-(4-(dimethylamino)butoxy)-5-propoxyphenoxy)-1,3-dimethyl-2-oxo-2,3-dihydro-1H-benzo[d]imidazol-5-yl)sulfamoyl)benzoate 2,2,2-trifluoroacetate (50 mg, 0.066 mmol) was dissolved in methanol (3.0 ml) and a solution of LiOH (7.9 mg, 0.33 mmol) in water (1.0 ml) was added. The reaction was stirred at 50° C. for 3 h and then the methanol was stripped off. The reaction was partitioned between water (25 mL) and EtOAc (25 mL) and the layers were separated. The aqueous layer was extracted with EtOAc (25 mL) and the combined organics were dried over Na₂SO₄, filtered, and concentrated to give methyl 3-(N-(6-(3-(4-(dimethylamino)butoxy)-5-propoxyphenoxy)-1,3-dimethyl-2-oxo-2,3-dihydro-1H-benzo[d]imidazol-5-yl)sulfamoyl)benzoate 2,2,2-trifluoroacetate as a white solid. MS (ES+) C₃₁H₃₈N₄O₈S requires: 626. found 627 [M+H]⁺. ¹H NMR (600 MHz, CD₃OD) δ 8.26 (s, 1H), 8.04 (d, J=7.8 Hz, 1H), 7.79 (d, J=7.1 Hz, 1H), 7.41-7.35 (m, 2H), 6.63 (s, 1H), 6.28-6.23 (m, 1H), 6.10-6.06 (m, 1H), 5.66-5.62 (m, 1H), 5.58-5.54 (m, 1H), 3.88 (t, J=5.8 Hz, 2H), 3.76 (t, J=6.4 Hz, 2H), 3.44 (s, 3H), 3.26 (s, 3H), 3.23-3.17 (m, 2H), 2.91 (s, 6H), 1.94-1.78 (m, 4H), 1.79-1.70 (m, 2H), 1.02 (t, J=7.4 Hz, 3H).

### Example 254

### 4-(3-((6-(3,4-dimethoxyphenylsulfonamido)-1,3-dimethyl-2-oxo-2,3-dihydro-1H-benzo[d]imidazol-5-yl)oxy)-5-propoxyphenoxy)-N,N,N-trimethylbutan-1-aminium 2,2,2-trifluoroacetate

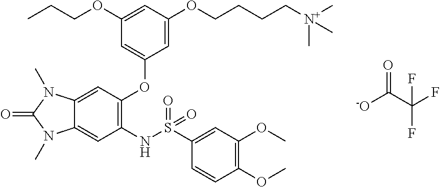

To a 0° C. solution of N-(6-(3-(4-(dimethylamino)butoxy)-5-propoxyphenoxy)-1,3-dimethyl-2-oxo-2,3-dihydro-1H-benzo[d]imidazol-5-yl)-3,4-dimethoxybenzenesulfonamide (9.2 mg, 0.014 mmol)(IACS-009571-001-4) in THF (0.5 ml) were added sodium hydride (10 mg, 0.417 mmol) and iodomethane (10 μl, 0.160 mmol) and the resulting mixture was warmed to room temp, then quenched with methanol, concentrated, took up into DMSO and purified by mass-triggered preparative HPLC (Mobile phase: A=0.1% TFA/H₂O, B=0.1% TFA/MeCN; Gradient: B=20-60%; 12 min; Column: C18) to give 4-(3-((6-(3,4-dimethoxyphenylsulfonamido)-1,3-dimethyl-2-oxo-2,3-dihydro-1H-benzo[d]imidazol-5-yl)oxy)-5-propoxyphenoxy)-N,N,N-trimethylbutan-1-aminium 2,2,2-trifluoroacetate (4.5 mg, 41%) as a pale yellow solid. MS (ES+) C₃₃H₄₅N₄O₈S⁺ requires: 657. found: 658 [M+H]⁺. ¹H NMR (600 MHz, d₆-DMSO) δ: 9.49 (s, 1H), 7.20-7.17 (m, 2H), 7.07 (s, 1H), 6.90 (m, 1H), 6.76 (s, 1H), 6.14 (t, J=2.1H, 1H), 5.72 (t, J=2.1 Hz, 1H), 5.66 (t, J=2.1 Hz, 1H), 3.86 (t, J=6.2 Hz, 2H), 3.79-3.75 (m, 5H), 3.62 (s, 3H), 3.33 (m, 2H), 3.29 (s, 3H), 3.20 (s, 3H), 3.05 (s, 9H), 1.80 (m, 2H), 1.68 (m, 4H), 0.95 (t, J=7.4 Hz, 3H).

### Biological Activity Assays

The following are assays that may be used to evaluate the biological efficacy of compounds of Formula (I).

### Trim24 AlphaScreen Assay

Recombinant human His-tagged Trim24-PHD-bromodomain (produced in-house), biotinylated H3K23ac (1-33aa) peptide (AnaSpec, Inc.), and test compound were added to a 384-well OptiPlate (Perkin Elmer) and incubated at room temperature for one hour. The assay buffer consisted of 50 mM Hepes, pH 7.5, 100 mM NaCl, and 0.12 mM Triton X-100. Final concentrations for this reaction were as follows: 5 nM Trim24 protein, 15 nM peptide, variable concentrations of compound (3-fold serial dilutions), and 0.5% (v/v) DMSO.

Streptavidin donor beads and nickel chelate acceptor beads, both from a Perkin Elmer AlphaScreen Histidine Detection Kit, were then added to the Optiplate to a final concentration of 10 ug/mL each. Following a 2 hour incubation at room temperature, the microplate was read on an Envision Plate Reader (Perkin Elmer). Percent of control (POC) values were calculated from the following formula:

POC=[sample signal-average background signal]/[average maximum signal−average background signal]*100

The average maximum signal was obtained from wells containing all assay components except test compound. The average background signal pertained to wells with all assay components except Trim24 and test compound. IC₅₀ values were calculated using a four-parameter logistic curve fit.

The following are table shows the Trim24 AlphaScreen Assay results for the synthesized compounds. Letter codes represent IC₅₀ activity ranges: 1-10 nM=“A”, 10-100 nM=“B”, 100-1,000 nM=“C”, and >1000 nM=“D”. “ND” indicates no data.

TABLE 2
Trim24 Assay Data

| | TRIM24 IC₅₀ |
|---|---|
| Example No. | Activity |
| 1 | C |
| 2 | C |
| 3 | D |
| 4 | C |
| 5 | C |
| 6 | C |
| 7 | B |
| 8 | A |
| 9 | D |
| 10 | A |
| 11 | D |
| 12 | D |
| 13 | D |
| 14 | C |
| 15 | D |
| 16 | D |
| 17 | C |
| 18 | C |
| 19 | C |
| 20 | D |
| 21 | D |
| 22 | C |
| 23 | C |
| 24 | D |
| 25 | C |
| 26 | C |
| 27 | D |
| 28 | C |
| 29 | C |
| 30 | C |
| 31 | C |
| 32 | D |
| 33 | D |
| 34 | C |
| 35 | D |
| 36 | C |
| 37 | D |
| 38 | D |
| 39 | C |
| 40 | D |
| 41 | C |
| 42 | C |
| 43 | C |
| 44 | C |
| 45 | B |
| 46 | C |
| 47 | C |
| 48 | C |
| 49 | C |
| 50 | C |
| 51 | C |
| 52 | C |
| 53 | C |
| 54 | C |
| 55 | C |
| 56 | D |
| 57 | C |
| 58 | B |
| 59 | C |
| 60 | B |
| 61 | B |
| 62 | C |
| 63 | C |
| 64 | C |
| 65 | C |
| 66 | B |
| 67 | B |
| 68 | C |
| 69 | B |
| 70 | B |
| 71 | B |
| 72 | B |
| 73 | D |
| 74 | C |
| 75 | B |
| 76 | C |
| 77 | C |
| 78 | C |
| 79 | D |
| 80 | D |
| 81 | D |
| 82 | C |
| 83 | B |
| 84 | C |
| 85 | C |
| 86 | B |
| 87 | C |
| 88 | B |
| 89 | C |
| 90 | B |
| 91 | B |
| 92 | B |
| 93 | C |
| 94 | B |
| 95 | C |
| 96 | B |
| 97 | C |
| 98 | B |
| 99 | B |
| 100 | B |
| 101 | B |
| 102 | B |
| 103 | D |
| 104 | D |
| 105 | B |
| 106 | C |
| 107 | B |
| 108 | B |
| 109 | B |
| 110 | B |
| 111 | D |
| 112 | C |
| 113 | C |
| 114 | D |
| 115 | C |
| 116 | C |
| 117 | B |
| 118 | B |
| 119 | C |
| 120 | C |
| 121 | C |
| 122 | B |
| 123 | B |
| 124 | B |
| 125 | B |
| 126 | B |
| 127 | B |
| 128 | B |
| 129 | C |
| 130 | D |
| 131 | C |
| 132 | C |
| 133 | C |
| 134 | B |
| 135 | B |
| 136 | B |
| 137 | B |
| 138 | B |
| 139 | B |
| 140 | B |
| 141 | B |
| 142 | C |
| 143 | C |
| 144 | C |
| 145 | B |
| 146 | C |
| 147 | B |
| 148 | B |
| 149 | B |
| 150 | C |
| 151 | B |
| 152 | B |
| 153 | B |
| 154 | B |
| 155 | C |
| 156 | C |
| 157 | C |
| 158 | B |
| 159 | B |
| 160 | C |
| 161 | C |
| 162 | C |
| 163 | C |
| 164 | C |
| 165 | B |
| 166 | C |
| 167 | C |
| 168 | B |
| 169 | C |
| 170 | C |
| 171 | C |
| 172 | C |
| 173 | C |
| 174 | B |
| 175 | C |
| 176 | B |
| 177 | C |
| 178 | B |
| 179 | B |
| 180 | B |
| 181 | B |
| 182 | B |
| 183 | A |
| 184 | B |
| 185 | C |
| 186 | A |
| 187 | B |
| 188 | B |
| 189 | A |
| 190 | B |
| 191 | B |
| 192 | B |
| 193 | C |
| 194 | B |
| 195 | B |
| 196 | A |
| 197 | B |
| 198 | B |
| 199 | B |
| 200 | A |
| 201 | A |
| 202 | A |
| 203 | B |
| 204 | B |
| 205 | A |
| 206 | B |
| 207 | B |
| 208 | B |
| 209 | A |
| 210 | B |
| 211 | A |
| 212 | A |
| 213 | B |
| 214 | B |
| 215 | A |
| 216 | B |
| 217 | B |
| 218 | B |
| 219 | A |
| 220 | C |
| 221 | B |
| 222 | B |
| 223 | B |
| 224 | B |
| 225 | B |
| 226 | B |
| 227 | D |
| 228 | C |
| 229 | C |
| 230 | C |
| 231 | C |
| 232 | C |
| 233 | C |
| 234 | D |
| 235 | D |
| 236 | C |
| 237 | C |
| 238 | C |
| 239 | C |
| 240 | C |
| 241 | B |
| 242 | B |
| 243 | B |
| 244 | C |
| 245 | C |
| 246 | B |
| 247 | B |
| 248 | B |
| 249 | B |
| 250 | B |
| 251 | A |
| 252 | B |
| 253 | B |
| 254 | A |

### BROMOscan DiscoveRx Binding Constant Assay for BRPF1, BRPF2, BRPF3, and TRIM24

### Assay Principle

Compounds that bind the bromodomain active site and directly (sterically) or indirectly (allosterically) prevent bromodomain binding to the immobilized ligand, will reduce the amount of protein captured on the solid support (Panels A & B). Conversely, test molecules that do not bind the bromodomain have no effect on the amount of bromodomain captured on the solid support (Panel C). Screening “hits” are identified by measuring the amount of bromodomain captured in test versus control samples by using a quantitative, precise and ultra-sensitive qPCR method that detects the associated DNA label. In a similar manner, dissociation constants (K_{d}'s) for test compound-bromodomain interactions are calculated by measuring the amount of bromodomain captured on the solid support as a function of the test compound concentration.

### Protocol Description

Bromodomain assays. T7 phage strains displaying bromodomains were grown in parallel in 24-well blocks in an E. coli host derived from the BL21 strain. E. coli were grown to log-phase and infected with T7 phage from a frozen stock (multiplicity of infection=0.4) and incubated with shaking at 32° C. until lysis (90-150 minutes). The lysates were centrifuged (5,000×g) and filtered (0.2 μm) to remove cell debris. Streptavidin-coated magnetic beads were treated with biotinylated small molecule or acetylated peptide ligands for 30 minutes at room temperature to generate affinity resins for bromodomain assays. The liganded beads were blocked with excess biotin and washed with blocking buffer (SeaBlock (Pierce), 1% BSA, 0.05% Tween 20, 1 mM DTT) to remove unbound ligand and to reduce non-specific phage binding. Binding reactions were assembled by combining bromodomains, liganded affinity beads, and test compounds in 1× binding buffer (17% SeaBlock, 0.33×PBS, 0.04% Tween 20, 0.02% BSA, 0.004% Sodium azide, 7.4 mM DTT). Test compounds were prepared as 1000× stocks in 100% DMSO and subsequently diluted 1:10 in monoethylene glycol (MEG) to create stocks at 100× the screening concentration (resulting stock solution is 10% DMSO/90% MEG). The compounds were then diluted directly into the assays such that the final concentration of DMSO and MEG were 0.1% and 0.9%, respectively. All reactions were performed in polystyrene 96-well plates in a final volume of 0.135 ml.

The assay plates were incubated at room temperature with shaking for 1 hour and the affinity beads were washed with wash buffer (1×PBS, 0.05% Tween 20). The beads were then re-suspended in elution buffer (lx PBS, 0.05% Tween 20, 2 μM nonbiotinylated affinity ligand) and incubated at room temperature with shaking for 30 minutes. The bromodomain concentration in the eluates was measured by qPCR.

### Compound Handling

An 11-point 3-fold serial dilution of each test compound was prepared in 100% DMSO at 1000× final test concentration. This serial is then diluted to 100× in ethylene glycol and subsequently diluted to 1× in the assay (final DMSO concentration=0.1%, Ethylene glycol concentration=0.9%). Most K_{d}'s were determined using a compound top concentration=10,000 nM. If the initial K_{d} determined was <0.169 nM (the lowest concentration tested), the measurement was repeated with a serial dilution starting at a lower top concentration.

### Binding Constants (K_{d}'s)

Binding constants (K_{d}'s) were calculated with a standard dose-response curve using the Hill equation: Response=Background+Signal−Background 1+(K_{d} Hill Slope/DoseHill Slope) The Hill Slope was set to −1. Curves were fitted using a non-linear least square fit with the Levenberg-Marquardt algorithm.

The following are table shows the Binding Constant Assay results for the synthesized compounds. Letter codes represent K_{d} activity ranges: <1 nM=“A”, 1-10 nM=“B”, 11-100 nM=“C”, and >100 nM=“D”.

TABLE 3
Binding Constant Data

| Example No. | TRIM24 K_{d} | BRPF1 K_{d} | BRPF2 K_{d} | BRPF3 K_{d} |
|---|---|---|---|---|
| 8 | A | B | B | C |
| 45 | B | C | C | C |
| 46 | C | C | C | D |
| 110 | B | B | C | C |
| 125 | B | B | B | C |

### OTHER EMBODIMENTS

The detailed description set-forth above is provided to aid those skilled in the art in practicing the present disclosure. However, the disclosure described and claimed herein is not to be limited in scope by the specific embodiments herein disclosed because these embodiments are intended as illustration of several aspects of the disclosure. Any equivalent embodiments are intended to be within the scope of this disclosure. Indeed, various modifications of the disclosure in addition to those shown and described herein will become apparent to those skilled in the art from the foregoing description, which do not depart from the spirit or scope of the present inventive discovery. Such modifications are also intended to fall within the scope of the appended claims.

What is claimed is:

1. A compound of structural Formula I 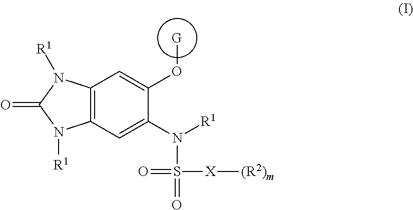
or a salt thereof, wherein:
m is chosen from the integers 0, 1, 2, and 3;
X is chosen from alkyl, aryl, cycloalkyl, heteroaryl, heteroarylalkyl, heterocycloalkyl, and heterocycloalkylalkyl;
each R¹ is independently chosen from alkenyl, alkyl, H, and haloalkyl;
each R² is independently chosen from alkenyl, alkoxy, alkyl, aryl, arylalkyl, cyano, cycloalkyl, H, halo, haloalkoxy, heteroaryl, heteroarylalkyl, heterocycloalkyl, hydroxyl, N(R³)₂, C(O)OH, N(R³)C(O)C(R³)₃, N(R³)C(O)OC(R³)₃, C(O)OC(R³)₃, and C(O)N(R³)₂, wherein two R² groups together with the atoms to which they are attached optionally form an aryl, cycloalkyl, heteroaryl, or heterocycloalkyl ring, which may be optionally substituted with between 0 and 3 R³ groups;
each R³ is independently chosen from alkenyl, alkoxy, alkyl, aminoalkyl, aryl, arylalkyl, cyano, cycloalkyl, cycloalkylalkyl, cycloalkylalkoxy, H, halo, haloalkyl, heteroaryl, heteroarylalkyl, heterocycloalkyl, heterocycloalkylalkyl, and hydroxyl; and
G is phenyl, which may be optionally substituted.

2. The compound as recited in claim 1, wherein the compound has structural Formula II: 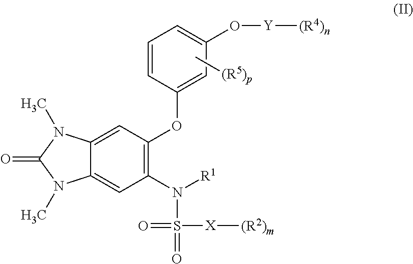
or a salt thereof, wherein:
m, n, and q are independently chosen from the integers 0, 1, 2, and 3;
p is chosen from the integers 0, 1, 2, 3, and 4;
X, Y, and Z are independently chosen from alkyl, aryl, cycloalkyl, heteroaryl, heteroarylalkyl, heterocycloalkyl, and heterocycloalkylalkyl;
R¹ is chosen from alkenyl, alkyl, H, and haloalkyl;
each R², R⁴, and R⁶ is independently chosen from alkenyl, alkoxy, alkyl, aryl, arylalkyl, cyano, cycloalkyl, H, halo, haloalkoxy, heteroaryl, heteroarylalkyl, heterocycloalkyl, hydroxyl, N(R³)₂, C(O)OH, N(R³)C(O)C(R³)₃, N(R³)C(O)OC(R³)₃, C(O)OC(R³)₃, and C(O)N(R³)₂, wherein two R² groups together with the atoms to which they are attached optionally form an aryl, cycloalkyl, heteroaryl, or heterocycloalkyl ring, which may be optionally substituted with between 0 and 3 R³ groups, wherein two R⁴ groups together with the atoms to which they are attached optionally form an aryl, cycloalkyl, heteroaryl, or heterocycloalkyl ring, which may be optionally substituted with between 0 and 3 R³ groups, wherein two R⁶ groups together with the atoms to which they are attached optionally form an aryl, cycloalkyl, heteroaryl, or heterocycloalkyl ring, which may be optionally substituted with between 0 and 3 R³ groups;
each R³ is independently chosen from alkenyl, alkoxy, alkyl, aminoalkyl, aryl, arylalkyl, cyano, cycloalkyl, cycloalkylalkyl, cycloalkylalkoxy, H, halo, haloalkyl, heteroaryl, heteroarylalkyl, heterocycloalkyl, heterocycloalkylalkyl, and hydroxyl; and
each R⁵ is independently chosen from O—Z—(R⁶)_{q}, alkyl, cyano, H, and halo.

3. The compound as recited in claim 1, wherein the compound has structural Formula III: 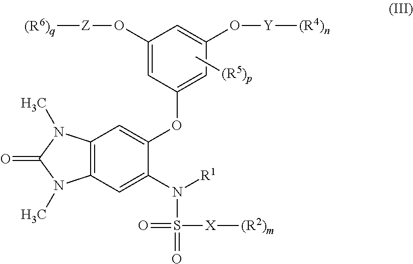
or a salt thereof, wherein:
m, n, p, and q are independently chosen from the integers 0, 1, 2, and 3;
X, Y, and Z are independently chosen from alkyl, aryl, cycloalkyl, heteroaryl, heteroarylalkyl, heterocycloalkyl, and heterocycloalkylalkyl;
R¹ is chosen from alkenyl, alkyl, H, and haloalkyl;
each R², R⁴, and R⁶ is independently chosen from alkenyl, alkoxy, alkyl, aryl, arylalkyl, cyano, cycloalkyl, H, halo, haloalkoxy, heteroaryl, heteroarylalkyl, heterocycloalkyl, hydroxyl, N(R³)₂, C(O)OH, N(R³)C(O)C(R³)₃, N(R³)C(O)OC(R³)₃, C(O)OC(R³)₃, and C(O)N(R³)₂, wherein two R² groups together with the atoms to which they are attached optionally form an aryl, cycloalkyl, heteroaryl, or heterocycloalkyl ring, which may be optionally substituted with between 0 and 3 R³ groups, wherein two R⁴ groups together with the atoms to which they are attached optionally form an aryl, cycloalkyl, heteroaryl, or heterocycloalkyl ring, which may be optionally substituted with between 0 and 3 R³ groups, wherein two R⁶ groups together with the atoms to which they are attached optionally form an aryl, cycloalkyl, heteroaryl, or heterocycloalkyl ring, which may be optionally substituted with between 0 and 3 R³ groups;
each R³ is independently chosen from alkenyl, alkoxy, alkyl, aminoalkyl, aryl, arylalkyl, cyano, cycloalkyl, cycloalkylalkyl, cycloalkylalkoxy, H, halo, haloalkyl, heteroaryl, heteroarylalkyl, heterocycloalkyl, heterocycloalkylalkyl, and hydroxyl; and
each R⁵ is independently chosen from H and halo.

4. The compound as recited in claim 3, wherein:
X is chosen from imidazole, phenyl, pyridine, and pyrazole.

5. The compound as recited in claim 3, wherein:
Y and Z are alkyl.

6. The compound as recited in claim 3, wherein:
R¹ is H.

7. The compound as recited in claim 3, wherein:
R² is chosen from alkyl and alkoxy.

8. The compound as recited in claim 3, wherein:
R⁴ and R⁶ are independently chosen from alkoxy, H, halo, and N(R³)₂.

9. A composition comprising a compound of claim 1 and a pharmaceutically acceptable carrier, adjuvant, or vehicle.

10. A method for inhibiting activity of a bromodomain-containing protein, or a mutant thereof, in a biological sample comprising the step of contacting said biological sample with a compound according to claim 1.

11. The method according to claim 10, wherein the bromodomain-containing protein is chosen from BRPF1, BRPF2, BRPF3, and TRIM24.

12. The method according to claim 11, wherein the bromodomain-containing protein is TRIM24.

13. A method for treating a bromodomain-containing protein-mediated disorder in a subject in need thereof, comprising the step of administering to said subject a compound according to claim 1.

14. The method according to claim 13, wherein the subject is a human.

15. The method according to claim 13, wherein the bromodomain-containing protein is chosen from BRPF1, BRPF2, BRPF3, and TRIM24.

16. The method according to claim 13, wherein the bromodomain-containing protein is TRIM24.

17. The method according to claim 13, wherein the bromodomain-containing protein-mediated disorder is a proliferative disorder, inflammatory disease, sepsis, neurological disease, autoimmune disease, or viral infection.

18. The method according to claim 17, wherein the proliferative disorder is cancer.

19. The method according to claim 18, wherein the cancer is chosen from adenocarcinoma, adult T-cell leukemia/lymphoma, bladder cancer, blastoma, bone cancer, breast cancer, brain cancer, carcinoma, myeloid sarcoma, cervical cancer, colorectal cancer, esophageal cancer, gastrointestinal cancer, glioblastoma multiforme, glioma, gallbladder cancer, gastric cancer, head and neck cancer, Hodgkin's lymphoma, non-Hodgkin's lymphoma, intestinal cancer, kidney cancer, laryngeal cancer, leukemia, lung cancer, lymphoma, liver cancer, small cell lung cancer, non-small cell lung cancer, mesothelioma, multiple myeloma, ocular cancer, optic nerve tumor, oral cancer, ovarian cancer, pituitary tumor, primary central nervous system lymphoma, prostate cancer, pancreatic cancer, pharyngeal cancer, renal cell carcinoma, rectal cancer, sarcoma, skin cancer, spinal tumor, small intestine cancer, stomach cancer, T-cell lymphoma, testicular cancer, thyroid cancer, throat cancer, urogenital cancer, urothelial carcinoma, uterine cancer, vaginal cancer, and Wilms' tumor.

20. The method according to claim 18, wherein the cancer is chosen from acute myelogenous leukemia and Burkitt's lymphoma.

21. The method according to claim 17, wherein the autoimmune and inflammatory diseases involve an inflammatory response to infections with bacteria, viruses, fungi, parasites or their toxins.

22. The method according to claim 17, wherein the autoimmune and inflammatory diseases or conditions are chosen from sepsis, sepsis syndrome, septic shock, endotoxemia, systemic inflammatory response syndrome (SIRS), multi-organ dysfunction syndrome, toxic shock syndrome, acute lung injury, ARDS (adult respiratory distress syndrome), acute renal failure, fulminant hepatitis, bums, acute pancreatitis, post-surgical syndromes, sarcoidosis, Herxheimer reactions, encephalitis, myelitis, meningitis, malaria and SIRS associated with viral infections such as influenza, herpes zoster, herpes simplex and coronavirus.

23. The method according to claim 17, wherein the neurological diseases or conditions are chosen from Alzheimer's disease, Parkinson's disease, Huntington disease, bipolar disorder, schizophrenia, Rubinstein-Taybi syndrome, and epilepsy.

24. A method of treating a bromodomain-containing protein-mediated disorder in a subject in need thereof, comprising the sequential or co-administration of a compound of claim 1 or a pharmaceutically acceptable salt thereof, and another therapeutic agent.

25. The method according to claim 24, wherein the therapeutic agent is chosen from a BET family bromodomain inhibitor.

26. The method according to claim 25, wherein the BET family bromodomain is chosen from BRD2, BRD3, and BRD4.

27. The method according to claim 24, wherein the therapeutic agent is chosen from a protein kinase inhibitor.

28. The method according to claim 27, wherein the protein kinase inhibitor is chosen from Aurora B, EGFR, PLK-1, and CDKs inhibitors.

29. The method according to claim 24, wherein the therapeutic agent is chosen from an antimetabolite, bcr-abl inhibitor, DNA damaging agent, EGFR inhibitor, microtubule stabilizing inhibitor, mitotic arrest inhibitor, S-phase inhibitor, and taxane.

30. The method according to claim 29, wherein the DNA damaging agent is chosen from an alkylating agent, anthracycline, antimetabolite agent, crosslinking agent, DNA replication inhibitor, intercalator, microtubule disruptor, PARP inhibitor, radiomimetic agent, radiosensitizer, strand break agent, and topoisomerase II inhibitor.

31. The method according to claim 24, wherein the therapeutic agent is chosen from aminoglutethimide, amsacrine, anastrozole, asparaginase, barasertib, bcg, bicalutamide, bleomycin, buserelin, busulfan, campothecin, capecitabine, carboplatin, carmustine, chlorambucil, chloroquine, cisplatin, cladribine, clodronate, colchicine, cyclophosphamide, cyproterone, cytarabine, dacarbazine, dactinomycin, daunorubicin, demethoxyviridin, dichloroacetate, dienestrol, diethylstilbestrol, docetaxel, doxorubicin, epirubicin, estradiol, estramustine, etoposide, everolimus, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, lonidamine, mechlorethamine, medroxyprogesterone, megestrol, melphalan, mercaptopurine, mesna, metformin, methotrexate, mitomycin, mitotane, mitoxantrone, nilutamide, nocodazole, olaparib, octreotide, oxaliplatin, paclitaxel, pamidronate, pentostatin, perifosine, plicamycin, porfimer, procarbazine, raltitrexed, rituximab, sorafenib, streptozocin, sunitinib, suramin, tamoxifen, temozolomide, temsirolimus, teniposide, testosterone, thioguanine, thiotepa, titanocene dichloride, topotecan, trastuzumab, tretinoin, vinblastine, vincristine, vindesine, and vinorelbine.

32. The method of claim 18, wherein the method further comprises administering non-chemical methods of cancer treatment.

33. The method of claim 32, wherein the method further comprises administering radiation therapy.

34. The method of claim 32, wherein the method further comprises administering surgery, thermoablation, focused ultrasound therapy, cryotherapy, or any combination thereof.

35. The method of claim 24, wherein the method further comprises administering non-chemical methods of cancer treatment.

36. The method of claim 35, wherein the method further comprises administering radiation therapy.

37. The method of claim 35, wherein the method further comprises administering surgery, thermoablation, focused ultrasound therapy, cryotherapy, or any combination thereof.
